(19) United States
(12) Patent Application Publication
(10) Pub. No.: US 2004/0023257 A1
(43) Pub. Date: Feb. 5, 2004

(54) Enzymes having secondary amidases activity and methods of use thereof

(75) Inventors: Nelson R. Barton, San Diego, CA (US); David Paul Weiner, Del Mar, CA (US); William Greenberg, San Diego, CA (US); Samantha Luu, San Diego, CA (US); Kristine Chang, San Diego, CA (US); Elizabeth Waters, San Diego, CA (US)

Correspondence Address: FISH & RICHARDSON, PC, 12390 EL CAMINO REAL, SAN DIEGO, CA, 92130-2081, US

(73) Assignee: Diversa Corporation, San Diego, CA (US)

(21) Appl. No.: 10/354,437

(22) Filed: Jan. 28, 2003

Related U.S. Application Data
(60) Provisional application No. 60/352,895, filed on Jan. 28, 2002.

(51) Int. Cl. C12Q 1/68; G06F 19/00; G01N 33/48; G01N 33/50; C07H 21/04; C12N 9/80

(52) U.S. Cl. 435/6; 435/69.1; 435/228; 435/320.1; 435/325; 536/23.2; 702/19

(57) ABSTRACT

This invention provides amidases, polynucleotides encoding the amidases, methods of making and using these polynucleotides and polypeptides. In one aspect, the invention provides enzymes having secondary amidase activity, e.g., having activity in the hydrolysis of amides, including enzymes having peptidase, protease and/or hydantoinase activity. In alternative aspects, the enzymes of the invention can be used to used to increase flavor in food (e.g., enzyme ripened cheese), promote bacterial and fungal killing, modify and de-protect fine chemical intermediates, synthesize peptide bonds, carry out chiral resolutions, hydrolyze Cephalosporin C. The enzymes of the invention can be used to generate 7-aminocephalosporanic acid (7-ACA) and semi-synthetic cephalosporin antibiotics, including caphalothin, cephaloridine and cefuroxime. The enzymes of the invention can be used as antimicrobial agents, e.g., as cell wall hydrolytic agents. The invention also provides a fluorescent amidase substrate comprising 7-(ε-D-2-aminoadipoylamido)-4-methylcoumarin.

### TECHNICAL FIELD

This invention relates generally to molecular and cellular biology and biochemistry. This invention provides amidases, polynucleotides encoding the amidases, the use of such polynucleotides and polypeptides, and in one aspect, enzymes having secondary amidase activity, e.g., having activity in the hydrolysis of amides, including enzymes having peptidase, protease and/or hydantoinase activity. In alternative aspects, the enzymes of the invention can be used to process foods, e.g., to increase flavor in food (e.g., enzyme ripened cheese), promote bacterial and fungal killing, modify and de-protect fine chemical intermediates, synthesize peptide bonds, carry out chiral resolutions, hydrolyze cephalosporin C. The enzymes of the invention can be used to generate 7-aminocephalosporanic acid (7-ACA) and semi-synthetic cephalosporin antibiotics, including caphalothin, cephaloridine and cefuroxime. The enzymes of the invention can be used as antimicrobial agents, e.g., as cell wall hydrolytic agents.

### BACKGROUND

Secondary amidases include a variety of useful enzymes including peptidases, proteases, and hydantoinases. This class of enzymes can be used in a range of commercial applications. For example, secondary amidases can be used to: 1) increase flavor in food, in particular cheese (known as enzyme ripened cheese); 2) promote bacterial and fungal killing; 3) modify and de-protect fine chemical intermediates 4) synthesize peptide bonds; 5) and carry out chiral resolutions. Particularly, there is a need in the art for an enzyme capable of hydrolyzing Cephalosporin C.

Cephalosporin C is the fermentation product of the cephalosporin biosynthesis pathway and although it has been shown to have some activity against gram-negative microorganisms as an antibiotic itself, the major commercial use of cephalosporin C is as a building block for other cephalosporin-like antibiotics. For example, the D-alpha-aminoadipoyl side chain may be removed to generate 7-aminocephalosporanic acid (7-ACA). 7-ACA is a precursor to a wide range of semi-synthetic cephalosporin antibiotics including caphalothin, cephaloridine and cefuroxime.

Semisynthetic cephalosporins are among the most widely used antibiotics. These antibiotics are synthesized from 7-aminocephalosporanic acid (7-ACA), a compound obtained through the deacylation of Cephalosporin C (Ceph C). Traditionally, this deacylation has been carried out using a chemical process. However, the chemical process involves the use of numerous toxic compounds that generate a costly chemical waste stream. For this reason, an enzymatic route for the production of 7-ACA from Ceph C is very appealing. Currently, enzymatic production of 7-ACA from Ceph C is accomplished using a two-enzyme process (FIG. 7). The first enzyme, D-amino acid oxidase, is used to oxidize Ceph C to a keto acid intermediate that is then decarboxylated by hydrogen peroxide to yield glutaryl-7-ACA. Glutaryl-7-ACA is then deacylated to 7-ACA through the action of the second enzyme, Glutaryl-7-ACA acylase. Although some Glutaryl-7-ACA acylases can directly convert Ceph C to 7-ACA, they do so with very poor efficiency. Nonetheless, glutaryl-7-ACA acylases with measurable activity on Ceph C are classified as cephalosporin C acylases.

The search for a secondary amidases has been limited by the absence of a substrate/assay combination suitable for high throughput screening. Previous discovery efforts have utilized substrates or assays that suffer from low throughput, lack of sensitivity, and/or lack of specificity.

The publications discussed herein are provided solely for their disclosure prior to the filing date of the present application. Nothing herein is to be construed as an admission that the invention is not entitled to antedate such disclosure by virtue of prior invention.

### SUMMARY

The invention provides polypeptides having an amidase activity, including a secondary amidase activity, e.g., catalyzing the hydrolysis of amides, such as enzymes having a peptidase, a protease and/or a hydantoinase activity. In alternative aspects, the enzymes of the invention can be used to process foods, e.g., to increase flavors in food (e.g., enzyme ripened cheeses), promote bacterial and fungal killing, modify and de-protect fine chemical intermediates, synthesize peptide bonds, carry out chiral resolutions, hydrolyze Cephalosporin C. The enzymes of the invention can be used to generate 7-aminocephalosporanic acid (7-ACA) and semi-synthetic cephalosporin antibiotics, including caphalothin, cephaloridine and cefuroxime. The enzymes of the invention can be used as antimicrobial agents, e.g., as cell wall hydrolytic agents.

The invention provides an isolated or recombinant nucleic acid comprising a nucleic acid sequence having at least 50% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 31, SEQ ID NO: 33, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 91, SEQ ID NO: 93, SEQ ID NO: 95, SEQ ID NO: 97, SEQ ID NO: 101, SEQ ID NO: 103, SEQ ID NO: 105, SEQ ID NO: 107, SEQ ID NO: 109, SEQ ID NO: 111, at least 55% sequence identity to SEQ ID NO: 35, SEQ ID NO: 73, SEQ ID NO: 89, SEQ ID NO: 113, at least 60% sequence identity to SEQ ID NO: 27, SEQ ID NO: 29, SEQ ID NO: 57, at least 65% sequence identity to SEQ ID NO: 99, at least 90% sequence identity to SEQ ID NO: 55, at least 99% sequence identity to SEQ ID NO: 37, over a region of at least about 100 residues, wherein the nucleic acid encodes at least one polypeptide having an amidase activity, and the sequence identities are determined by analysis with a sequence comparison algorithm or by a visual inspection.

In one aspect, the isolated or recombinant nucleic acid comprises a nucleic acid sequence having at least 55% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 31, SEQ ID NO: 33, SEQ ID NO: 35, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 101, SEQ ID NO: 103, SEQ ID NO: 105, SEQ ID NO: 107, SEQ ID NO: 109, SEQ ID NO: 111, SEQ ID NO: 113, over a region of at least about 100 residues.

In one aspect, the nucleic acid comprises a sequence having at least 60% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 100 residues.

In one aspect, the nucleic acid comprises a sequence having at least 65% sequence identity to SEQ ID NO: 1, SEQ ID NO: 3, SEQ ID NO: 5, SEQ ID NO: 7, SEQ ID NO: 9, SEQ ID NO: 1, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 100 residues.

In one aspect, the nucleic acid comprises a sequence having at least 70% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ I) NO: 27, SEQ ID NO: 29, SEQ ID NO: 31, SEQ ID NO: 33, SEQ ID NO: 35, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 100 residues.

In one aspect, the nucleic acid comprises a sequence having at least 75% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 100 residues.

In one aspect, the nucleic acid comprises a sequence having at least 80% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 100 residues.

In one aspect, wherein the nucleic acid comprises a sequence having at least 85% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 100 residues.

In one aspect, the nucleic acid comprises a sequence having at least 90% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 100 residues.

In one aspect, the nucleic acid comprises a sequence having at least 95% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 100 residues.

In one aspect, the nucleic acid comprises a sequence having at least 99% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 100 residues.

In one aspect, the nucleic acid sequence comprises a sequence as set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113.

The invention provides an isolated or recombinant nucleic acid encoding a polypeptide comprising a sequence as set forth in SEQ ID NO: 2, SEQ ID NO: 4, SEQ ID NO: 6, ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 113, SEQ ID NO: 114.

In one aspect, wherein the sequence comparison algorithm is a BLAST version 2.2.2 algorithm where a filtering setting is set to blastall -p blastp -d “nr pataa”-F F, and all other options are set to default.

In one aspect, the nucleic acid comprises a sequence having at least 50% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 200, 300, 400, 500, 550, 600, or 650 residues.

In one aspect, the nucleic acid comprises a sequence having at least 50% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 700 residues.

In one aspect, the nucleic acid comprises a sequence having at least 50% sequence identity to SEQ ID NO: 1, SEQ ID NO: 3, SEQ ID NO: 5, ID NO: 7, SEQ ID NO: 9, SEQ ID NO: 13, SEQ ID NO: 15, SEQ ID NO: 17, SEQ ID NO: 19, SEQ ID NO: 21, SEQ ID NO: 25, SEQ ID NO: 27, SEQ ID NO: 31, SEQ ID NO: 33, SEQ ID NO: 35, SEQ ID NO: 39, SEQ ID NO: 41, SEQ ID NO: 43, SEQ ID NO: 47, SEQ ID NO: 49, SEQ ID NO: 51, SEQ ID NO: 53, SEQ ID NO: 59, SEQ ID NO: 61, SEQ ID NO: 63, SEQ ID NO: 65, SEQ ID NO: 67, SEQ ID NO: 69, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 111, SEQ ID NO: 113 over a region of at least about 800 residues.

In one aspect, the nucleic acid comprises a sequence having at least 50% sequence identity to SEQ ID NO: 3, SEQ ID NO: 5, SEQ ID NO: 9, SEQ ID NO: 13, SEQ ID NO: 15, SEQ ID NO: 17, SEQ ID NO: 19, SEQ ID NO: 21, SEQ ID NO: 31, SEQ ID NO: 35, SEQ I) NO: 39, SEQ ID NO: 47, SEQ ID NO: 49, SEQ ID NO: 53, SEQ ID NO: 61, SEQ ID NO: 63, SEQ ID NO: 65, SEQ ID NO: 73, SEQ ID NO: 75, SEQ ID NO: 81, SEQ ID NO: 83, SEQ ID NO: 87, SEQ ID NO: 89, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 111, SEQ ID NO: 113 over a region of at least about 900 residues.

In one aspect, the nucleic acid comprises a sequence having at least 50% sequence identity to SEQ ID NO: 3, SEQ ID NO: 5, SEQ ID NO: 9, SEQ ID NO: 13, SEQ ID NO: 15, SEQ ID NO: 17, SEQ ID NO: 19, SEQ ID NO: 21, SEQ ID NO: 31, SEQ ID NO: 35, SEQ ID NO: 39, SEQ ID NO: 47, SEQ ID NO: 49, SEQ ID NO: 53, SEQ ID NO: 61, SEQ ID NO: 63, SEQ ID NO: 65, SEQ ID NO: 73, SEQ ID NO: 75, SEQ ID NO: 81, SEQ ID NO: 83, SEQ ID NO: 87, SEQ ID NO: 89, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 111, SEQ ID NO: 113 over a region of at least about 900 residues.

In one aspect, the nucleic acid comprises a sequence having at least 50% sequence identity to SEQ ID NO: 15, SEQ ID NO: 17, SEQ ID NO: 19, SEQ ID NO: 21, SEQ ID NO: 31, SEQ ID NO: 35, SEQ ID NO: 47, SEQ ID NO: 49, SEQ ID NO: 53, SEQ ID NO: 61, SEQ ID NO: 63, SEQ ID NO: 65, SEQ ID NO: 73, SEQ ID NO: 75, SEQ ID NO: 81, SEQ ID NO: 87, SEQ ID NO: 89, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 111, SEQ ID NO: 113 over a region of at least about 1000 residues.

In one aspect, the nucleic acid comprises a sequence having at least 50% sequence identity to SEQ ID NO: 31, SEQ ID NO: 35, SEQ ID NO: 47, SEQ ID NO: 49, SEQ ID NO: 53, SEQ ID NO: 61, SEQ ID NO: 63, SEQ ID NO: 65, SEQ ID NO: 73, SEQ ID NO: 75, SEQ ID NO: 81, SEQ ID NO: 87, SEQ ID NO: 89, SEQ ID NO: 97, SEQ ID NO: 99, SEQ ID NO: 101, SEQ ID NO: 105, SEQ ID NO: 107, SEQ ID NO: 111 over a region of at least about 1200 residues.

The invention provides an isolated or recombinant nucleic acid, wherein the nucleic acid comprises a sequence that hybridizes under stringent conditions to a nucleic acid comprising a sequence a s set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, wherein the nucleic acid encodes a polypeptide having an amidase activity.

In one aspect, the nucleic acid is at least about 50, 100, 150, 200, 300, 400 or 500 residues in length. In one aspect, the nucleic acid is at least about 600, 700, 800, 900, 1000, 1100 or 1200 residues in length or the full length of the gene or transcript.

In one aspect, the stringent conditions comprise a wash step comprising a wash in 0.2×SSC at a temperature of about 65° C. for about 15 minutes.

In one aspect, the amidase activity comprises hydrolyzing an amide bond. The amidase activity can comprise a secondary amidase activity. In one aspect, the amidase activity comprises an internal amidase activity. In one aspect, the amidase activity comprises a C-terminal amidase activity. The amidase activity can comprise an N-terminal amidase activity. The amidase activity can comprise hydrolyzing amide bonds in a protein. The amidase activity can comprise hydrolyzing an amide bond in a drug or pharmaceutical composition, e.g., a cephalosporin, such as cephalosporin C. In one aspect, the amidase activity comprises an hydrolyzing amide bond in cephalosporin C to produce 7-aminocephalosporanic acid (7-ACA).

In one aspect, the amidase activity is enantioselective. The amidase can generate enantiomerically pure L-amino acids from racemic mixtures. The amidase can generate peptides by the enzymatic conversion of amino acid alkyl esters or N-protected peptide alkyl esters.

In one aspect, the amidase retains activity under conditions comprising a temperature range of between about 37° C. to about 95° C. The amidase can retain activity under conditions comprising a temperature range of between about 55° C. to about 85° C.

In one aspect, the amidase activity is thermotolerant. The amidase activity can be thermotolerant after exposure to a temperature in the range from greater than 37° C. to about 95° C.

The invention provides nucleic acid probes for identifying a nucleic acid encoding a polypeptide with an amidase activity, wherein the probe comprises at least 10 consecutive bases of a nucleic acid sequence of the invention. The invention provides nucleic acid probes for identifying a nucleic acid encoding a polypeptide having an amidase activity, wherein the probe comprises a nucleic acid comprising a nucleic acid sequence having at least 50%, 55%, 60%, 65%, 70%, 75%, 80%, 85%, 90%, 95%, 98%, 99%, or more, sequence identity to nucleic acid sequence of the invention, wherein the sequence identities are determined by analysis with a sequence comparison algorithm or by visual inspection. In one aspect, the probe can comprise an oligonucleotide comprising at least about 10 to 50, about 20 to 60, about 30 to 70, about 40 to 80, or about 60 to 100, or about 70 to 150 consecutive bases of a nucleic acid sequence.

The invention provides amplification primer sequence pairs for amplifying a nucleic acid encoding a polypeptide of the invention, e.g., a polypeptide having an amidase activity, wherein the primer pair is capable of amplifying a nucleic acid sequence of the invention. One or each member of the amplification primer sequence pair comprises an oligonucleotide can comprise at least about 10 to 50 consecutive bases of the sequence. The invention provides methods of amplifying a nucleic acid encoding a polypeptide having an amidase activity comprising amplification of a template nucleic acid with an amplification primer sequence pair capable of amplifying a nucleic acid sequence of the invention.

The invention provides expression cassette comprising a nucleic acid of the invention. In one aspect, the nucleic acid can be operably linked to an animal or a plant promoter. In one aspect, the expression cassette can further comprise a plant expression vector. The plant expression vector can comprise a plant virus. In one aspect, the plant promoter can comprise a potato, rice, corn, wheat, or barley promoter. In one aspect, the promoter can comprise a promoter derived from T-DNA of Agrobacterium tumefaciens. In one aspect, the promoter can be a constitutive promoter. The constitutive promoter can be CaMV35S. In another aspect, the promoter can be an inducible promoter. In one aspect, the promoter can be a tissue-specific promoter. The tissue-specific promoter can be a seed-specific, a leaf-specific, a root-specific, a stem-specific or an abscission-induced promoter.

The invention provides vectors comprising a nucleic acid of the invention. The invention provides cloning vehicle comprising a vector of the invention or a nucleic acid of the invention. In one aspect, the cloning vehicle can comprise a viral vector, a plasmid, a phage, a phagemid, a cosmid, a fosmid, a bacteriophage or an artificial chromosome. In one aspect, the viral vector can comprise an adenovirus vector, a retroviral vector or an adeno-associated viral vector. In another aspect, the cloning vehicle can comprise a bacterial artificial chromosome (BAC), a plasmid, a bacteriophage P1-derived vector (PAC), a yeast artificial chromosome (YAC), or a mammalian artificial chromosome (MAC).

The invention provides transformed cells comprising a vector of the invention, a nucleic acid of the invention or a cloning vehicle of the invention. In one aspect, the cell can be a bacterial cell, a mammalian cell, a fungal cell, a yeast cell, an insect cell or a plant cell. In one aspect, the plant cell can be a potato, rice, corn, wheat, tobacco or barley cell.

The invention provides transgenic non-human animals comprising a vector of the invention, a nucleic acid of the invention or a cloning vehicle of the invention. In one aspect, the animal can be a mouse.

The invention provides transgenic plant comprising a vector of the invention, a nucleic acid of the invention or a cloning vehicle of the invention. In one aspect, the plant can be a corn plant, a sorghum plant, a potato plant, a tomato plant, a wheat plant, an oilseed plant, a rapeseed plant, a soybean plant, a rice plant, a barley plant, a grass, or a tobacco plant.

The invention provides transgenic seeds comprising a nucleic acid of the invention. In one aspect, the seed can be a corn seed, a wheat kernel, an oilseed, a rapeseed, a soybean seed, a palm kernel, a sunflower seed, a sesame seed, a rice, a barley, a peanut or a tobacco plant seed.

The invention provides antisense oligonucleotides comprising a nucleic acid sequence of the invention or a subsequence thereof. In one aspect, the antisense oligonucleotide can be between about 10 to 50, about 20 to 60, about 30 to 70, about 40 to 80, or about 60 to 100 bases in length.

The invention provides methods of inhibiting the translation of an amidase message in a cell comprising administering to the cell or expressing in the cell an antisense oligonucleotide comprising a nucleic acid sequence complementary to or capable of hybridizing under stringent conditions to a nucleic acid of the invention.

The invention provides isolated or recombinant polypeptides comprising (a) a sequence comprising at least 50% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 32, SEQ ID NO: 34, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 76, SEQ ID NO: 78, SEQ ID NO: 80, SEQ ID NO: 82, SEQ ID NO: 85, SEQ ID NO: 87, SEQ ID NO: 88, SEQ ID NO: 92, SEQ ID NO: 94, SEQ ID NO: 96, SEQ ID NO: 98, SEQ ID NO: 102, SEQ ID NO: 104, SEQ ID NO: 106, SEQ ID NO: 108, SEQ ID NO: 110, SEQ ID NO: 112, at least 55% sequence identity to SEQ ID NO: 36, SEQ ID NO: 74, SEQ ID NO: 90, SEQ ID NO: 114, at least 60% sequence identity to SEQ ID NO: 28, SEQ ID NO: 30, SEQ ID NO: 58, at least 65% sequence identity to SEQ ID NO: 100, at least 90% sequence identity to SEQ ID NO: 56, at least 99% sequence identity to SEQ ID NO: 38, over a region of at least about 100 residues; or; (b) a polypeptide encoded by a nucleic acid of the invention.

In one aspect, the polypeptide can have an amidase activity.

In one aspect, the polypeptide comprises an amino acid sequence having at least 50% identity a sequence region of at least about 150, 200 250, 300, 350, 400, 450 or 500 residues. In one aspect, the polypeptide comprises an amino acid sequence having at least 55%, 60%, 65%, 70%, 75%, 80%, 85%, 90%, 95% or 99% identity over a region of at least about 100 residues.

In one aspect, the isolated or recombinant polypeptide can comprise an amino acid sequence as set forth in SEQ ID NO: 2, SEQ ID NO: 4, SEQ ID NO: 6, ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 113, SEQ ID NO: 114.

In one aspect, the amidase activity comprises hydrolyzing an amide bond. The amidase activity can comprise a secondary amidase activity. In one aspect, the amidase activity comprises an internal amidase activity. In one aspect, the amidase activity comprises a C-terminal amidase activity. The amidase activity can comprise an N-terminal amidase activity. The amidase activity can comprise hydrolyzing amide bonds in a protein. The amidase activity can comprise hydrolyzing an amide bond in a drug or pharmaceutical composition, e.g., a cephalosporin, such as cephalosporin C. In one aspect, the amidase activity comprises an hydrolyzing amide bond in cephalosporin C to produce 7-aminocephalosporanic acid (7-ACA).

In one aspect, the amidase activity is enantioselective. The amidase can generate enantiomerically pure L-amino acids from racemic mixtures. The amidase can generate peptides by the enzymatic conversion of amino acid alkyl esters or N-protected peptide alkyl esters.

In one aspect, the invention provides isolated or recombinant polypeptide, wherein the amidase activity is thermostable. In one aspect, the polypeptide can retain an amidase activity under conditions comprising a temperature range of between about 37° C. to about 95° C. In another aspect, the polypeptide can retain an amidase activity under conditions comprising a temperature range of between about 55° C. to about 85° C., a temperature range of between about 70° C. to about 95° C., or a temperature range of between about 90° C. to about 95° C.

In another aspect, the invention provides isolated or recombinant polypeptide, wherein the amidase activity is thermotolerant. In one aspect, the polypeptide can retain an amidase activity after exposure to a temperature in the range from greater than 37° C. to about 95° C., in the range from greater than 55° C. to about 85° C., or in the range from greater than 90° C. to about 95° C.

The invention provides isolated or recombinant polypeptide comprising the polypeptide of the invention and lacking a signal sequence.

In one aspect, the invention provides isolated or recombinant polypeptides of the invention, wherein the amidase activity comprises a specific activity at about 37° C. in the range from about 100 to about 1000 units per milligram of protein. In one aspect, the amidase activity can comprise a specific activity at 37° C. from about 500 to about 750 units per milligram of protein, in the range from about 500 to about 1200 units per milligram of protein, or in the range from about 750 to about 1000 units per milligram of protein. In one aspect, the thermotolerance can comprise retention of at least half of the specific activity of the amidase at 37° C. after being heated to an elevated temperature. In another aspect, the thermotolerance can comprise retention of specific activity at 37° C. in the range from about 500 to about 1200 units per milligram of protein after being heated to an elevated temperature.

In one aspect, the isolated or recombinant polypeptide of the invention can comprise at least one glycosylation site. In one aspect, glycosylation can be an N-linked glycosylation. In one aspect, the polypeptide of the invention can be glycosylated after being expressed in a P. pastoris or a S. pombe.

The invention provides the isolated or recombinant polypeptide of the invention, wherein the polypeptide retains an amidase activity under conditions comprising about pH 4.5 or pH 5 or less. In one aspect, the polypeptide can retain an amidase activity under conditions comprising about pH 8.0, pH 8.5, pH 9, pH 9.5, pH 10 or pH 10.5 or more.

The invention provides protein preparations comprising a polypeptide of the invention, wherein the protein preparation comprises a liquid, a solid or a gel.

In one aspect, the invention provides heterodimers comprising a polypeptide of the invention and a second domain. In one aspect, the second domain can be a polypeptide and the heterodimer is a fusion protein. In another aspect, the second domain can be an epitope or a tag. The invention provides homodimers comprising a polypeptide of the invention.

The invention provides immobilized polypeptides having an amidase activity, wherein the polypeptide comprises a polypeptide or a heterodimer comprising a polypeptide of the invention. In one aspect, the polypeptide can be immobilized on a cell, a metal, a resin, a polymer, a ceramic, a glass, a microelectrode, a graphitic particle, a bead, a gel, a plate, an array or a capillary tube. The invention provides arrays comprising an immobilized polypeptide of the invention. In one aspect, an array can comprise an immobilized nucleic acid of the invention.

The invention provides isolated or recombinant antibodies that specifically bind to a polypeptide of the invention or to a polypeptide encoded by a nucleic acid of the invention. In one aspect, the antibody can be a monoclonal or a polyclonal antibody. The invention provides hybridomas comprising an antibody of the invention.

The invention provides food supplements for an animal comprising a polypeptide of the invention. The invention provides edible enzyme delivery matrices comprising a polypeptide of the invention.

The invention provides methods of isolating or identifying a polypeptide with an amidase activity comprising the steps of: (a) providing an antibody of the invention; (b) providing a sample comprising polypeptides; and (c) contacting the sample of step (b) with the antibody of step (a) under conditions wherein the antibody can specifically bind to the polypeptide, thereby isolating or identifying a polypeptide having an amidase activity.

The invention provides methods of making an anti-amidase antibody comprising administering to a non-human animal a nucleic acid of the invention, a polypeptide of the invention, or a polypeptide encoded by a nucleic acid of the invention in an amount sufficient to generate a humoral immune response, thereby making an anti-amidase antibody.

The invention provides methods of producing a recombinant polypeptide comprising the steps of: (a) providing a nucleic acid operably linked to a promoter, wherein the nucleic acid comprises a nucleic acid of the invention; and (b) expressing the nucleic acid of step (a) under conditions that allow expression of the polypeptide, thereby producing a recombinant polypeptide. In one aspect, the method can further comprise transforming a host cell with the nucleic acid of step (a) followed by expressing the nucleic acid of step (a), thereby producing a recombinant polypeptide in a transformed cell.

The invention provides methods for identifying a polypeptide having an amidase activity comprising the following steps: (a) providing a polypeptide of the invention; (b) providing an amidase substrate; and (c) contacting the polypeptide with the substrate of step (b) and detecting a decrease in the amount of substrate or an increase in the amount of a reaction product, wherein a decrease in the amount of the substrate or an increase in the amount of the reaction product detects a polypeptide having an amidase activity. In one aspect, the substrate can be a protein or amide. In another aspect, the substrate can be a cephalosporin C.

The invention provides methods for identifying an amidase substrate comprising the following steps: (a) providing a polypeptide of the invention; (b) providing a test substrate; and (c) contacting the polypeptide of step (a) with the test substrate of step (b) and detecting a decrease in the amount of substrate or an increase in the amount of reaction product, wherein a decrease in the amount of the substrate or an increase in the amount of a reaction product identifies the test substrate as an amidase substrate.

The invention provides methods of determining whether a test compound specifically binds to a polypeptide comprising the following steps: (a) expressing a nucleic acid or a vector comprising the nucleic acid under conditions permissive for translation of the nucleic acid to a polypeptide, wherein the nucleic acid comprises a nucleic acid of the invention, or, providing a polypeptide of the invention; (b) providing a test compound; (c) contacting the polypeptide with the test compound; and (d) determining whether the test compound of step (b) specifically binds to the polypeptide.

The invention provides methods for identifying a modulator of an amidase activity comprising the following steps: (a) providing a polypeptide of the invention or a polypeptide encoded by a nucleic acid of the invention; (b) providing a test compound; (c) contacting the polypeptide of step (a) with the test compound of step (b) and measuring an activity of the amidase, wherein a change in the amidase activity measured in the presence of the test compound compared to the activity in the absence of the test compound provides a determination that the test compound modulates the amidase activity. In one aspect, the amidase activity can be measured by providing an amidase substrate and detecting a decrease in the amount of the substrate or an increase in the amount of a reaction product, or, an increase in the amount of the substrate or a decrease in the amount of a reaction product. In one aspect, a decrease in the amount of the substrate or an increase in the amount of the reaction product with the test compound as compared to the amount of substrate or reaction product without the test compound identifies the test compound as an activator of amidase activity. In another aspect, an increase in the amount of the substrate or a decrease in the amount of the reaction product with the test compound as compared to the amount of substrate or reaction product without the test compound identifies the test compound as an inhibitor of amidase activity.

The invention provides computer systems comprising a processor and a data storage device wherein said data storage device has stored thereon a polypeptide a polypeptide of the invention, or a polypeptide encoded by a nucleic acid of the invention. In one aspect, the computer system can further comprise a sequence comparison algorithm and a data storage device having at least one reference sequence stored thereon. The sequence comparison algorithm can comprise a computer program that indicates polymorphisms. In another aspect, the computer system can further comprise an identifier that identifies one or more features in said sequence.

The invention provides computer readable mediums having stored thereon a polypeptide sequence or a nucleic acid sequence, wherein the polypeptide sequence comprises a polypeptide of the invention or a polypeptide encoded by a nucleic acid of the invention.

The invention provides methods for identifying a feature in a sequence comprising the steps of: (a) reading the sequence using a computer program which identifies one or more features in a sequence, wherein the sequence comprises a polypeptide sequence or a nucleic acid sequence, wherein the polypeptide sequence comprises a polypeptide of the invention or a polypeptide encoded by a nucleic acid of the invention; and (b) identifying one or more features in the sequence with the computer program.

The invention provides methods for comparing a first sequence to a second sequence comprising the steps of: (a) reading the first sequence and the second sequence through use of a computer program which compares sequences, wherein the first sequence comprises a polypeptide sequence or a nucleic acid sequence, wherein the polypeptide sequence comprises a polypeptide of the invention or a polypeptide encoded by a nucleic acid of the invention; and (b) determining differences between the first sequence and the second sequence with the computer program. In one aspect, the step of determining differences between the first sequence and the second sequence can further comprise the step of identifying polymorphisms. In one aspect, the method can further comprise an identifier that identifies one or more features in a sequence. In another aspect, the method can comprise reading the first sequence using a computer program and identifying one or more features in the sequence.

The invention provides methods for isolating or recovering a nucleic acid encoding a polypeptide with an amidase activity from an environmental sample comprising the steps of: (a) providing an amplification primer sequence pair for amplifying a nucleic acid encoding a polypeptide with an amidase activity, wherein the primer pair is capable of amplifying a nucleic acid of the invention, or a subsequence thereof; (b) isolating a nucleic acid from the environmental sample or treating the environmental sample such that nucleic acid in the sample is accessible for hybridization to the amplification primer pair; and, (c) combining the nucleic acid of step (b) with the amplification primer pair of step (a) and amplifying nucleic acid from the environmental sample, thereby isolating or recovering a nucleic acid encoding a polypeptide with an amidase activity from an environmental sample. In one aspect, one or each member of the amplification primer sequence pair can comprise an oligonucleotide comprising at least about 10 to 50 consecutive bases of a sequence of the invention, or a subsequence thereof.

The invention provides methods for isolating or recovering a nucleic acid encoding a polypeptide with an amidase activity from an environmental sample comprising the steps of: (a) providing a polynucleotide probe comprising a nucleic acid of the invention; (b) isolating a nucleic acid from the environmental sample or treating the environmental sample such that nucleic acid in the sample is accessible for hybridization to a polynucleotide probe of step (a); (c) combining the isolated nucleic acid or the treated environmental sample of step (b) with the polynucleotide probe of step (a); and (d) isolating a nucleic acid that specifically hybridizes with the polynucleotide probe of step (a), thereby isolating or recovering a nucleic acid encoding a polypeptide with an amidase activity from an environmental sample. In one aspect, the environmental sample can comprise a water sample, a liquid sample, a soil sample, an air sample or a biological sample. In one aspect, the biological sample can be derived from a bacterial cell, a protozoan cell, an insect cell, a yeast cell, a plant cell, a fungal cell or a mammalian cell.

The invention provides methods of generating a variant of a nucleic acid encoding a polypeptide with an amidase activity comprising the steps of: (a) providing a template nucleic acid comprising a nucleic acid of the invention; and (b) modifying, deleting or adding one or more nucleotides in the template sequence, or a combination thereof, to generate a variant of the template nucleic acid. In one aspect, the method can further comprise expressing the variant nucleic acid to generate a variant amidase polypeptide. In one aspect, the modifications, additions or deletions can be introduced by a method comprising error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturated mutagenesis (GSSM), synthetic ligation reassembly (SLR) and a combination thereof. In another aspect, the modifications, additions or deletions can be introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof.

In one aspect, the method can be iteratively repeated until an amidase having an altered or different activity or an altered or different stability from that of a polypeptide encoded by the template nucleic acid is produced. In one aspect, the variant amidase polypeptide can be thermotolerant, and retains some activity after being exposed to an elevated temperature. In another aspect, the variant amidase polypeptide can have increased glycosylation as compared to the amidase encoded by a template nucleic acid. In one aspect, the variant amidase polypeptide can have an amidase activity under a high temperature, wherein the amidase encoded by the template nucleic acid is not active under the high temperature. In one aspect, the method can be iteratively repeated until an amidase coding sequence having an altered codon usage from that of the template nucleic acid is produced. In another aspect, the method can be iteratively repeated until an amidase gene having higher or lower level of message expression or stability from that of the template nucleic acid is produced.

The invention provides methods for modifying codons in a nucleic acid encoding a polypeptide with an amidase activity to increase its expression in a host cell, the method comprising the following steps: (a) providing a nucleic acid encoding a polypeptide with an amidase activity comprising a nucleic acid of the invention; and (b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon under-represented in coding sequences in genes in the host cell, thereby modifying the nucleic acid to increase its expression in a host cell.

The invention provides methods for modifying codons in a nucleic acid encoding an amidase polypeptide, the method comprising the following steps: (a) providing a nucleic acid encoding a polypeptide with an amidase activity comprising a nucleic acid of the invention; and, (b) identifying a codon in the nucleic acid of step (a) and replacing it with a different codon encoding the same amino acid as the replaced codon, thereby modifying codons in a nucleic acid encoding an amidase.

The invention provides methods for modifying codons in a nucleic acid encoding an amidase polypeptide to increase its expression in a host cell, the method comprising the following steps: (a) providing a nucleic acid encoding an amidase polypeptide and comprising a nucleic acid of the invention; and, (b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon under-represented in coding sequences in genes in the host cell, thereby modifying the nucleic acid to increase its expression in a host cell.

The invention provides methods for modifying a codon in a nucleic acid encoding a polypeptide having an amidase activity to decrease its expression in a host cell, the method comprising the following steps: (a) providing a nucleic acid of the invention; and (b) identifying at least one preferred codon in the nucleic acid of step (a) and replacing it with a non-preferred or less preferred codon encoding the same amino acid as the replaced codon, wherein a preferred codon is a codon over-represented in coding sequences in genes in a host cell and a non-preferred or less preferred codon is a codon under-represented in coding sequences in genes in the host cell, thereby modifying the nucleic acid to decrease its expression in a host cell. In one aspect, the host cell can be a bacterial cell, a fungal cell, an insect cell, a yeast cell, a plant cell or a mammalian cell.

The invention provides methods for producing a library of nucleic acids encoding a plurality of modified amidase active sites or substrate binding sites, wherein the modified active sites or substrate binding sites are derived from a first nucleic acid comprising a sequence encoding a first active site or a first substrate binding site the method comprising the following steps: (a) providing a first nucleic acid encoding a first active site or first substrate binding site, wherein the first nucleic acid sequence comprises a sequence that hybridizes under stringent conditions to a sequence of the invention, or a subsequence thereof, and the nucleic acid encodes an amidase active site or an amidase substrate binding site; (b) providing a set of mutagenic oligonucleotides that encode naturally-occurring amino acid variants at a plurality of targeted codons in the first nucleic acid; and, (c) using the set of mutagenic oligonucleotides to generate a set of active site-encoding or substrate binding site-encoding variant nucleic acids encoding a range of amino acid variations at each amino acid codon that was mutagenized, thereby producing a library of nucleic acids encoding a plurality of modified amidase active sites or substrate binding sites. In one aspect, the method can further comprise mutagenizing the first nucleic acid of step (a) by a method comprising an optimized directed evolution system, error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturated mutagenesis (GSSM), synthetic ligation reassembly (SLR) and a combination thereof. In another aspect, the method can further comprise mutagenizing the first nucleic acid of step (a) or variants by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof.

The invention provides methods for making a small molecule comprising the following steps: (a) providing a plurality of biosynthetic enzymes capable of synthesizing or modifying a small molecule, wherein one of the enzymes comprises an amidase enzyme encoded by a nucleic acid comprising a nucleic acid of the invention; (b) providing a substrate for at least one of the enzymes of step (a); and (c) reacting the substrate of step (b) with the enzymes under conditions that facilitate a plurality of biocatalytic reactions to generate a small molecule by a series of biocatalytic reactions.

The invention provides methods for modifying a small molecule comprising the following steps: (a) providing an amidase enzyme, wherein the enzyme comprises a of the invention or a polypeptide encoded by a nucleic acid of the invention; (b) providing a small molecule; and (c) reacting the enzyme of step (a) with the small molecule of step (b) under conditions that facilitate an enzymatic reaction catalyzed by the amidase enzyme, thereby modifying a small molecule by an amidase enzymatic reaction. In one aspect, the method can comprise a plurality of small molecule substrates for the enzyme of step (a), thereby generating a library of modified small molecules produced by at least one enzymatic reaction catalyzed by the amidase enzyme. In another aspect, the method can comprise a plurality of additional enzymes under conditions that facilitate a plurality of biocatalytic reactions by the enzymes to form a library of modified small molecules produced by the plurality of enzymatic reactions. Alternatively, the method can further comprise the step of testing the library to determine if a particular modified small molecule which exhibits a desired activity is present within the library. The step of testing the library can further comprise the steps of systematically eliminating all but one of the biocatalytic reactions used to produce a portion of the plurality of the modified small molecules within the library by testing the portion of the modified small molecule for the presence or absence of the particular modified small molecule with a desired activity, and identifying at least one specific biocatalytic reaction that produces the particular modified small molecule of desired activity.

The invention provides methods for determining a functional fragment of an amidase enzyme comprising the steps of: (a) providing an amidase enzyme, wherein the enzyme comprises a polypeptide of the invention or a polypeptide encoded by a nucleic acid of the invention; and (b) deleting a plurality of amino acid residues from the sequence of step (a) and testing the remaining subsequence for an amidase activity, thereby determining a functional fragment of an amidase enzyme. In one aspect, the amidase activity can be measured by providing an amidase substrate and detecting a decrease in the amount of the substrate or an increase in the amount of a reaction product.

The invention provides methods for whole cell engineering of new or modified phenotypes by using real-time metabolic flux analysis, the method comprising the following steps: (a) making a modified cell by modifying the genetic composition of a cell, wherein the genetic composition is modified by addition to the cell of a nucleic acid comprising a nucleic acid of the invention; (b) culturing the modified cell to generate a plurality of modified cells; (c) measuring at least one metabolic parameter of the cell by monitoring the cell culture of step (b) in real time; and, (d) analyzing the data of step (c) to determine if the measured parameter differs from a comparable measurement in an unmodified cell under similar conditions, thereby identifying an engineered phenotype in the cell using real-time metabolic flux analysis. In one aspect, the genetic composition of the cell can be modified by a method comprising deletion of a sequence or modification of a sequence in the cell, or, knocking out the expression of a gene. In one aspect, the method can further comprise selecting a cell comprising a newly engineered phenotype. In another aspect, the method can further comprise culturing the selected cell, thereby generating a new cell strain comprising a newly engineered phenotype.

The invention provides methods for hydrolyzing a amide bond comprising the following steps: (a) providing a polypeptide having an amidase activity, wherein the polypeptide comprises a polypeptide of the invention or a polypeptide encoded by a nucleic acid of the invention; (b) providing a composition comprising an amide bond; and (c) contacting the polypeptide of step (a) with the composition of step (b) under conditions wherein the polypeptide hydrolyzes the amide bond. In one aspect, the composition can comprise an internal amide bond. In another aspect, the composition can comprise a C-terminal amide bond or an N-terminal amide bond.

The invention provides methods for liquefying or removing a compound containing an amide bond from a composition comprising the following steps: (a) providing a polypeptide having an amidase activity, wherein the polypeptide comprises a polypeptide of the invention or a polypeptide encoded by a nucleic acid of the invention; (b) providing a composition comprising a compound containing an amide bond; and (c) contacting the polypeptide of step (a) with the composition of step (b) under conditions wherein the polypeptide removes or liquefies or removes the compound containing an amide bond.

The invention provides methods of increasing thermotolerance or thermostability of an amidase polypeptide, the method comprising glycosylating an amidase polypeptide, wherein the polypeptide comprises at least thirty contiguous amino acids of a polypeptide of the invention or a polypeptide encoded by a nucleic acid of the invention, thereby increasing the thermotolerance or thermostability of the amidase polypeptide. In one aspect, the amidase specific activity can be thermostable or thermotolerant at a temperature in the range from greater than about 37° C. to about 95° C.

The invention provides methods for overexpressing a recombinant amidase polypeptide in a cell comprising expressing a vector comprising a nucleic acid of the invention, wherein overexpression is effected by use of a high activity promoter, a dicistronic vector or by gene amplification of the vector.

The invention provides detergent compositions comprising a polypeptide of the invention or a polypeptide encoded by a nucleic acid of the invention, wherein the polypeptide comprises an amidase activity. In one aspect, the amidase can be a nonsurface-active amidase. In another aspect, the amidase can be a surface-active amidase.

The invention provides methods for washing an object comprising the following steps: (a) providing a composition comprising a polypeptide having an amidase activity, wherein the polypeptide comprises a polypeptide of the invention or a polypeptide encoded by a nucleic acid of the invention; (b) providing an object; and (c) contacting the polypeptide of step (a) and the object of step (b) under conditions wherein the composition can wash the object.

The invention provides methods for hydrolyzing a protein or an amide in a feed or a food prior to consumption by an animal comprising the following steps: (a) obtaining a feed material comprising a protein, wherein the protein can be hydrolyzed by a polypeptide having an amidase activity, wherein the polypeptide comprises a polypeptide of the invention or a polypeptide encoded by a nucleic acid of the invention; and (b) adding the polypeptide of step (a) to the feed or food material in an amount sufficient for a sufficient time period to cause hydrolysis of the protein and formation of a treated food or feed, thereby hydrolyzing the protein in the food or the feed prior to consumption by the animal. In one aspect, the food or feed comprises rice, corn, barley, wheat, legumes, or potato.

The invention provides methods for resolution of racemic mixtures of optically active compounds comprising the following steps: (a) providing a polypeptide comprising a polypeptide of the invention or a polypeptide encoded by a nucleic acid of the invention, wherein the polypeptide is selective for one enantiomer of optically active compounds; (b) providing a racemic mixture of optically active compounds, and (c) contacting the polypeptide of step (a) with the mixture of step (b) under conditions wherein the polypeptide can selectively convert only one enantiomer of optically active compound thereby resulting in a resolution of racemic mixtures. In one aspect, the polypeptide can be selective for a L enantiomer. In another aspect, the polypeptide can be selective for a R enantiomer. In one aspect, the polypeptide is stereospecific.

The invention provides methods for synthesizing a compound comprising an amide bond comprising the following steps: (a) providing a polypeptide comprising a polypeptide of the invention or a polypeptide encoded by a nucleic acid of the invention, wherein the polypeptide comprises an amidase activity; (b) providing precursors; and (c) contacting the polypeptide of step (a) with the precursor of step (b) under conditions wherein the polypeptide can catalyze the synthesis of the amide bond. In one aspect, the polypeptide can be stereoselective or stereospecific and the compound comprising an amide bond can be chiral. In one aspect, the precursors can be poorly water-soluble. In another aspect, the precursors can be achiral and the compound comprising an amide bond is chiral. In one aspect, the compound comprising an amide bond can be an amino acid or amino amid. In one aspect, the compound can be methyl dopa.

The invention provides methods for hydrolysis of a penicillin comprising the following steps: (a) providing a polypeptide comprising a polypeptide of the invention or a polypeptide encoded by a nucleic acid of the invention; (b) providing a composition comprising a penicillin; (c) combining the polypeptide of step (a) with the composition of the step (b) under conditions wherein the polypeptide can hydrolyze the penicillin.

The invention provides methods for hydrolysis of a cephalosporin comprising the following steps: (a) providing a polypeptide comprising a polypeptide of the invention or a polypeptide encoded by a nucleic acid of the invention; (b) providing a composition comprising a cephalosporin; (c) combining the polypeptide of step (a) with the composition of the step (b) under conditions wherein the polypeptide can hydrolyze the cephalosporin. In one aspect, the cephalosporin can be Cephalosporin C.

The invention provides methods for synthesis of a 7-aminocephalosporanic acid (7-ACA) comprising the following steps: (a) providing a polypeptide comprising a polypeptide of the invention or a polypeptide encoded by a nucleic acid of the invention; (b) providing a composition comprising a cephalosporin C; (c) combining the polypeptide of step (a) with the composition of the step (b) under conditions wherein the polypeptide can convert the cephalosporin C to 7-aminocephalosporanic acid (7-ACA).

The invention provides methods for cell wall hydrolysis comprising the following steps: (a) providing a polypeptide comprising a polypeptide of the invention or a polypeptide encoded by a nucleic acid of the invention; (b) providing a composition comprising a cell wall; and (c) contacting the polypeptide of step (a) with the composition of step (b) wherein the polypeptide can hydrolyze the cell wall.

The invention provides methods for influencing fermentation in food processing comprising the following steps: (a) providing a polypeptide comprising a polypeptide of the invention or a polypeptide encoded by a nucleic acid of the invention; (b) providing a composition comprising bacterial used in food processing; and (c) contacting the polypeptide of step (a) with the composition of step (b) under conditions wherein the polypeptide can change the fermentation characteristics of the bacteria. In one aspect, the fermentation characteristics of bacteria can comprise speed of growth, acid production or survival.

The invention provides methods for cheese ripening and flavor development comprising the following steps: (a) providing a polypeptide comprising a polypeptide of the invention or a polypeptide encoded by a nucleic acid of the invention; (b) providing a composition comprising cheese; (c) contacting the polypeptide of step (a) with the composition of step (b) under conditions wherein the polypeptide hydrolyze milk casein thereby assisting in cheese ripening and the development of cheese flavor.

The invention provides methods of making a transgenic plant comprising the following steps: (a) introducing a heterologous nucleic acid sequence into the cell, wherein the heterologous nucleic sequence comprises a nucleic acid of the invention, thereby producing a transformed plant cell; (b) producing a transgenic plant from the transformed cell. In one aspect, the step (a) can further comprise introducing the heterologous nucleic acid sequence by electroporation or microinjection of plant cell protoplasts. In another aspect, the step (a) can further comprise introducing the heterologous nucleic acid sequence directly to plant tissue by DNA particle bombardment. Alternatively, the step (a) can further comprise introducing the heterologous nucleic acid sequence into the plant cell DNA using an Agrobacterium tumefaciens host.

The invention provides methods of expressing a heterologous nucleic acid sequence in a cell, e.g., an animal or plant cell comprising the following steps: (a) transforming the cell with a heterologous nucleic acid sequence operably linked to a promoter, wherein the heterologous nucleic sequence comprises a nucleic acid of the invention; (b) growing the cell under conditions wherein the heterologous nucleic acids sequence is expressed in the cell.

The invention provides methods promoting bacterial or fungal killing comprising providing a polypeptide comprising a polypeptide of the invention or a polypeptide encoded by a nucleic acid of the invention, and contacting the polypeptide of step (a) with a composition, thereby promoting bacterial or fungal killing. The invention provides antimicrobial compositions comprising a polypeptide of the invention or a polypeptide encoded by a nucleic acid of the invention. The antimicrobial composition can be a bacteriocide or a fungicide.

The invention provides food products comprising a polypeptide of the invention or a polypeptide encoded by a nucleic acid of the invention. The invention provides cheeses comprising a polypeptide of the invention or a polypeptide encoded by a nucleic acid as of the invention. The invention provides dairy products comprising a polypeptide of the invention or a polypeptide encoded by a nucleic acid of the invention.

The invention provides pharmaceutical compositions comprising a polypeptide of the invention or a polypeptide encoded by a nucleic acid of the invention.

The invention provides consumer products and food products comprising a polypeptide of the invention or a polypeptide encoded by a nucleic acid of the invention, including edible products, cosmetic products, products for cleaning fabrics, hard surfaces and human skin and the like, bread and bread improvers, butter, margarine, low calorie substitutes of butter, cheeses, dressings, mayonnaise-like products, meat products, food ingredients containing peptides, shampoos, creams or lotions, e.g., for treatment of the human skin, soap and soap-replacement products, washing powders or liquids, and/or products for cleaning food production equipment and kitchen utensils.

The invention provides an isolated nucleic acid having a sequence as set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 and variants thereof having at least 50% sequence identity to the sequence of the invention and encoding polypeptides having an amidase activity, including secondary amidase activity, e.g., catalyzing the hydrolysis of amides, including enzymes having peptidase, protease and/or hydantoinase activity.

One aspect of the invention is an isolated nucleic acid having a sequence as set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ I) NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, sequences substantially identical thereto, and sequences complementary thereto.

Another aspect of the invention is an isolated nucleic acid including at least 10 consecutive bases of a sequence as set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, sequences substantially identical thereto, and the sequences complementary thereto.

In yet another aspect, the invention provides an isolated nucleic acid encoding a polypeptide having a sequence as set forth in SEQ ID NO: 2, SEQ ID NO: 4, SEQ ID NO: 6, ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 113, SEQ ID NO: 114, and variants thereof encoding a polypeptide having an amidase activity, including secondary amidase activity, and having at least 50% sequence identity to such sequences. In one aspect, the amidase activity includes the hydrolysis of amides, e.g., enzymes having peptidase, protease and/or hydantoinase activity.

Another aspect of the invention is an isolated nucleic acid encoding a polypeptide of the invention, and sequences substantially identical thereto.

Another aspect of the invention is an isolated nucleic acid encoding a polypeptide of the invention and sequences substantially identical thereto.

In yet another aspect, the invention provides a purified polypeptide having a sequence as set forth in SEQ ID NO: 2, SEQ ID NO: 4, SEQ ID NO: 6, ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 113, SEQ ID NO: 114, and sequences substantially identical thereto. In one aspect, the polypeptide has an amidase activity, including a secondary amidase activity, including e.g., the hydrolysis of amides, including enzymes having peptidase, protease and/or hydantoinase activity.

Another aspect of the invention is an isolated or purified antibody that specifically binds to a polypeptide of the invention, and sequences substantially identical thereto.

Another aspect of the invention is an isolated or purified antibody or binding fragment thereof, which specifically binds to a polypeptide of the invention, and sequences substantially identical thereto.

Another aspect of the invention is a method of making a polypeptide of the invention and sequences substantially identical thereto. The method includes introducing a nucleic acid encoding the polypeptide into a host cell, wherein the nucleic acid is operably linked to a promoter, and culturing the host cell under conditions that allow expression of the nucleic acid.

Another aspect of the invention is a method of making a polypeptide having at least 10 amino acids of a polypeptide of the invention, and sequences substantially identical thereto. The method includes introducing a nucleic acid encoding the polypeptide into a host cell, wherein the nucleic acid is operably linked to a promoter, and culturing the host cell under conditions that allow expression of the nucleic acid, thereby producing the polypeptide.

Another aspect of the invention is a method of generating a variant including obtaining a nucleic acid of the invention, e.g., a polynucleotide having a sequence as set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ED NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, sequences substantially identical thereto, sequences complementary to the sequence of the invention, fragments comprising at least 30 consecutive nucleotides of the foregoing sequence, and changing one or more nucleotides in the sequence to another nucleotide, deleting one or more nucleotides in the sequence, or adding one or more nucleotides to the sequence.

Another aspect of the invention is a computer readable medium having stored thereon a nucleic acid of the invention, and sequences substantially identical thereto, or a polypeptide of the invention and sequences substantially identical thereto. Another aspect of the invention is a computer system including a processor and a data storage device wherein the data storage device has stored thereon a nucleic acid of the invention and sequences substantially identical thereto, or a polypeptide of the invention and sequences substantially identical thereto. Another aspect of the invention is a method for comparing a first sequence to a reference sequence wherein the first sequence is a nucleic acid of the invention, and sequences substantially identical thereto, or a polypeptide of the invention and sequences substantially identical thereto. The method includes reading the first sequence and the reference sequence through use of a computer program which compares sequences; and determining differences between the first sequence and the reference sequence with the computer program. Another aspect of the invention is a method for identifying a feature in a nucleic acid of the invention and sequences substantially identical thereto, or a polypeptide of the invention and sequences substantially identical thereto, including reading the sequence through the use of a computer program which identifies features in sequences; and identifying features in the sequence with the computer program.

Another aspect of the invention is an assay for identifying fragments or variants of a polypeptide of the invention and sequences substantially identical thereto, which retain the enzymatic function of a polypeptide of the invention and sequences substantially identical thereto. The assay includes contacting a polypeptide of the invention and sequences substantially identical thereto, or polypeptide fragment or variant with a substrate molecule under conditions which allow the polypeptide fragment or variant to function, and detecting either a decrease in the level of substrate or an increase in the level of the specific reaction product of the reaction between the polypeptide and substrate thereby identifying a fragment or variant of such sequences.

Table 1 is a table of the sequences of the present invention.

The invention provides a fluorescent amidase, e.g., a secondary amidase, substrate, 7-ε-D-2-aminoadipoylamido)-4-methylcoumarin. The invention provides a composition comprising 7-ε-D-2-aminoadipoylamido)-4-methylcoumarin. In one aspect, the 7-ε-D-2-aminoadipoylamido)-4-methylcoumarin of the invention is used in high throughput (HT) activity-based, whole cell screening, e.g., for the discovery of an amidase activity. In one aspect, the 7-ε-D-2-aminoadipoylamido)-4-methylcoumarin of the invention is used for HT screening of environmental libraries. In one aspect, 7-ε-D-2-aminoadipoylamido)-4-methylcoumarin is used as a substrate for the discovery of cephalosporin C amidases. The invention provides methods for the discovery of cephalosporin C amidases using 7-ε-D-2-aminoadipoylamido)-4-methylcoumarin.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

The following drawings are illustrative of embodiments of the invention and are not meant to limit the scope of the invention as encompassed by the claims.

FIG. 1 is a block diagram of a computer system.

FIG. 2 is a flow diagram illustrating one aspect of a process for comparing a new nucleotide or protein sequence with a database of sequences in order to determine the homology levels between the new sequence and the sequences in the database.

FIG. 3 is a flow diagram illustrating one aspect of a process in a computer for determining whether two sequences are homologous.

FIG. 4 is a flow diagram illustrating one aspect of an identifier process 300 for detecting the presence of a feature in a sequence.

FIG. 5 is an illustration of an exemplary method of the invention, a two-step synthetic procedure to synthesize the fluorescent amidase substrate 7-ε-D-2-aminoadipoylamido)-4-methylcoumarin, as exemplified in Example 3.

FIG. 6 is a graph of the relatedness of amino acid sequences of secondary amidases discovered in Example 3, due to their ability to cleave the fluorogenic substrate 7-ε-D-2-aminoadipoylamido)-4-methylcoumarin.

FIG. 7 is an illustration of the two enzyme deacylation of Cephalosporin C.

FIG. 8 is a graph of the activity of SEQ ID NO: 9 and SEQ ID NO: 10 with DTT and L-Cysteine.

FIG. 9 is an illustration of the structures of various fluorescent substrates used in Example 5, including the synthesized 7-ε-D-2-aminoadipoylamido)-4-methylcoumarin.

FIG. 10 shows three novel clones that have been identified and subcloned both for enzyme characterization and for use in sequence based screening.

FIG. 11 is an illustration of the exemplary approach of Example 5.

FIG. 12 illustrates reaction samples analyzed by using High Performance Liquid Chromatography (HPLC), as described in Example 8, below.

Like reference symbols in the various drawings indicate like elements.

### DETAILED DESCRIPTION

The invention provides amidase enzymes, polynucleotides encoding the enzymes, methods of making and using these polynucleotides and polypeptides. The invention is directed to novel polypeptides having an amidase activity, nucleic acids encoding them and antibodies that bind to them. The polypeptides of the invention can be used in a variety of diagnostic, therapeutic, and industrial contexts. The amidases of the invention can have secondary amidase activity, e.g., having activity in the hydrolysis of amides, including enzymes having peptidase, protease and/or hydantoinase activity. In alternative aspects, the enzymes of the invention can be used to process foods, e.g., to increase flavors in foods (e.g., enzyme ripened cheeses), promote bacterial and fungal killing, modify and de-protect fine chemical intermediates, synthesize peptide bonds, carry out chiral resolutions, hydrolyze antibiotics, e.g., cephalosporin C.

The amidases of the invention can be active at a high and/or at a low temperature, or, over a wide range of temperature. For example, they can be active in the temperatures ranging between 20° C. to 90° C., between 30° C. to 80° C., or between 40° C. to 70° C. The invention also provides amidases that have activity at alkaline pHs or at acidic pHs, e.g., low water acidity. In alternative aspects, the amidases of the invention can have activity in acidic pHs as low as pH 5.0, pH 4.5, pH 4.0, and pH 3.5. In alternative aspects, the amidases of the invention can have activity in alkaline pHs as high as pH 9.5, pH 10, pH 10.5, and pH 11. In one aspect, the amidases of the invention are active in the temperature range of between about 40° C. to about 70° C. under conditions of low water activity (low water content).

The invention also provides methods for further modifying the exemplary amidases of the invention to generate proteins with desirable properties. For example, amidases generated by the methods of the invention can have altered enzymatic activity, thermal stability, pH/activity profile, pH/stability profile (such as increased stability at low, e.g. pH<6 or pH<5, or high, e.g. pH>9, pH values), stability towards oxidation, Ca²⁺ dependency, specific activity and the like. The invention provides for altering any property of interest. For instance, the alteration may result in a variant which, as compared to a parent enzyme, has altered enzymatic activity, or, pH or temperature activity profiles.

Definitions

The term “amidase” includes all polypeptides, e.g., enzymes, having an amidase activity, e.g., that catalyze the hydrolysis of amides. For example, the term amidase includes polypeptides having secondary amidase activity, e.g., having activity in the hydrolysis of amides. The term includes enzymes having a peptidase, a protease and/or a hydantoinase activity. The term includes enzymes that can be used to increase flavor in food (e.g., enzyme ripened cheese), promote bacterial and fungal killing, modify and de-protect fine chemical intermediates, synthesize peptide bonds, carry out chiral resolutions. The term includes enzymes that can hydrolyze an amide antibiotic, e.g., cephalosporin C. An “amidase variant” comprises an amino acid sequence which is derived from the amino acid sequence of a “precursor amidase”. The precursor amidase can include naturally-occurring amidases and recombinant amidases. The amino acid sequence of the amidase variant can be “derived” from the precursor amidase amino acid sequence by the substitution, deletion or insertion of one or more amino acids of the precursor amino acid sequence. Such modification can be of the “precursor DNA sequence” which encodes the amino acid sequence of the precursor amidase rather than manipulation of the precursor amidase enzyme per se. Suitable methods for such manipulation of the precursor DNA sequence include methods disclosed herein, as well as methods known to those skilled in the art. The amidases of the invention also can have activities as described in U.S. Pat. Nos. 6,500,659; 6,465,204; 6,429,004. In addition to the screening methods described herein, see U.S. Pat. No. 6,333,176, for an alternative method to routinely test if a polypeptide has amidase activity.

The term “antibody” includes a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

The term “fragments” as used herein includes portions of a polypeptide, e.g., a naturally occurring protein, which can exist in at least two different conformations. Fragments can have the same or substantially the same amino acid sequence as the polypeptide, e.g., the naturally occurring protein. “Substantially the same” can mean that an amino acid sequence is largely, but not entirely, the same, but retains at least one functional activity of the sequence to which it is related. In one aspect, amino acid sequences can be “substantially the same” or “substantially homologous” if they are at least about at least about 50%, 55%, 60%, 65%, 70%, 75%, 80%, 85%, 90%, 95%, 98% or 99% or more identical. Fragments which have different three dimensional structures as the naturally occurring protein are also included. An example of this, is a “pro-form” molecule, such as a low activity proprotein that can be modified by cleavage to produce a mature enzyme with significantly higher activity.

The terms “array” or “microarray” or “biochip” or “chip” as used herein is a plurality of target elements, each target element comprising a defined amount of one or more polypeptides (including antibodies) or nucleic acids immobilized onto a defined area of a substrate surface, as discussed in further detail, below.

As used herein, the terms “computer,” “computer program” and “processor” are used in their broadest general contexts and incorporate all such devices, as described in detail, below. A “coding sequence of” or a “sequence encodes” a particular polypeptide or protein, is a nucleic acid sequence which is transcribed and translated into a polypeptide or protein when placed under the control of appropriate regulatory sequences.

The term “expression cassette” as used herein refers to a nucleotide sequence which is capable of affecting expression of a structural gene (i.e., a protein coding sequence, such as an amidase of the invention) in a host compatible with such sequences. Expression cassettes include at least a promoter operably linked with the polypeptide coding sequence; and, optionally, with other sequences, e.g., transcription termination signals. Additional factors necessary or helpful in effecting expression may also be used, e.g., enhancers. Thus, expression cassettes also include plasmnids, expression vectors, recombinant viruses, any form of recombinant “naked DNA” vector, and the like.

“Operably linked” as used herein refers to a functional relationship between two or more nucleic acid (e.g., DNA) segments. Typically, it refers to the functional relationship of transcriptional regulatory sequence to a transcribed sequence. For example, a promoter is operably linked to a coding sequence, such as a nucleic acid of the invention, if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

As used herein, the term “promoter” includes all sequences capable of driving transcription of a coding sequence in a cell, e.g., a plant cell. Thus, promoters used in the constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription. “Constitutive” promoters are those that drive expression continuously under most environmental conditions and states of development or cell differentiation. “Inducible” or “regulatable” promoters direct expression of the nucleic acid of the invention under the influence of environmental conditions or developmental conditions. Examples of environmental conditions that may affect transcription by inducible promoters include anaerobic conditions, elevated temperature, drought, or the presence of light.

“Tissue-specific” promoters are transcriptional control elements that are only active in particular cells or tissues or organs, e.g., in plants or animals. Tissue-specific regulation may be achieved by certain intrinsic factors which ensure that genes encoding proteins specific to a given tissue are expressed. Such factors are known to exist in mammals and plants so as to allow for specific tissues to develop.

The term “plant” includes whole plants, plant parts (e.g., leaves, stems, flowers, roots, etc.), plant protoplasts, seeds and plant cells and progeny of same. The class of plants which can be used in the method of the invention is generally as broad as the class of higher plants amenable to transformation techniques, including angiosperms (monocotyledonous and dicotyledonous plants), as well as gymnosperms. It includes plants of a variety of ploidy levels, including polyploid, diploid, haploid and hemizygous states. As used herein, the term “transgenic plant” includes plants or plant cells into which a heterologous nucleic acid sequence has been inserted, e.g., the nucleic acids and various recombinant constructs (e.g., expression cassettes) of the invention.

“Plasmids” can be commercially available, publicly available on an unrestricted basis, or can be constructed from available plasmids in accord with published procedures. Equivalent plasmids to those described herein are known in the art and will be apparent to the ordinarily skilled artisan.

The term “gene” includes a nucleic acid sequence comprising a segment of DNA involved in producing a transcription product (e.g., a message), which in turn is translated to produce a polypeptide chain, or regulates gene transcription, reproduction or stability. Genes can include regions preceding and following the coding region, such as leader and trailer, promoters and enhancers, as well as, where applicable, intervening sequences (introns) between individual coding segments (exons).

The phrases “nucleic acid” or “nucleic acid sequence” includes oligonucleotide, nucleotide, polynucleotide, or to a fragment of any of these, to DNA or RNA (e.g., mRNA, rRNA, tRNA) of genomic or synthetic origin which may be single-stranded or double-stranded and may represent a sense or antisense strand, to peptide nucleic acid (PNA), or to any DNA-like or RNA-like material, natural or synthetic in origin, including, e.g., iRNA, ribonucleoproteins (e.g., iRNPs). The term encompasses nucleic acids, i.e., oligonucleotides, containing known analogues of natural nucleotides. The term also encompasses nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156.

“Amino acid” or “amino acid sequence” include an oligopeptide, peptide, polypeptide, or protein sequence, or to a fragment, portion, or subunit of any of these, and to naturally occurring or synthetic molecules. The terms “polypeptide” and “protein” include amino acids joined to each other by peptide bonds or modified peptide bonds, i.e., peptide isosteres, and may contain modified amino acids other than the 20 gene-encoded amino acids. The term “polypeptide” also includes peptides and polypeptide fragments, motifs and the like. The term also includes glycosylated polypeptides. The peptides and polypeptides of the invention also include all “mimetic” and “peptidomimetic” forms, as described in further detail, below.

The term “isolated” includes a material removed from its original environment, e.g., the natural environment if it is naturally occurring. For example, a naturally occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. As used herein, an isolated material or composition can also be a “purified” composition, i.e., it does not require absolute purity; rather, it is intended as a relative definition. Individual nucleic acids obtained from a library can be conventionally purified to electrophoretic homogeneity. In alternative aspects, the invention provides nucleic acids which have been purified from genomic DNA or from other sequences in a library or other environment by at least one, two, three, four, five or more orders of magnitude.

As used herein, the term “recombinant” can include nucleic acids adjacent to a “backbone” nucleic acid to which it is not adjacent in its natural environment. In one aspect, nucleic acids represent 5% or more of the number of nucleic acid inserts in a population of nucleic acid “backbone molecules.” “Backbone molecules” according to the invention include nucleic acids such as expression vectors, self-replicating nucleic acids, viruses, integrating nucleic acids, and other vectors or nucleic acids used to maintain or manipulate a nucleic acid insert of interest. In one aspect, the enriched nucleic acids represent 10%, 15%, 20%, 30%, 40%, 50%, 60%, 70%, 80%, 90%, 95%, 98% or more of the number of nucleic acid inserts in the population of recombinant backbone molecules. “Recombinant” polypeptides or proteins refer to polypeptides or proteins produced by recombinant DNA techniques; e.g., produced from cells transformed by an exogenous DNA construct encoding the desired polypeptide or protein. “Synthetic” polypeptides or protein are those prepared by chemical synthesis, as described in further detail, below.

A promoter sequence can be “operably linked to” a coding sequence when RNA polymerase which initiates transcription at the promoter will transcribe the coding sequence into mRNA, as discussed further, below.

“Oligonucleotide” includes either a single stranded polydeoxynucleotide or two complementary polydeoxynucleotide strands which may be chemically synthesized. Such synthetic oligonucleotides have no 5′ phosphate and thus will not ligate to another oligonucleotide without adding a phosphate with an ATP in the presence of a kinase. A synthetic oligonucleotide can ligate to a fragment that has not been dephosphorylated.

The phrase “substantially identical” in the context of two nucleic acids or polypeptides, can refer to two or more sequences that have, e.g., at least about 50%, 55%, 60%, 65%, 70%, 75%, 80%, 85%, 90%, 95%, 98% or 99% or more nucleotide or amino acid residue (sequence) identity, when compared and aligned for maximum correspondence, as measured using one any known sequence comparison algorithm, as discussed in detail below, or by visual inspection. In alternative aspects, the invention provides nucleic acid and polypeptide sequences having substantial identity to an exemplary sequence of the invention, SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113; and, SEQ ID NO: 2, SEQ ID NO: 4, SEQ ID NO: 6, ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 113, SEQ ID NO: 114, respectively, over a region of at least about 10, 20, 30, 40, 50, 100, 150, 200, 250, 300, 350, 400, 450, 500, 550, 600, 650, 700, 750, 800, 850, 900, 950, 1000 or more residues, or a region ranging from between about 50 residues to the full length of the nucleic acid or polypeptide. Nucleic acid sequences of the invention can be substantially identical over the entire length of a polypeptide coding region.

A “substantially identical” amino acid sequence also can include a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from an amidase, resulting in modification of the structure of the polypeptide, without significantly altering its biological activity. For example, amino- or carboxyl-terminal amino acids that are not required for amidase activity can be removed.

“Hybridization” includes the process by which a nucleic acid strand joins with a complementary strand through base pairing. Hybridization reactions can be sensitive and selective so that a particular sequence of interest can be identified even in samples in which it is present at low concentrations. Stringent conditions can be defined by, for example, the concentrations of salt or formamide in the prehybridization and hybridization solutions, or by the hybridization temperature, and are well known in the art. For example, stringency can be increased by reducing the concentration of salt, increasing the concentration of formamide, or raising the hybridization temperature, altering the time of hybridization, as described in detail, below. In alternative aspects, nucleic acids of the invention are defined by their ability to hybridize under various stringency conditions (e.g., high, medium, and low), as set forth herein.

“Variant” includes polynucleotides or polypeptides of the invention modified at one or more base pairs, codons, introns, exons, or amino acid residues (respectively) yet still retain the biological activity of an amidase of the invention. Variants can be produced by any number of means included methods such as, for example, error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, GSSM and any combination thereof. Techniques for producing variant amidase having activity at a pH or temperature, for example, that is different from a wild-type amidase, are included herein.

The term “saturation mutagenesis” or “GSSM” includes a method that uses degenerate oligonucleotide primers to introduce point mutations into a polynucleotide, as described in detail, below.

The term “optimized directed evolution system” or “optimized directed evolution” includes a method for reassembling fragments of related nucleic acid sequences, e.g., related genes, and explained in detail, below.

The term “synthetic ligation reassembly” or “SLR” includes a method of ligating oligonucleotide fragments in a non-stochastic fashion, and explained in detail, below.

Generating and Manipulating Nucleic Acids

The invention provides nucleic acids, including expression cassettes such as expression vectors, encoding the amidase polypeptides of the invention. The invention also includes methods for discovering new amidase sequences using the nucleic acids of the invention. The invention also includes methods for inhibiting the expression of amidase genes, transcripts and polypeptides using the nucleic acids of the invention. Also provided are methods for modifying the nucleic acids of the invention by, e.g., synthetic ligation reassembly, optimized directed evolution system and/or saturation mutagenesis. The nucleic acids of the invention can be made, isolated and/or manipulated by, e.g., cloning and expression of cDNA libraries, amplification of message or genomic DNA by PCR, and the like. In practicing the methods of the invention, homologous genes can be modified by manipulating a template nucleic acid, as described herein. The invention can be practiced in conjunction with any method or protocol or device known in the art, which are well described in the scientific and patent literature.

General Techniques

The nucleic acids used to practice this invention, whether RNA, iRNA, antisense nucleic acid, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

In one aspect, a nucleic acid encoding a polypeptide of the invention is assembled in appropriate phase with a leader sequence capable of directing secretion of the translated polypeptide or fragment thereof.

The invention provides fusion proteins and nucleic acids encoding them. A polypeptide of the invention can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-414). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll (1993) DNA Cell. Biol., 12:441-53.

Transcriptional and Translational Control Sequences

The invention provides nucleic acid (e.g., DNA) sequences of the invention operatively linked to expression (e.g., transcriptional or translational) control sequence(s), e.g., promoters or enhancers, to direct or modulate RNA synthesis/ expression. The expression control sequence can be in an expression vector. Exemplary bacterial promoters include lacd, lacZ, T3, T7, gpt, lambda PR, PL and trp. Exemplary eukaryotic promoters include CMV immediate early, HSV thymidine kinase, early and late SV40, LTRs from retrovirus, and mouse metallothionein I.

Promoters suitable for expressing a polypeptide in bacteria include the E. coli lac or trp promoters, the lacd promoter, the lacZ promoter, the T3 promoter, the T7 promoter, the glit promoter, the lambda PR promoter, the lambda PL promoter, promoters from operons encoding glycolytic enzymes such as 3-phosphoglycerate kinase (PGK), and the acid phosphatase promoter. Eukaryotic promoters include the CMV immediate early promoter, the HSV thymidine kinase promoter, heat shock promoters, the early and late SV40 promoter, LTRs from retroviruses, and the mouse metallothionein-I promoter. Other promoters known to control expression of genes in prokaryotic or eukaryotic cells or their viruses may also be used.

Tissue-Specific Plant Promoters

The invention provides expression cassettes that can be expressed in a tissue-specific manner, e.g., that can express an amidase of the invention in a tissue-specific manner. The invention also provides plants or seeds that express an amidase of the invention in a tissue-specific manner. The tissue-specificity can be seed specific, stem specific, leaf specific, root specific, fruit specific and the like.

In one aspect, a constitutive promoter such as the CaMV 35S promoter can be used for expression in specific parts of the plant or seed or throughout the plant. For example, for overexpression, a plant promoter fragment can be employed which will direct expression of a nucleic acid in some or all tissues of a plant, e.g., a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumefaciens, and other transcription initiation regions from various plant genes known to those of skill. Such genes include, e.g., ACT11 from Arabidopsis (Huang (1996) Plant Mol. Biol. 33:125-139); Cat3 from Arabidopsis (GenBank No. U43147, Zhong (1996) Mol. Gen. Genet. 251:196-203); the gene encoding stearoyl-acyl carrier protein desaturase from Brassica napus (Genbank No. X74782, Solocombe (1994) Plant Physiol. 104:1167-1176); GPc1 from maize (GenBank No. X15596; Martinez (1989) J. Mol. Biol 208:551-565); the Gpc2 from maize (GenBank No. U45855, Manjunath (1997) Plant Mol. Biol. 33:97-112); plant promoters described in U.S. Pat. Nos. 4,962,028; 5,633,440.

The invention uses tissue-specific or constitutive promoters derived from viruses which can include, e.g., the tobamovirus subgenomic promoter (Kumagai (1995) Proc. Natl. Acad. Sci. USA 92:1679-1683; the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer (1996) Plant Mol. Biol. 31:1129-1139).

Alternatively, the plant promoter may direct expression of amidase-expressing nucleic acid in a specific tissue, organ or cell type (i.e. tissue-specific promoters) or may be otherwise under more precise environmental or developmental control or under the control of an inducible promoter. Examples of environmental conditions that may affect transcription include anaerobic conditions, elevated temperature, the presence of light, or sprayed with chemicals/hormones. For example, the invention incorporates the drought-inducible promoter of maize (Busk (1997) supra); the cold, drought, and high salt inducible promoter from potato (Kirch (1997) Plant Mol. Biol. 33:897 909).

Tissue-specific promoters can promote transcription only within a certain time frame of developmental stage within that tissue. See, e.g., Blazquez (1998) Plant Cell 10:791-800, characterizing the Arabidopsis LEAFY gene promoter. See also Cardon (1997) Plant J 12:367-77, describing the transcription factor SPL3, which recognizes a conserved sequence motif in the promoter region of the A. thaliana floral meristem identity gene AP1; and Mandel (1995) Plant Molecular Biology, Vol. 29, pp 995-1004, describing the meristem promoter eIF4. Tissue specific promoters which are active throughout the life cycle of a particular tissue can be used. In one aspect, the nucleic acids of the invention are operably linked to a promoter active primarily only in cotton fiber cells. In one aspect, the nucleic acids of the invention are operably linked to a promoter active primarily during the stages of cotton fiber cell elongation, e.g., as described by Rinehart (1996) supra. The nucleic acids can be operably linked to the Fb12A gene promoter to be preferentially expressed in cotton fiber cells (Ibid). See also, John (1997) Proc. Natl. Acad. Sci. USA 89:5769-5773; John, et al., U.S. Pat. Nos. 5,608,148 and 5,602,321, describing cotton fiber-specific promoters and methods for the construction of transgenic cotton plants. Root-specific promoters may also be used to express the nucleic acids of the invention. Examples of root-specific promoters include the promoter from the alcohol dehydrogenase gene (DeLisle (1990) Int. Rev. Cytol. 123:39-60). Other promoters that can be used to express the nucleic acids of the invention include, e.g., ovule-specific, embryo-specific, endosperm-specific, integument-specific, seed coat-specific promoters, or some combination thereof; a leaf-specific promoter (see, e.g., Busk (1997) Plant J. 11:1285 1295, describing a leaf-specific promoter in maize); the ORF13 promoter from Agrobacterium rhizogenes (which exhibits high activity in roots, see, e.g., Hansen (1997) supra); a maize pollen specific promoter (see, e.g., Guerrero (1990) Mol. Gen. Genet. 224:161168); a tomato promoter active during fruit ripening, senescence and abscission of leaves and, to a lesser extent, of flowers can be used (see, e.g., Blume (1997) Plant J. 12:731 746); a pistil-specific promoter from the potato SK2 gene (see, e.g., Ficker (1997) Plant Mol. Biol. 35:425 431); the Blec4 gene from pea, which is active in epidermal tissue of vegetative and floral shoot apices of transgenic alfalfa making it a useful tool to target the expression of foreign genes to the epidermal layer of actively growing shoots or fibers; the ovule-specific BEL1 gene (see, e.g., Reiser (1995) Cell 83:735-742, GenBank No. U39944); and/or, the promoter in Klee, U.S. Pat. No. 5,589,583, describing a plant promoter region is capable of conferring high levels of transcription in meristematic tissue and/or rapidly dividing cells.

Alternatively, plant promoters which are inducible upon exposure to plant hormones, such as auxins, are used to express the nucleic acids of the invention. For example, the invention can use the auxin-response elements E1 promoter fragment (AuxREs) in the soybean (Glycine max L.) (Liu (1997) Plant Physiol. 115:397-407); the auxin-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37:906-913); a plant biotin response element (Streit (1997) Mol. Plant Microbe Interact. 10:933-937); and, the promoter responsive to the stress hormone abscisic acid (Sheen (1996) Science 274:1900-1902).

The nucleic acids of the invention can also be operably linked to plant promoters which are inducible upon exposure to chemicals reagents which can be applied to the plant, such as herbicides or antibiotics. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. Coding sequence can be under the control of, e.g., a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324). Using chemically- (e.g., hormone- or pesticide-) induced promoters, i.e., promoter responsive to a chemical which can be applied to the transgenic plant in the field, expression of a polypeptide of the invention can be induced at a particular stage of development of the plant. Thus, the invention also provides for transgenic plants containing an inducible gene encoding for polypeptides of the invention whose host range is limited to target plant species, such as corn, rice, barley, wheat, potato or other crops, inducible at any stage of development of the crop.

One of skill will recognize that a tissue-specific plant promoter may drive expression of operably linked sequences in tissues other than the target tissue. Thus, a tissue-specific promoter is one that drives expression preferentially in the target tissue or cell type, but may also lead to some expression in other tissues as well.

The nucleic acids of the invention can also be operably linked to plant promoters which are inducible upon exposure to chemicals reagents. These reagents include, e.g., herbicides, synthetic auxins, or antibiotics which can be applied, e.g., sprayed, onto transgenic plants. Inducible expression of the amidase-producing nucleic acids of the invention will allow selection of plants with the desired amidase synthesis or activity. The development of plant parts can thus controlled. In this way the invention provides the means to facilitate the harvesting of plants and plant parts. For example, in various embodiments, the maize In2-2 promoter, activated by benzenesulfonamide herbicide safeners, is used (De Veylder (1997) Plant Cell Physiol. 38:568-577); application of different herbicide safeners induces distinct gene expression patterns, including expression in the root, hydathodes, and the shoot apical meristem. Coding sequences of the invention are also under the control of a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324).

If proper polypeptide expression is desired, a polyadenylation region at the 3′-end of the coding region should be included. The polyadenylation region can be derived from the natural gene, from a variety of other plant genes, or from genes in the Agrobacterial T-DNA.

Expression Vectors and Cloning Vehicles

The invention provides expression vectors and cloning vehicles comprising nucleic acids of the invention, e.g., sequences encoding the amidases and antibodies of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors of the invention can include chromosomal, non-chromosomal and synthetic DNA sequences. Large numbers of suitable vectors are known to those of skill in the art, and are commercially available. Exemplary vectors are include: bacterial: pQE vectors (Qiagen), pBluescript plasmids, pNH vectors, (lambda-ZAP vectors (Stratagene); ptrc99a, pKK223-3, pDR540, pRIT2T (Pharmacia); Eukaryotic: pXT1, pSG5 (Stratagene), pSVK3, pBPV, pMSG, pSVLSV40 (Pharmacia). However, any other plasmid or other vector may be used so long as they are replicable and viable in the host. Low copy number or high copy number vectors may be employed with the present invention.

The expression vector can comprise a promoter, a ribosome binding site for translation initiation and a transcription terminator. The vector may also include appropriate sequences for amplifying expression. Mammalian expression vectors can comprise an origin of replication, any necessary ribosome binding sites, a polyadenylation site, splice donor and acceptor sites, transcriptional termination sequences, and 5′ flanking non-transcribed sequences. In some aspects, DNA sequences derived from the SV40 splice and polyadenylation sites may be used to provide the required non-transcribed genetic elements. In one aspect, the expression vectors contain one or more selectable marker genes to permit selection of host cells containing the vector. Such selectable markers include genes encoding dihydrofolate reductase or genes conferring neomycin resistance for eukaryotic cell culture, genes conferring tetracycline or ampicillin resistance in E. coli, and the S. cerevisiae TRP1 gene. Promoter regions can be selected from any desired gene using chloramphenicol transferase (CAT) vectors or other vectors with selectable markers.

Vectors for expressing the polypeptide or fragment thereof in eukaryotic cells can also contain enhancers to increase expression levels. Enhancers are cis-acting elements of DNA, usually from about 10 to about 300 bp in length that act on a promoter to increase its transcription. Examples include the SV40 enhancer on the late side of the replication origin bp 100 to 270, the cytomegalovirus early promoter enhancer, the polyoma enhancer on the late side of the replication origin, and the adenovirus enhancers.

A nucleic acid sequence can be inserted into a vector by a variety of procedures. In general, the sequence is ligated to the desired position in the vector following digestion of the insert and the vector with appropriate restriction endonucleases. Alternatively, blunt ends in both the insert and the vector may be ligated. A variety of cloning techniques are known in the art, e.g., as described in Ausubel and Sambrook. Such procedures and others are deemed to be within the scope of those skilled in the art.

The vector can be in the form of a plasmid, a viral particle, or a phage. Other vectors include chromosomal, non-chromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by, e.g., Sambrook.

Particular bacterial vectors which can be used include the commercially available plasmids comprising genetic elements of the well known cloning vector pBR322 (ATCC 37017), pKK223-3 (Pharmacia Fine Chemicals, Uppsala, Sweden), GEM1 (Promega Biotec, Madison, Wis., USA) pQE70, pQE60, pQE-9 (Qiagen), pD10, psiX174 pBluescript II KS, pNH8A, pNH16a, pNH18A, pNH46A (Stratagene), ptrc99a, pKK223-3, pKY233-3, DR540, pRIT5 (Pharmacia), pKK232-8 and pCM7. Particular eukaryotic vectors include pSV2CAT, pOG44, pXT1, pSG (Stratagene) pSVK3, pBPV, pMSG, and pSVL (Pharmacia). However, any other vector may be used as long as it is replicable and viable in the host cell.

The nucleic acids of the invention can be expressed in expression cassettes, vectors or viruses and transiently or stably expressed in plant cells and seeds. One exemplary transient expression system uses episomal expression systems, e.g., cauliflower mosaic virus (CaMV) viral RNA generated in the nucleus by transcription of an episomal mini-chromosome containing supercoiled DNA, see, e.g., Covey (1990) Proc. Natl. Acad. Sci. USA 87:1633-1637. Alternatively, coding sequences, i.e., all or sub-fragments of sequences of the invention can be inserted into a plant host cell genome becoming an integral part of the host chromosomal DNA. Sense or antisense transcripts can be expressed in this manner. A vector comprising the sequences (e.g., promoters or coding regions) from nucleic acids of the invention can comprise a marker gene that confers a selectable phenotype on a plant cell or a seed. For example, the marker may encode biocide resistance, particularly antibiotic resistance, such as resistance to kanamycin, G418, bleomycin, hygromycin, or herbicide resistance, such as resistance to chlorosulfuron or Basta.

Expression vectors capable of expressing nucleic acids and proteins in plants are well known in the art, and can include, e.g., vectors from Agrobacterium spp., potato virus X (see, e.g., Angell (1997) EMBO J. 16:3675-3684), tobacco mosaic virus (see, e.g., Casper (1996) Gene 173:69-73), tomato bushy stunt virus (see, e.g., Hillman (1989) Virology 169:42-50), tobacco etch virus (see, e.g., Dolja (1997) Virology 234:243-252), bean golden mosaic virus (see, e.g., Morinaga (1993) Microbiol Immunol. 37:471-476), cauliflower mosaic virus (see, e.g., Cecchini (1997) Mol. Plant Microbe Interact. 10:1094-1101), maize Ac/Ds transposable element (see, e.g., Rubin (1997) Mol. Cell. Biol. 17:6294-6302; Kunze (1996) Curr. Top. Microbiol. Immunol. 204:161-194), and the maize suppressor-mutator (Spm) transposable element (see, e.g., Schlappi (1996) Plant Mol. Biol. 32:717-725); and derivatives thereof.

In one aspect, the expression vector can have two replication systems to allow it to be maintained in two organisms, for example in mammalian or insect cells for expression and in a prokaryotic host for cloning and amplification. Furthermore, for integrating expression vectors, the expression vector can contain at least one sequence homologous to the host cell genome. It can contain two homologous sequences which flank the expression construct. The integrating vector can be directed to a specific locus in the host cell by selecting the appropriate homologous sequence for inclusion in the vector. Constructs for integrating vectors are well known in the art.

Expression vectors of the invention may also include a selectable marker gene to allow for the selection of bacterial strains that have been transformed, e.g., genes which render the bacteria resistant to drugs such as ampicillin, chloramphenicol, erythromycin, kanamycin, neomycin and tetracycline. Selectable markers can also include biosynthetic genes, such as those in the histidine, tryptophan and leucine biosynthetic pathways.

Host Cells and Transformed Cells

The invention also provides a transformed cell comprising a nucleic acid sequence of the invention, e.g., a sequence encoding an amidase or antibody of the invention, or a vector of the invention. The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera S19. Exemplary animal cells include CHO, COS or Bowes melanoma or any mouse or human cell line. The selection of an appropriate host is within the abilities of those skilled in the art. Techniques for transforming a wide variety of higher plant species are well known and described in the technical and scientific literature. See, e.g., Weising (1988) Ann. Rev. Genet. 22:421-477, U.S. Pat. No. 5,750,870.

The vector can be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, I., Basic Methods in Molecular Biology, (1986)).

In one aspect, the nucleic acids or vectors of the invention are introduced into the cells for screening, thus, the nucleic acids enter the cells in a manner suitable for subsequent expression of the nucleic acid. The method of introduction is largely dictated by is the targeted cell type. Exemplary methods include CaPO₄ precipitation, liposome fusion, lipofection (e.g., LIPOFECTIN™), electroporation, viral infection, etc. The candidate nucleic acids may stably integrate into the genome of the host cell (for example, with retroviral introduction) or may exist either transiently or stably in the cytoplasm (i.e. through the use of traditional plasmids, utilizing standard regulatory sequences, selection markers, etc.). As many pharmaceutically important screens require human or model mammalian cell targets, retroviral vectors capable of transfecting such targets are preferred.

Where appropriate, the engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment thereof can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

Amplification of Nucleic Acids

In practicing the invention, nucleic acids of the invention and nucleic acids encoding the polypeptides of the invention, or modified nucleic acids of the invention, can be reproduced by amplification. Amplification can also be used to clone or modify the nucleic acids of the invention. Thus, the invention provides amplification primer sequence pairs for amplifying nucleic acids of the invention. In alternative aspects, where the primer pairs are capable of amplifying nucleic acid sequences of the invention, or a subsequence thereof. One of skill in the art can design amplification primer sequence pairs for any part of or the full length of these sequences; for example:

The exemplary SEQ ID NO: 1 is

atgaactcaaccttagcctacttcacggaacagggacccatgtctgacccgggaacctatcgttcgctttttgaagatcttcccacatcca
tcccagatctggtgaagcttgtgcagggagtcaccctacatatcttttggacggagcgatatggactcaaagttcccccgcaacgaatgga
ggaactgcagctccgttcgatggagaaacggctggcgcgcacgctcgaattagatccgcgtccacttgttgagccgcgtccgctagagaac
aagttgctcggcaattgtcgggatcattctctattgcttaccgcgctgctgcgtcatcagggagttccggctcgcgcccgctgtgggtttg
gtgcctacttcctgccagaccattttgaggaccactgggtcgttgagtactggaatcaggagcaatcccgctgggtacttgtggacgcaca
gttggatgcctcacagcgcgaggtgttgaagatcgactttgacactttggatgtcccccgtgatcaattcatcgtcggcggcaaagcctgg
caaatgtgccgttctggcgagcaagaccctggcaaattcggcattttcgatatgaatggattgggcttcgtgcggggggatcttgtacgtg
atgtcgcctcgctcaataaaatggaattgctgccctgggattgctggggtgttattctcgttgagaaactcgatgacccggctgacctttc
cgtgcttgatcgagtcgcttcgctcaccgcgagagatgtccccgattttgaagtgctgcgcgcctgttatgagtctgatccgcgactgcgt
gtgaacgactcattgctgagctacgtcaacgggaacatggtggaagtccagatcgcttaa

Thus, an exemplary amplification primer sequence pair is residues 1 to 21 of SEQ ID NO: 1 and the complementary strand of the last 21 residues of SEQ ID NO: 1.

The exemplary SEQ ID NO: 3 is

gtgccgagcctcgacgagtacgcgacccacagcgccttcaccgaccccggccggcaccgggacctgctcggcgcgaccgggac
gtcgcccgacgacctgcaccgtgcggcgacaggcgtcgtcctgcactaccgcggccagcgcgaccggctcacggacgagcagc
tgcccgacgtcgacctgcgctggttctccgcccagctcgaggtcgttcggcaccgcgcggcgctcccgctcggcgcgcaccgg
acggacgcgcagcacctcgcggggtgctgccgcgaccacacgctgctcgccgtcgccgtcctgcgcgagcacggcatccccgc
gcgcagccgcgtcggcttcgccgactacttcgagcccgacttccaccacgaccacgtcgtcgtcgagcggtgggacggcgcgc
ggtgggtgcgcttcgactcggcgctggacccggcggaccacctgttcgacgtggacgacatgccggcgggggagggcatgccg
ttcgagacggccgccgaggtctggctcgccgcgcgggcgggccgcgtcgacccccggcggtacggcgtggacaaggcgatgcc
gcacctgatcggcatcccgttcctgctcggcgaggtcttcctcgagctcgcgcaccggcagcgcgacgagatcctgctgtggg
acgtgtggggcgtcggcatcccgccgttcgcgcggccggacggcctggcacccgtgaccatgtcggacgacgagatggcggag
ctcgccgacgaggtggcgcggctcgtcgtcgcggcggacgacggcgacgacgcggctgacgcggcgctcgacgcccgctacgc
cgccgatccccgcctgcggccgacggccaacccgctcgtggcgctctcgccgctcgaacgcatcggggacgtcgacctgacgg
cgcggacgacgacctggcggtga

Thus, an exemplary amplification primer sequence pair is residues 1 to 21 of SEQ ID NO: 3 and the complementary strand of the last 21 residues of SEQ ID NO: 3.

The exemplary SEQ ID NO: 5 is

atgaccaatcagccggagcgcagcaccgcacggtcatactacgccgccccggcggcgatgaccgacttgagcgcgcatcgcgcg
cgcttgcgcgacctgccgaccgatctggccgggctctgccgcgtcattcagggactgctggtgcatccctttctcgcgcacctc
tacggcctgccgtcgagcgcgctgcgcctcggcgagttggagttgcgccgcgcctcggcgatgctcgatcacgcgttgaccctc
gacgcgcgcccgctcgtcgaggcgcgcccgccggagcgacgcctggtgggcaactgccgccacttttcggtgctgttctgcgcc
ttactgcgcgcccagggcgttccggcgcgcgcccgctgcggattcggcgcctacttcaatccggcgcgtttcgaggatcactgg
gtcggcgaagtctgggactcgacgcgcggcgcctggcgcctcgtcgacgcacagctcgatgccgagcagcgccaggcgctgcgc
atctcgttcgatccgctcgacgtgccgcgcagcgagttcgtggtagccggcgaggcgtggcgacggtgccggagcggcgcggcc
gctcccgaactgttcggcatcctcgatctgcgcggtctctggttcgtgcgcggcaacgtggtgcgcgacctcgccgcgttcagc
aagcgcgaactgctgccgtgggacggctggggtctgatggcgacgcgcgaggacagcagtcctgccgagctggcgctactcgac
cacgtcgccgagctgactctggccggcgacgagcgccacgacgagcgcctgcatctgcaggatgccgaacccggcctgcgcgtg
cctcgcgtcgttctcagcttcaacctgaacggcgccgaggtcgatctcggccccggcgttgcgaactga

Thus, an exemplary amplification primer sequence pair is residues 1 to 21 of SEQ ID NO: 5 and the complementary strand of the last 21 residues of SEQ ID NO: 5.

The exemplary SEQ ID NO: 7 is

atgcgcagcgacctcgcattctatcaaacacaggggatcatcaccgatcccggccaacatcacgacctgctgaccggcctgccggg
cgacctgcccggcctggtcaaagtcgtccagggcctggtggtgcacgtcttctggctggagcgctacggcttgaagctgaaggaga
cgcgcaaggccgaggtgcagttgcgctgggctgaaaagcagctcgagcgcatccgcgcgctcgacccgcgcccgctggccgaagcc
cggcccctggagaagcgcctggtgggcaactgccgggatttcaccgtcctgctggtatgcctgctgcgcgcccggggcatcccggc
ccgcgcgcgctgcggtttcgccaagtacttcgaggcggggcggcacatggatcactgggtggccgaggtttggaacgccgagctgc
aacgctggactttggtcgacgcacaactcgacgacctgcagcgcaaggcgctcgcgataccgttcaacccgctggacgtgccgcgc
gtgcagttcctgaccggcggcgaagcctggctgcgctgccgcaaggggcaggccgaccccgagaccttcggcatcttcgacctgaa
ggggttgtggttcgtgcgcggggacttcgtgcgcgacgtggccgcgctcaacaaggttgagctgctgccctgggatgcatggggca
tcgccgatgtgcaggaaaaggatatctccggggaagacctggttttcctggacgaggtggccgagctctcacatggcgacgtggag
cgcttcgagcaggtgaaggggctgtatgaaaccgacccccggctgcacgtgccggaggtgatcaacagttacacacaggcaggggt
gctgcgcgtcgatctccaagcacattcgtag

Thus, an exemplary amplification primer sequence pair is residues 1 to 21 of SEQ ID NO: 7 and the complementary strand of the last 21 residues of SEQ ID NO: 7.

The exemplary SEQ ID NO: 9 is

atgaccgatcgtgcgccgtacgccgcccagagtcccatctccgatccgggcgatatgtccaggtggcttactggcttgccagcagatt
tcgcggccctgcgggcgctggccaggccgctggtcgcacactaccgggccgatgacctggcggcgttcggcattcccgaggagcgcgt
ggaggagatcgacacgcggtttgcggagcggatgctggcgcggctgcacgagatggagagcggtccgctcacgccggagcgcacgccg
gccaaccgcctcgtgggctgctgccgggacttcaccctgctctacctgaccatgctgcgccacgccggcatcccggcacggtcacgcg
tgggctttgccggctactttgccgctggctggttcatcgaccacgtggtggctgaggtctgggacgaggccaacgggcgctggcgcct
ggtcgatccccagttggcggatgtgcgcactgaccccaacgacggcttccccatcgatacgctcgatatcccgcgcgaccgtttcctg
gttgcgggcatggcgtggcaggcttgccggagtgaggaactgcagccagagcagttcgtggttgacccagatctcgatatcccggtga
cgcgcggctggctgcaactgcggcacaacctggtgcaggacctcgccgcactgacgaagcgggagatgatcctctgggatacgtgggg
catcctgggtgacgagccggtggcggaggatacgctgcccttgctggacagcatcgcggctgtcaccgccgatcccgatgtcacgtac
gcggacgccctcaatctctacgagcgggagccgggggtgcaggtgccgccagaggtgatgagcttcaacatgctggcgaacgagccaa
ggatggtggcgtcgggggtgtag

Thus, an exemplary amplification primer sequence pair is residues 1 to 21 of SEQ ID NO: 9 and the complementary strand of the last 21 residues of SEQ ID NO: 9.

The exemplary SEQ ID NO: 11 is

atgcttgcagccggggtaccaggacgacttgtaggccttcaccggattgttgaactcgatctcgagcgtgaaacgctcgggcagctgc
agcaggcacttcttcaggtcgccctgcagtgcctgcctgacgccctcgcggatctgcgcgcaggcggccttgggcgcgaggctgacgg
tcgacggcccgatgccctcgctgaccgcgacggcggtgatgttggggttggtctccttgatgtcggtgcagagctgccagtcgccgga
gacgaacaccaccgggacgccgaccatggcggcggcataggcgtgcagcaggaattccgaagtcgccacgccgttgatgcgcatgcgc
atgacctcacccgtcagcgtgtgcgccaagggattggtctcgtcgccggccttcgagtgatagccgatgaacatcgcggcatcgaagc
tcttgtccagctcctgcaccatgctcatcggatggccgctccagccgcggatcaggcgcacattctccggcagtcggcctgcaggatg
ttgcgcccggtcgcgtgtgcgtccttgatcaggatctccttggcccccgccgcgttggcgccgtcgcacgccgccagcacttcgcgcg
tcatctgctcgcgatgctcgggatagtcggcgtgcggcttgcgcgcctcgtcccagttggtgatgccggcggtgccctcgatgtcggc
gctgatgaagatcttcatgccactcctctttgcaaacgcgcgccactctag

Thus, an exemplary amplification primer sequence pair is residues 1 to 21 of SEQ ID NO: 11 and the complementary strand of the last 21 residues of SEQ ID NO: 11.

The exemplary SEQ ID NO: 13 is

ttgccgcaaggcgtgtgcgccgcttcactgcgtcggtatcggcaacgaaaggaacagtacctcatgacgatacaccaacagattctc
gacttctatacgcgccctgccgggatgacgtccgccggccaattcgcgcccttattcgacgcgctgccgagcgagtgggcgaactcg
tccgcatcatccagggccttggggtgtatgaccttgtggcgtccggcttctacggcttcacgatcccggacgagcgccagggcgaga
tccacctccgccccgtagagaaaatgctgggccgcctcctcgccctcgacgaccggccgctccgtgtcgcccggccggtcgacaggc
gtctggtcggccgctgccgtcacttcgtgctgctactcgtcgccatgttgcgggccaagggtgttccggcgcgggcgcgctgcgggt
tcggctcctactttagacgcgggttctttgaggaccactgggtgtgcgagtactggaacgccgccgaagcccgctgggtgcttgtcg
atccacagttcgacgaggtttggcgggagacactacagatagatcacgacattcttgatgtgccgcgcgaccgtttcctggagcggg
cgacgcctgggcgcaatgccgcgcgggtgcggccgacccggcgaagttcgaaatcgttttcgccgacctgagcggactgtggttcat
cgccgggaacctggtgcgcgacgtggcggcgctcaacaagacggagatgctgccgtgggacgtctggggcgcccagccccgcccgca
cgaagcgctcgacgacgaccaactgaccttcttcgacaaactcgccgcgctcacgcgcgagcctgacgcgtcgttcgcggaactgcg
caccctctacgaaggagatgatcgcctgcgtgtgccggcgaccgtcttcaacgcgatgcgcaacgcgcccgaaacgatcgcgggctg
a

Thus, an exemplary amplification primer sequence pair is residues 1 to 21 of SEQ ID NO 013 and the complementary strand of the last 21 residues of SEQ ID NO: 13.

The exemplary SEQ ID NO: 15 is

gtggaccaaaccggagcaaatgacgcactggtggggcatggccggcggcccgcgtccgccggtcgccgagaccgacctgcgcg
tcggcggccgcttcaaggtgcagttctgggatcccaagaacgagcatcacagcgtcagcggcatctacaaggaggcgtgccca
accggaagctcgccttctcgtgggcctggcagagcacgcccgagcgcgaatcgctggtgacgatcgagctcaacccggtcacc
gagggcaccatgctgacgctgacccacgagcagttcttcgacgagaaggcgcgcgacgaccacggccgcggctggaacgtcgc
cctcgaccgcctggagagcttcctcacatgacccccggccaggccgtggaccgggcgttcgccggcttgccgggcgatcccgc
gtcgctggccggcgtcgtgcagggccttttgatgcacgagcatatcgcgccggcctacggcctcaccctgagcgaggcccagc
acgcggaggcgcacacccggccggtcgaggagatcgtgcgccagatcgtggcgcacgatcctcgtccgctcgccgagccgcgc
gcgcccggcgaacgccaggtcggcaattgccggcacttcaccctgctgcacgtcacgatgctgcgccgcgccggcgtgcgggc
gcgcgcccgctgcggcttcggcggctacttcgagccgggcaagttcctcgaccactgggtcaccgaatactggaacgagcggc
gccaggcgtgggttctggtcgatgcccagctcgatgcccgccagcgcgagctcttcaagatcgccttcgaccccctcgacgtg
ccgcgcgacaagttcctggtcgcgggcgacgcctggcagcgctgccgcgccggcaccgccgatccgaacgcgttcggcatcct
cgacatgcacgggctgtggttcgtcgccggcaatttgatccgcgacgtcgccgcgctcaacgaccacgtgatgctgccgtggg
acgtgtggggcgcgatgacccagaacgacgcggagctcgaccaaccgttcctcgacaagctggccgcgctgaccgtcgagccc
gaccgccatttcggcgagctgcgcgccgtctaccaggatccgcgcgtgaaagtgccggcgaccgtgttcaacgccatccgcaa
ccgccccgaaaccctttga

Thus, an exemplary amplification primer sequence pair is residues 1 to 21 of SEQ ID NO: 15 and the complementary strand of the last 21 residues of SEQ ID NO: 15.

Amplification reactions can also be used to quantify the amount of nucleic acid in a sample (such as the amount of message in a cell sample), label the nucleic acid (e.g., to apply it to an array or a blot), detect the nucleic acid, or quantify the amount of a specific nucleic acid in a sample. In one aspect of the invention, message isolated from a cell or a cDNA library are amplified.

The skilled artisan can select and design suitable oligonucleotide amplification primers. Amplification methods are also well known in the art, and include, e.g., polymerase chain reaction, PCR (see, e.g., PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564.

Determining the Degree of Sequence Identity

The invention provides nucleic acids and polypeptides having at least 50% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, 30 SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, and, SEQ ID NO: 2, SEQ ID NO: 4, SEQ ID NO: 6, ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 113, SEQ ID NO: 114. In one aspect, the invention provides nucleic acids and polypeptides having at least 99%, 98%, 97%, 96%, 95%, 90%, 85%, 80%, 75%, 70%, 65%, 60%, 55% or 50% sequence identity (homology) to a sequence of the invention. In one aspect, the invention provides nucleic acids and polypeptide having sequence as set forth in a sequence of the invention. In alternative aspects, the sequence identity can be over a region of at least about 5, 10, 20, 30, 40, 50, 100, 150, 200, 250, 300, 350, 400, 450, 500, 550, 600, 650, 700, 750, 800, 850, 900, 950, 1000, or more consecutive residues, or the full length of the nucleic acid or polypeptide. The extent of sequence identity (homology) may be determined using any computer program and associated parameters, including those described herein, such as BLAST 2.2.2. or FASTA version 3.0t78, with the default parameters.

Homologous sequences also include RNA sequences in which uridines replace the thymines in the nucleic acid sequences. The homologous sequences may be obtained using any of the procedures described herein or may result from the correction of a sequencing error. It will be appreciated that the nucleic acid sequences as set forth herein can be represented in the traditional single character format (see, e.g., Stryer, Lubert. Biochemistry, 3rd Ed., W. H Freeman & Co., New York) or in any other format which records the identity of the nucleotides in a sequence.

Various sequence comparison programs identified herein are used in this aspect of the invention. Protein and/or nucleic acid sequence identities (homologies) may be evaluated using any of the variety of sequence comparison algorithms and programs known in the art. Such algorithms and programs include, but are not limited to, TBLASTN, BLASTP, FASTA, TFASTA, and CLUSTALW (Pearson and Lipman, Proc. Natl. Acad. Sci. USA 85(8):2444-2448, 1988; Altschul et al., J. Mol. Biol. 215(3):403-410, 1990; Thompson et al., Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993).

Homology or identity can be measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various deletions, substitutions and other modifications. The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence (a sequence of the invention), to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the numbers of contiguous residues. For example, in alternative aspects of the invention, contiguous residues ranging anywhere from 20 to the full length of an exemplary polypeptide or nucleic acid sequence of the invention are compared to a reference sequence of the same number of contiguous positions after the two sequences are optimally aligned. If the reference sequence has the requisite sequence identity to an exemplary polypeptide or nucleic acid sequence of the invention, e.g., 50%, 55%, 60%, 65%, 70%, 75%, 80%, 90% or 95%, 98%, 99% or more sequence identity to a sequence of the invention, that sequence is within the scope of the invention. In alternative embodiments, subsequences ranging from about 20 to 600, about 50 to 200, and about 100 to 150 are compared to a reference sequence of the same number of contiguous positions after the two sequences are optimally aligned. Methods of alignment of sequence for comparison are well known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of person & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. Other algorithms for determining homology or identity include, for example, in addition to a BLAST program (Basic Local Alignment Search Tool at the National Center for Biological Information), ALIGN, AMAS (Analysis of Multiply Aligned Sequences), AMPS (Protein Multiple Sequence Alignment), ASSET (Aligned Segment Statistical Evaluation Tool), BANDS, BESTSCOR, BIOSCAN (Biological Sequence Comparative Analysis Node), BLIMPS (BLocks IMProved Searcher), FASTA, Intervals & Points, BMB, CLUSTAL V, CLUSTAL W, CONSENSUS, LCONSENSUS, WCONSENSUS, Smith-Waterman algorithm, DARWIN, Las Vegas algorithm, FNAT (Forced Nucleotide Alignment Tool), Framealign, Framesearch, DYNAMIC, FILTER, FSAP (Fristensky Sequence Analysis Package), GAP (Global Alignment Program), GENAL, GIBBS, GenQuest, ISSC (Sensitive Sequence Comparison), LALIGN (Local Sequence Alignment), LCP (Local Content Program), MACAW (Multiple Alignment Construction & Analysis Workbench), MAP (Multiple Alignment Program), MBLKP, MBLKN, PIMA (Pattern-Induced Multi-sequence Alignment), SAGA (Sequence Alignment by Genetic Algorithm) and WHAT-IF. Such alignment programs can also be used to screen genome databases to identify polynucleotide sequences having substantially identical sequences. A number of genome databases are available, for example, a substantial portion of the human genome is available as part of the Human Genome Sequencing Project (Gibbs, 1995). Several genomes have been sequenced, e.g., M. genitalium (Fraser et al., 1995), M. jannaschii (Bult et al., 1996), H. influenzae (Fleischmann et al., 1995), E. coli (Blattner et al., 1997), and yeast (S. cerevisiae) (Mewes et al., 1997), and D. melanogaster (Adams et al., 2000). Significant progress has also been made in sequencing the genomes of model organism, such as mouse, C. elegans, and Arabadopsis sp. Databases containing genomic information annotated with some functional information are maintained by different organization, and are accessible via the internet.

BLAST, BLAST 2.0 and BLAST 2.2.2 algorithms are also used to practice the invention. They are described, e.g., in Altschul (1977) Nuc. Acids Res. 25:3389-3402; Altschul (1990) J. Mol. Biol. 215:403-410. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul (1990) supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectations (E) of 10, and the BLOSUM62 scoring matrix (see Henikoff & Henikoff (1989) Proc. Natl. Acad. Sci. USA 89:10915) alignments (B) of 50, expectation (E) of 10, M=5, N=−4, and a comparison of both strands. The BLAST algorithm also performs a statistical analysis of the similarity between two sequences (see, e.g., Karlin & Altschul (1993) Proc. Natl. Acad. Sci. USA 90:5873). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more preferably less than about 0.01, and most preferably less than about 0.001. In one aspect, protein and nucleic acid sequence homologies are evaluated using the Basic Local Alignment Search Tool (“BLAST”). For example, five specific BLAST programs can be used to perform the following task: (1) BLASTP and BLAST3 compare an amino acid query sequence against a protein sequence database; (2) BLASTN compares a nucleotide query sequence against a nucleotide sequence database; (3) BLASTX compares the six-frame conceptual translation products of a query nucleotide sequence (both strands) against a protein sequence database; (4) TBLASTN compares a query protein sequence against a nucleotide sequence database translated in all six reading frames (both strands); and, (5) TBLASTX compares the six-frame translations of a nucleotide query sequence against the six-frame translations of a nucleotide sequence database. The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., Science 256:1443-1445, 1992; Henikoff and Henikoff, Proteins 17:49-61, 1993). Less preferably, the PAM or PAM250 matrices may also be used (see, e.g., Schwartz and Dayhoff, eds., 1978, Matrices for Detecting Distance Relationships: Atlas of Protein Sequence and Structure, Washington: National Biomedical Research Foundation).

In one aspect of the invention, to determine if a nucleic acid has the requisite sequence identity to be within the scope of the invention, the NCBI BLAST 2.2.2 programs is used, default options to blastp. There are about 38 setting options in the BLAST 2.2.2 program. In this exemplary aspect of the invention, all default values are used except for the default filtering setting (i.e., all parameters set to default except filtering which is set to OFF); in its place a “−F F” setting is used, which disables filtering. Use of default filtering often results in Karlin-Altschul violations due to short length of sequence.

The default values used in this exemplary aspect of the invention include:

“Filter for low complexity: ON

Word Size: 3

Matrix: Blosum62

Gap Costs: Existence: 11

Extension: 1”

Other default settings can be: filter for low complexity OFF, word size of 3 for protein, BLOSUM62 matrix, gap existence penalty of −11 and a gap extension penalty of −1. An exemplary NCBI BLAST 2.2.2 program setting has the “−W” option default to 0. This means that, if not set, the word size defaults to 3 for proteins and 11 for nucleotides.

Computer Systems and Computer Program Products

To determine and identify sequence identities, structural homologies, motifs and the like in silico, the sequence of the invention can be stored, recorded, and manipulated on any medium which can be read and accessed by a computer. Accordingly, the invention provides computers, computer systems, computer readable mediums, computer programs products and the like recorded or stored thereon the nucleic acid and polypeptide sequences of the invention. As used herein, the words “recorded” and “stored” refer to a process for storing information on a computer medium. A skilled artisan can readily adopt any known methods for recording information on a computer readable medium to generate manufactures comprising one or more of the nucleic acid and/or polypeptide sequences of the invention.

Another aspect of the invention is a computer readable medium having recorded thereon at least one nucleic acid and/or polypeptide sequence of the invention. Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of other media known to those skilled in the art.

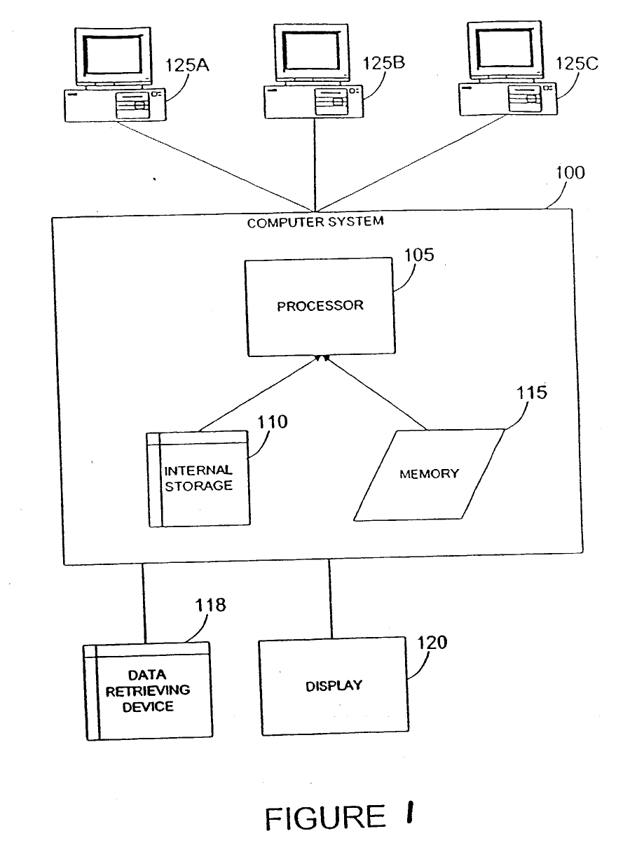

Aspects of the invention include systems (e.g., internet based systems), particularly computer systems, which store and manipulate the sequences and sequence information described herein. One example of a computer system 100 is illustrated in block diagram form in FIG. 1. As used herein, “a computer system” refers to the hardware components, software components, and data storage components used to analyze a nucleotide or polypeptide sequence of the invention. The computer system 100 can include a processor for processing, accessing and manipulating the sequence data. The processor 105 can be any well-known type of central processing unit, such as, for example, the Pentium III from Intel Corporation, or similar processor from Sun, Motorola, Compaq, AMD or International Business Machines. The computer system 100 is a general purpose system that comprises the processor 105 and one or more internal data storage components 110 for storing data, and one or more data retrieving devices for retrieving the data stored on the data storage components. A skilled artisan can readily appreciate that any one of the currently available computer systems are suitable.

In one aspect, the computer system 100 includes a processor 105 connected to a bus which is connected to a main memory 115 (preferably implemented as RAM) and one or more internal data storage devices 110, such as a hard drive and/or other computer readable media having data recorded thereon. The computer system 100 can further include one or more data retrieving device 118 for reading the data stored on the internal data storage devices 110. The data retrieving device 118 may represent, for example, a floppy disk drive, a compact disk drive, a magnetic tape drive, or a modem capable of connection to a remote data storage system (e.g., via the internet) etc. In some embodiments, the internal data storage device 110 is a removable computer readable medium such as a floppy disk, a compact disk, a magnetic tape, etc. containing control logic and/or data recorded thereon. The computer system 100 may advantageously include or be programmed by appropriate software for reading the control logic and/or the data from the data storage component once inserted in the data retrieving device. The computer system 100 includes a display 120 which is used to display output to a computer user. It should also be noted that the computer system 100 can be linked to other computer systems 125a-c in a network or wide area network to provide centralized access to the computer system 100. Software for accessing and processing the nucleotide or amino acid sequences of the invention can reside in main memory 115 during execution. In some aspects, the computer system 100 may further comprise a sequence comparison algorithm for comparing a nucleic acid sequence of the invention. The algorithm and sequence(s) can be stored on a computer readable medium. A “sequence comparison algorithm” refers to one or more programs which are implemented (locally or remotely) on the computer system 100 to compare a nucleotide sequence with other nucleotide sequences and/or compounds stored within a data storage means. For example, the sequence comparison algorithm may compare the nucleotide sequences of the invention stored on a computer readable medium to reference sequences stored on a computer readable medium to identify homologies or structural motifs.

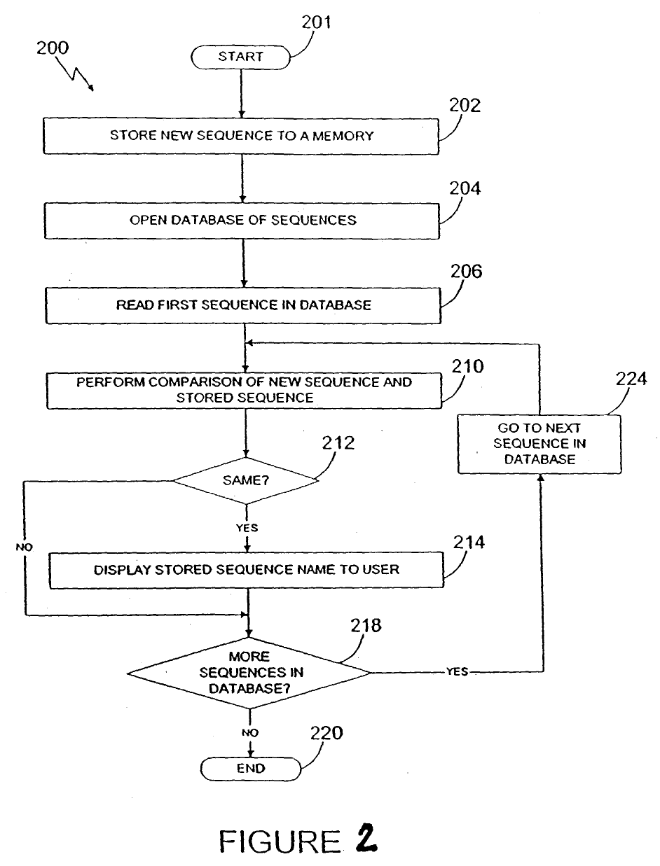

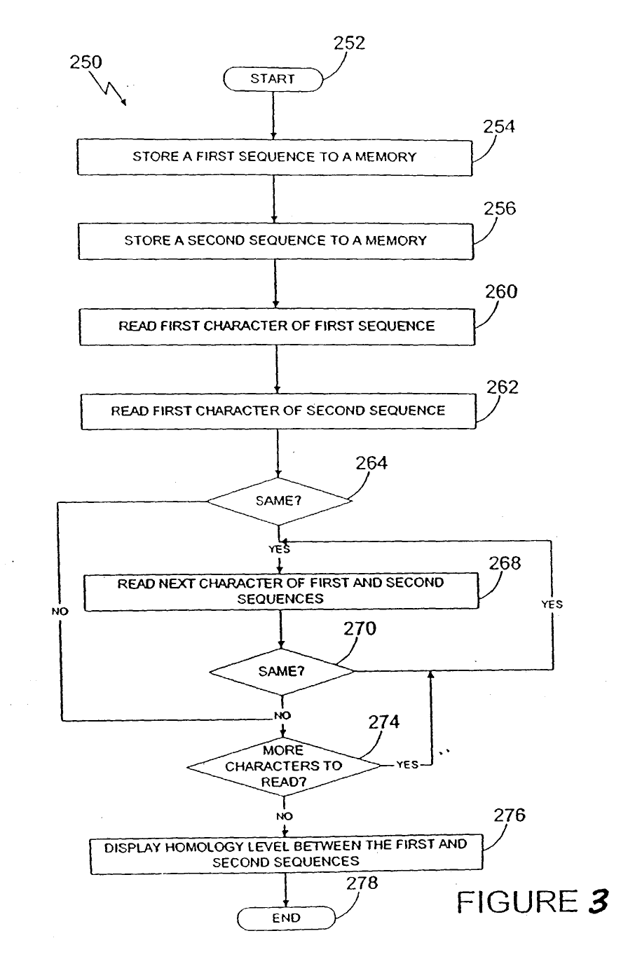

The parameters used with the above algorithms may be adapted depending on the sequence length and degree of homology studied. In some aspects, the parameters may be the default parameters used by the algorithms in the absence of instructions from the user. FIG. 2 is a flow diagram illustrating one aspect of a process 200 for comparing a new nucleotide or protein sequence with a database of sequences in order to determine the homology levels between the new sequence and the sequences in the database. The database of sequences can be a private database stored within the computer system 100, or a public database such as GENBANK that is available through the Internet. The process 200 begins at a start state 201 and then moves to a state 202 wherein the new sequence to be compared is stored to a memory in a computer system 100. As discussed above, the memory could be any type of memory, including RAM or an internal storage device. The process 200 then moves to a state 204 wherein a database of sequences is opened for analysis and comparison. The process 200 then moves to a state 206 wherein the first sequence stored in the database is read into a memory on the computer. A comparison is then performed at a state 210 to determine if the first sequence is the same as the second sequence. It is important to note that this step is not limited to performing an exact comparison between the new sequence and the first sequence in the database. Well-known methods are known to those of skill in the art for comparing two nucleotide or protein sequences, even if they are not identical. For example, gaps can be introduced into one sequence in order to raise the homology level between the two tested sequences. The parameters that control whether gaps or other features are introduced into a sequence during comparison are normally entered by the user of the computer system. Once a comparison of the two sequences has been performed at the state 210, a determination is made at a decision state 210 whether the two sequences are the same. Of course, the term “same” is not limited to sequences that are absolutely identical. Sequences that are within the homology parameters entered by the user will be marked as “same” in the process 200. If a determination is made that the two sequences are the same, the process 200 moves to a state 214 wherein the name of the sequence from the database is displayed to the user. This state notifies the user that the sequence with the displayed name fulfills the homology constraints that were entered. Once the name of the stored sequence is displayed to the user, the process 200 moves to a decision state 218 wherein a determination is made whether more sequences exist in the database. If no more sequences exist in the database, then the process 200 terminates at an end state 220. However, if more sequences do exist in the database, then the process 200 moves to a state 224 wherein a pointer is moved to the next sequence in the database so that it can be compared to the new sequence. In this manner, the new sequence is aligned and compared with every sequence in the database. It should be noted that if a determination had been made at the decision state 212 that the sequences were not homologous, then the process 200 would move immediately to the decision state 218 in order to determine if any other sequences were available in the database for comparison. Accordingly, one aspect of the invention is a computer system comprising a processor, a data storage device having stored thereon a nucleic acid sequence of the invention and a sequence comparer for conducting the comparison. The sequence comparer may indicate a homology level between the sequences compared or identify structural motifs, or it may identify structural motifs in sequences which are compared to these nucleic acid codes and polypeptide codes. FIG. 3 is a flow diagram illustrating one embodiment of a process 250 in a computer for determining whether two sequences are homologous. The process 250 begins at a start state 252 and then moves to a state 254 wherein a first sequence to be compared is stored to a memory. The second sequence to be compared is then stored to a memory at a state 256. The process 250 then moves to a state 260 wherein the first character in the first sequence is read and then to a state 262 wherein the first character of the second sequence is read. It should be understood that if the sequence is a nucleotide sequence, then the character would normally be either A, T, C, G or U. If the sequence is a protein sequence, then it can be a single letter amino acid code so that the first and sequence sequences can be easily compared. A determination is then made at a decision state 264 whether the two characters are the same. If they are the same, then the process 250 moves to a state 268 wherein the next characters in the first and second sequences are read. A determination is then made whether the next characters are the same. If they are, then the process 250 continues this loop until two characters are not the same. If a determination is made that the next two characters are not the same, the process 250 moves to a decision state 274 to determine whether there are any more characters either sequence to read. If there are not any more characters to read, then the process 250 moves to a state 276 wherein the level of homology between the first and second sequences is displayed to the user. The level of homology is determined by calculating the proportion of characters between the sequences that were the same out of the total number of sequences in the first sequence. Thus, if every character in a first 100 nucleotide sequence aligned with an every character in a second sequence, the homology level would be 100%.

Alternatively, the computer program can compare a reference sequence to a sequence of the invention to determine whether the sequences differ at one or more positions. The program can record the length and identity of inserted, deleted or substituted nucleotides or amino acid residues with respect to the sequence of either the reference or the invention. The computer program may be a program which determines whether a reference sequence contains a single nucleotide polymorphism (SNP) with respect to a sequence of the invention, or, whether a sequence of the invention comprises a SNP of a known sequence. Thus, in some aspects, the computer program is a program which identifies SNPs. The method may be implemented by the computer systems described above and the method illustrated in FIG. 3. The method can be performed by reading a sequence of the invention and the reference sequences through the use of the computer program and identifying differences with the computer program.

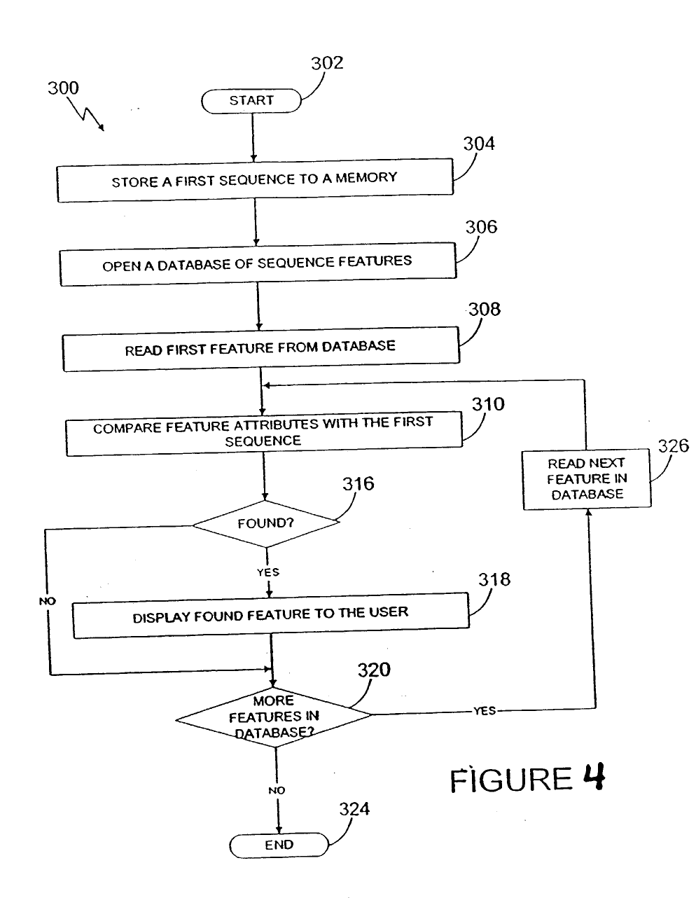

In other aspects the computer based system comprises an identifier for identifying features within a nucleic acid or polypeptide of the invention. An “identifier” refers to one or more programs which identifies certain features within a nucleic acid sequence. For example, an identifier may comprise a program which identifies an open reading frame (ORF) in a nucleic acid sequence. FIG. 4 is a flow diagram illustrating one aspect of an identifier process 300 for detecting the presence of a feature in a sequence. The process 300 begins at a start state 302 and then moves to a state 304 wherein a first sequence that is to be checked for features is stored to a memory 115 in the computer system 100. The process 300 then moves to a state 306 wherein a database of sequence features is opened. Such a database would include a list of each feature's attributes along with the name of the feature. For example, a feature name could be “Initiation Codon” and the attribute would be “ATG”. Another example would be the feature name “TAATAA Box” and the feature attribute would be “TAATAA”. An example of such a database is produced by the University of Wisconsin Genetics Computer Group. Alternatively, the features may be structural polypeptide motifs such as alpha helices, beta sheets, or functional polypeptide motifs such as enzymatic active sites, helix-turn-helix motifs or other motifs known to those skilled in the art. Once the database of features is opened at the state 306, the process 300 moves to a state 308 wherein the first feature is read from the database. A comparison of the attribute of the first feature with the first sequence is then made at a state 310. A determination is then made at a decision state 316 whether the attribute of the feature was found in the first sequence. If the attribute was found, then the process 300 moves to a state 318 wherein the name of the found feature is displayed to the user. The process 300 then moves to a decision state 320 wherein a determination is made whether move features exist in the database. If no more features do exist, then the process 300 terminates at an end state 324. However, if more features do exist in the database, then the process 300 reads the next sequence feature at a state 326 and loops back to the state 310 wherein the attribute of the next feature is compared against the first sequence. If the feature attribute is not found in the first sequence at the decision state 316, the process 300 moves directly to the decision state 320 in order to determine if any more features exist in the database. Thus, in one aspect, the invention provides a computer program that identifies open reading frames (ORFs).

A polypeptide or nucleic acid sequence of the invention can be stored and manipulated in a variety of data processor programs in a variety of formats. For example, a sequence can be stored as text in a word processing file, such as MicrosoftWORD or WORDPERFECT or as an ASCII file in a variety of database programs familiar to those of skill in the art, such as DB2, SYBASE, or ORACLE. In addition, many computer programs and databases may be used as sequence comparison algorithms, identifiers, or sources of reference nucleotide sequences or polypeptide sequences to be compared to a nucleic acid sequence of the invention. The programs and databases used to practice the invention include, but are not limited to: MacPattern (EMBL), DiscoveryBase (Molecular Applications Group), GeneMine (Molecular Applications Group), Look (Molecular Applications Group), MacLook (Molecular Applications Group), BLAST and BLAST2 (NCBI), BLASTN and BLASTX (Altschul et al, J. Mol. Biol. 215: 403, 1990), FASTA (Pearson and Lipman, Proc. Natl. Acad. Sci. USA, 85: 2444, 1988), FASTDB (Brutlag et al. Comp. App. Biosci. 6:237-245, 1990), Catalyst (Molecular Simulations Inc.), Catalyst/SHAPE (Molecular Simulations Inc.), Cerius2.DBAccess (Molecular Simulations Inc.), HypoGen (Molecular Simulations Inc.), Insight II, (Molecular Simulations Inc.), Discover (Molecular Simulations Inc.), CHARMm (Molecular Simulations Inc.), Felix (Molecular Simulations Inc.), DelPhi, (Molecular Simulations Inc.), QuanteMM, (Molecular Simulations Inc.), Homology (Molecular Simulations Inc.), Modeler (Molecular Simulations Inc.), ISIS (Molecular Simulations Inc.), Quanta/Protein Design (Molecular Simulations Inc.), WebLab (Molecular Simulations Inc.), WebLab Diversity Explorer (Molecular Simulations Inc.), Gene Explorer (Molecular Simulations Inc.), SeqFold (Molecular Simulations Inc.), the MDL Available Chemicals Directory database, the MDL Drug Data Report data base, the Comprehensive Medicinal Chemistry database, Derwent's World Drug Index database, the BioByteMasterFile database, the Genbank database, and the Genseqn database. Many other programs and data bases would be apparent to one of skill in the art given the present disclosure.

Motifs which may be detected using the above programs include sequences encoding leucine zippers, helix-turn-helix motifs, glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites.

Hybridization of Nucleic Acids

The invention provides isolated or recombinant nucleic acids that hybridize under stringent conditions to an exemplary sequence of the invention, or a nucleic acid that encodes a polypeptide of the invention. The stringent conditions can be highly stringent conditions, medium stringent conditions, low stringent conditions, including the high and reduced stringency conditions described herein. In one aspect, it is the stringency of the wash conditions that set forth the conditions which determine whether a nucleic acid is within the scope of the invention, as discussed below.

In alternative embodiments, nucleic acids of the invention as defined by their ability to hybridize under stringent conditions can be between about five residues and the full length of nucleic acid of the invention; e.g., they can be at least 5, 10, 15, 20, 25, 30, 35, 40, 50, 55, 60, 65, 70, 75, 80, 90, 100, 150, 200, 250, 300, 350, 400, 450, 500, 550, 600, 650, 700, 750, 800, 850, 900, 950, 1000, or more, residues in length. Nucleic acids shorter than full length are also included. These nucleic acids can be useful as, e.g., hybridization probes, labeling probes, PCR oligonucleotide probes, iRNA, antisense or sequences encoding antibody binding peptides (epitopes), motifs, active sites and the like.

In one aspect, nucleic acids of the invention are defined by their ability to hybridize under high stringency comprises conditions of about 50% formamide at about 37° C. to 42° C. In one aspect, nucleic acids of the invention are defined by their ability to hybridize under reduced stringency comprising conditions in about 35% to 25% formamide at about 30° C. to 35° C.

Alternatively, nucleic acids of the invention are defined by their ability to hybridize under high stringency comprising conditions at 42° C. in 50% formamide, 5×SSPE, 0.3% SDS, and a repetitive sequence blocking nucleic acid, such as cot-1 or salmon sperm DNA (e.g., 200 n/ml sheared and denatured salmon sperm DNA). In one aspect, nucleic acids of the invention are defined by their ability to hybridize under reduced stringency conditions comprising 35% formamide at a reduced temperature of 35° C.

Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide.

The temperature range corresponding to a particular level of stringency can be further narrowed by calculating the purine to pyrimidine ratio of the nucleic acid of interest and adjusting the temperature accordingly. Nucleic acids of the invention are also defined by their ability to hybridize under high, medium, and low stringency conditions as set forth in Ausubel and Sambrook. Variations on the above ranges and conditions are well known in the art. Hybridization conditions are discussed further, below.

The above procedure may be modified to identify nucleic acids having decreasing levels of homology to the probe sequence. For example, to obtain nucleic acids of decreasing homology to the detectable probe, less stringent conditions may be used. For example, the hybridization temperature may be decreased in increments of 5° C. from 68° C. to 42° C. in a hybridization buffer having a Na⁺ concentration of approximately 1M. Following hybridization, the filter may be washed with 2×SSC, 0.5% SDS at the temperature of hybridization. These conditions are considered to be “moderate” conditions above 50° C. and “low” conditions below 50° C. A specific example of “moderate” hybridization conditions is when the above hybridization is conducted at 55° C. A specific example of “low stringency” hybridization conditions is when the above hybridization is conducted at 45° C.

Alternatively, the hybridization may be carried out in buffers, such as 6×SSC, containing formamide at a temperature of 42° C. In this case, the concentration of formamide in the hybridization buffer may be reduced in 5% increments from 50% to 0% to identify clones having decreasing levels of homology to the probe. Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide.

However, the selection of a hybridization format is not critical—it is the stringency of the wash conditions that set forth the conditions which determine whether a nucleic acid is within the scope of the invention. Wash conditions used to identify nucleic acids within the scope of the invention include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. See Sambrook, Tijssen and Ausubel for a description of SSC buffer and equivalent conditions.

These methods may be used to isolate nucleic acids of the invention.

Oligonucleotides Probes and Methods for Using Them

The invention also provides nucleic acid probes that can be used, e.g., for identifying nucleic acids encoding a polypeptide with an amidase activity or fragments thereof or for identifying amidase genes. In one aspect, the probe comprises at least 10 consecutive bases of a nucleic acid of the invention. Alternatively, a probe of the invention can be at least about 5, 6, 7, 8, 9, 10, 15, 20, 25, 30, 35, 40, 45, 50, 60, 70, 80, 90, 100, 110, 120, 130, 150 or about 10 to 50, about 20 to 60 about 30 to 70, consecutive bases of a sequence as set forth in a nucleic acid of the invention. The probes identify a nucleic acid by binding and/or hybridization. The probes can be used in arrays of the invention, see discussion below, including, e.g., capillary arrays. The probes of the invention can also be used to isolate other nucleic acids or polypeptides.

The probes of the invention can be used to determine whether a biological sample, such as a soil sample, contains an organism having a nucleic acid sequence of the invention or an organism from which the nucleic acid was obtained. In such procedures, a biological sample potentially harboring the organism from which the nucleic acid was isolated is obtained and nucleic acids are obtained from the sample. The nucleic acids are contacted with the probe under conditions which permit the probe to specifically hybridize to any complementary sequences present in the sample. Where necessary, conditions which permit the probe to specifically hybridize to complementary sequences may be determined by placing the probe in contact with complementary sequences from samples known to contain the complementary sequence, as well as control sequences which do not contain the complementary sequence. Hybridization conditions, such as the salt concentration of the hybridization buffer, the formamide concentration of the hybridization buffer, or the hybridization temperature, may be varied to identify conditions which allow the probe to hybridize specifically to complementary nucleic acids (see discussion on specific hybridization conditions).

If the sample contains the organism from which the nucleic acid was isolated, specific hybridization of the probe is then detected. Hybridization may be detected by labeling the probe with a detectable agent such as a radioactive isotope, a fluorescent dye or an enzyme capable of catalyzing the formation of a detectable product. Many methods for using the labeled probes to detect the presence of complementary nucleic acids in a sample are familiar to those skilled in the art. These include Southern Blots, Northern Blots, colony hybridization procedures, and dot blots. Protocols for each of these procedures are provided in Ausubel and Sambrook.

Alternatively, more than one probe (at least one of which is capable of specifically hybridizing to any complementary sequences which are present in the nucleic acid sample), may be used in an amplification reaction to determine whether the sample contains an organism containing a nucleic acid sequence of the invention (e.g., an organism from which the nucleic acid was isolated). In one aspect, the probes comprise oligonucleotides. In one aspect, the amplification reaction may comprise a PCR reaction. PCR protocols are described in Ausubel and Sambrook (see discussion on amplification reactions). In such procedures, the nucleic acids in the sample are contacted with the probes, the amplification reaction is performed, and any resulting amplification product is detected. The amplification product may be detected by performing gel electrophoresis on the reaction products and staining the gel with an intercalator such as ethidium bromide. Alternatively, one or more of the probes may be labeled with a radioactive isotope and the presence of a radioactive amplification product may be detected by autoradiography after gel electrophoresis.

Probes derived from sequences near the 3′ or 5′ ends of a nucleic acid sequence of the invention can also be used in chromosome walking procedures to identify clones containing additional, e.g., genomic sequences. Such methods allow the isolation of genes which encode additional proteins of interest from the host organism.

In one aspect, nucleic acid sequences of the invention are used as probes to identify and isolate related nucleic acids. In some aspects, the so-identified related nucleic acids may be cDNAs or genomic DNAs from organisms other than the one from which the nucleic acid of the invention was first isolated. In such procedures, a nucleic acid sample is contacted with the probe under conditions which permit the probe to specifically hybridize to related sequences. Hybridization of the probe to nucleic acids from the related organism is then detected using any of the methods described above.

In nucleic acid hybridization reactions, the conditions used to achieve a particular level of stringency can vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter. Hybridization can be carried out under conditions of low stringency, moderate stringency or high stringency. As an example of nucleic acid hybridization, a polymer membrane containing immobilized denatured nucleic acids is first prehybridized for 30 minutes at 45° C. in a solution consisting of 0.9 M NaCl, 50 mM NaH₂PO4, pH 7.0, 5.0 mM Na₂EDTA, 0.5% SDS, 10× Denhardt's, and 0.5 mg/ml polyriboadenylic acid. Approximately 2×10⁷ cpm (specific activity 4-9×10⁸ cpm/ug) of ³²P end-labeled oligonucleotide probe can then added to the solution. After 12-16 hours of incubation, the membrane is washed for 30 minutes at room temperature (RT) in 1× SET (150 mM NaCl, 20 mM Tris hydrochloride, pH 7.8, 1 mM Na₂EDTA) containing 0.5% SDS, followed by a 30 minute wash in fresh 1× SET at Tm-10° C. for the oligonucleotide probe. The membrane is then exposed to auto-radiographic film for detection of hybridization signals.

By varying the stringency of the hybridization conditions used to identify nucleic acids, such as cDNAs or genomic DNAs, which hybridize to the detectable probe, nucleic acids having different levels of homology to the probe can be identified and isolated. Stringency may be varied by conducting the hybridization at varying temperatures below the melting temperatures of the probes. The melting temperature, Tm, is the temperature (under defined ionic strength and pH) at which 50% of the target sequence hybridizes to a perfectly complementary probe. Very stringent conditions are selected to be equal to or about 5° C. lower than the Tm for a particular probe. The melting temperature of the probe may be calculated using the following exemplary formulas. For probes between 14 and 70 nucleotides in length the melting temperature (Tm) is calculated using the formula: Tm=81.5+16.6(log [Na+])+0.41(fraction G+C)−(600/N) where N is the length of the probe. If the hybridization is carried out in a solution containing formamide, the melting temperature may be calculated using the equation: Tm=81.5+16.6(log [Na+])+0.41(fraction G+C)−(0.63% formamide)−(600/N) where N is the length of the probe. Prehybridization may be carried out in 6×SSC, 5× Denhardt's reagent, 0.5% SDS, 100 μg denatured fragmented salmon sperm DNA or 6×SSC, 5× Denhardt's reagent, 0.5% SDS, 100 μg denatured fragmented salmon sperm DNA, 50% formamide. Formulas for SSC and Denhardt's and other solutions are listed, e.g., in Sambrook.

Hybridization is conducted by adding the detectable probe to the prehybridization solutions listed above. Where the probe comprises double stranded DNA, it is denatured before addition to the hybridization solution. The filter is contacted with the hybridization solution for a sufficient period of time to allow the probe to hybridize to cDNAs or genomic DNAs containing sequences complementary thereto or homologous thereto. For probes over 200 nucleotides in length, the hybridization may be carried out at 15-25° C. below the Tm. For shorter probes, such as oligonucleotide probes, the hybridization may be conducted at 5-10° C. below the Tm. In one aspect, hybridizations in 6×SSC are conducted at approximately 68° C. In one aspect, hybridizations in 50% formamide containing solutions are conducted at approximately 42° C. All of the foregoing hybridizations would be considered to be under conditions of high stringency.

Following hybridization, the filter is washed to remove any non-specifically bound detectable probe. The stringency used to wash the filters can also be varied depending on the nature of the nucleic acids being hybridized, the length of the nucleic acids being hybridized, the degree of complementarity, the nucleotide sequence composition (e.g., GC v. AT content), and the nucleic acid type (e.g., RNA v. DNA). Examples of progressively higher stringency condition washes are as follows: 2×SSC, 0.1% SDS at room temperature for 15 minutes (low stringency); 0.1×SSC, 0.5% SDS at room temperature for 30 minutes to 1 hour (moderate stringency); 0.1×SSC, 0.5% SDS for 15 to 30 minutes at between the hybridization temperature and 68° C. (high stringency); and 0.1 SM NaCl for 15 minutes at 72° C. (very high stringency). A final low stringency wash can be conducted in 0.1×SSC at room temperature. The examples above are merely illustrative of one set of conditions that can be used to wash filters. One of skill in the art would know that there are numerous recipes for different stringency washes.

Nucleic acids which have hybridized to the probe can be identified by autoradiography or other conventional techniques. The above procedure may be modified to identify nucleic acids having decreasing levels of homology to the probe sequence. For example, to obtain nucleic acids of decreasing homology to the detectable probe, less stringent conditions may be used. For example, the hybridization temperature may be decreased in increments of 5° C. from 68° C. to 42° C. in a hybridization buffer having a Na+ concentration of approximately 1M. Following hybridization, the filter may be washed with 2×SSC, 0.5% SDS at the temperature of hybridization. These conditions are considered to be “moderate” conditions above 50° C. and “low” conditions below 50° C. An example of “moderate” hybridization conditions is when the above hybridization is conducted at 55° C. An example of “low stringency” hybridization conditions is when the above hybridization is conducted at 45° C.

Alternatively, the hybridization may be carried out in buffers, such as 6×SSC, containing formamide at a temperature of 42° C. In this case, the concentration of formamide in the hybridization buffer may be reduced in 5% increments from 50% to 0% to identify clones having decreasing levels of homology to the probe. Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide.

These probes and methods of the invention can be used to isolate nucleic acids having a sequence with at least about 99%, 98%, 97%, at least 95%, at least 90%, at least 85%, at least 80%, at least 75%, at least 70%, at least 65%, at least 60%, at least 55%, or at least 50% homology to a nucleic acid sequence of the invention comprising at least about 10, 15, 20, 25, 30, 35, 40, 50, 75, 100, 150, 200, 250, 300, 350, 400, 500, 550, 600, 650, 700, 750, 800, 850, 900, 950, 1000, or more consecutive bases thereof, and the sequences complementary thereto. Homology may be measured using an alignment algorithm, as discussed herein. For example, the homologous polynucleotides may have a coding sequence which is a naturally occurring allelic variant of one of the coding sequences described herein. Such allelic variants may have a substitution, deletion or addition of one or more nucleotides when compared to a nucleic acid of the invention.

Additionally, the probes and methods of the invention can be used to isolate nucleic acids which encode polypeptides having at least about 99%, at least 95%, at least 90%, at least 85%, at least 80%, at least 75%, at least 70%, at least 65%, at least 60%, at least 55%, or at least 50% sequence identity (homology) to a polypeptide of the invention comprising at least 5, 10, 15, 20, 25, 30, 35, 40, 50, 75, 100, or 150 consecutive amino acids, as determined using a sequence alignment algorithm (e.g., such as the FASTA version 3.0t78 algorithm with the default parameters, or a BLAST 2.2.2 program with exemplary settings as set forth herein).

Inhibiting Expression of Amidases of the Invention

The invention provides nucleic acids complementary to (e.g., antisense sequences to) the nucleic acid sequences of the invention. Antisense sequences are capable of inhibiting the transport, splicing or transcription of amidase-encoding genes. The inhibition can be effected through the targeting of genomic DNA or messenger RNA. The transcription or function of targeted nucleic acid can be inhibited, for example, by hybridization and/or cleavage. One particularly useful set of inhibitors provided by the present invention includes oligonucleotides which are able to either bind amidase gene or message, in either case preventing or inhibiting the production or function of amidase. The association can be through sequence specific hybridization. Another useful class of inhibitors includes oligonucleotides which cause inactivation or cleavage of amidase message. The oligonucleotide can have enzyme activity which causes such cleavage, such as ribozymes. The oligonucleotide can be chemically modified or conjugated to an enzyme or composition capable of cleaving the complementary nucleic acid. A pool of many different such oligonucleotides can be screened for those with the desired activity.

Antisense Oligonucleotides

The invention provides antisense oligonucleotides capable of binding amidase message which can inhibit proteolytic activity by targeting mRNA. Strategies for designing antisense oligonucleotides are well described in the scientific and patent literature, and the skilled artisan can design such amidase oligonucleotides using the novel reagents of the invention. For example, gene walking/RNA mapping protocols to screen for effective antisense oligonucleotides are well known in the art, see, e.g., Ho (2000) Methods Enzymol. 314:168-183, describing an RNA mapping assay, which is based on standard molecular techniques to provide an easy and reliable method for potent antisense sequence selection. See also Smith (2000) Eur. J. Pharm. Sci. 11:191-198.

Naturally occurring nucleic acids are used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as described in WO 97/03211; WO 96/39154; Mata (1997) Toxicol Appl Pharmacol 144:189-197; Antisense Therapeutics, ed. Agrawal (Humana Press, Totowa, N.J., 1996). Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense amidase sequences of the invention (see, e.g., Gold (1995) J. of Biol. Chem. 270:13581-13584).

Inhibitory Ribozymes

The invention provides ribozymes capable of binding amidase message. These ribozymes can inhibit amidase activity by, e.g., targeting mRNA. Strategies for designing ribozymes and selecting the amidase-specific antisense sequence for targeting are well described in the scientific and patent literature, and the skilled artisan can design such ribozymes using the novel reagents of the invention. Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it can be released from that RNA to bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The ribozyme of the invention, e.g., an enzymatic ribozyme RNA molecule, can be formed in a hammerhead motif, a hairpin motif, as a hepatitis delta virus motif, a group I intron motif and/or an RNaseP-like RNA in association with an RNA guide sequence. Examples of hammerhead motifs are described by, e.g., Rossi (1992) Aids Research and Human Retroviruses 8:183; hairpin motifs by Hampel (1989) Biochemistry 28:4929, and Hampel (1990) Nuc. Acids Res. 18:299; the hepatitis delta virus motif by Perrotta (1992) Biochemistry 31:16; the RNaseP motif by Guerrier-Takada (1983) Cell 35:849; and the group I intron by Cech U.S. Pat. No. 4,987,071. The recitation of these specific motifs is not intended to be limiting. Those skilled in the art will recognize that a ribozyme of the invention, e.g., an enzymatic RNA molecule of this invention, can have a specific substrate binding site complementary to one or more of the target gene RNA regions. A ribozyme of the invention can have a nucleotide sequence within or surrounding that substrate binding site which imparts an RNA cleaving activity to the molecule.

Modification of Nucleic Acids

The invention provides methods of generating variants of the nucleic acids of the invention, e.g., those encoding an amidase of the invention or an antibody of the invention. These methods can be repeated or used in various combinations to generate amidases having an altered or different activity or an altered or different stability from that of an amidase encoded by the template nucleic acid. These methods also can be repeated or used in various combinations, e.g., to generate variations in gene/ message expression, message translation or message stability. In another aspect, the genetic composition of a cell is altered by, e.g., modification of a homologous gene ex vivo, followed by its reinsertion into the cell.

A nucleic acid of the invention can be altered by any means. For example, random or stochastic methods, or, non-stochastic, or “directed evolution,” methods, see, e.g., U.S. Pat. No. 6,361,974. Methods for random mutation of genes are well known in the art, see, e.g., U.S. Pat. No. 5,830,696. For example, mutagens can be used to randomly mutate a gene. Mutagens include, e.g., ultraviolet light or gamma irradiation, or a chemical mutagen, e.g., mitomycin, nitrous acid, photoactivated psoralens, alone or in combination, to induce DNA breaks amenable to repair by recombination. Other chemical mutagens include, for example, sodium bisulfite, nitrous acid, hydroxylamine, hydrazine or formic acid. Other mutagens are analogues of nucleotide precursors, e.g., nitrosoguanidine, 5-bromouracil, 2-aminopurine, or acridine. These agents can be added to a PCR reaction in place of the nucleotide precursor thereby mutating the sequence. Intercalating agents such as proflavine, acriflavine, quinacrine and the like can also be used.

Any technique in molecular biology can be used, e.g., random PCR mutagenesis, see, e.g., Rice (1992) Proc. Natl. Acad. Sci. USA 89:5467-5471; or, combinatorial multiple cassette mutagenesis, see, e.g., Crameri (1995) Biotechniques 18:194-196. Alternatively, nucleic acids, e.g., genes, can be reassembled after random, or “stochastic,” fragmentation, see, e.g., U.S. Pat. Nos. 6,291,242; 6,287,862; 6,287,861; 5,955,358; 5,830,721; 5,824,514; 5,811,238; 5,605,793. In alternative aspects, modifications, additions or deletions are introduced by error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturated mutagenesis (GSSM), synthetic ligation reassembly (SLR), recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, and/or a combination of these and other methods.

The following publications describe a variety of recursive recombination procedures and/or methods which can be incorporated into the methods of the invention: Stemmer (1999) “Molecular breeding of viruses for targeting and other clinical properties” Tumor Targeting 4:1-4; Ness (1999) Nature Biotechnology 17:893-896; Chang (1999) “Evolution of a cytokine using DNA family shuffling” Nature Biotechnology 17:793-797; Minshull (1999) “Protein evolution by molecular breeding” Current Opinion in Chemical Biology 3:284-290; Christians (1999) “Directed evolution of thymidine kinase for AZT phosphorylation using DNA family shuffling” Nature Biotechnology 17:259-264; Crameri (1998) “DNA shuffling of a family of genes from diverse species accelerates directed evolution” Nature 391:288-291; Crameri (1997) “Molecular evolution of an arsenate detoxification pathway by DNA shuffling,” Nature Biotechnology 15:436-438; Zhang (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp.447-457; Crameri and Stemmer (1995) “Combinatorial multiple cassette mutagenesis creates all the permutations of mutant and wildtype cassettes” BioTechniques 18:194-195; Stemmer et al. (1995) “Single-step assembly of a gene and entire plasmid form large numbers of oligodeoxyribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) Oligonucleotide-directed mutagenesis: a simple method using two oligonucleotide primers and a single-stranded DNA template“Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional protocols that can be used to practice the invention include point mismatch repair (Kramer (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh (1986) “Use of oligonucleotides to generate large deletions” Nucl. Acids Res. 14: 5115), restriction-selection and restriction-selection and restriction-purification (Wells et al. (1986) “Importance of hydrogen-bond formation in stabilizing the transition state of subtilisin” Phil. Trans. R. Soc. Lond. A 317:415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundstrom et al. (1985) “Oligonucleotide-directed mutagenesis by microscale ‘shot-gun’ gene synthesis” Nucl. Acids Res. 13: 3305-3316), double-strand break repair (Mandecki (1986); Arnold (1993) “Protein engineering for unusual environments” Current Opinion in Biotechnology 4:450-455. “Oligonucleotide-directed double-strand break repair in plasmids of Escherichia coli: a method for site-specific mutagenesis” Proc. Natl. Acad. Sci. USA, 83:7177-7181). Additional details on many of the above methods can be found in Methods in Enzymology Volume 154, which also describes useful controls for trouble-shooting problems with various mutagenesis methods.

Protocols that can be used to practice the invention are described, e.g., in U.S. Pat. Nos. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination.”

Protocols that can be used to practice the invention (providing details regarding various diversity generating methods) are described, e.g., in U.S. patent application Ser. No. 09/407,800, “SHUFFLING OF CODON ALTERED GENES” by Patten et al. filed Sep. 28, 1999; “EVOLUTION OF WHOLE CELLS AND ORGANISMS BY RECURSIVE SEQUENCE RECOMBINATION” by del Cardayre et al., U.S. Pat. No. 6,379,964; “OLIGONUCLEOTIDE MEDIATED NUCLEIC ACID RECOMBINATION” by Crameri et al., U.S. Pat. Nos. 6,319,714; 6,368,861; 6,376,246; 6,423,542; 6,426,224 and PCT/US00/01203; “USE OF CODON-VARIED OLIGONUCLEOTIDE SYNTHESIS FOR SYNTHETIC SHUFFLING” by Welch et al., U.S. Pat. No. 6,436,675; “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) and, e.g. “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579); “METHODS OF POPULATING DATA STRUCTURES FOR USE IN EVOLUTIONARY SIMULATIONS” by Selifonov and Stemmer, filed Jan. 18, 2000 (PCT/US00/01138); and “SINGLE-STRANDED NUCLEIC ACID TEMPLATE-MEDIATED RECOMBINATION AND NUCLEIC ACID FRAGMENT ISOLATION” by Affholter, filed Sep. 6, 2000 (U.S. Ser. No. 09/656,549); and U.S. Pat. Nos. 6,177,263; 6,153,410.

Non-stochastic, or “directed evolution,” methods include, e.g., saturation mutagenesis (GSSM), synthetic ligation reassembly (SLR), or a combination thereof are used to modify the nucleic acids of the invention to generate amidases with new or altered properties (e.g., activity under highly acidic or alkaline conditions, high temperatures, and the like). Polypeptides encoded by the modified nucleic acids can be screened for an activity before testing for proteolytic or other activity. Any testing modality or protocol can be used, e.g., using a capillary array platform. See, e.g., U.S. Pat. Nos. 6,361,974; 6,280,926; 5,939,250.

Saturation Mutagenesis, or, GSSM

In one aspect, codon primers containing a degenerate N,N,G/T sequence are used to introduce point mutations into a polynucleotide, e.g., an amidase or an antibody of the invention, so as to generate a set of progeny polypeptides in which a full range of single amino acid substitutions is represented at each amino acid position, e.g., an amino acid residue in an enzyme active site or ligand binding site targeted to be modified. These oligonucleotides can comprise a contiguous first homologous sequence, a degenerate N,N,G/T sequence, and, optionally, a second homologous sequence. The downstream progeny translational products from the use of such oligonucleotides include all possible amino acid changes at each amino acid site along the polypeptide, because the degeneracy of the N,N,G/T sequence includes codons for all 20 amino acids. In one aspect, one such degenerate oligonucleotide (comprised of, e.g., one degenerate N,N,G/T cassette) is used for subjecting each original codon in a parental polynucleotide template to a full range of codon substitutions. In another aspect, at least two degenerate cassettes are used—either in the same oligonucleotide or not, for subjecting at least two original codons in a parental polynucleotide template to a full range of codon substitutions. For example, more than one N,N,G/T sequence can be contained in one oligonucleotide to introduce amino acid mutations at more than one site. This plurality of N,N,G/T sequences can be directly contiguous, or separated by one or more additional nucleotide sequence(s). In another aspect, oligonucleotides serviceable for introducing additions and deletions can be used either alone or in combination with the codons containing an N,N,G/T sequence, to introduce any combination or permutation of amino acid additions, deletions, and/or substitutions.

In one aspect, simultaneous mutagenesis of two or more contiguous amino acid positions is done using an oligonucleotide that contains contiguous N,N,G/T triplets, i.e. a degenerate (N,N,G/T)n sequence. In another aspect, degenerate cassettes having less degeneracy than the N,N,G/T sequence are used. For example, it may be desirable in some instances to use (e.g. in an oligonucleotide) a degenerate triplet sequence comprised of only one N, where said N can be in the first second or third position of the triplet. Any other bases including any combinations and permutations thereof can be used in the remaining two positions of the triplet. Alternatively, it may be desirable in some instances to use (e.g. in an oligo) a degenerate N,N,N triplet sequence.

In one aspect, use of degenerate triplets (e.g., N,N,G/T triplets) allows for systematic and easy generation of a full range of possible natural amino acids (for a total of 20 amino acids) into each and every amino acid position in a polypeptide (in alternative aspects, the methods also include generation of less than all possible substitutions per amino acid residue, or codon, position). For example, for a 100 amino acid polypeptide, 2000 distinct species (i.e. 20 possible amino acids per position×100 amino acid positions) can be generated. Through the use of an oligonucleotide or set of oligonucleotides containing a degenerate N,N,G/T triplet, 32 individual sequences can code for all 20 possible natural amino acids. Thus, in a reaction vessel in which a parental polynucleotide sequence is subjected to saturation mutagenesis using at least one such oligonucleotide, there are generated 32 distinct progeny polynucleotides encoding 20 distinct polypeptides. In contrast, the use of a non-degenerate oligonucleotide in site-directed mutagenesis leads to only one progeny polypeptide product per reaction vessel. Nondegenerate oligonucleotides can optionally be used in combination with degenerate primers disclosed; for example, nondegenerate oligonucleotides can be used to generate specific point mutations in a working polynucleotide. This provides one means to generate specific silent point mutations, point mutations leading to corresponding amino acid changes, and point mutations that cause the generation of stop codons and the corresponding expression of polypeptide fragments.

In one aspect, each saturation mutagenesis reaction vessel contains polynucleotides encoding at least 20 progeny polypeptide (e.g., amidases) molecules such that all 20 natural amino acids are represented at the one specific amino acid position corresponding to the codon position mutagenized in the parental polynucleotide (other aspects use less than all 20 natural combinations). The 32-fold degenerate progeny polypeptides generated from each saturation mutagenesis reaction vessel can be subjected to clonal amplification (e.g. cloned into a suitable host, e.g., E. coli host, using, e.g., an expression vector) and subjected to expression screening. When an individual progeny polypeptide is identified by screening to display a favorable change in property (when compared to the parental polypeptide, such as increased proteolytic activity under alkaline or acidic conditions), it can be sequenced to identify the correspondingly favorable amino acid substitution contained therein.

In one aspect, upon mutagenizing each and every amino acid position in a parental polypeptide using saturation mutagenesis as disclosed herein, favorable amino acid changes may be identified at more than one amino acid position. One or more new progeny molecules can be generated that contain a combination of all or part of these favorable amino acid substitutions. For example, if 2 specific favorable amino acid changes are identified in each of 3 amino acid positions in a polypeptide, the permutations include 3 possibilities at each position (no change from the original amino acid, and each of two favorable changes) and 3 positions. Thus, there are 3×3×3 or 27 total possibilities, including 7 that were previously examined—6 single point mutations (i.e. 2 at each of three positions) and no change at any position.

In another aspect, site-saturation mutagenesis can be used together with another stochastic or non-stochastic means to vary sequence, e.g., synthetic ligation reassembly (see below), shuffling, chimerization, recombination and other mutagenizing processes and mutagenizing agents. This invention provides for the use of any mutagenizing process(es), including saturation mutagenesis, in an iterative manner.

Synthetic Ligation Reassembly (SLR)

The invention provides a non-stochastic gene modification system termed “synthetic ligation reassembly,” or simply “SLR,” a “directed evolution process,” to generate polypeptides, e.g., amidases or antibodies of the invention, with new or altered properties. SLR is a method of ligating oligonucleotide fragments together non-stochastically. This method differs from stochastic oligonucleotide shuffling in that the nucleic acid building blocks are not shuffled, concatenated or chimerized randomly, but rather are assembled non-stochastically. See, e.g., U.S. patent application Ser. No. 09/332,835 entitled “Synthetic Ligation Reassembly in Directed Evolution” and filed on Jun. 14, 1999 (“U.S. Ser. No. 09/332,835”). In one aspect, SLR comprises the following steps: (a) providing a template polynucleotide, wherein the template polynucleotide comprises sequence encoding a homologous gene; (b) providing a plurality of building block polynucleotides, wherein the building block polynucleotides are designed to cross-over reassemble with the template polynucleotide at a predetermined sequence, and a building block polynucleotide comprises a sequence that is a variant of the homologous gene and a sequence homologous to the template polynucleotide flanking the variant sequence; (c) combining a building block polynucleotide with a template polynucleotide such that the building block polynucleotide cross-over reassembles with the template polynucleotide to generate polynucleotides comprising homologous gene sequence variations.

SLR does not depend on the presence of high levels of homology between polynucleotides to be rearranged. Thus, this method can be used to non-stochastically generate libraries (or sets) of progeny molecules comprised of over 10100 different chimeras. SLR can be used to generate libraries comprised of over 101000 different progeny chimeras. Thus, aspects of the present invention include non-stochastic methods of producing a set of finalized chimeric nucleic acid molecule shaving an overall assembly order that is chosen by design. This method includes the steps of generating by design a plurality of specific nucleic acid building blocks having serviceable mutually compatible ligatable ends, and assembling these nucleic acid building blocks, such that a designed overall assembly order is achieved.

The mutually compatible ligatable ends of the nucleic acid building blocks to be assembled are considered to be “serviceable” for this type of ordered assembly if they enable the building blocks to be coupled in predetermined orders. Thus, the overall assembly order in which the nucleic acid building blocks can be coupled is specified by the design of the ligatable ends. If more than one assembly step is to be used, then the overall assembly order in which the nucleic acid building blocks can be coupled is also specified by the sequential order of the assembly step(s). In one aspect, the annealed building pieces are treated with an enzyme, such as a ligase (e.g. T4 DNA ligase), to achieve covalent bonding of the building pieces.

In one aspect, the design of the oligonucleotide building blocks is obtained by analyzing a set of progenitor nucleic acid sequence templates that serve as a basis for producing a progeny set of finalized chimeric polynucleotides. These parental oligonucleotide templates thus serve as a source of sequence information that aids in the design of the nucleic acid building blocks that are to be mutagenized, e.g., chimerized or shuffled. In one aspect of this method, the sequences of a plurality of parental nucleic acid templates are aligned in order to select one or more demarcation points. The demarcation points can be located at an area of homology, and are comprised of one or more nucleotides. These demarcation points are preferably shared by at least two of the progenitor templates. The demarcation points can thereby be used to delineate the boundaries of oligonucleotide building blocks to be generated in order to rearrange the parental polynucleotides. The demarcation points identified and selected in the progenitor molecules serve as potential chimerization points in the assembly of the final chimeric progeny molecules. A demarcation point can be an area of homology (comprised of at least one homologous nucleotide base) shared by at least two parental polynucleotide sequences. Alternatively, a demarcation point can be an area of homology that is shared by at least half of the parental polynucleotide sequences, or, it can be an area of homology that is shared by at least two thirds of the parental polynucleotide sequences. Even more preferably a serviceable demarcation points is an area of homology that is shared by at least three fourths of the parental polynucleotide sequences, or, it can be shared by at almost all of the parental polynucleotide sequences. In one aspect, a demarcation point is an area of homology that is shared by all of the parental polynucleotide sequences.

In one aspect, a ligation reassembly process is performed exhaustively in order to generate an exhaustive library of progeny chimeric polynucleotides. In other words, all possible ordered combinations of the nucleic acid building blocks are represented in the set of finalized chimeric nucleic acid molecules. At the same time, in another aspect, the assembly order (i.e. the order of assembly of each building block in the 5′ to 3 sequence of each finalized chimeric nucleic acid) in each combination is by design (or non-stochastic) as described above. Because of the non-stochastic nature of this invention, the possibility of unwanted side products is greatly reduced.

In another aspect, the ligation reassembly method is performed systematically. For example, the method is performed in order to generate a systematically compartmentalized library of progeny molecules, with compartments that can be screened systematically, e.g. one by one. In other words this invention provides that, through the selective and judicious use of specific nucleic acid building blocks, coupled with the selective and judicious use of sequentially stepped assembly reactions, a design can be achieved where specific sets of progeny products are made in each of several reaction vessels. This allows a systematic examination and screening procedure to be performed. Thus, these methods allow a potentially very large number of progeny molecules to be examined systematically in smaller groups. Because of its ability to perform chimerizations in a manner that is highly flexible yet exhaustive and systematic as well, particularly when there is a low level of homology among the progenitor molecules, these methods provide for the generation of a library (or set) comprised of a large number of progeny molecules. Because of the non-stochastic nature of the instant ligation reassembly invention, the progeny molecules generated preferably comprise a library of finalized chimeric nucleic acid molecules having an overall assembly order that is chosen by design. The saturation mutagenesis and optimized directed evolution methods also can be used to generate different progeny molecular species. It is appreciated that the invention provides freedom of choice and control regarding the selection of demarcation points, the size and number of the nucleic acid building blocks, and the size and design of the couplings. It is appreciated, furthermore, that the requirement for intermolecular homology is highly relaxed for the operability of this invention. In fact, demarcation points can even be chosen in areas of little or no intermolecular homology. For example, because of codon wobble, i.e. the degeneracy of codons, nucleotide substitutions can be introduced into nucleic acid building blocks without altering the amino acid originally encoded in the corresponding progenitor template. Alternatively, a codon can be altered such that the coding for an originally amino acid is altered. This invention provides that such substitutions can be introduced into the nucleic acid building block in order to increase the incidence of intermolecular homologous demarcation points and thus to allow an increased number of couplings to be achieved among the building blocks, which in turn allows a greater number of progeny chimeric molecules to be generated.

In another aspect, the synthetic nature of the step in which the building blocks are generated allows the design and introduction of nucleotides (e.g., one or more nucleotides, which may be, for example, codons or introns or regulatory sequences) that can later be optionally removed in an in vitro process (e.g. by mutagenesis) or in an in vivo process (e.g. by utilizing the gene splicing ability of a host organism). It is appreciated that in many instances the introduction of these nucleotides may also be desirable for many other reasons in addition to the potential benefit of creating a serviceable demarcation point.

In one aspect, a nucleic acid building block is used to introduce an intron. Thus, functional introns are introduced into a man-made gene manufactured according to the methods described herein. -The artificially introduced intron(s) can be functional in a host cells for gene splicing much in the way that naturally-occurring introns serve functionally in gene splicing.

Optimized Directed Evolution System

The invention provides a non-stochastic gene modification system termed “optimized directed evolution system” to generate polypeptides, e.g., amidases or antibodies of the invention, with new or altered properties. Optimized directed evolution is directed to the use of repeated cycles of reductive reassortment, recombination and selection that allow for the directed molecular evolution of nucleic acids through recombination. Optimized directed evolution allows generation of a large population of evolved chimeric sequences, wherein the generated population is significantly enriched for sequences that have a predetermined number of crossover events.

A crossover event is a point in a chimeric sequence where a shift in sequence occurs from one parental variant to another parental variant. Such a point is normally at the juncture of where oligonucleotides from two parents are ligated together to form a single sequence. This method allows calculation of the correct concentrations of oligonucleotide sequences so that the final chimeric population of sequences is enriched for the chosen number of crossover events. This provides more control over choosing chimeric variants having a predetermined number of crossover events.

In addition, this method provides a convenient means for exploring a tremendous amount of the possible protein variant space in comparison to other systems. Previously, if one generated, for example, 10¹³ chimeric molecules during a reaction, it would be extremely difficult to test such a high number of chimeric variants for a particular activity. Moreover, a significant portion of the progeny population would have a very high number of crossover events which resulted in proteins that were less likely to have increased levels of a particular activity. By using these methods, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

One method for creating a chimeric progeny polynucleotide sequence is to create oligonucleotides corresponding to fragments or portions of each parental sequence. Each oligonucleotide preferably includes a unique region of overlap so that mixing the oligonucleotides together results in a new variant that has each oligonucleotide fragment assembled in the correct order. Additional information can also be found, e.g., in U.S. Ser. No. 09/332,835; U.S. Pat. No. 6,361,974. The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonueleotides from each parental variant in alternating order is very low. If each oligonuclootide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a 1/3 chance (assuming 3 parents) that an oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determnining the PDF is described below. By utilizing these methods, one can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events. These methods are directed to the use of repeated cycles of reductive reassortment, recombination and selection that allow for the directed molecular evolution of a nucleic acid encoding a polypeptide through recombination. This system allows generation of a large population of evolved chimeric sequences, wherein the generated population is significantly enriched for sequences that have a predetermined number of crossover events. A crossover event is a point in a chimeric sequence where a shift in sequence occurs from one parental variant to another parental variant. Such a point is normally at the juncture of where oligonucleotides from two parents are ligated together to form a single sequence. The method allows calculation of the correct concentrations of oligonucleotide sequences so that the final chimeric population of sequences is enriched for the chosen number of crossover events. This provides more control over choosing chimeric variants having a predetermined number of crossover events.

In addition, these methods provide a convenient means for exploring a tremendous amount of the possible protein variant space in comparison to other systems. By using the methods described herein, the population of chimerics molecules can be enriched for those variants that have a particular number of crossover events. Thus, although one can still generate 1013 chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

In one aspect, the method creates a chimeric progeny polynucleotide sequence by creating oligonucleotides corresponding to fragments or portions of each parental sequence. Each oligonucleotide preferably includes a unique region of overlap so that mixing the oligonucleotides together results in a new variant that has each oligonucleotide fragment assembled in the correct order. See also U.S. Ser. No. 09/332,835.

The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a 1/3 chance (assuming 3 parents) that an oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. One can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events.

Determining Crossover Events

Aspects of the invention include a system and software that receive a desired crossover probability density function (PDF), the number of parent genes to be reassembled, and the number of fragments in the reassembly as inputs. The output of this program is a “fragment PDF” that can be used to determine a recipe for producing reassembled genes, and the estimated crossover PDF of those genes. The processing described herein is preferably performed in MATLABâ (The Mathworks, Natick, Mass.) a programming language and development environment for technical computing.

Iterative Processes

In practicing the invention, these processes can be iteratively repeated. For example, a nucleic acid (or, the nucleic acid) responsible for an altered or new amidase phenotype is identified, re-isolated, again modified, re-tested for activity. This process can be iteratively repeated until a desired phenotype is engineered. For example, an entire biochemical anabolic or catabolic pathway can be engineered into a cell, including, e.g., amide hydrolysis activity, generation of 7-aminocephalosporanic acid (7-ACA), synthesis of a semi-synthetic cephalosporin antibiotic, for example, caphalothin, cephaloridine and cefuroxime.

Similarly, if it is determined that a particular oligonucleotide has no affect at all on the desired trait (e.g., a new amidase phenotype), it can be removed as a variable by synthesizing larger parental oligonucleotides that include the sequence to be removed. Since incorporating the sequence within a larger sequence prevents any crossover events, there will no longer be any variation of this sequence in the progeny polynucleotides. This iterative practice of determining which oligonucleotides are most related to the desired trait, and which are unrelated, allows more efficient exploration all of the possible protein variants that might be provide a particular trait or activity.

In Vivo Shuffling

In vivo shuffling of molecules is use in methods of the invention that provide variants of polypeptides of the invention, e.g., antibodies, amidases, and the like. In vivo shuffling can be performed utilizing the natural property of cells to recombine multimers. While recombination in vivo has provided the major natural route to molecular diversity, genetic recombination remains a relatively complex process that involves 1) the recognition of homologies; 2) strand cleavage, strand invasion, and metabolic steps leading to the production of recombinant chiasma; and finally 3) the resolution of chiasma into discrete recombined molecules. The formation of the chiasma requires the recognition of homologous sequences.

In one aspect, the invention provides a method for producing a hybrid polynucleotide from at least a first polynucleotide (e.g., an amidase of the invention) and a second polynucleotide (e.g., an enzyme, such as an amidase of the invention or any other amidase, or, a tag or an epitope). The invention can be used to produce a hybrid polynucleotide by introducing at least a first polynucleotide and a second polynucleotide which share at least one region of partial sequence homology into a suitable host cell. The regions of partial sequence homology promote processes which result in sequence reorganization producing a hybrid polynucleotide. The term “hybrid polynucleotide”, as used herein, is any nucleotide sequence which results from the method of the present invention and contains sequence from at least two original polynucleotide sequences. Such hybrid polynucleotides can result from intermolecular recombination events which promote sequence integration between DNA molecules. In addition, such hybrid polynucleotides can result from intramolecular reductive reassortment processes which utilize repeated sequences to alter a nucleotide sequence within a DNA molecule.

In Vivo Reassortment

The invention provides in vivo reassortment using the nucleic acids of the invention. These methods comprise “inter-molecular” processes collectively referred to as “recombination” which in bacteria, is generally viewed as a “RecA-dependent” phenomenon. The methods of the invention can utilize recombination processes of a host cell to recombine and re-assort sequences, or the cells' ability to mediate reductive processes to decrease the complexity of quasi-repeated sequences in the cell by deletion. This process of “reductive reassortment” can occur by an “intra-molecular”, RecA-independent process.

In another aspect of the invention, novel polynucleotides can be generated by the process of reductive reassortment. The method involves the generation of constructs containing consecutive sequences (original encoding sequences), their insertion into an appropriate vector, and their subsequent introduction into an appropriate host cell. The reassortment of the individual molecular identities occurs by combinatorial processes between the consecutive sequences in the construct possessing regions of homology, or between quasi-repeated units. The reassortment process recombines and/or reduces the complexity and extent of the repeated sequences, and results in the production of novel molecular species. Various treatments may be applied to enhance the rate of reassortment. These could include treatment with ultra-violet light, or DNA damaging chemicals, and/or the use of host cell lines displaying enhanced levels of “genetic instability”. Thus the reassortment process may involve homologous recombination or the natural property of quasi-repeated sequences to direct their own evolution.

Repeated or “quasi-repeated” sequences play a role in genetic instability. In the present invention, “quasi-repeats” are repeats that are not restricted to their original unit structure. Quasi-repeated units can be presented as an array of sequences in a construct; consecutive units of similar sequences. Once ligated, the junctions between the consecutive sequences become essentially invisible and the quasi-repetitive nature of the resulting construct is now continuous at the molecular level. The deletion process the cell performs to reduce the complexity of the resulting construct operates between the quasi-repeated sequences. The quasi-repeated units provide a practically limitless repertoire of templates upon which slippage events can occur. The constructs containing the quasi-repeats thus effectively provide sufficient molecular elasticity that deletion (and potentially insertion) events can occur virtually anywhere within the quasi-repetitive units.

When the quasi-repeated sequences are all ligated in the same orientation, for instance head to tail or vice versa, the cell cannot distinguish individual units. Consequently, the reductive process can occur throughout the sequences. In contrast, when for example, the units are presented head to head, rather than head to tail, the inversion delineates the endpoints of the adjacent unit so that deletion formation will favor the loss of discrete units. Thus, it is preferable with the present method that the sequences are in the same orientation. Random orientation of quasi-repeated sequences will result in the loss of reassortment efficiency, while consistent orientation of the sequences will offer the highest efficiency. However, while having fewer of the contiguous sequences in the same orientation decreases the efficiency, it may still provide sufficient elasticity for the effective recovery of novel molecules. Constructs can be made with the quasi-repeated sequences in the same orientation to allow higher efficiency.

Sequences can be assembled in a head to tail orientation using any of a variety of methods, including the following:

a) Primers that include a poly-A head and poly-T tail which when made single-stranded would provide orientation can be utilized. This is accomplished by having the first few bases of the primers made from RNA and hence easily removed RNaseH.

b) Primers that include unique restriction cleavage sites can be utilized. Multiple sites, a battery of unique sequences, and repeated synthesis and ligation steps would be required.

c) The inner few bases of the primer could be thiolated and an exonuclease used to produce properly tailed molecules.

The recovery of the re-assorted sequences relies on the identification of cloning vectors with a reduced repetitive index (RI). The re-assorted encoding sequences can then be recovered by amplification. The products are re-cloned and expressed. The recovery of cloning vectors with reduced RI can be affected by:

1) The use of vectors only stably maintained when the construct is reduced in complexity.

2) The physical recovery of shortened vectors by physical procedures. In this case, the cloning vector would be recovered using standard plasmid isolation procedures and size fractionated on either an agarose gel, or column with a low molecular weight cut off utilizing standard procedures.

3) The recovery of vectors containing interrupted genes which can be selected when insert size decreases.

4) The use of direct selection techniques with an expression vector and the appropriate selection.

Encoding sequences (for example, genes) from related organisms may demonstrate a high degree of homology and encode quite diverse protein products. These types of sequences can be useful in the present invention as quasi-repeats. However, while the examples illustrated below demonstrate the reassortment of nearly identical original encoding sequences (quasi-repeats), this process is not limited to such nearly identical repeats.

The following example demonstrates an exemplary method of the invention. Encoding nucleic acid sequences (quasi-repeats) derived from three (3) unique species are described. Each sequence encodes a protein with a distinct set of properties. Each of the sequences differs by a single or a few base pairs at a unique position in the sequence. The quasi-repeated sequences are separately or collectively amplified and ligated into random assemblies such that all possible permutations and combinations are available in the population of ligated molecules. The number of quasi-repeat units can be controlled by the assembly conditions. The average number of quasi-repeated units in a construct is defined as the repetitive index (RI).

Once formed, the constructs may, or may not be size fractionated on an agarose gel according to published protocols, inserted into a cloning vector, and transfected into an appropriate host cell. The cells are then propagated and “reductive reassortment” is effected. The rate of the reductive reassortment process may be stimulated by the introduction of DNA damage if desired. Whether the reduction in RI is mediated by deletion formation between repeated sequences by an “intra-molecular” mechanism, or mediated by recombination-like events through “inter-molecular” mechanisms is immaterial. The end result is a reassortment of the molecules into all possible combinations.

Optionally, the method comprises the additional step of screening the library members of the shuffled pool to identify individual shuffled library members having the ability to bind or otherwise interact, or catalyze a particular reaction (e.g., such as catalytic domain of an enzyme) with a predetermined macromolecule, such as for example a proteinaceous receptor, an oligosaccharide, virion, or other predetermined compound or structure.

The polypeptides that are identified from such libraries can be used for therapeutic, diagnostic, research and related purposes (e.g., catalysts, solutes for increasing osmolarity of an aqueous solution, and the like), and/or can be subjected to one or more additional cycles of shuffling and/or selection.

In another aspect, it is envisioned that prior to or during recombination or reassortment, polynucleotides generated by the method of the invention can be subjected to agents or processes which promote the introduction of mutations into the original polynucleotides. The introduction of such mutations would increase the diversity of resulting hybrid polynucleotides and polypeptides encoded therefrom. The agents or processes which promote mutagenesis can include, but are not limited to: (+)-CC-1065, or a synthetic analog such as (+)-CC-1065-(N3-Adenine (See Sun and Hurley, (1992); an N-acetylated or deacetylated 4′-fluoro-4-aminobiphenyl adduct capable of inhibiting DNA synthesis (See, for example, van de Poll et al. (1992)); or a N-acetylated or deacetylated 4-aminobiphenyl adduct capable of inhibiting DNA synthesis (See also, van de Poll et al. (1992), pp. 751-758); trivalent chromium, a trivalent chromium salt, a polycyclic aromatic hydrocarbon (PAH) DNA adduct capable of inhibiting DNA replication, such as 7-bromomethyl-benz[α]anthracene (“BMA”), tris(2,3-dibromopropyl)phosphate (“Tris-BP”), 1,2-dibromo-3-chloropropane (“DBCP”), 2-bromoacrolein (2BA), benzo[α]pyrene-7,8-dihydrodiol-9-10-epoxide (“BPDE”), a platinum(II) halogen salt, N-hydroxy-2-amino-3-methylimidazo[4,5-f]-quinoline (“N-hydroxy-IQ”), and N-hydroxy-2-amino-1-methyl-6-phenylimidazo[4,5-f]-pyridine (“N-hydroxy-PhIP”). Especially preferred means for slowing or halting PCR amplification consist of UV light (+)-CC-1065 and (+)-CC-1065-(N3-Adenine). Particularly encompassed means are DNA adducts or polynucleotides comprising the DNA adducts from the polynucleotides or polynucleotides pool, which can be released or removed by a process including heating the solution comprising the polynucleotides prior to further processing.

Producing Sequence Variants

The invention also provides additional methods for making sequence variants of the nucleic acid (e.g., amidase) sequences of the invention. The invention also provides additional methods for isolating amidases using the nucleic acids and polypeptides of the invention. In one aspect, the invention provides for variants of an amidase coding sequence (e.g., a gene, cDNA or message) of the invention, which can be altered by any means, including, e.g., random or stochastic methods, or, non-stochastic, or “directed evolution,” methods, as described above.

The isolated variants may be naturally occurring. Variant can also be created in vitro. Variants may be created using genetic engineering techniques such as site directed mutagenesis, random chemical mutagenesis, Exonuclease III deletion procedures, and standard cloning techniques. Alternatively, such variants, fragments, analogs, or derivatives may be created using chemical synthesis or modification procedures. Other methods of making variants are also familiar to those skilled in the art. These include procedures in which nucleic acid sequences obtained from natural isolates are modified to generate nucleic acids which encode polypeptides having characteristics which enhance their value in industrial or laboratory applications. In such procedures, a large number of variant sequences having one or more nucleotide differences with respect to the sequence obtained from the natural isolate are generated and characterized. These nucleotide differences can result in amino acid changes with respect to the polypeptides encoded by the nucleic acids from the natural isolates.

For example, variants may be created using error prone PCR. In error prone PCR, PCR is performed under conditions where the copying fidelity of the DNA polymerase is low, such that a high rate of point mutations is obtained along the entire length of the PCR product. Error prone PCR is described, e.g., in Leung, D. W., et al., Technique, 1:11-15, 1989) and Caldwell, R. C. & Joyce G. F., PCR Methods Applic., 2:28-33, 1992. Briefly, in such procedures, nucleic acids to be mutagenized are mixed with PCR primers, reaction buffer, MgCl₂, MnCl₂, Taq polymerase and an appropriate concentration of dNTPs for achieving a high rate of point mutation along the entire length of the PCR product. For example, the reaction may be performed using 20 fmoles of nucleic acid to be mutagenized, 30 pmole of each PCR primer, a reaction buffer comprising 50 mM KCl, 10 mM Tris HCl (pH 8.3) and 0.01% gelatin, 7 mM MgCl₂, 0.5 mM MnCl₂, 5 units of Taq polymerase, 0.2 mM dGTP, 0.2 mM dATP, 1 mM dCTP, and 1 mM dTTP. PCR may be performed for 30 cycles of 94° C. for 1 min, 45° C. for 1 min, and 72° C. for 1 min. However, it will be appreciated that these parameters may be varied as appropriate. The mutagenized nucleic acids are cloned into an appropriate vector and the activities of the polypeptides encoded by the mutagenized nucleic acids is evaluated.

Variants may also be created using oligonucleotide directed mutagenesis to generate site-specific mutations in any cloned DNA of interest. Oligonucleotide mutagenesis is described, e.g., in Reidhaar-Olson (1988) Science 241:53-57. Briefly, in such procedures a plurality of double stranded oligonucleotides bearing one or more mutations to be introduced into the cloned DNA are synthesized and inserted into the cloned DNA to be mutagenized. Clones containing the mutagenized DNA are recovered and the activities of the polypeptides they encode are assessed.

Another method for generating variants is assembly PCR. Assembly PCR involves the assembly of a PCR product from a mixture of small DNA fragments. A large number of different PCR reactions occur in parallel in the same vial, with the products of one reaction priming the products of another reaction. Assembly PCR is described in, e.g., U.S. Pat. No. 5,965,408.

Still another method of generating variants is sexual PCR mutagenesis. In sexual PCR mutagenesis, forced homologous recombination occurs between DNA molecules of different but highly related DNA sequence in vitro, as a result of random fragmentation of the DNA molecule based on sequence homology, followed by fixation of the crossover by primer extension in a PCR reaction. Sexual PCR mutagenesis is described, e.g., in Stemmer (1994) Proc. Natl. Acad. Sci. USA 91:10747-10751. Briefly, in such procedures a plurality of nucleic acids to be recombined are digested with DNase to generate fragments having an average size of 50-200 nucleotides. Fragments of the desired average size are purified and resuspended in a PCR mixture. PCR is conducted under conditions which facilitate recombination between the nucleic acid fragments. For example, PCR may be performed by resuspending the purified fragments at a concentration of 10-30 ng/:l in a solution of 0.2 mM of each dNTP, 2.2 mM MgCl₂, 50 mM KCL, 10 mM Tris HCl, pH 9.0, and 0.1% Triton X-100. 2.5 units of Taq polymerase per 100:1 of reaction mixture is added and PCR is performed using the following regime: 94° C. for 60 seconds, 94° C. for 30 seconds, 50-55° C. for 30 seconds, 72° C. for 30 seconds (30-45 times) and 72° C. for 5 minutes. However, it will be appreciated that these parameters may be varied as appropriate. In some aspects, oligonucleotides may be included in the PCR reactions. In other aspects, the Klenow fragment of DNA polymerase I may be used in a first set of PCR reactions and Taq polymerase may be used in a subsequent set of PCR reactions. Recombinant sequences are isolated and the activities of the polypeptides they encode are assessed.

Variants may also be created by in vivo mutagenesis. In some aspects, random mutations in a sequence of interest are generated by propagating the sequence of interest in a bacterial strain, such as an E. coli strain, which carries mutations in one or more of the DNA repair pathways. Such “mutator” strains have a higher random mutation rate than that of a wild-type parent. Propagating the DNA in one of these strains will eventually generate random mutations within the DNA. Mutator strains suitable for use for in vivo mutagenesis are described, e.g., in PCT Publication No. WO 91/16427.

Variants may also be generated using cassette mutagenesis. In cassette mutagenesis a small region of a double stranded DNA molecule is replaced with a synthetic oligonucleotide “cassette” that differs from the native sequence. The oligonucleotide often contains completely and/or partially randomized native sequence.

Recursive ensemble mutagenesis may also be used to generate variants. Recursive ensemble mutagenesis is an algorithm for protein engineering (protein mutagenesis) developed to produce diverse populations of phenotypically related mutants whose members differ in amino acid sequence. This method uses a feedback mechanism to control successive rounds of combinatorial cassette mutagenesis. Recursive ensemble mutagenesis is described, e.g., in Arkin (1992) Proc. Natl. Acad. Sci. USA 89:7811-7815.

In some aspects, variants are created using exponential ensemble mutagenesis. Exponential ensemble mutagenesis is a process for generating combinatorial libraries with a high percentage of unique and functional mutants, wherein small groups of residues are randomized in parallel to identify, at each altered position, amino acids which lead to functional proteins. Exponential ensemble mutagenesis is described, e.g., in Delegrave (1993) Biotechnology Res. 11:1548-1552. Random and site-directed mutagenesis are described, e.g., in Arnold (1993) Current Opinion in Biotechnology 4:450-455.

In some aspects, the variants are created using shuffling procedures wherein portions of a plurality of nucleic acids which encode distinct polypeptides are fused together to create chimeric nucleic acid sequences which encode chimeric polypeptides as described in, e.g., U.S. Pat. Nos. 5,965,408; 5,939,250 (see also discussion, above).

The invention also provides variants of polypeptides of the invention (e.g., amidases) comprising sequences in which one or more of the amino acid residues (e.g., of an exemplary polypeptide, such as SEQ ID NO: 2, SEQ ID NO: 4, SEQ ID NO: 6, ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 113, SEQ ID NO: 114) are substituted with a conserved or non-conserved amino acid residue (e.g., a conserved amino acid residue) and such substituted amino acid residue may or may not be one encoded by the genetic code. Conservative substitutions are those that substitute a given amino acid in a polypeptide by another amino acid of like characteristics. Thus, polypeptides of the invention include those with conservative substitutions of sequences of the invention, including but not limited to the following replacements: replacements of an aliphatic amino acid such as Alanine, Valine, Leucine and Isoleucine with another aliphatic amino acid; replacement of a Serine with a Threonine or vice versa; replacement of an acidic residue such as Aspartic acid and Glutamic acid with another acidic residue; replacement of a residue bearing an amide group, such as Asparagine and Glutamine, with another residue bearing an amide group; exchange of a basic residue such as Lysine and Arginine with another basic residue; and replacement of an aromatic residue such as Phenylalanine, Tyrosine with another aromatic residue. Other variants are those in which one or more of the amino acid residues of the polypeptides of the invention includes a substituent group.

Other variants within the scope of the invention are those in which the polypeptide is associated with another compound, such as a compound to increase the half-life of the polypeptide, for example, polyethylene glycol.

Additional variants within the scope of the invention are those in which additional amino acids are fused to the polypeptide, such as a leader sequence, a secretory sequence, a proprotein sequence or a sequence which facilitates purification, enrichment, or stabilization of the polypeptide.

In some aspects, the variants, fragments, derivatives and analogs of the polypeptides of the invention retain the same biological function or activity as the exemplary polypeptides, e.g., amidase activity, as described herein. In other aspects, the variant, fragment, derivative, or analog includes a proprotein, such that the variant, fragment, derivative, or analog can be activated by cleavage of the proprotein portion to produce an active polypeptide.

Optimizing Codons to Achieve High Levels of Protein Expression in Host Cells

The invention provides methods for modifying amidase-encoding nucleic acids to modify codon usage. In one aspect, the invention provides methods for modifying codons in a nucleic acid encoding an amidase to increase or decrease its expression in a host cell. The invention also provides nucleic acids encoding an amidase modified to increase its expression in a host cell, amidase so modified, and methods of making the modified amidases. The method comprises identifying a “non-preferred” or a “less preferred” codon in amidase-encoding nucleic acid and replacing one or more of these non-preferred or less preferred codons with a “preferred codon” encoding the same amino acid as the replaced codon and at least one non-preferred or less preferred codon in the nucleic acid has been replaced by a preferred codon encoding the same amino acid. A preferred codon is a codon over-represented in coding sequences in genes in the host cell and a non-preferred or less preferred codon is a codon under-represented in coding sequences in genes in the host cell.

Host cells for expressing the nucleic acids, expression cassettes and vectors of the invention include bacteria, yeast, fungi, plant cells, insect cells and mammalian cells. Thus, the invention provides methods for optimizing codon usage in all of these cells, codon-altered nucleic acids and polypeptides made by the codon-altered nucleic acids. Exemplary host cells include gram negative bacteria, such as Escherichia coli and Pseudomonas fluorescens; gram positive bacteria, such as Streptomyces diversa, Lactobacillus gasseri, Lactococcus lactis, Lactococcus cremoris, Bacillus subtilis. Exemplary host cells also include eukaryotic organisms, e.g., various yeast, such as Saccharomyces sp., including Saccharomyces cerevisiae, Schizosaccharomyces pombe, Pichia pastoris, and Kluyveromyces lactis, Hansenula polymorpha, Aspergillus niger, and mammalian cells and cell lines and insect cells and cell lines. Thus, the invention also includes nucleic acids and polypeptides optimized for expression in these organisms and species.

For example, the codons of a nucleic acid encoding an amidase isolated from a bacterial cell are modified such that the nucleic acid is optimally expressed in a bacterial cell different from the bacteria from which the amidase was derived, a yeast, a fungi, a plant cell, an insect cell or a mammalian cell. Methods for optimizing codons are well known in the art, see, e.g., U.S. Pat. No. 5,795,737; Baca (2000) Int. J. Parasitol. 30:113-118; Hale (1998) Protein Expr. Purif. 12:185-188; Narum (2001) Infect. Immun. 69:7250-7253. See also Narum (2001) Infect. Immun. 69:7250-7253, describing optimizing codons in mouse systems; Outchkourov (2002) Protein Expr. Purif. 24:18-24, describing optimizing codons in yeast; Feng (2000) Biochemistry 39:15399-15409, describing optimizing codons in E. coli; Humphreys (2000) Protein Expr. Purif. 20:252-264, describing optimizing codon usage that affects secretion in E. coli.

Transgenic Non-Human Animals

The invention provides transgenic non-human animals comprising a nucleic acid, a polypeptide (an amidase or an antibody of the invention), an expression cassette or vector or a transfected or transformed cell of the invention. The invention also provides methods of making and using these transgenic non-human animals.

The transgenic non-human animals can be, e.g., goats, rabbits, sheep, pigs, cows, rats and mice, comprising the nucleic acids of the invention. These animals can be used, e.g., as in vivo models to study amidase activity, or, as models to screen for agents that change the amidase activity in vivo. The coding sequences for the polypeptides to be expressed in the transgenic non-human animals can be designed to be constitutive, or, under the control of tissue-specific, developmental-specific or inducible transcriptional regulatory factors. Transgenic non-human animals can be designed and generated using any method known in the art; see, e.g., U.S. Pat. Nos. 6,211,428; 6,187,992; 6,156,952; 6,118,044; 6,111,166; 6,107,541; 5,959,171; 5,922,854; 5,892,070; 5,880,327; 5,891,698; 5,639,940; 5,573,933; 5,387,742; 5,087,571, describing making and using transformed cells and eggs and transgenic mice, rats, rabbits, sheep, pigs and cows. See also, e.g., Pollock (1999) J. Immunol. Methods 231:147-157, describing the production of recombinant proteins in the milk of transgenic dairy animals; Baguisi (1999) Nat. Biotechnol. 17:456-461, demonstrating the production of transgenic goats. U.S. Pat. No. 6,211,428, describes making and using transgenic non-human mammals which express in their brains a nucleic acid construct comprising a DNA sequence. U.S. Pat. No. 5,387,742, describes injecting cloned recombinant or synthetic DNA sequences into fertilized mouse eggs, implanting the injected eggs in pseudo-pregnant females, and growing to term transgenic mice whose cells express proteins related to the pathology of Alzheimer's disease. U.S. Pat. No. 6,187,992, describes making and using a transgenic mouse whose genome comprises a disruption of the gene encoding amyloid precursor protein (APP).

“Knockout animals” can also be used to practice the methods of the invention. For example, in one aspect, the transgenic or modified animals of the invention comprise a “knockout animal,” e.g., a “knockout mouse,” engineered not to express an endogenous gene, which is replaced with a gene expressing an amidase of the invention, or, a fusion protein comprising an amidase of the invention.

Transgenic Plants and Seeds

The invention provides transgenic plants and seeds comprising a nucleic acid, a polypeptide (an amidase or an antibody of the invention), an expression cassette or vector or a transfected or transformed cell of the invention. The transgenic plant can be dicotyledonous (a dicot) or monocotyledonous (a monocot). The invention also provides methods of making and using these transgenic plants and seeds. The transgenic plant or plant cell expressing a polypeptide of the present invention may be constructed in accordance with any method known in the art. See, for example, U.S. Pat. No. 6,309,872.

Nucleic acids and expression constructs of the invention can be introduced into a plant cell by any means. For example, nucleic acids or expression constructs can be introduced into the genome of a desired plant host, or, the nucleic acids or expression constructs can be episomes. Introduction into the genome of a desired plant can be such that the host's α-amidase production is regulated by endogenous transcriptional or translational control elements. The invention also provides “knockout plants” where insertion of gene sequence by, e.g., homologous recombination, has disrupted the expression of the endogenous gene. Means to generate “knockout” plants are well-known in the art, see, e.g., Strepp (1998) Proc Natl. Acad. Sci. USA 95:4368-4373; Miao (1995) Plant J 7:359-365. See discussion on transgenic plants, below.

The nucleic acids of the invention can be used to confer desired traits on essentially any plant, e.g., on starch-producing plants, such as potato, wheat, rice, barley, and the like. Nucleic acids of the invention can be used to manipulate metabolic pathways of a plant in order to optimize or alter host's expression of amidase. Amidases of the invention can be used in production of a transgenic plant to produce a compound not naturally produced by that plant. This can lower production costs or create a novel product.

In one aspect, the first step in production of a transgenic plant involves making an expression construct for expression in a plant cell. These techniques are well known in the art. They can include selecting and cloning a promoter, a coding sequence for facilitating efficient binding of ribosomes to mRNA and selecting the appropriate gene terminator sequences. One exemplary constitutive promoter is CaMV35S, from the cauliflower mosaic virus, which generally results in a high degree of expression in plants. Other promoters are more specific and respond to cues in the plant's internal or external environment. An exemplary light-inducible promoter is the promoter from the cab gene, encoding the major chlorophyll a/b binding protein.

In one aspect, the nucleic acid is modified to achieve greater expression in a plant cell. For example, a sequence of the invention is likely to have a higher percentage of A-T nucleotide pairs compared to that seen in a plant, some of which prefer G-C nucleotide pairs. Therefore, A-T nucleotides in the coding sequence can be substituted with G-C nucleotides without significantly changing the amino acid sequence to enhance production of the gene product in plant cells.

Selectable marker gene can be added to the gene construct in order to identify plant cells or tissues that have successfully integrated the transgene. This may be necessary because achieving incorporation and expression of genes in plant cells is a rare event, occurring in just a few percent of the targeted tissues or cells. Selectable marker genes encode proteins that provide resistance to agents that are normally toxic to plants, such as antibiotics or herbicides. Only plant cells that have integrated the selectable marker gene will survive when grown on a medium containing the appropriate antibiotic or herbicide. As for other inserted genes, marker genes also require promoter and termination sequences for proper function.

In one aspect, making transgenic plants or seeds comprises incorporating sequences of the invention and, optionally, marker genes into a target expression construct (e.g., a plasmid), along with positioning of the promoter and the terminator sequences. This can involve transferring the modified gene into the plant through a suitable method. For example, a construct may be introduced directly into the genomic DNA of the plant cell using techniques such as electroporation and microinjection of plant cell protoplasts, or the constructs can be introduced directly to plant tissue using ballistic methods, such as DNA particle bombardment. For example, see, e.g., Christou (1997) Plant Mol. Biol. 35:197-203; Pawlowski (1996) Mol. Biotechnol. 6:17-30; Klein (1987) Nature 327:70-73; Takumi (1997) Genes Genet. Syst. 72:63-69, discussing use of particle bombardment to introduce transgenes into wheat; and Adam (1997) supra, for use of particle bombardment to introduce YACs into plant cells. For example, Rinehart (1997) supra, used particle bombardment to generate transgenic cotton plants. Apparatus for accelerating particles is described U.S. Pat. No. 5,015,580; and, the commercially available BioRad (Biolistics) PDS-2000 particle acceleration instrument; see also, John, U.S. Pat. No. 5,608,148; and Ellis, U.S. Pat. No. 5,681,730, describing particle-mediated transformation of gymnosperms.

In one aspect, protoplasts can be immobilized and injected with a nucleic acids, e.g., an expression construct. Although plant regeneration from protoplasts is not easy with cereals, plant regeneration is possible in legumes using somatic embryogenesis from protoplast derived callus. Organized tissues can be transformed with naked DNA using gene gun technique, where DNA is coated on tungsten microprojectiles, shot 1/100th the size of cells, which carry the DNA deep into cells and organelles. Transformed tissue is then induced to regenerate, usually by somatic embryogenesis. This technique has been successful in several cereal species including maize and rice.

Nucleic acids, e.g., expression constructs, can also be introduced in to plant cells using recombinant viruses. Plant cells can be transformed using viral vectors, such as, e.g., tobacco mosaic virus derived vectors (Rouwendal (1997) Plant Mol. Biol. 33:989-999), see Porta (1996) “Use of viral replicons for the expression of genes in plants,” Mol. Biotechnol. 5:209-221.

Alternatively, nucleic acids, e.g., an expression construct, can be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Agrobacterium tumefaciens-mediated transformation techniques, including disarming and use of binary vectors, are well described in the scientific literature. See, e.g., Horsch (1984) Science 233:496-498; Fraley (1983) Proc. Natl. Acad. Sci. USA 80:4803 (1983); Gene Transfer to Plants, Potrykus, ed. (Springer-Verlag, Berlin 1995). The DNA in an A. tumefaciens cell is contained in the bacterial chromosome as well as in another structure known as a Ti (tumor-inducing) plasmid. The Ti plasmid contains a stretch of DNA termed T-DNA (˜20 kb long) that is transferred to the plant cell in the infection process and a series of vir (virulence) genes that direct the infection process. A. tumefaciens can only infect a plant through wounds: when a plant root or stem is wounded it gives off certain chemical signals, in response to which, the vir genes of A. tumefaciens become activated and direct a series of events necessary for the transfer of the T-DNA from the Ti plasmid to the plant's chromosome. The T-DNA then enters the plant cell through the wound. One speculation is that the T-DNA waits until the plant DNA is being replicated or transcribed, then inserts itself into the exposed plant DNA. In order to use A. tumefaciens as a transgene vector, the tumor-inducing section of T-DNA have to be removed, while retaining the T-DNA border regions and the vir genes. The transgene is then inserted between the T-DNA border regions, where it is transferred to the plant cell and becomes integrated into the plant's chromosomes.

The invention provides for the transformation of monocotyledonous plants using the nucleic acids of the invention, including important cereals, see Hiei (1997) Plant Mol. Biol. 35:205-218. See also, e.g., Horsch, Science (1984) 233:496; Fraley (1983) Proc. Natl Acad. Sci USA 80:4803; Thykjaer (1997) supra; Park (1996) Plant Mol. Biol. 32:1135-1148, discussing T-DNA integration into genomic DNA. See also D'Halluin, U.S. Pat. No. 5,712,135, describing a process for the stable integration of a DNA comprising a gene that is functional in a cell of a cereal, or other monocotyledonous plant.

In one aspect, the third step can involve selection and regeneration of whole plants capable of transmitting the incorporated target gene to the next generation. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee (1987) Ann. Rev. of Plant Phys. 38:467-486. To obtain whole plants from transgenic tissues such as immature embryos, they can be grown under controlled environmental conditions in a series of media containing nutrients and hormones, a process known as tissue culture. Once whole plants are generated and produce seed, evaluation of the progeny begins.

After the expression cassette is stably incorporated in transgenic plants, it can be introduced into other plants by sexual crossing. Any of a number of standard breeding techniques can be used, depending upon the species to be crossed. Since transgenic expression of the nucleic acids of the invention leads to phenotypic changes, plants comprising the recombinant nucleic acids of the invention can be sexually crossed with a second plant to obtain a final product. Thus, the seed of the invention can be derived from a cross between two transgenic plants of the invention, or a cross between a plant of the invention and another plant. The desired effects (e.g., expression of the polypeptides of the invention to produce a plant in which flowering behavior is altered) can be enhanced when both parental plants express the polypeptides of the invention. The desired effects can be passed to future plant generations by standard propagation means.

The nucleic acids and polypeptides of the invention are expressed in or inserted in any plant or seed. Transgenic plants of the invention can be dicotyledonous or monocotyledonous. Examples of monocot transgenic plants of the invention are grasses, such as meadow grass (blue grass, Poa), forage grass such as festuca, lolium, temperate grass, such as Agrostis, and cereals, e.g., wheat, oats, rye, barley, rice, sorghum, and maize (corn). Examples of dicot transgenic plants of the invention are tobacco, legumes, such as lupins, potato, sugar beet, pea, bean and soybean, and cruciferous plants (family Brassicaceae), such as cauliflower, rape seed, and the closely related model organism Arabidopsis thaliana. Thus, the transgenic plants and seeds of the invention include a broad range of plants, including, but not limited to, species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, and Zea.

In alternative embodiments, the nucleic acids of the invention are expressed in plants which contain fiber cells, including, e.g., cotton, silk cotton tree (Kapok, Ceiba pentandra), desert willow, creosote bush, winterfat, balsa, ramie, kenaf, hemp, roselle, jute, sisal abaca and flax. In alternative embodiments, the transgenic plants of the invention can be members of the genus Gossypium, including members of any Gossypium species, such as G. arboreum; G. herbaceum, G. barbadense, and G. hirsutum.

The invention also provides for transgenic plants to be used for producing large amounts of the polypeptides of the invention. For example, see Palmgren (1997) Trends Genet. 13:348; Chong (1997) Transgenic Res. 6:289-296 (producing human milk protein beta-casein in transgenic potato plants using an auxin-inducible, bidirectional mannopine synthase (mas1′,2′) promoter with Agrobacterium tumefaciens-mediated leaf disc transformation methods).

Using known procedures, one of skill can screen for plants of the invention by detecting the increase or decrease of transgene mRNA or protein in transgenic plants. Means for detecting and quantitation of mRNAs or proteins are well known in the art.

Polypeptides and Peptides

The invention provides isolated or recombinant polypeptides having a sequence identity to an exemplary sequence of the invention, e.g., SEQ ID NO: 2, SEQ ID NO: 4, SEQ ID NO: 6, ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 113, SEQ ID NO: 114. As discussed above, the identity can be over the full length of the polypeptide, or, the identity can be over a region of at least about 50, 60, 70, 80, 90, 100, 150, 200, 250, 300, 350, 400, 450, 500, 550, 600, 650, 700, 750, 800, 850, 900, 950, 1000 or 1100 or more residues. Polypeptides of the invention can also be shorter than the full length of exemplary polypeptides. In alternative aspects, the invention provides polypeptides (peptides, fragments) ranging in size between about 5 and the full length of a polypeptide, e.g., an enzyme, such as an amidase; exemplary sizes being of about 5, 10, 15, 20, 25, 30, 35, 40, 45, 50, 55, 60, 65, 70, 75, 80, 85, 90, 100, 125, 150, 175, 200, 250, 300, 350, 400, 450, 500, 550, 600, 650, 700, or more residues, e.g., contiguous residues of an exemplary amidase of the invention. Peptides of the invention can be useful as, e.g., labeling probes, antigens, toleragens, motifs, amidase active sites.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer.

The peptides and polypeptides of the invention can also be glycosylated. The glycosylation can be added post-translationally either chemically or by cellular biosynthetic mechanisms, wherein the later incorporates the use of known glycosylation motifs, which can be native to the sequence or can be added as a peptide or added in the nucleic acid coding sequence. The glycosylation can be O-linked or N-linked.

The peptides and polypeptides of the invention include all polymers comprising amino acids joined to each other by peptide bonds or modified peptide bonds, i.e., peptide isosteres, and may contain modified amino acids other than the 20 gene-encoded amino acids. The polypeptides may be modified by either natural processes, such as post-translational processing, or by chemical modification techniques which are well known in the art. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also a given polypeptide may have many types of modifications. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphytidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. (See Creighton, T. E., Proteins—Structure and Molecular Properties 2nd Ed., W. H. Freeman and Company, New York (1993); Posttranslational Covalent Modification of Proteins, B. C. Johnson, Ed., Academic Press, New York, pp. 1-12 (1983)).

The peptides and polypeptides of the invention, as defined above, include all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, in one aspect, a mimetic composition is within the scope of the invention if it has an amidase activity.

Polypeptide mimetic compositions of the invention can contain any combination of non-natural structural components. In alternative aspect, mimetic compositions of the invention include one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide of the invention can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

A polypeptide of the invention can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; D- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholinyl-(4-ethyl)carbodiimide or 1-ethyl-3(4-azonia-4,4-dimetholpentyl)carbodiimide. Aspartyl or glutamyl can also be converted to asparaginyl and glutaminyl residues by reaction with ammonium ions. Mimetics of basic amino acids can be generated by substitution with, e.g., (in addition to lysine and arginine) the amino acids omithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl)propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A residue, e.g., an amino acid, of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but also can be referred to as the R- or S-form.

The invention also provides methods for modifying the polypeptides of the invention by either natural processes, such as post-translational processing (e.g., phosphorylation, acylation, etc), or by chemical modification techniques, and the resulting modified polypeptides. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also a given polypeptide may have many types of modifications. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. See, e.g., Creighton, T. E., Proteins—Structure and Molecular Properties 2nd Ed., W. H. Freeman and Company, New York (1993); Posttranslational Covalent Modification of Proteins, B. C. Johnson, Ed., Academic Press, New York, pp. 1-12 (1983).

Solid-phase chemical peptide synthesis methods can also be used to synthesize the polypeptide or fragments of the invention. Such method have been known in the art since the early 1960's (Merrifield, R. B., J. Am. Chem. Soc., 85:2149-2154, 1963) (See also Stewart, J. M. and Young, J. D., Solid Phase Peptide Synthesis, 2nd Ed., Pierce Chemical Co., Rockford, Ill., pp. 11-12)) and have recently been employed in commercially available laboratory peptide design and synthesis kits (Cambridge Research Biochemicals). Such commercially available laboratory kits have generally utilized the teachings of H. M. Geysen et al, Proc. Natl. Acad. Sci., USA, 81:3998 (1984) and provide for synthesizing peptides upon the tips of a multitude of “rods” or “pins” all of which are connected to a single plate. When such a system is utilized, a plate of rods or pins is inverted and inserted into a second plate of corresponding wells or reservoirs, which contain solutions for attaching or anchoring an appropriate amino acid to the pin's or rod's tips. By repeating such a process step, i.e., inverting and inserting the rod's and pin's tips into appropriate solutions, amino acids are built into desired peptides. In addition, a number of available FMOC peptide synthesis systems are available. For example, assembly of a polypeptide or fragment can be carried out on a solid support using an Applied Biosystems, Inc. Model 431A™ automated peptide synthesizer. Such equipment provides ready access to the peptides of the invention, either by direct synthesis or by synthesis of a series of fragments that can be coupled using other known techniques.

The invention provides novel amidases, including the exemplary enzymes having sequences as set forth in SEQ ID NO: 2, SEQ ID NO: 4, SEQ ID NO: 6, ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 113, SEQ ID NO: 114, nucleic acids encoding them, antibodies that bind them, and methods for making and using them. In one aspect, the polypeptides of the invention have an amidase activity, as described herein, including, e.g., the ability to hydrolyze amides, including enzymes having secondary amidase activity, including a peptidase, a protease and/or a hydantoinase activity.

In alternative aspects, the amidases of the invention have activities that have been modified from those of the exemplary amidases described herein. The invention includes amidases with and without signal sequences and the signal sequences themselves. The invention includes immobilized amidases, anti-amidase antibodies and fragments thereof. The invention provides methods for inhibiting amidase activity, e.g., using dominant negative mutants or anti-amidase antibodies of the invention. The invention includes heterocomplexes, e.g., fusion proteins, heterodimers, etc., comprising the amidases of the invention.

Amidases of the invention can be used in laboratory and industrial settings to hydrolyze amide compounds for a variety of purposes. These amidases can be used alone to provide specific hydrolysis or can be combined with other amidases to provide a “cocktail” with a broad spectrum of activity. Exemplary uses of the amidases of the invention include their use to increase flavor in food (e.g., enzyme ripened cheese), promote bacterial and fungal killing, modify and de-protect fine chemical intermediates, synthesize peptide bonds, carry out chiral resolutions, hydrolyze amide-containing antibiotics or other drugs, e.g., cephalosporin C.

Amidases of the invention can have an amidase activity under various conditions, e.g., extremes in pH and/or temperature, oxidizing agents, and the like. The invention provides methods leading to alternative amidase preparations with different catalytic efficiencies and stabilities, e.g., towards temperature, oxidizing agents and changing wash conditions. In one aspect, amidase variants can be produced using techniques of site-directed mutagenesis and/or random mutagenesis. In one aspect, directed evolution can be used to produce a great variety of amidase variants with alternative specificities and stability.

The proteins of the invention are also useful as research reagents to identify amidase modulators, e.g., activators or inhibitors of amidase activity. Briefly, test samples (compounds, broths, extracts, and the like) are added to amidase assays to determine their ability to inhibit hydrolysis. Inhibitors identified in this way can be used in industry and research to reduce or prevent undesired hydrolysis, e.g., proteolysis. As with amidases, inhibitors can be combined to increase the spectrum of activity.

The invention also provides methods of discovering new amidases using the nucleic acids, polypeptides and antibodies of the invention. In one aspect, lambda phage libraries are screened for expression-based discovery of amidases. In one aspect, the invention uses lambda phage libraries in screening to allow detection of toxic clones; improved access to substrate; reduced need for engineering a host, by-passing the potential for any bias resulting from mass excision of the library; and, faster growth at low clone densities. Screening of lambda phage libraries can be in liquid phase or in solid phase. In one aspect, the invention provides screening in liquid phase. This gives a greater flexibility in assay conditions; additional substrate flexibility; higher sensitivity for weak clones; and ease of automation over solid phase screening.

The invention provides screening methods using the proteins and nucleic acids of the invention and robotic automation to enable the execution of many thousands of biocatalytic reactions and screening assays in a short period of time, e.g., per day, as well as ensuring a high level of accuracy and reproducibility (see discussion of arrays, below). As a result, a library of derivative compounds can be produced in a matter of weeks. For further teachings on modification of molecules, including small molecules, see PCT/US94/09174.

The present invention includes amidase enzymes which are non-naturally occurring amidase variants having a different proteolytic activity, stability, substrate specificity, pH profile and/or performance characteristic as compared to the precursor amidase from which the amino acid sequence of the variant is derived. Specifically, such amidase variants have an amino acid sequence not found in nature, which is derived by substitution of a plurality of amino acid residues of a precursor amidase with different amino acids. The precursor amidase may be a naturally-occurring amidase or a recombinant amidase. The useful amidase variants encompass the substitution of any of the naturally occurring L-amino acids at the designated amino acid residue positions.

Exemplary has the sequence:

SEQ ID NO:2

Met Asn Ser Thr Leu Ala Tyr Phe Thr Glu Gln Gly Pro Met Ser Asp Pro Gly Thr Tyr Arg Ser Leu Phe Glu Asp
Leu Pro Thr Ser Ile Pro Asp Leu Val Lys Leu Val Gln Gly Val Thr Leu His Ile Phe Trp Thr Glu Arg Tyr Gly
Leu Lys Val Pro Pro Gln Arg Met Glu Glu Leu Gln Leu Arg Ser Met Glu Lys Arg Leu Ala Arg Thr Leu Glu Leu
Asp Pro Arg Pro Leu Val Glu Pro Arg Pro Leu Glu Asn Lys Leu Leu Gly Asn Cys Arg Asp His Ser Leu Leu Leu
Thr Ala Leu Leu Arg His Gln Gly Val Pro Ala Arg Ala Arg Cys Gly Phe Gly Ala Tyr Phe Leu Pro Asp His Phe
Glu Asp His Trp Val Val Glu Tyr Trp Asn Gln Glu Gln Ser Arg Trp Val Leu Val Asp Ala Gln Leu Asp Ala Ser
Gln Arg Glu Val Leu Lys Ile Asp Phe Asp Thr Leu Asp Val Pro Arg Asp Gln Phe Ile Val Gly Gly Lys Ala Trp
Gln Met Cys Arg Ser Gly Glu Gln Asp Pro Gly Lys Phe Gly Ile Phe Asp Met Asn Gly Leu Gly Phe Val Arg Gly
Asp Leu Val Arg Asp Val Ala Ser Leu Asn Lys Met Glu Leu Leu Pro Trp Asp Cys Trp Gly Val Ile Leu Val Glu
Lys Leu Asp Asp Pro Ala Asp Leu Ser Val Leu Asp Arg Val Ala Ser Leu Thr Ala Arg Asp Val Pro Asp Phe Glu
Val Leu Arg Ala Cys Tyr Glu Ser Asp Pro Arg Leu Arg Val Asn Asp Ser Leu Leu Ser Tyr Val Asn Gly Asn Met
Val Glu Val Gln Ile Ala
Exemplary has the sequence

SEQ ID NO:4

Val Pro Ser Leu Asp Glu Tyr Ala Thr His Ser Ala Phe Thr Asp Pro Gly Arg His Arg Asp Leu Leu Gly
Ala Thr Gly Thr Ser Pro Asp Asp Leu His Arg Ala Ala Thr Gly Val Val Leu His Tyr Arg Gly Gln Arg Asp Arg
Leu Thr Asp Glu Gln Leu Pro Asp Val Asp Leu Arg Trp Phe Ser Ala Gln Leu Glu Val Val Arg His Arg Ala Ala
Leu Pro Leu Gly Ala His Arg Thr Asp Ala Gln His Leu Ala Gly Cys Cys Arg Asp His Thr Leu Leu Ala Val Ala
Val Leu Arg Glu His Gly Ile Pro Ala Arg Ser Arg Val Gly Phe Ala Asp Tyr Phe Glu Pro Asp Phe His His Asp
His Val Val Val Glu Arg Trp Asp Gly Ala Arg Trp Val Arg Phe Asp Ser Ala Leu Asp Pro Ala Asp His Leu Phe
Asp Val Asp Asp Met Pro Ala Gly Glu Gly Met Pro Phe Glu Thr Ala Ala Glu Val Trp Leu Ala Ala Arg Ala Gly
Arg Val Asp Pro Arg Arg Tyr Gly Val Asp Lys Ala Met Pro His Leu Ile Gly Ile Pro Phe Leu Leu Gly Glu Val
Phe Leu Glu Leu Ala His Arg Gln Arg Asp Glu Ile Leu Leu Trp Asp Val Trp Gly Val Gly Ile Pro Pro Phe Ala
Arg Pro Asp Gly Leu Ala Pro Val Thr Met Ser Asp Asp Glu Met Ala Glu Leu Ala Asp Gln Val Ala Arg Leu Val
Val Ala Ala Asp Asp Gly Asp Asp Ala Ala Asp Ala Ala Leu Asp Ala Arg Tyr Ala Ala Asp Pro Arg Leu Arg Pro
Thr Ala Asn Pro Leu Val Ala Leu Ser Pro Leu Glu Arg Ile Gly Asp Val Asp Leu Thr Ala Arg Thr Thr Thr Trp
Arg
Exemplary has the sequence:

SEQ ID NO:6

Met Thr Asn Gln Pro Glu Arg Ser Thr Ala Arg Ser Tyr Tyr Ala Ala Pro Ala Ala Met Thr Asp Leu Ser Ala His
Arg Ala Arg Leu Arg Asp Leu Pro Thr Asp Leu Ala Gly Leu Cys Arg Val Ile Gln Gly Leu Leu Val His Pro Phe
Leu Ala His Leu Tyr Gly Leu Pro Ser Ser Ala Leu Arg Leu Gly Glu Leu Glu Leu Arg Arg Ala Ser Ala Met Leu
Asp His Ala Leu Thr Leu Asp Ala Arg Pro Leu Val Glu Ala Arg Pro Pro Glu Arg Arg Leu Val Gly Asn Cys Arg
His Phe Ser Val Leu Phe Cys Ala Leu Leu Arg Ala Gln Gly Val Pro Ala Arg Ala Arg Cys Gly Phe Gly Ala Tyr
Phe Asn Pro Ala Arg Phe Glu Asp His Trp Val Gly Glu Val Trp Asp Ser Thr Arg Gly Ala Trp Arg Leu Val Asp
Ala Gln Leu Asp Ala Glu Gln Arg Gln Ala Leu Arg Ile Ser Phe Asp Pro Leu Asp Val Pro Arg Ser Glu Phe Val
Val Ala Gly Glu Ala Trp Arg Arg Cys Arg Ser Gly Ala Ala Ala Pro Glu Leu Phe Gly Ile Leu Asp Leu Arg Gly
Leu Trp Phe Val Arg Gly Asn Val Val Arg Asp Leu Ala Ala Phe Ser Lys Arg Glu Leu Leu Pro Trp Asp Gly Trp
Gly Leu Met Ala Thr Arg Glu Asp Ser Ser Pro Ala Glu Leu Ala Leu Leu Asp His Val Ala Glu Leu Thr Leu Ala
Gly Asp Glu Arg His Asp Glu Arg Leu His Leu Gln Asp Ala Glu Pro Gly Leu Arg Val Pro Arg Val Val Leu Set
Phe Asn Leu Asn Gly Ala Glu Val Asp Leu Gly Pro Gly Val Ala Asn
Exemplary has the sequence:

SEQ ID NO:8

Met Arg Ser Asp Leu Ala Phe Tyr Gln Thr Gln Gly Ile Ile Thr Asp Pro Gly Gln His His Asp Leu Leu Thr Gly
Leu Pro Gly Asp Leu Pro Gly Leu Val Lys Val Val Gln Gly Leu Val Val His Val Phe Trp Leu Glu Arg Tyr Gly
Leu Lys Leu Lys Glu Thr Arg Lys Ala Gln Val Gln Leu Arg Trp Ala Glu Lys Gln Leu Glu Arg Ile Arg Ala Leu
Asp Pro Arg Pro Leu Ala Glu Ala Arg Pro Leu Glu Lys Arg Leu Val Gly Asn Cys Arg Asp Phe Thr Val Leu Leu
Val Cys Leu Leu Arg Ala Arg Gly Ile Pro Ala Arg Ala Arg Cys Gly Phe Ala Lys Tyr Phe Glu Ala Gly Arg His
Met Asp His Trp Val Ala Glu Val Trp Asn Ala Glu Leu Gln Arg Trp Thr Leu Val Asp Ala Gln Leu Asp Asp Leu
Gln Arg Lys Ala Leu Ala Ile Pro Phe Asn Pro Leu Asp Val Pro Arg Val Gln Phe Leu Thr Gly Gly Glu Ala Trp
Leu Arg Cys Arg Lys Gly Gln Ala Asp Pro Glu Thr Phe Gly Ile Phe Asp Leu Lys Gly Leu Trp Phe Val Arg Gly
Asp Phe Val Arg Asp Val Ala Ala Leu Asn Lys Val Glu Leu Leu Pro Trp Asp Ala Trp Gly Ile Ala Asp Val Gln
Glu Lys Asp Ile Ser Gly Glu Asp Leu Val Phe Leu Asp Glu Val Ala Glu Leu Ser His Gly Asp Val Glu Arg Phe
Glu Gln Val Lys Gly Leu Tyr Glu Thr Asp Pro Arg Leu His Val Pro Glu Val Ile Asn Ser Tyr Thr Gln Ala Gly
Val Leu Arg Val Asp Leu Gln Ala His Ser
Exemplary has the sequence

SEQ ID NO:10

Met Thr Asp Arg Ala Pro Tyr Ala Ala Gln Ser Pro Ile Ser Asp Pro Gly Asp Met Ser Arg Trp Leu Thr Gly Leu
Pro Ala Asp Phe Ala Ala Leu Arg Ala Leu Ala Arg Pro Leu Val Ala His Tyr Arg Ala Asp Asp Leu Ala Ala Phe
Gly Ile Pro Glu Glu Arg Val Glu Glu Ile Asp Thr Arg Phe Ala Glu Arg Met Leu Ala Arg Leu His Glu Met Glu
Ser Gly Pro Leu Thr Pro Glu Arg Thr Pro Ala Asn Arg Leu Val Gly Cys Cys Arg Asp Phe Thr Leu Leu Tyr Leu
Thr Met Leu Arg His Ala Gly Ile Pro Ala Arg Ser Arg Val Gly Phe Ala Gly Tyr Phe Ala Ala Gly Trp Phe Ile
Asp His Val Val Ala Glu Val Trp Asp Glu Ala Asn Gly Arg Trp Arg Leu Val Asp Pro Gln Leu Ala Asp Val Arg
Thr Asp Pro Asn Asp Gly Phe Pro Ile Asp Thr Leu Asp Ile Pro Arg Asp Arg Phe Leu Val Ala Gly Met Ala Trp
Gln Ala Cys Arg Ser Glu Glu Leu Gln Pro Glu Gln Phe Val Val Asp Pro Asp Leu Asp Ile Pro Val Thr Arg Gly
Trp Leu Gln Leu Arg His Asn Leu Val Gln Asp Leu Ala Ala Leu Thr Lys Arg Glu Met Ile Leu Trp Asp Thr Trp
Gly Ile Leu Gly Asp Glu Pro Val Ala Glu Asp Thr Leu Pro Leu Leu Asp Ser Ile Ala Ala Val Thr Ala Asp Pro
Asp Val Thr Tyr Ala Asp Ala Leu Asn Leu Tyr Glu Arg Glu Pro Gly Val Gln Val Pro Pro Glu Val Met Ser Phe
Asn Met Leu Ala Asn Glu Pro Arg Met Val Ala Ser Gly Val
Exemplary has the sequence

SEQ ID NO:12

Met Leu Ala Ala Gly Val Pro Gly Arg Leu Val Gly Leu His Arg Ile Val Glu Leu Asp Leu Glu Arg Glu Thr Leu
Gly Gln Leu Gln Gln Ala Leu Leu Gln Val Ala Leu Gln Cys Leu Pro Asp Ala Leu Ala Asp Leu Arg Ala Gly Gly
Leu Gly Arg Glu Ala Asp Gly Arg Arg Pro Asp Ala Leu Ala Asp Arg Asp Gly Gly Asp Val Gly Val Gly Leu Leu
Asp Val Gly Ala Glu Leu Pro Val Ala Gly Asp Glu His His Arg Asp Ala Asp His Gly Gly Gly Ile Gly Val Gln
Gln Glu Phe Arg Ser Arg His Ala Val Asp Ala His Ala His Asp Leu Thr Arg Gln Arg Val Arg Gln Gly Ile Gly
Leu Val Ala Gly Leu Arg Val Ile Ala Asp Glu His Arg Gly Ile Glu Ala Leu Val Gln Leu Leu His His Ala His
Arg Met Ala Ala Pro Ala Ala Asp Gln Ala His Ile Leu Arg Gln Val Gly Leu Gln Asp Val Ala Pro Gly Arg Val
Cys Val Leu Asp Gln Asp Leu Leu Gly Pro Arg Arg Val Gly Ala Val Ala Arg Arg Gln His Phe Ala Arg His Leu
Leu Ala Met Leu Gly Ile Val Gly Val Arg Leu Ala Arg Leu Val Pro Val Gly Asp Ala Gly Gly Ala Leu Asp Val
Gly Ala Asp Glu Asp Leu His Ala Thr Pro Leu Cys Lys Arg Ala Pro Leu
Exemplary has the sequence:

SEQ ID NO:14

Met Pro Gln Gly Val Cys Ala Ala Ser Leu Arg Arg Tyr Arg Gln Arg Lys Glu Gln Tyr Leu Met Thr Ile His Gln
Gln Ile Leu Asp Phe Tyr Thr Arg Pro Ala Gly Met Thr Ser Ala Gly Gln Phe Ala Pro Leu Phe Asp Ala Leu Pro
Ser Asp Val Gly Glu Leu Val Arg Ile Ile Gln Gly Leu Gly Val Tyr Asp Leu Val Ala Ser Gly Phe Tyr Gly Phe
Thr Ile Pro Asp Glu Arg Gln Gly Glu Ile His Leu Arg Pro Val Glu Lys Met Leu Gly Arg Leu Leu Ala Leu Asp
Asp Arg Pro Leu Arg Val Ala Arg Pro Val Asp Arg Arg Leu Val Gly Arg Cys Arg His Phe Val Leu Leu Leu Val
Ala Met Leu Arg Ala Lys Gly Val Pro Ala Arg Ala Arg Cys Gly Phe Gly Ser Tyr Phe Arg Arg Gly Phe Phe Glu
Asp His Trp Val Cys Glu Tyr Trp Asn Ala Ala Glu Ala Arg Trp Val Leu Val Asp Pro Gln Phe Asp Glu Val Trp
Arg Glu Thr Leu Gln Ile Asp His Asp Ile Leu Asp Val Pro Arg Asp Arg Phe Leu Val Ala Gly Asp Ala Trp Ala
Gln Cys Arg Ala Gly Ala Ala Asp Pro Ala Lys Phe Glu Ile Val Phe Ala Asp Leu Ser Gly Leu Trp Phe Ile Ala
Gly Asn Leu Val Arg Asp Val Ala Ala Leu Asn Lys Thr Glu Met Leu Pro Trp Asp Val Trp Gly Ala Gln Pro Arg
Pro His Glu Ala Leu Asp Asp Asp Gln Leu Thr Phe Phe Asp Lys Leu Ala Ala Leu Thr Arg Glu Pro Asp Ala Ser
Phe Ala Glu Leu Arg Thr Leu Tyr Glu Gly Asp Asp Arg Leu Arg Val Pro Ala Thr Val Phe Asn Ala Met Arg Asn
Ala Pro Glu Thr Ile Ala Gly
Exemplary has the sequence:

SEQ ID NO:16

Val Asp Gln Thr Gly Ala Asn Asp Ala Leu Val Gly His Gly Arg Arg Pro Ala Ser Ala Gly Arg Arg Asp Arg Pro
Ala Arg Arg Arg Pro Leu Gln Gly Ala Val Leu Gly Ser Gln Glu Arg Ala Ser Gln Arg Gln Arg His Leu Gln Gly
Gly Arg Ala Gln Pro Glu Ala Arg Leu Leu Val Gly Leu Ala Glu His Ala Arg Ala Arg Ile Ala Gly Asp Asp Arg
Ala Gln Pro Gly His Arg Gly His His Ala Asp Ala Asp Pro Arg Ala Val Leu Arg Arg Glu Gly Ala Arg Arg Pro
Arg Pro Arg Leu Glu Arg Arg Pro Arg Pro Pro Gly Glu Leu Pro His Met Thr Pro Gly Gln Ala Val Asp Arg Ala
Phe Ala Gly Leu Pro Gly Asp Pro Ala Ser Leu Ala Gly Val Val Gln Gly Leu Leu Met His Glu His Ile Ala Pro
Ala Tyr Gly Leu Thr Leu Ser Glu Ala Gln His Ala Glu Ala His Thr Arg Pro Val Glu Glu Ile Val Arg Gln Ile
Val Ala His Asp Pro Arg Pro Leu Ala Glu Pro Arg Ala Pro Gly Glu Arg Gln Val Gly Asn Cys Arg His Phe Thr
Leu Leu His Val Thr Met Leu Arg Arg Ala Gly Val Arg Ala Arg Ala Arg Cys Gly Phe Gly Gly Tyr Phe Glu Pro
Gly Lys Phe Leu Asp His Trp Val Thr Glu Tyr Trp Asn Glu Arg Arg Gln Ala Trp Val Leu Val Asp Ala Gln Leu
Asp Ala Arg Gln Arg Glu Leu Phe Lys Ile Ala Phe Asp Pro Leu Asp Val Pro Arg Asp Lys Phe Leu Val Ala Gly
Asp Ala Trp Gln Arg Cys Arg Ala Gly Thr Ala Asp Pro Asn Ala Phe Gly Ile Leu Asp Met His Gly Leu Trp Phe
Val Ala Gly Asn Leu Ile Arg Asp Val Ala Ala Leu Asn Asp His Val Met Leu Pro Trp Asp Val Trp Gly Ala Met
Thr Gln Asn Asp Ala Glu Leu Asp Gln Pro Phe Leu Asp Lys Leu Ala Ala Leu Thr Val Glu Pro Asp Arg His Phe
Gly Glu Leu Arg Ala Val Tyr Gln Asp Pro Arg Val Lys Val Pro Ala Thr Val Phe Asn Ala Ile Arg Asn Arg Pro
Gln Thr Leu

Amidase Signal Sequences

The invention also provides amidase-encoding nucleic acids comprising signal sequences. In one aspect, the signal sequences of the invention are identified following identification of novel amidase polypeptides.

The pathways by which proteins are sorted and transported to their proper cellular location are often referred to as protein targeting pathways. One of the most important elements in all of these targeting systems is a short amino acid sequence at the amino terminus of a newly synthesized polypeptide called the signal sequence. This signal sequence directs a protein to its appropriate location in the cell and is removed during transport or when the protein reaches its final destination. Most lysosomal, membrane, or secreted proteins have an amino-terminal signal sequence that marks them for translocation into the lumen of the endoplasmic reticulum. More than 100 signal sequences for proteins in this group have been determined. The sequences vary in length from 13 to 36 amino acid residues. Various methods of recognition of signal sequences are known to those of skill in the art. For example, in one aspect, novel amidase signal peptides are identified by a method referred to as SignalP. SignalP uses a combined neural network which recognizes both signal peptides and their cleavage sites. (Nielsen, et al., “Identification of prokaryotic and eukaryotic signal peptides and prediction of their cleavage sites.” Protein Engineering, vol. 10, no.1, p. 1-6(1997).

It should be understood that in some aspects amidases of the invention may not have signal sequences. It may be desirable to include a nucleic acid sequence encoding a signal sequence from one amidase operably linked to a nucleic acid sequence of a different amidase or, optionally, a signal sequence from a non-amidase protein may be desired.

Amidases as Selective Catalysts

The invention provides amidases having stereo-, enantio-, regio-, and chemo-selective activity. Enzymes are highly selective catalysts. Their hallmark is the ability to catalyze reactions with exquisite stereo-, regio-, and chemo-selectivities that are unparalleled in conventional synthetic chemistry. Moreover, enzymes are remarkably versatile. In one aspect, the enzymes of the invention are tailored to function in organic solvents, operate at extreme pHs (for example, high pHs and low pHs), extreme temperatures (for example, high temperatures and low temperatures), extreme salinity levels (for example, high salinity and low salinity), and catalyze reactions with compounds that are structurally unrelated to their natural, physiological substrates.

In one aspect, the enzymes of the invention are reactive toward a wide range of natural and unnatural substrates, thus enabling the modification of virtually any organic lead compound. Moreover, unlike traditional chemical catalysts, enzymes are highly enantio- and regio-selective. The high degree of functional group specificity exhibited by enzymes enables one to keep track of each reaction in a synthetic sequence leading to a new active compound. Enzymes are also capable of catalyzing many diverse reactions unrelated to their physiological function in nature. For example, peroxidases catalyze the oxidation of phenols by hydrogen peroxide. Peroxidases can also catalyze hydroxylation reactions that are not related to the native function of the enzyme. Other examples are proteases which catalyze the breakdown of polypeptides. In organic solution some proteases can also acylate sugars, a function unrelated to the native function of these enzymes.

The present invention exploits the unique catalytic properties of enzymes. Whereas the use of biocatalysts (i.e., purified or crude enzymes, non-living or living cells) in chemical transformations normally requires the identification of a particular biocatalyst that reacts with a specific starting compound, the present invention uses selected biocatalysts and reaction conditions that are specific for functional groups that are present in many starting compounds. In one aspect, each biocatalyst is specific for one functional group, or several related functional groups, and can react with many starting compounds containing this functional group.

The biocatalytic reactions of the invention can produce a population of derivatives from a single starting compound. These derivatives can be subjected to another round of biocatalytic reactions to produce a second population of derivative compounds. Thousands of variations of the original compound can be produced with each iteration of biocatalytic derivatization.

The enzymes of the invention can react at specific sites of a starting compound without affecting the rest of the molecule, a process which is very difficult to achieve using traditional chemical methods. This high degree of biocatalytic specificity provides the means to identify a single active compound within the library. The library is characterized by the series of biocatalytic reactions used to produce it, a so-called “biosynthetic history”. Screening the library for biological activities and tracing the biosynthetic history identifies the specific reaction sequence producing the active compound. The reaction sequence is repeated and the structure of the synthesized compound determined. This mode of identification, unlike other synthesis and screening approaches, does not require immobilization technologies, and compounds can be synthesized and tested free in solution using virtually any type of screening assay. In one aspect, the high degree of specificity of enzyme reactions on functional groups allows for the “tracking” of specific enzymatic reactions that make up the biocatalytically produced library.

In alternative aspect, many of the procedural steps are performed using robotic automation. This can enable the execution of many thousands of biocatalytic reactions and screening assays per day. It can also ensure a high level of accuracy and reproducibility. As a result, using the methods of the invention a library of derivative compounds can be produced in a matter of weeks. For further teachings on modification of molecules, including small molecules, see, e.g., PCT/US94/09174.

Uncultivated Organisms (“Environmental Samples”)

In various aspect, the invention provides isolated nucleic acids having at least 50% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 110, SEQ ID NO: 113, and polypeptides having at least 50% sequence identity to a sequence as set forth in SEQ ID NO: 2, SEQ ID NO: 4, SEQ ID NO: 6, ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 113, SEQ ID NO: 114. Sources of the polynucleotides may be isolated from individual organisms (“isolates”), collections of organisms that have been grown in defined media (“enrichment cultures”), or, uncultivated organisms (“environmental samples”). The use of a culture-independent approach to derive polynucleotides encoding novel bioactivities from environmental samples is most preferable since it allows one to access untapped resources of biodiversity.

In one aspect, the invention isolates amidases from “environmental libraries,” which can be generated from environmental samples and can represent the collective genomes of naturally occurring organisms. These “environmental libraries” can be archived in cloning vectors that can be propagated in suitable prokaryotic hosts. In one aspect, because the cloned DNA is initially extracted directly from environmental samples, the libraries are not limited to the small fraction of prokaryotes that can be grown in pure culture. In one aspect, a normalization of the environmental DNA present in these samples allows more equal representation of the DNA from all of the species present in the original sample. This can dramatically increase the efficiency of finding interesting genes from minor constituents of the sample which may be under-represented by several orders of magnitude compared to the-dominant species.

In one aspect, gene libraries generated from one or more uncultivated microorganisms are screened for an activity of interest. Potential pathways encoding bioactive molecules of interest are first captured in prokaryotic cells in the form of gene expression libraries. Polynucleotides encoding activities of interest are isolated from such libraries and introduced into a host cell. The host cell is grown under conditions which promote recombination and/or reductive reassortment creating potentially active biomolecules with novel or enhanced activities. Expression in a host cell may be improved (e.g., yield) by evolving or modifying a polynucleotide of interest.

The microorganisms from which the polynucleotide may be prepared include prokaryotic microorganisms, such as Eubacteria and Archaebacteria, and lower eukaryotic microorganisms such as fungi, some algae and protozoa. Polynucleotides may be isolated from environmental samples in which case the nucleic acid may be recovered without culturing of an organism or recovered from one or more cultured organisms. In one aspect, such microorganisms may be extremophiles, such as hyperthermophiles, psychrophiles, psychrotrophs, halophiles, barophiles and acidophiles. Polynucleotides encoding enzymes isolated from extremophilic microorganisms are particularly preferred. Such enzymes may function at temperatures above 100° C. in terrestrial hot springs and deep sea thermal vents, at temperatures below 0° C. in arctic waters, in the saturated salt environment of the Dead Sea, at pH values around 0 in coal deposits and geothermal sulfur-rich springs, or at pH values greater than 11 in sewage sludge. For example, several esterases and lipases cloned and expressed from extremophilic organisms show high activity throughout a wide range of temperatures and pHs.

In one aspect of the invention complex environmental libraries are screened using high throughput screening methods to identify novel enzymes with secondary amidase activity.

Fluorescent Amidase Substrates

In another aspect of the invention, commercially available fluorescent substrates (e.g. CBZ-L-ALA-AMC, CBZ-L-ARG-AMC, CBZ-L-ASP-AMC, CBZ-L-LEU-AMC, (BZ-L-PHE-AMC) are used for discovery or a novel substrate for secondary amidase discovery. In another aspect of the invention, the libraries are screened for secondary amidase activity using 7-(ε-D-2-aminoadipoylamido)-4-methylcoumarin.

In the present invention the novel fluorescent secondary amidase substrate, 7-(ε-D-2-aminoadipoylamido)-4-methylcoumarin has been designed, synthesized, and demonstrated. The substrate was specifically designed for use in high throughput (HT) activity-based, whole cell screening for the discovery of a secondary amidase activity that can directly convert the antibiotic cephalosporin C to 7-aminocephalosporanic acid (7-ACA). The substrate utilizes the D-2-aminoadipoyl side chain found on cephalosporin C attached to the fluorescent reporter 7-amino-4-methylcoumarin through an amide linkage. Therefore, enzymes that can cleave this substrate are likely recognizing the D-2-aminoadipoyl side chain and cleaving the fluorescent substrate at a position equivalent to the desired site of cleavage in cephalosporin C. The substrate has been used for HT screening of environmental libraries and it has identified a novel secondary amidase that can convert cephalosporin C to 7-ACA. However, 7-(ε-D-2-aminoadipoylamido)-4-methylcoumarin is not limited to use as a substrate for the discovery of cephalosporin C amidases and the substrate has identified a number of novel hydrolases with secondary amidase activity. In addition, the substrate has proved useful for the kinetic characterization of crude or purified enzyme preparations in assays designed to determine Km and specific activity values for these enzymes.

The substrate, 7-(ε-D-2-aminoadipoylamido)-4-methylcoumarin, is suitable for high throughput screening and it has been used to identify secondary amidases in both conventional 1536 well and 100,000 well GIGAMATRIX™ format (Diversa Corporation, San Diego, Calif.). In addition, the substrate is very sensitive because it utilizes a fluorescent reporter, 7-amino-4-methylcoumarin. Finally, the substrate is specific, containing the D-2-aminoadipoyl side chain found in cephalosporin C.

Secondary amidases of the invention can be screened using the methods and substrates described herein. For example, amidase activity can be identified using a cephalosporin C or a hydantoin as the amidase specific substrate.

Polynucleotides selected and isolated as described herein are introduced into a suitable host cell. A suitable host cell is any cell which is capable of promoting recombination and/or reductive reassortment. The selected polynucleotides can be already in a vector which includes appropriate control sequences. The host cell can be a higher eukaryotic cell, such as a mammalian cell, or a lower eukaryotic cell, such as a yeast cell, or preferably, the host cell can be a prokaryotic cell, such as a bacterial cell. Introduction of the construct into the host cell can be effected by calcium phosphate transfection, DEAE-Dextran mediated transfection, or electroporation (Davis et al., 1986).

Exemplary appropriate hosts include bacterial cells, such as E. coli, Streptomyces, Salmonella typhimurium; fungal cells, such as yeast; insect cells such as Drosophila S2 and Spodoptera Sf9; animal cells such as CHO, COS or Bowes melanoma; adenoviruses; and plant cells. The selection of an appropriate host is deemed to be within the scope of those skilled in the art from the teachings herein.

With particular references to various mammalian cell culture systems that can be employed to express recombinant protein, examples of mammalian expression systems include the COS-7 lines of monkey kidney fibroblasts, described in “SV40-transformed simian cells support the replication of early SV40 mutants” (Gluzman, 1981), and other cell lines capable of expressing a compatible vector, for example, the C127, 3T3, CHO, HeLa and B14K cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide the required nontranscribed genetic elements.

Host cells containing the polynucleotides of interest can be cultured in conventional nutrient media modified as appropriate for activating promoters, selecting transformants or amplifying genes. The culture conditions, such as temperature, pH and the like, are those previously used with the host cell selected for expression, and will be apparent to the ordinarily skilled artisan. The clones which are identified as having the specified enzyme activity may then be sequenced to identify the polynucleotide sequence encoding an enzyme having the enhanced activity.

Generating Biochemical Pathways

In another aspect, the methods of the invention can be used to generate novel polynucleotides encoding biochemical pathways from one or more operons or gene clusters or portions thereof. For example, bacteria and many eukaryotes have a coordinated mechanism for regulating genes whose products are involved in related processes. The genes are clustered, in structures referred to as “gene clusters,” on a single chromosome and are transcribed together under the control of a single regulatory sequence, including a single promoter which initiates transcription of the entire cluster. Thus, a gene cluster is a group of adjacent genes that are either identical or related, usually as to their function. An example of a biochemical pathway encoded by gene clusters are polyketides.

Gene cluster DNA can be isolated from different organisms and ligated into vectors, particularly vectors containing expression regulatory sequences which can control and regulate the production of a detectable protein or protein-related array activity from the ligated gene clusters. Use of vectors which have an exceptionally large capacity for exogenous DNA introduction are particularly appropriate for use with such gene clusters and are described by way of example herein to include the f-factor (or fertility factor) of E. coli. This f-factor of E. coli is a plasmid which affect high-frequency transfer of itself during conjugation and is ideal to achieve and stably propagate large DNA fragments, such as gene clusters from mixed microbial samples. A particularly preferred embodiment is to use cloning vectors, referred to as “fosmids” or bacterial artificial chromosome (BAC) vectors. These are derived from E. coli f-factor which is able to stably integrate large segments of genomic DNA. When integrated with DNA from a mixed uncultured environmental sample, this makes it possible to achieve large genomic fragments in the form of a stable “environmental DNA library.” Another type of vector for use in the present invention is a cosmid vector. Cosmid vectors were originally designed to clone and propagate large segments of genomic DNA. Cloning into cosmid vectors is described in detail in Sambrook et al., Molecular Cloning: A Laboratory Manual, 2nd Ed., Cold Spring Harbor Laboratory Press (1989). Once ligated into an appropriate vector, two or more vectors containing different polyketide synthase gene clusters can be introduced into a suitable host cell. Regions of partial sequence homology shared by the gene clusters will promote processes which result in sequence reorganization resulting in a hybrid gene cluster. The novel hybrid gene cluster can then be screened for enhanced activities not found in the original gene clusters.

In a one aspect, the invention provides a method for producing a biologically active hybrid amidase polypeptide and screening such a polypeptide for enhanced activity by:

1) introducing at least a first polynucleotide in operable linkage and a second polynucleotide in operable linkage, said at least first polynucleotide and second polynucleotide sharing at least one region of partial sequence homology, into a suitable host cell;

2) growing the host cell under conditions which promote sequence reorganization resulting in a hybrid polynucleotide in operable linkage;

3) expressing a hybrid polypeptide encoded by the hybrid polynucleotide;

4) screening the hybrid polypeptide under conditions which promote identification of enhanced biological activity; and

5) isolating the a polynucleotide encoding the hybrid polypeptide.

Methods for screening for various enzyme activities are known to those of skill in the art and are discussed throughout the present specification. Such methods may be employed when isolating the polypeptides and polynucleotides of the invention.

Hybrid Amidases, Antibodies and Peptide Libraries

In one aspect, the invention provides hybrid amidases, antibodies and fusion proteins, including peptide libraries, comprising sequences of the invention. The peptide libraries of the invention can be used to isolate peptide modulators (e.g., activators or inhibitors) of targets, such as amidase substrates, receptors, enzymes. The peptide libraries of the invention can be used to identify formal binding partners of targets, such as ligands, e.g., cytokines, hormones and the like.

In one aspect, the fusion proteins of the invention (e.g., the peptide moiety) are conformationally stabilized (relative to linear peptides) to allow a higher binding affinity for targets. The invention provides fusions of amidases and antibodies of the invention and other peptides, including known and random peptides. They can be fused in such a manner that the structure of the amidases is not significantly perturbed and the peptide is metabolically or structurally conformationally stabilized. This allows the creation of a peptide library that is easily monitored both for its presence within cells and its quantity.

Amino acid sequence variants of the invention can be characterized by a predetermined nature of the variation, a feature that sets them apart from a naturally occurring form, e.g., an allelic or interspecies variation of an amidase sequence. In one aspect, the variants of the invention exhibit the same qualitative biological activity as the naturally occurring analogue. Alternatively, the variants can be selected for having modified characteristics. In one aspect, while the site or region for introducing an amino acid sequence variation is predetermined, the mutation per se need not be predetermined. For example, in order to optimize the performance of a mutation at a given site, random mutagenesis may be conducted at the target codon or region and the expressed amidase variants screened for the optimal combination of desired activity. Techniques for making substitution mutations at predetermined sites in DNA having a known sequence are well known, as discussed herein for example, M13 primer mutagenesis and PCR mutagenesis. Screening of the mutants can be done using assays of proteolytic activities. In alternative aspects, amino acid substitutions can be single residues; insertions can be on the order of from about 1 to 20 amino acids, although considerably larger insertions can be done. Deletions can range from about 1 to about 20, 30, 40, 50, 60, 70 residues or more. To obtain a final derivative with the optimal properties, substitutions, deletions, insertions or any combination thereof may be used. Generally, these changes are done on a few amino acids to minimize the alteration of the molecule. However, larger changes may be tolerated in certain circumstances.

The invention provides amidases and antibodies where the structure of the polypeptide backbone, the secondary or the tertiary structure, e.g., an alpha-helical or beta-sheet structure, has been modified. In one aspect, the charge or hydrophobicity has been modified. In one aspect, the bulk of a side chain has been modified. Substantial changes in function or immunological identity are made by selecting substitutions that are less conservative. For example, substitutions can be made which more significantly affect: the structure of the polypeptide backbone in the area of the alteration, for example a alpha-helical or a beta-sheet structure; a charge or a hydrophobic site of the molecule, which can be at an active site; or a side chain. The invention provides substitutions in polypeptide of the invention where (a) a hydrophilic residues, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine. The variants can exhibit the same qualitative biological activity (i.e. amidase activity) although variants can be selected to modify the characteristics of the amidases as needed.

In one aspect, amidases and antibodies of the invention comprise epitopes or purification tags, signal sequences or other fusion sequences, etc. In one aspect, the amidases and antibodies of the invention can be fused to a random peptide to form a fusion polypeptide. By “fused” or “operably linked” herein is meant that the random peptide and the amidase are linked together, in such a manner as to minimize the disruption to the stability of the amidase structure, e.g., it retains amidase activity. The fusion polypeptide (or fusion polynucleotide encoding the fusion polypeptide) can comprise further components as well, including multiple peptides at multiple loops.

In one aspect, the peptides and nucleic acids encoding them are randomized, either fully randomized or they are biased in their randomization, e.g. in nucleotide/residue frequency generally or per position. “Randomized” means that each nucleic acid and peptide consists of essentially random nucleotides and amino acids, respectively. In one aspect, the nucleic acids which give rise to the peptides can be chemically synthesized, and thus may incorporate any nucleotide at any position. Thus, when the nucleic acids are expressed to form peptides, any amino acid residue may be incorporated at any position. The synthetic process can be designed to generate randomized nucleic acids, to allow the formation of all or most of the possible combinations over the length of the nucleic acid, thus forming a library of randomized nucleic acids. The library can provide a sufficiently structurally diverse population of randomized expression products to affect a probabilistically sufficient range of cellular responses to provide one or more cells exhibiting a desired response. Thus, the invention provides an interaction library large enough so that at least one of its members will have a structure that gives it affinity for some molecule, protein, or other factor.

Screening Methodologies and “On-Line” Monitoring Devices

In practicing the methods of the invention, a variety of apparatus and methodologies can be used to in conjunction with the polypeptides and nucleic acids of the invention, e.g., to screen polypeptides for amidase or antibody activity, to screen compounds as potential modulators, e.g., activators or inhibitors, of an amidase activity, for antibodies that bind to a polypeptide of the invention, for nucleic acids that hybridize to a nucleic acid of the invention, to screen for cells expressing a polypeptide of the invention and the like.

Capillary Arrays

Capillary arrays, such as the GIGAMATRIX™, Diversa Corporation, San Diego, Calif., can be used to in the methods of the invention. Nucleic acids or polypeptides of the invention can be immobilized to or applied to an array, including capillary arrays. Arrays can be used to screen for or monitor libraries of compositions (e.g., small molecules, antibodies, nucleic acids, etc.) for their ability to bind to or modulate the activity of a nucleic acid or a polypeptide of the invention. Capillary arrays provide another system for holding and screening samples. For example, a sample screening apparatus can include a plurality of capillaries formed into an array of adjacent capillaries, wherein each capillary comprises at least one wall defining a lumen for retaining a sample. The apparatus can further include interstitial material disposed between adjacent capillaries in the array, and one or more reference indicia formed within of the interstitial material. A capillary for screening a sample, wherein the capillary is adapted for being bound in an array of capillaries, can include a first wall defining a lumen for retaining the sample, and a second wall formed of a filtering material, for filtering excitation energy provided to the lumen to excite the sample. A polypeptide or nucleic acid, e.g., a ligand, can be introduced into a first component into at least a portion of a capillary of a capillary array. Each capillary of the capillary array can comprise at least one wall defining a lumen for retaining the first component. An air bubble can be introduced into the capillary behind the first component. A second component can be introduced into the capillary, wherein the second component is separated from the first component by the air bubble. A sample of interest can be introduced as a first liquid labeled with a detectable particle into a capillary of a capillary array, wherein each capillary of the capillary array comprises at least one wall defining a lumen for retaining the first liquid and the detectable particle, and wherein the at least one wall is coated with a binding material for binding the detectable particle to the at least one wall. The method can further include removing the first liquid from the capillary tube, wherein the bound detectable particle is maintained within the capillary, and introducing a second liquid into the capillary tube. The capillary array can include a plurality of individual capillaries comprising at least one outer wall defining a lumen. The outer wall of the capillary can be one or more walls fused together. Similarly, the wall can define a lumen that is cylindrical, square, hexagonal or any other geometric shape so long as the walls form a lumen for retention of a liquid or sample. The capillaries of the capillary array can be held together in close proximity to form a planar structure. The capillaries can be bound together, by being fused (e.g., where the capillaries are made of glass), glued, bonded, or clamped side-by-side. The capillary array can be formed of any number of individual capillaries, for example, a range from 100 to 4,000,000 capillaries. A capillary array can form a micro titer plate having about 100,000 or more individual capillaries bound together.

Arrays, or “Biochips”

Nucleic acids or polypeptides of the invention can be immobilized to or applied to an array. Arrays can be used to screen for or monitor libraries of compositions (e.g., small molecules, antibodies, nucleic acids, etc.) for their ability to bind to or modulate the activity of a nucleic acid or a polypeptide of the invention. For example, in one aspect of the invention, a monitored parameter is transcript expression of an amidase gene. One or more, or, all the transcripts of a cell can be measured by hybridization of a sample comprising transcripts of the cell, or, nucleic acids representative of or complementary to transcripts of a cell, by hybridization to immobilized nucleic acids on an array, or “biochip.” By using an “array” of nucleic acids on a microchip, some or all of the transcripts of a cell can be simultaneously quantified. Alternatively, arrays comprising genomic nucleic acid can also be used to determine the genotype of a newly engineered strain made by the methods of the invention. Polypeptide arrays” can also be used to simultaneously quantify a plurality of proteins. The present invention can be practiced with any known “array,” also referred to as a “microarray” or “nucleic acid array” or “polypeptide array” or “antibody array” or “biochip,” or variation thereof. Arrays are generically a plurality of “spots” or “target elements,” each target element comprising a defined amount of one or more biological molecules, e.g., oligonucleotides, immobilized onto a defined area of a substrate surface for specific binding to a sample molecule, e.g., mRNA transcripts.

In practicing the methods of the invention, any known array and/or method of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32. See also published U.S. patent applications Ser. Nos. 20010018642; 20010019827; 20010016322; 20010014449; 20010014448; 20010012537; 20010008765.

Antibodies and Antibody-Based Screening Methods

The invention provides isolated or recombinant antibodies that specifically bind to an amidase of the invention. These antibodies can be used to isolate, identify or quantify the amidases of the invention or related polypeptides. These antibodies can be used to isolate other polypeptides within the scope the invention or other related amidases. The antibodies can be designed to bind to an active site of an amidase. Thus, the invention provides methods of inhibiting amidases using the antibodies of the invention.

The antibodies can be used in immunoprecipitation, staining, immunoaffinity columns, and the like. If desired, nucleic acid sequences encoding for specific antigens can be generated by immunization followed by isolation of polypeptide or nucleic acid, amplification or cloning and immobilization of polypeptide onto an array of the invention. Alternatively, the methods of the invention can be used to modify the structure of an antibody produced by a cell to be modified, e.g., an antibody's affinity can be increased or decreased. Furthermore, the ability to make or modify antibodies can be a phenotype engineered into a cell by the methods of the invention.

Methods of immunization, producing and isolating antibodies (olyclonal and monoclonal) are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

Polypeptides or peptides can be used to generate antibodies which bind specifically to the polypeptides, e.g., the amidases, of the invention. The resulting antibodies may be used in immunoaffinity chromatography procedures to isolate or purify the polypeptide or to determine whether the polypeptide is present in a biological sample. In such procedures, a protein preparation, such as an extract, or a biological sample is contacted with an antibody capable of specifically binding to one of the polypeptides of the invention.

In immunoaffinity procedures, the antibody is attached to a solid support, such as a bead or other column matrix. The protein preparation is placed in contact with the antibody under conditions in which the antibody specifically binds to one of the polypeptides of the invention. After a wash to remove non-specifically bound proteins, the specifically bound polypeptides are eluted.

The ability of proteins in a biological sample to bind to the antibody may be determined using any of a variety of procedures familiar to those skilled in the art. For example, binding may be determined by labeling the antibody with a detectable label such as a fluorescent agent, an enzymatic label, or a radioisotope. Alternatively, binding of the antibody to the sample may be detected using a secondary antibody having such a detectable label thereon. Particular assays include ELISA assays, sandwich assays, radioimmunoassays, and Western Blots.

Polyclonal antibodies generated against the polypeptides of the invention can be obtained by direct injection of the polypeptides into an animal or by administering the polypeptides to a non-human animal. The antibody so obtained will then bind the polypeptide itself. In this manner, even a sequence encoding only a fragment of the polypeptide can be used to generate antibodies which may bind to the whole native polypeptide. Such antibodies can then be used to isolate the polypeptide from cells expressing that polypeptide.

For preparation of monoclonal antibodies, any technique which provides antibodies produced by continuous cell line cultures can be used. Examples include the hybridoma technique, the trioma technique, the human B-cell hybridoma technique, and the EBV-hybridoma technique (see, e.g., Cole (1985) in Monoclonal Antibodies and Cancer Therapy, Alan R. Liss, Inc., pp. 77-96).

Techniques described for the production of single chain antibodies (see, e.g., U.S. Pat. No. 4,946,778) can be adapted to produce single chain antibodies to the polypeptides of the invention. Alternatively, transgenic mice may be used to express humanized antibodies to these polypeptides or fragments thereof.

Antibodies generated against the polypeptides of the invention may be used in screening for similar polypeptides (e.g., amidases) from other organisms and samples. In such techniques, polypeptides from the organism are contacted with the antibody and those polypeptides which specifically bind the antibody are detected. Any of the procedures described above may be used to detect antibody binding.

Kits

The invention provides kits comprising the compositions, e.g., nucleic acids, expression cassettes, vectors, cells, transgenic seeds or plants or plant parts, polypeptides (e.g., amidases) and/or antibodies of the invention. The kits also can contain instructional material teaching the methodologies and industrial uses of the invention, as described herein. For example, the kits can be for increasing flavors in food (e.g., enzyme ripened cheeses), promoting bacterial and fungal killing, modifying and de-protecting fine chemical intermediates, synthesizing peptide bonds, carrying out chiral resolutions, hydrolyzing cephalosporin C using the enzymes of the invention.

Measuring Metabolic Parameters

The methods of the invention provide whole cell evolution, or whole cell engineering, of a cell to develop a new cell strain having a new phenotype, e.g., a new or modified amidase activity, by modifying the genetic composition of the cell. The genetic composition can be modified by addition to the cell of a nucleic acid of the invention. To detect the new phenotype, at least one metabolic parameter of a modified cell is monitored in the cell in a “real time” or “on-line” time frame. In one aspect, a plurality of cells, such as a cell culture, is monitored in “real time” or “on-line.” In one aspect, a plurality of metabolic parameters is monitored in “real time” or “on-line.” Metabolic parameters can be monitored using the amidases of the invention.

Metabolic flux analysis (MFA) is based on a known biochemistry framework. A linearly independent metabolic matrix is constructed based on the law of mass conservation and on the pseudo-steady state hypothesis (PSSH) on the intracellular metabolites. In practicing the methods of the invention, metabolic networks are established, including the:

identity of all pathway substrates, products and intermediary metabolites

identity of all the chemical reactions interconverting the pathway metabolites, the stoichiometry of the pathway reactions,

identity of all the enzymes catalyzing the reactions, the enzyme reaction kinetics,

the regulatory interactions between pathway components, e.g. allosteric interactions, enzyme-enzyme interactions etc,

intracellular compartmentalization of enzymes or any other supramolecular organization of the enzymes, and,

the presence of any concentration gradients of metabolites, enzymes or effector molecules or diffusion barriers to their movement.

Once the metabolic network for a given strain is built, mathematic presentation by matrix notion can be introduced to estimate the intracellular metabolic fluxes if the on-line metabolome data is available. Metabolic phenotype relies on the changes of the whole metabolic network within a cell. Metabolic phenotype relies on the change of pathway utilization with respect to environmental conditions, genetic regulation, developmental state and the genotype, etc. In one aspect of the methods of the invention, after the on-line MFA calculation, the dynamic behavior of the cells, their phenotype and other properties are analyzed by investigating the pathway utilization. For example, if the glucose supply is increased and the oxygen decreased during the yeast fermentation, the utilization of respiratory pathways will be reduced and/or stopped, and the utilization of the fermentative pathways will dominate. Control of physiological state of cell cultures will become possible after the pathway analysis. The methods of the invention can help determine how to manipulate the fermentation by determining how to change the substrate supply, temperature, use of inducers, etc. to control the physiological state of cells to move along desirable direction. In practicing the methods of the invention, the MFA results can also be compared with transcriptome and proteome data to design experiments and protocols for metabolic engineering or gene shuffling, etc.

In practicing the methods of the invention, any modified or new phenotype can be conferred and detected, including new or improved characteristics in the cell. Any aspect of metabolism or growth can be monitored.

Monitoring Expression of an mRNA Transcript

In one aspect of the invention, the engineered phenotype comprises increasing or decreasing the expression of an mRNA transcript (e.g., an amidase message) or generating new (e.g., amidase) transcripts in a cell. This increased or decreased expression can be traced by testing for the presence of an amidase of the invention or by amidase activity assays. mRNA transcripts, or messages, also can be detected and quantified by any method known in the art, including, e.g., Northern blots, quantitative amplification reactions, hybridization to arrays, and the like. Quantitative amplification reactions include, e.g., quantitative PCR, including, e.g., quantitative reverse transcription polymerase chain reaction, or RT-PCR; quantitative real time RT-PCR, or “real-time kinetic RT-PCR” (see, e.g., Kreuzer (2001) Br. J. Haematol. 114:313-318; Xia (2001) Transplantation 72:907-914). In one aspect of the invention, the engineered phenotype is generated by knocking out expression of a homologous gene. The gene's coding sequence or one or more transcriptional control elements can be knocked out, e.g., promoters or enhancers. Thus, the expression of a transcript can be completely ablated or only decreased.

In one aspect of the invention, the engineered phenotype comprises increasing the expression of a homologous gene. This can be effected by knocking out of a negative control element, including a transcriptional regulatory element acting in cis- or trans-, or, mutagenizing a positive control element. One or more, or, all the transcripts of a cell can be measured by hybridization of a sample comprising transcripts of the cell, or, nucleic acids representative of or complementary to transcripts of a cell, by hybridization to immobilized nucleic acids on an array.

Monitoring Expression of a Polypeptides, Peptides and Amino Acids

In one aspect of the invention, the engineered phenotype comprises increasing or decreasing the expression of a polypeptide (e.g., an amidase) or generating new polypeptides in a cell. This increased or decreased expression can be traced by determining the amount of amidase present or by amidase activity assays. Polypeptides, peptides and amino acids also can be detected and quantified by any method known in the art, including, e.g., nuclear magnetic resonance (NMR), spectrophotometry, radiography (protein radiolabeling), electrophoresis, capillary electrophoresis, high performance liquid chromatography (HPLC), thin layer chromatography (TLC), hyperdiffusion chromatography, various immunological methods, e.g. immunoprecipitation, immunodiffusion, immuno-electrophoresis, radioimmunoassays (RIAs), enzyme-linked immunosorbent assays (ELISAs), immuno-fluorescent assays, gel electrophoresis (e.g., SDS-PAGE), staining with antibodies, fluorescent activated cell sorter (FACS), pyrolysis mass spectrometry, Fourier-Transform Infrared Spectrometry, Raman spectrometry, GC-MS, and LC-Electrospray and cap-LC-tandem-electrospray mass spectrometries, and the like. Novel bioactivities can also be screened using methods, or variations thereof, described in U.S. Pat. No. 6,057,103. Furthermore, as discussed below in detail, one or more, or, all the polypeptides of a cell can be measured using a protein array.

Industrial, Food Processing and Pharmaceutical Applications

The enzymes of the invention can be used for a variety of industrial, food processing and pharmaceutical applications, e.g., to increase flavor in food (e.g., “enzyme ripened cheese”), to promote bacterial and fungal killing, to modify and/or de-protect fine chemical intermediates, to synthesize peptide bonds, to carry out chiral resolutions and/or to hydrolyze an amide-containing drug, e.g., an antibiotic, such as cephalosporin C. The enzymes of the invention can have secondary amidase activity, e.g., they can catalyze the hydrolysis of amide. Polypeptides of the invention include proteins having peptidase, protease and/or hydantoinase activity. In one aspect, amidases of the invention can catalyze the selective hydrolytic elimination of the free amino group on the C-terminal end of peptide amides, but do not cleave peptide bonds. In one aspect, amidases of the invention can catalyze the selective hydrolytic elimination of the free amino group at the C-terminal location of peptide amides.

Enantio-Selective Processes

The compositions and methods of the invention can be used for the resolution of racemic mixtures of optically active compounds, including the stereochemical purification of chiral amides. In one aspect, the enzymes of the invention are selective for the L, or “natural” enantiomer of amino acid derivatives. In one aspect, they are useful for the production of optically active compounds. These reactions can be performed in the presence of the chemically more reactive ester functionally.

The compositions and methods of the invention can be used in the enzymatic resolution and chiral synthesis of compounds that are poorly water-soluble or that are produced via product-inhibited reactions. Racemic mixtures that can be processed by the invention include mixtures of isomers of chiral amides and other compounds. A chiral precursors can be biotransformed into valuable chiral products, including amino nitrile compounds, which can stereoselectively be converted into such valuable products as amino acids (e.g., D-amino acids and methyl dopa) and amino amides.

In one aspect, the amidases of the invention are used to generate enantiomerically pure L-amino acids from racemic mixtures of N-protected amino acid amides. In one aspect, a racemic mixture of N-protected amino acid amides is incubated with the peptide amidase. It is reacted until the complete conversion of the N-protected L-amino acid amide. In one aspect, the N-protected L-amino acid is separated from the N-protected D-amino acid amide based on the differences of charge.

In one aspect, the amidases of the invention are used to obtain non-proteinaceous D-amino acids, e.g., using N-protected racemic amino acid amides, such as N-acetyl-neopentylglycine amide, N-acetyl-naphthylalanine amide, N-acetylphenylglycine amide or similar derivatives. In one aspect, the N-acetyl-L-amino acid amides are enzymatically hydrolyzed and the N-acetyl-D-amino acid amides separated from the reaction mixture by chromatography and converted by acid hydrolysis into the free D-amino acids.

In one aspect, the amidases of the invention are used to produce peptides by the enzymatic conversion of amino acid alkyl esters (optionally N-protected amino acid alkyl esters) or N-protected peptide alkyl esters (optionally N-protected peptide alkyl esters) with amino acid amides in aqueous phase or an aqueous-organic environment. In one aspect, the enzyme catalyzes peptidic bonding and enzymatic splitting off of the amide protective group. The synthesis can be allowed to take place in a continuous manner. The peptide amide can be hydrolyzed by the peptide amidase enzymatically to the peptide. In one aspect, the peptide is separated by its charge from the reaction mixture. The amidases of the invention can be used with any enantio-selective processing method, as described e.g. in U.S. Pat. No. 4,800,162.

Pharmaceutical Applications

The amidases of the invention can be used in pharmaceutical applications, for example, to process a drug, e.g., to hydrolyze cephalosporin C to 7-aminocephalosporanic acid or a corresponding derivative thereof The enzymes of the invention can be used to generate 7-aminocephalosporanic acid (7-ACA) and semi-synthetic cephalosporin antibiotics, including caphalothin, cephaloridine and cefuroxime. The invention provides drugs and pharmaceutical comprising a polypeptide of the invention.

In one aspect, the invention provides an enzyme process (using a polypeptide of the invention) for the one-step conversion of cephalosporin C or a derivative thereof into 7-aminocephalosporanic acid or a corresponding derivative thereof. This process can incorporate any pharmaceutical method, e.g., a described in U.S. Pat. No. 6,297,032.

Food Processing

Polypeptides of the invention, including proteins having peptidase, protease and/or hydantoinase activity, can be used in any aspect of food processing. For example, the enzymes of the invention can influence the fermentation characteristics of the bacteria, such as bacterial speed of growth, acid production and survival. In the manner the enzymes of the invention can be used to influence dairy product flavo, and functional and textural characteristics, as well as influencing the fermentation characteristics of the bacteria, such as is speed of growth, acid production and survival. The enzymes of the invention can be used as cell wall hydrolases.

The polypeptides of the invention can be used in the process of cheese ripening. The polypeptides of the invention can be used in the hydrolysis of milk caseins. Peptidases are important enzymes in the process of cheese ripening and the development of cheese flavor. The hydrolysis of milk caseins in cheese results in textural changes and the development of cheese flavors. Proteolytic enzymes can be added to starter cultures or any time during the cheese ripening process. In one aspect, a polypeptide of the invention used in cheese ripening has N-acetylnuramoyl-L-alanine amidase activity. The amidases of the invention can be used in conjunction with muramidases, lysozymes, including N-acetyl muramidase, muramidase and N-acetylglucosaminidase. The amidases of the invention can be used with any food processing (e.g., cheese processing or ripening) method, as described e.g. in U.S. Pat. No. 6,476,209.

Bacterial and Fungal Killing

The amidases of the invention can be used to promote bacterial and fungal killing. The enzymes of the invention can be used as cell wall hydrolases. The amidases of the invention can be used in methods for the enzymatic decontamination of specimens as a means to control and prevent microbiological contamination. The methods of the invention can utilize one or more amidases of the invention, or other lytic enzymes, to compromise the viability or structural integrity of contaminating microorganisms (e.g., non-gram negative microorganisms). The amidases and methods of the invention can be used to promote bacterial and fungal killing, e.g., used as a bacteriocide, a fungicide, on any surface or sample, solid or liquid. The composition and method of the invention can be used alone or in addition to other agents to facilitate microbiological contamination, including antibiotics. The amidases of the invention can be used with any antimicrobial method, product or device (e.g., bacteriocides, fungicides), as described e.g. in U.S. Pat. Nos. 5,985,593; 5,369,016; 5,955,258.

The amidases of the invention can also be used to improve the shelf life of a consumer product or a food product. The enzyme of the invention is incorporated into a food product or a consumer product in such amount that the growth of spoiling bacteria or pathogenic bacteria is inhibited or their viability is strongly reduced. Thus, the invention provides consumer products and food products comprising the amidases of the invention. Consumer products and food products of the invention comprise edible products, cosmetic products, products for cleaning fabrics, hard surfaces and human skin and the like. Food products and consumer products of the invention include bread and bread improvers, butter, margarine, low calorie substitutes of butter, cheeses, dressings, mayonnaise-like products, meat products, food ingredients containing peptides, shampoos, creams or lotions, e.g., for treatment of the human skin, soap and soap-replacement products, washing powders or liquids, and/or products for cleaning food production equipment and kitchen utensils.

Modifying Small Molecules

The invention provides methods for modifying small molecules using the amidases of the invention. In one aspect, the invention comprises contacting a polypeptide encoded by a polynucleotide of the invention (which include enzymatically active fragments thereof) with a small molecule to produce a modified small molecule. A library of modified small molecules is tested to determine if a modified small molecule is present within the library which exhibits a desired activity. A specific biocatalytic reaction which produces the modified small molecule of desired activity is identified by systematically eliminating each of the biocatalytic reactions used to produce a portion of the library, and then testing the small molecules produced in the portion of the library for the presence or absence of the modified small molecule with the desired activity. The specific biocatalytic reactions which produce the modified small molecule of desired activity is optionally repeated. The biocatalytic reactions are conducted with a group of biocatalysts that react with distinct structural moieties found within the structure of a small molecule, each biocatalyst is specific for one structural moiety or a group of related structural moieties; and each biocatalyst reacts with many different small molecules which contain the distinct structural moiety.

### EXAMPLES

The invention will be further described with reference to the following examples; however, it is to be understood that the invention is not limited to such examples.

### Example 1

Synthesis of Novel Fluorescent Substrate

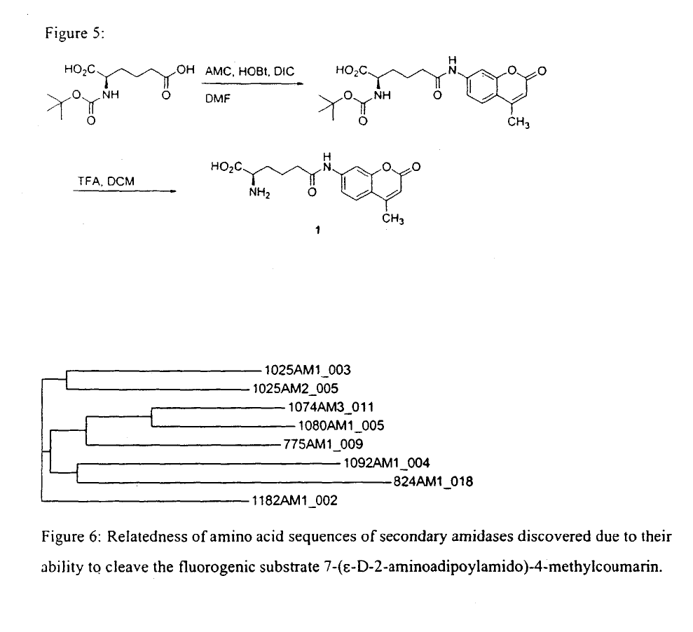

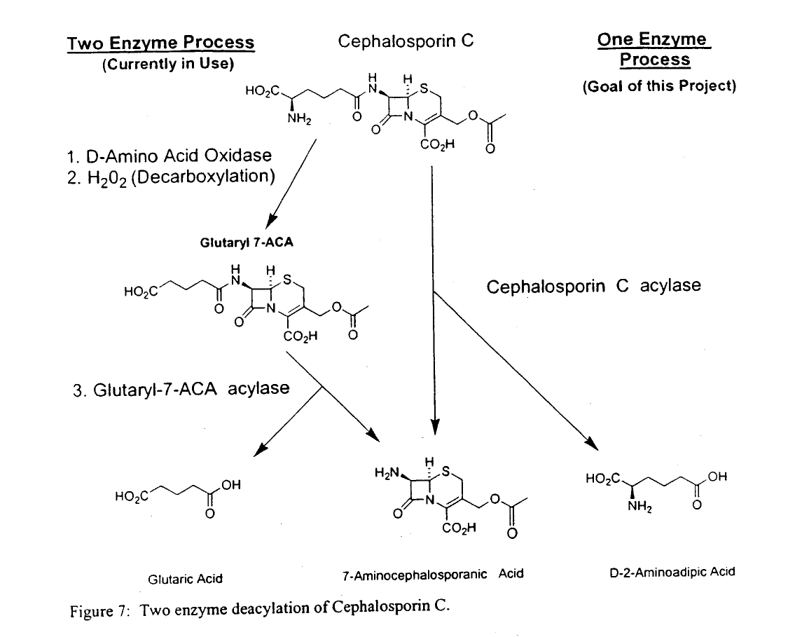

The fluorescent amidase substrate 7-(ε-D-2-aminoadipoylamido)-4-methylcoumarin (FIG. 7, structure “1”) was prepared by a two-step synthetic procedure (FIG. 5). 7-amino-4-methylcoumarin (350 mg, 2.0 mmol), N-Boc-D-2-aminoadipic acid (523 mg, 2.0 mmol) and N-hydroxybenzotriazole (270 mg, 2.0 mmol) were dissolved in 2.5 mL of dimethylformamide. Diisopropylcarbodiimide (313 μL, 2.0 mmol) was added, and the mixture was stirred at room temperature for 24 hours. It was then filtered to remove diisopropylurea by-product, and concentrated under reduced pressure. The residue was redissolved in 15 mL ethyl acetate and allowed to stand at 4° C. for 1 hour. The precipitate that formed, which consisted of a single regioisomer, was filtered, washed with ethyl acetate, and dried under reduced pressure to yield 440 mg (53%) of an off-white solid. This product was dissolved in 6 mL 1:1 dichloromethane: trifluoroacetic acid containing 0.1% triethylsilane and allowed to stand for 30 minutes before concentration under reduced pressure. The residue was dissolved in 2 mL water, and carefully neutralized by addition of saturated aqueous sodium bicarbonate, to a pH of 7.5 to 8.0. The precipitate that formed was filtered, and washed twice with 5 mL water, then four times with 10 mL ethyl acetate, and dried under reduced pressure. An off-white powder (308 mg, 92%) was obtained. The structure 1 assigned to the product based on proton, carbon-13 NMR and electrospray mass spectral analysis. The regiochemistry was confirmed by HMBC analysis.

### Example 2

Practice: Screening Environmental Libraries

The substrate 7-(ε-D-2-aminoadipoylamido)-4-methylcoumarin and the commercially available fluorescent substrates were used in high throughput, homogeneous assays to screen a collection of environmental libraries (Diversa Corporation, San Diego, Calif.). The substrates can be used to screen plasmid or phage libraries with whole cells or lysed cells. The substrate can be used for kinetic assays because fluorescence is generated upon cleavage of the amide bound, no secondary reagents are required to produce or enhance the fluorescent signal. In addition, the substrates can be used for quantitative kinetic characterization of enzymes present in crude lysates or purified preparations.

An exemplary liquid phase screen utilizing the 7-(ε-D-2-aminoadipoylamido)-4-methylcoumarin substrate would be carried out as follows:

The library of recombinant clones to be screened is introduced into the appropriate expression host cells, for example E. coli. The cells harboring library clones are diluted in growth media containing 7-(ε-D-2-aminoadipoylamido)-4-methylcoumarin (10-100 uM) to produce a clone density of 1-10 clones per well in either a 384 well, 1536 well, or 100,000 well screening format. The screening plates are placed at 25-37° C. to allow protein expression and substrate turnover. Expression of the recombinant proteins encoded by these clones is constitutive and therefore does not require a secondary addition or a change in growth conditions to induce protein expression. The presence of substrate during protein expression allows kinetic measurements to be taken. This is accomplished by reading liberated fluorescence, resulting from substrate cleavage over time, in a fluorescence plate reader (excitation at 360 nm and emission at 465 nm). Because the substrate can be used in a whole cell assay, no lysis steps are required to provide intracellular enzymes access to the substrate. Positive clones can be directly recovered from the well of the screening plate and further processed.

Results

Using the liquid screening protocol outlined above, 8 novel secondary amidases that have activity on 7-(ε-D-2-aminoadipoylamido)-4-methylcoumarin have been discovered. These secondary amidases and their corresponding nucleic acid sequences are set forth in Table 1. Analysis of the deduced amino acid sequences of these clones shows that they are related at the level of primary sequence (See FIG. 6).

### Example 3

Modifications/Variations

In addition to using 7-(ε-D-2-aminoadipoylamido)-4-methylcoumarin in liquid phase assays, the substrate has also been used in zymogram applications. In this application, crude lysates containing secondary amidase activity are fractionated on non-denaturing polyacrylamide gels. The gel is then soaked in buffer containing 7-(ε-D-2-aminoadipoylamido)-4-methylcoumarin. The location of secondary amidase activity in the gel is identified as bands of fluorescence that appear as the enzyme cleaves the substrate and the fluorescent signal is retained in the gel in close proximity to the active enzyme.

### Example 4

Enzymatic Activity of Isolated Enzymes

Assay Method. Reactions were performed in 384 well black plastic microtiter dishes (ScreenMates, Matrix Technologies, Hudson, N.H.). Reactions were prepared by combining the following:

50 mM Tris-HCl, pH 7.5

25 uM 7-(ε-D-2-aminoadipoylamido)-4-methylcoumarin

5 mM DTT or L-cysteine (or buffer for control)

1 ug purified SEQ ID NOS: 9 and 10

Final volume: 100 ul

Cleavage of 7-(ε-D-2-aminoadipoylamido)-4-methylcoumarin was recorded as an increase in fluorescence measured at an excitation of 360 nm and emission at 465 nm in a Molecular Devices SPECTRAMAX GEMINI XS™ fluorescence plate reader at 37° C. The Assay method was adapted from Arnon, R.: Papain, in Methods in Enzymology, XIX, Perlmann, G., and Lorand, L., eds., Academic Press, NY, 226 (1970).

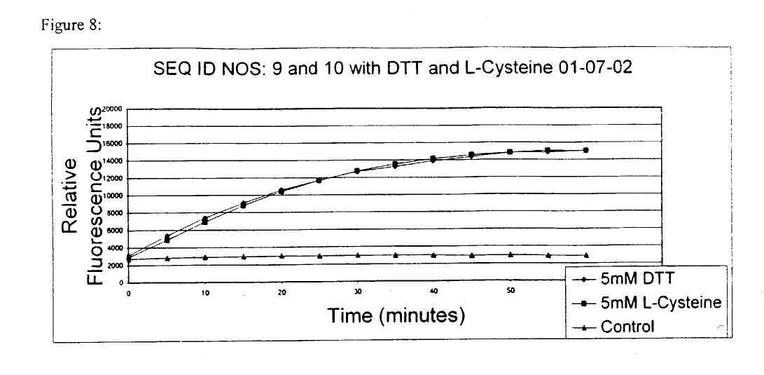

As can be seen in FIG. 8, the enzymatic activity of secondary amidase SEQ ID NOS: 9 and 10 is activated by incubation of the enzyme with dithiothreitol (DTT) or L-cysteine. 5 mM DTT shows activity of SEQ ID NOS: 9 and 10 against 7-(ε-D-2-aminoadipoylamido)-4-methylcoumarin in the presence of 5 mM DTT; 5 mM L-cysteine shows activity of SEQ ID NOS: 9 and 10 against 7-(ε-D-2-aminoadipoylamido)-4-methylcoumarin in the presence of 5 mM L-cysteine; Control shows activity of SEQ ID NOS: 9 and 10 against 7-(ε-D-2-aminoadipoylamido)-4-methylcoumarin in buffer alone.

### Example 5

One Enzyme Conversion of Cephalosporin C To 7-ACA

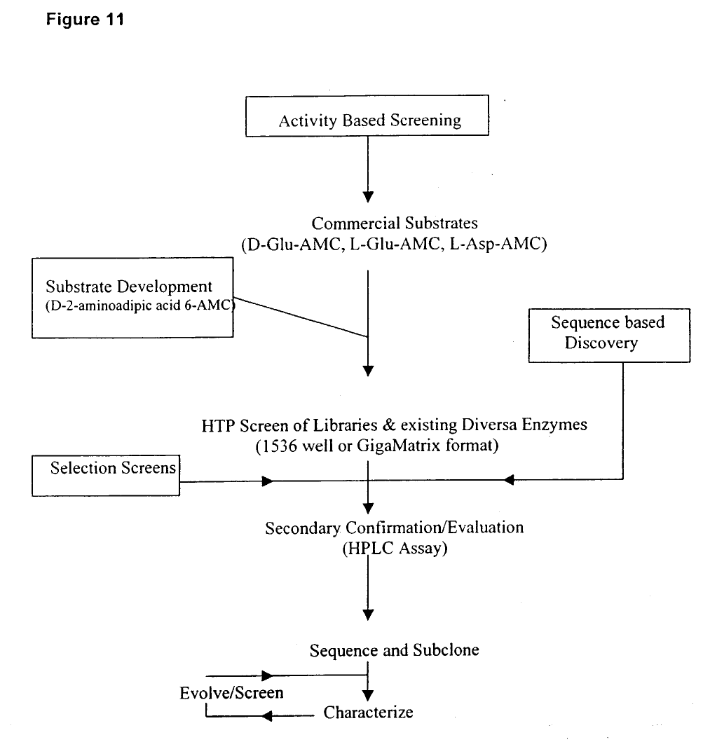

Activity Based Screening of Enzymes for Ceph C Acylase Activity (See FIG. 11)

Activity based screening includes the following:

Activity based screening of enzymes for ceph C acylase activity. Enzyme classes will include:

i. 1° and 2° amidases

ii. Shuffled amidases

iii. Esterases

iv. Amino Acid acylases

Activity based screening of libraries

Sequence based screening of libraries

Possible selection screening of libraries

Characterization of Discovered Clones

Liquid Based Activity Based Screening

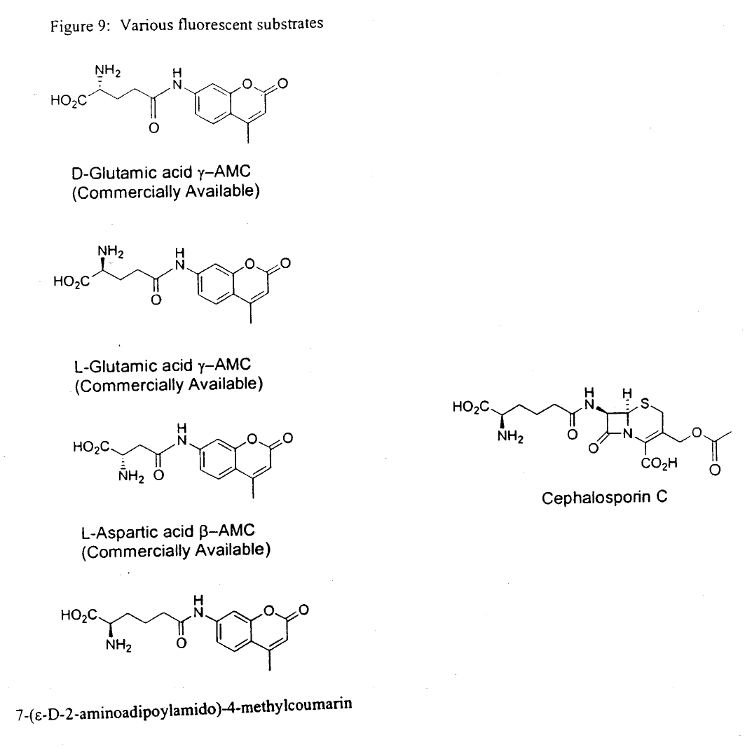

In one aspect, activity based screening is carried out as a homogeneous assay in a 1536 well format. Because it is a homogeneous assay, in another aspect GIGAMATRIX™ screening is used. Initial screening efforts will utilize commercially available fluorescent substrates that most closely mimic ceph C. In addition, a fluorescent substrate that more closely mimics ceph C has been synthesized (see FIG. 9).

Agar Based Activity Screen with 7-ACA Sensitive Indicator Strain

In one aspect, the invention provides a selection screen that takes advantage of an “uncharacterized” soil isolate that is sensitive to 7-ACA yet resistant to ceph C, as described, e.g., in Matsuda et al., 1987. J. Bacteriol., V. 169, pp. 5815-5820. This strain is used as an indicator strain in a clearing zone assay to identify clones that can convert ceph C to 7-ACA. Cells expressing library clones are overlayed on a lawn of indicator cells. Positive clones convert ceph C to 7-ACA thereby killing neighboring indicator cells to produce a clearing zone. In order to take advantage of this type of approach, it may be necessary to use a 7-ACA sensitive/ceph C resistant strain.

Sequence Based Screening

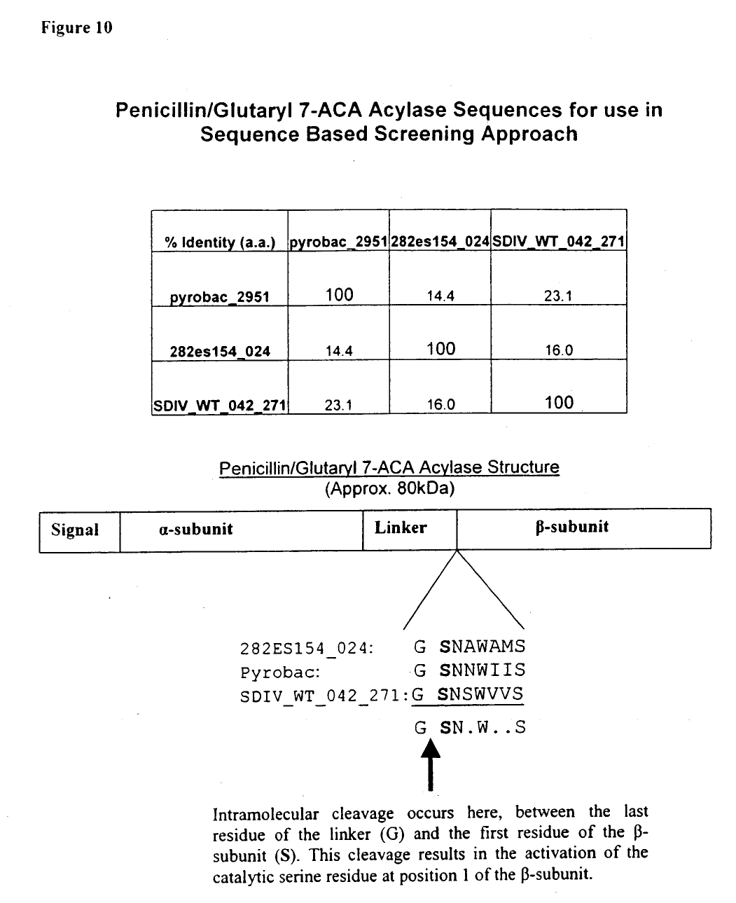

In one aspect, the invention provides sequence based screening involving biopanning and hybridization approaches that utilize ceph C (glutaryl-7-ACA) acylase sequences as probes. Three novel clones have been identified. These have been subcloned both for enzyme characterization and for use in sequence based screening (see FIG. 10). In one aspect, these sequences as well as published ceph C acylase sequences are compared, conserved regions identified, and degenerate oligonucleotide primers designed for use in a PCR screen other libraries.

### Example 6

Exemplary Selection Screens

Aminoadipic Acid Selection

The invention provides an aminoadipic acid selection method. In one aspect, the method produces/identifies a strain that is 1) resistant to ceph C; 2) unable to utilize ceph C as a sole carbon and/or nitrogen source; but 3) able to utilize D-2-aminoadipic acid as a sole carbon and/or nitrogen source. In one aspect, libraries are screened using selection in ceph C-containing minimal media and searched for cell growth. In one aspect, the growth reflects release of aminoadipic acid (and 7-ACA) from ceph C as an expressed activity is, thereby allowing the cells to metabolize the aminoadipic acid for growth.

“Caged ” Growth Source Selection

In one aspect, a carbon growth source is used as a “caged” growth source in an exemplary selection protocol. The carbon growth source is attached to D-2-Aminoadipic acid (AAA) and the substrate is supplied as the sole carbon and or nitrogen source for a culture that is auxotrophic for this “caged” carbon source. In one aspect, excised phagemid libraries are screened using a leucine auxotroph. Leucine can be attached to D-2-Aminoadipic acid to create the caged growth source. Libraries can be screened in leucine auxotrophs grown in a minimal media containing the caged leucine. Clones that express an activity that is able to release Leu from D-2-Aminoadipic acid-Leu can grow. The assumption is that enzymes that can cleave D-2-Aminoadipic acid-Leu can also cleave D-2-Aminoadipic acid-7ACA (cephalosporin C).

Characterization of Clones Expressing Amidases

In one aspect, secondary characterization of clones encoding an amidase discovered as described above involves the evaluation of the activity of the clones using ceph C as the substrate and an HPLC method to monitor the conversion of ceph C to 7-ACA.

### Example 7

Evolution/Optimization of Amidase Sequences

The invention provides methods of making amidases with altered activities comprising modification of the nucleic acids of the invention, as described herein. Because glutaryl-7-ACA acylases are able to directly convert ceph C to 7-ACA, albeit with low efficiency, in one aspect these enzymes are used for GSSM and reassembly (see above) to generate amidases of altered activity.

There is at least one published report demonstrating improvements in ceph C utilization efficiency (1.5 to 2.5 fold) through site-directed mutagenesis of glutaryl-7-ACA acylase from Pseudomonas strain N176 (see, e.g., Ishii, et al., 1995. Eur. J. Biochem., V. 230, pp. 773-778).

Liquid Activity Based Screening

In one aspect, the methods provide activity based screening, e.g., liquid activity based screening, of amidase-encoding nucleic acids. In one aspect, the fluorescent substrates represent suitable mimics for ceph C. The important determinant in substrate recognition may be the side chain D-2-aminoadipoyl group of ceph C; therefore, the 7-ACA group can be replaced with a fluorescent indicator.

It has already been shown that the primary determinant for substrate recognition is the side chain for penicillin acylases. A recent crystal structure analysis of a ceph C acylase suggests that the β-lactam nucleus may not be directly involved in specific interactions with the active site residues. In addition, assay development with a commercial penicillin acylase has shown that this enzyme has good activity on the commercial fluorescent substrates that are currently being used in the activity-based screen.

Agar based activity screening and aminoadipic acid selection screening In one aspect, the invention provides agar based activity screening and aminoadipic acid selection screening of amidases. In one aspect, the goal is to screen and identify strains that are either resistant or sensitive to components of the growth media. These screens can be considered secondary approaches with liquid activity screening and sequence based efforts as primary approaches.

“Caged” Growth Source Selection

In an exemplary “caged” growth selection method, D-2-Aminoadipic acid-Leu is synthesized. However, the final product contains two isomers. These isomers have proven difficult to separate by chromatography. In one aspect, screening is done using the mixed isomer. Any clone that grows will be re-screened in media containing pure D-2-Aminoadipic acid-Leu. The assumption is that an enzyme that can cleave D-2-Aminoadipic acid-Leu can also cleave cephalosporin C to 7-ACA.

### Example 8

Direct Conversion of Cephalosporin C to 7-Aminocephalosporanic Acid

This example demonstrates the use of secondary amidases of the invention for the direct conversion of cephalosporin C to 7-aminocephalosporanic acid.

Complex environmental libraries were screened using high throughput screening methods to identify the novel enzymes of the invention having a secondary amidase activity. In addition to using commercially available fluorescent substrates for discovery, novel substrates for secondary amidase discovery were synthesized, including 7-(ε-D-2-aminoadipoylamido)-4-methylcoumarin.

The effectiveness of the novel fluorescent secondary amidase substrate, 7-(ε-D-2-aminoadipoylamido)-4-methylcoumarin was demonstrated. 7-(ε-D-2-aminoadipoylamido)-4-methylcoumarin was specifically designed for use in high throughput (HT) activity-based, whole cell screening for the discovery of a secondary amidase activity that can directly convert the antibiotic cephalosporin C to 7-aminocephalosporanic acid (7-ACA). 7-(ε-D-2-aminoadipoylamido)-4-methylcoumarin utilizes the D-2-aminoadipoyl side chain found on cephalosporin C attached to the fluorescent reporter 7-amino-4-methylcoumarin through an amide linkage. Therefore, enzymes that can cleave this substrate can recognize the D-2-aminoadipoyl side chain and cleave the fluorescent substrate at a position equivalent to the desired site of cleavage in cephalosporin C. 7-(ε-D-2-aminoadipoylamido)-4-methylcoumarin was used for HT screening of environmental libraries and it identified novel secondary amidases that can convert cephalosporin C to 7-ACA. Sixteen sequences were discovered that possess activity on 7-(ε-D-2-aminoadipoylamido)-4-methylcoumarin. One of these enzymes, SEQ ID NO: 70, has been characterized with respect to its activity on cephalosporin C. SEQ ID NO: 70 can directly convert cephalosporin C to 7-ACA. In one aspect, this enzyme and related enzymes are useful for the large-scale production of 7-ACA from cephalosporin C using a one-enzyme process.

High throughput screening methods were used identify novel enzymes with secondary amidase activity against a novel substrate, 7-(ε-D-2-aminoadipoylamido)-4-methylcoumarin. Screening of environmental libraries, identified 16 unique secondary amidases, related to B. subtilis DppA D-amino peptidase, that have activity on 7-(ε-D-2-aminoadipoylamido)-4-methylcoumarin. Characterization of one of these enzymes, SEQ ID NO: 70, demonstrated that it can directly convert cephalosporin C to 7-ACA. The ability of DppA family members to carry out this direct enzymatic conversion of cephalosporin C to 7-ACA is a novel activity.

The substrate utilized here, 7-(ε-D-2-aminoadipoylamido)-4-methylcoumarin, is suitable for high throughput screening and has been used to identify secondary amidases in both conventional 1536 well and the 100,000 well GIGAMATRIX™ format (Diversa, San Diego, Calif.). In addition, the substrate is very sensitive because it utilizes a fluorescent reporter, 7-amino-4-methylcoumarin. Finally, the substrate is specific, containing the D-2-aminoadipoyl side chain found in cephalosporin C.

Using the liquid screening protocol outlined above, 16 novel DppA-related secondary amidases were discovered. These enzymes have activity on 7-(ε-D-2-aminoadipoylamido)-4-methylcoumarin.

Analysis of the deduced amino acid sequences shows that they are related at the level of primary sequence.

Direct Conversion of Cephalosporin C to 7-ACA by Amidase of the Invention

An E. coli strain engineered to over-express SEQ ID NO: 70 was used produce an active, recombinant SEQ ID NO: 70 enzyme of the invention. The purified enzyme has been characterized using cephalosporin C as the substrate.

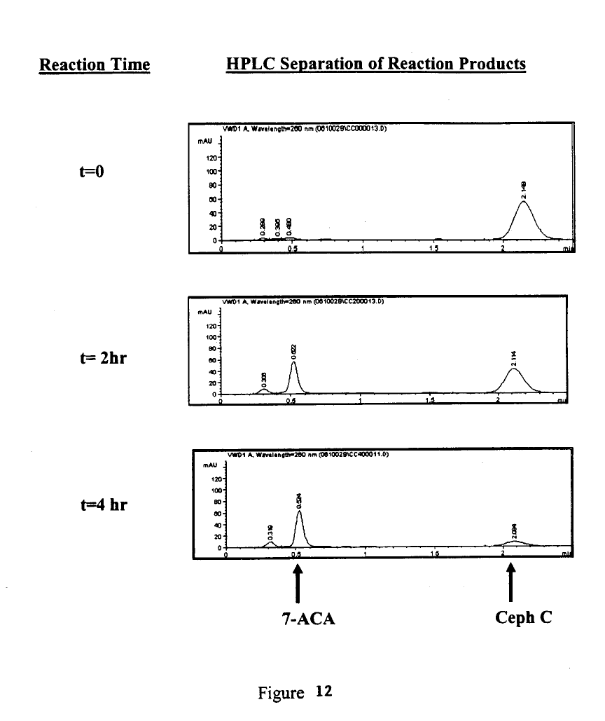

FIG. 12 illustrates reaction samples analyzed by using High Performance Liquid Chromatography (HPLC). Purified recombinant SEQ ID NO: 70 was assayed for its ability to directly convert cephalosporin C to 7-ACA. The reaction was performed with 7 mg/ml purified SEQ ID NO: 70 enzyme and 20 mM cephalosporin C in 50 mM MOPS buffer, pH 7.0 at 37° C. Samples of the reaction were taken at time 0, at 2 hours, and at 4 hours. The reaction samples were fractionated using HPLC in order to resolve the substrate (Ceph C) and the reaction product (7-ACA), as illustrated in FIG. 12. The results demonstrate that SEQ ID NO: 70 enzyme can directly convert cephalosporin C to 7-ACA.

Enzyme Activity Assay

Another exemplary routine assay to test for amidase activity comprises use of a substrate buffer of 50 mM sodium phosphate buffer pH 7.0 containing 13.5 mg/ml cloxacillin and cephalosporin C (10 mg/ml). Cell suspensions or semi-purified enzyme samples can be incubated in substrate buffer for up to 24 hours at 37° C. At a suitable time a 100 μl aliquot of fluram reagent was then added, mixed and the sample incubated at room temperature for 1 hour. Fluram reagent consists of fluroescamine (Sigma Chemical Co. St. Louis, Mo.) at 1 mg/ml in dry acetone. The presence of 7-ACA in the reaction mix can be detected after derivatization of the free primary amine group by using fluorescence spectroscopy at an excitation wave length of 378 nm and emission detection at 498 nm in a flow injection assay system with 10% acetone in water as carrier fluid. 7-ACA production also can be demonstrated in an HPLC system. The enzyme reaction mix can be derivatized as above a 20 μl sample applied in a mobile phase consisting of 35% acetonitrile, 0.1% trifluoroacetic acid in HPLC-grade water onto a 15 cm Hypersil ODS 5 mu column at 32° C. with a flow rate of 1.5 ml/min. Detection by fluorescence spectroscopy was as above. In this system authentic 7-ACA standard has a retention time of 6.7 minutes. See, e.g., U.S. Pat. No. 6,297,032. For other routine assays, see, e.g., EP0283218, EP0322032, EP0405846, EP0474652.

Modifications/Variations

The invention provides methods for making modifications and/or variations of the nucleic acids and polypeptides of the invention. In one aspect, the enzymes of the invention are modified, e.g., by directed evolution, using GSSM and gene reassembly technologies. These evolution strategies can be used to produce an enzyme that has improved biochemical characteristics for use in a one-enzyme biocatalytic process to be used for the direct conversion of ceph C to 7-ACA.

A number of embodiments of the invention have been described. Nevertheless, it will be understood that various modifications may be made without departing from the spirit and scope of the invention. Accordingly, other embodiments are within the scope of the following claims.

What is claimed is:

1. An isolated or recombinant nucleic acid comprising a nucleic acid sequence having
at least 50% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 31, SEQ ID NO: 33, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 91, SEQ ID NO: 93, SEQ ID NO: 95, SEQ ID NO: 97, SEQ ID NO: 101, SEQ ID NO: 103, SEQ ID NO: 105, SEQ ID NO: 107, SEQ ID NO: 109, SEQ ID NO: 111,
at least 55% sequence identity to SEQ ID NO: 35, SEQ ID NO: 73, SEQ ID NO: 89, SEQ ID NO: 113,
at least 60% sequence identity to SEQ ID NO: 27, SEQ ID NO: 29, SEQ ID NO: 57,
at least 65% sequence identity to SEQ ID NO: 99,
at least 90% sequence identity to SEQ ID NO: 55,
at least 99% sequence identity to SEQ ID NO: 37,
over a region of at least about 100 residues, wherein the nucleic acid encodes at least one polypeptide having an amidase activity, and the sequence identities are determined by analysis with a sequence comparison algorithm or by a visual inspection.

2. The isolated or recombinant nucleic acid of claim 1, wherein the isolated or recombinant nucleic acid comprises a nucleic acid sequence having at least 55% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 31, SEQ ID NO: 33, SEQ ID NO: 35, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 101, SEQ ID NO: 103, SEQ ID NO: 105, SEQ ID NO: 107, SEQ ID NO: 109, SEQ ID NO: 111, SEQ ID NO: 113, over a region of at least about 100 residues.

3. The isolated or recombinant nucleic acid of claim 2, wherein the nucleic acid comprises a sequence having at least 60% sequence identity to SEQ ID NO: 1, SEQ ID NO: 3, SEQ ID NO: 5, SEQ ID NO: 7, SEQ ID NO: 9, SEQ ID NO: 1, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 100 residues.

4. The isolated or recombinant nucleic acid of claim 3, wherein the nucleic acid comprises a sequence having at least 65% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 100 residues.

5. The isolated or recombinant nucleic acid of claim 4, wherein the nucleic acid comprises a sequence having at least 70% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 57, SEQ ID NO: 59, SEQ ID NO.61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 100 residues.

6. The isolated or recombinant nucleic acid of claim 5, wherein the nucleic acid comprises a sequence having at least 75% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ I:D NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 100 residues.

7. The isolated or recombinant nucleic acid of claim 6, wherein the nucleic acid comprises a sequence having at least 80% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 100 residues.

8. The isolated or recombinant nucleic acid of claim 7, wherein the nucleic acid comprises a sequence having at least 85% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 100 residues.

9. The isolated or recombinant nucleic acid of claim 8, wherein the nucleic acid comprises a sequence having at least 90% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ED NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 100 residues.

10. The isolated or recombinant nucleic acid of claim 9, wherein the nucleic acid comprises a sequence having at least 95% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 100 residues.

11. The isolated or recombinant nucleic acid of claim 10, wherein the nucleic acid comprises a sequence having at least 99% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ if NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 100 residues.

12. The isolated or recombinant nucleic acid of claim 11, wherein the nucleic acid sequence comprises a sequence as set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ED NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113.

13. An isolated or recombinant nucleic acid encoding a polypeptide comprising a sequence as set forth in SEQ ID NO: 2, SEQ ID NO: 4, SEQ ID NO: 6, ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ED NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 113, SEQ ID NO: 114.

14. The isolated or recombinant nucleic acid of claim 1, wherein the sequence comparison algorithm is a BLAST version 2.2.2 algorithm where a filtering setting is set to blastall-p blastp-d “nr pataa”-F F, and all other options are set to default.

15. The isolated or recombinant nucleic acid of claim 1, wherein the nucleic acid comprises a sequence having at least 50% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 200, 300, 400, 500, 550, 600, or 650 residues.

16. The isolated or recombinant nucleic acid of claim 15, wherein the nucleic acid comprises a sequence having at least 50% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113 over a region of at least about 700 residues.

17. The isolated or recombinant nucleic acid of claim 16, wherein the nucleic acid comprises a sequence having at least 50% sequence identity to SEQ ID NO: 1, SEQ ID NO: 3, SEQ ID NO: 5, ID NO: 7, SEQ ID NO: 9, SEQ ID NO: 13, SEQ ID NO: 15, SEQ ID NO: 17, SEQ ID NO: 19, SEQ ID NO: 21, SEQ ID NO: 25, SEQ ID NO: 27, SEQ ID NO: 31, SEQ ID NO: 33, SEQ ID NO: 35, SEQ ID NO: 39, SEQ ID NO: 41, SEQ ID NO: 43, SEQ ID NO: 47, SEQ ID NO: 49, SEQ ID NO: 51, SEQ ID NO: 53, SEQ ID NO: 59, SEQ ID NO: 61, SEQ ID NO: 63, SEQ ID NO: 65, SEQ ID NO: 67, SEQ ID NO: 69, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 111, SEQ ID NO: 113 over a region of at least about 800 residues.

18. The isolated or recombinant nucleic acid of claim 17, wherein the nucleic acid comprises a sequence having at least 50% sequence identity to SEQ ID NO: 3, SEQ ID NO: 5, SEQ ID NO: 9, SEQ ID NO: 13, SEQ ID NO: 15, SEQ ID NO: 17, SEQ ID NO: 19, SEQ ID NO: 21, SEQ ID NO: 31, SEQ ID NO: 35, SEQ ID NO: 39, SEQ ID NO: 47, SEQ ID NO: 49, SEQ ID NO: 53, SEQ ID NO: 61, SEQ ID NO: 63, SEQ ID NO: 65, SEQ ID NO: 73, SEQ ID NO: 75, SEQ ID NO: 81, SEQ ID NO: 83, SEQ ID NO: 87, SEQ ID NO: 89, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 111, SEQ ID NO: 113 over a region of at least about 900 residues.

19. The isolated or recombinant nucleic acid of claim 18, wherein the nucleic acid comprises a sequence having at least 50% sequence identity to SEQ ID NO: 3, SEQ ID NO: 5, SEQ ID NO: 9, SEQ ID NO: 13, SEQ ID NO: 15, SEQ ID NO: 17, SEQ ID NO: 19, SEQ ID NO: 21, SEQ ID NO: 31, SEQ ID NO: 35, SEQ ID NO: 39, SEQ ID NO: 47, SEQ ID NO: 49, SEQ ID NO: 53, SEQ ID NO: 61, SEQ ID NO: 63, SEQ ID NO: 65, SEQ ID NO: 73, SEQ ID NO: 75, SEQ ID NO: 81, SEQ ID NO: 83, SEQ ID NO: 87, SEQ ID NO: 89, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 111, SEQ ID NO: 113 over a region of at least about 900 residues.

20. The isolated or recombinant nucleic acid of claim 19, wherein the nucleic acid comprises a sequence having at least 50% sequence identity to SEQ ID NO: 15, SEQ ID NO: 17, SEQ ID NO: 19, SEQ ID NO: 21, SEQ ID NO: 31, SEQ ID NO: 35, SEQ ID NO: 47, SEQ ID NO: 49, SEQ ID NO: 53, SEQ ID NO: 61, SEQ ID NO: 63, SEQ ID NO: 65, SEQ ID NO: 73, SEQ ID NO: 75, SEQ ID NO: 81, SEQ ID NO: 87, SEQ ID NO: 89, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 111, SEQ ID NO: 113 over a region of at least about 1000 residues.

21. The isolated or recombinant nucleic acid of claim 20, wherein the nucleic acid comprises a sequence having at least 50% sequence identity to SEQ ID NO: 31, SEQ ID NO: 35, SEQ ID NO: 47, SEQ ID NO: 49, SEQ ID NO: 53, SEQ ID NO: 61, SEQ ID NO: 63, SEQ ID NO: 65, SEQ ID NO: 73, SEQ ID NO: 75, SEQ ID NO: 81, SEQ ID NO: 87, SEQ ID NO: 89, SEQ ID NO: 97, SEQ ID NO: 99, SEQ ED NO: 101, SEQ ID NO: 105, SEQ ID NO: 107, SEQ ID NO: 111 over a region of at least about 1200 residues.

22. An isolated or recombinant nucleic acid, wherein the nucleic acid comprises a sequence that hybridizes under stringent conditions to a nucleic acid comprising a sequence as set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, wherein the nucleic acid encodes a polypeptide having an amidase activity.

23. The isolated or recombinant nucleic acid of claim 22, wherein the nucleic acid is at least about 50, 100, 150, 200, 300, 400 or 500 residues in length.

24. The isolated or recombinant nucleic acid of claim 23, wherein the nucleic acid is at least about 600, 700, 800, 900, 1000, 1100 or 1200 residues in length or the full length of the gene or transcript.

25. The isolated or recombinant nucleic acid of claim 22, wherein the stringent conditions comprise a wash step comprising a wash in 0.2×SSC at a temperature of about 65° C. for about 15 minutes.

26. The isolated or recombinant nucleic acid of claim 1, wherein the amidase activity comprises hydrolyzing an amide bond.

27. The isolated or recombinant nucleic acid of claim 1, wherein the amidase activity comprises a secondary amidase activity.

28. The isolated or recombinant nucleic acid of claim 1, wherein the amidase activity comprises an internal amidase activity.

29. The isolated or recombinant nucleic acid of claim 1, wherein the amidase activity comprises a C-terminal amidase activity.

30. The isolated or recombinant nucleic acid of claim 1, wherein the amidase activity comprises an N-terminal amidase activity.

31. The isolated or recombinant nucleic acid of claim 26, wherein the amidase activity comprises hydrolyzing amide bonds in a protein.

32. The isolated or recombinant nucleic acid of claim 1, wherein the amidase activity comprises hydrolyzing an amide bond in a cephalosporin.

33. The isolated or recombinant nucleic acid of claim 32, wherein the cephalosporin comprises cephalosporin C.

34. The isolated or recombinant nucleic acid of claim 33, wherein the amidase activity comprises an hydrolyzing amide bond in cephalosporin C to produce 7-aminocephalosporanic acid (7-ACA).

35. The isolated or recombinant nucleic acid of claim 1, wherein the amidase activity is enantioselective.

36. The isolated or recombinant nucleic acid of claim 35, wherein the amidase generates enantiomerically pure L-amino acids from racemic mixtures.

37. The isolated or recombinant nucleic acid of claim 1, wherein amidase generates peptides by the enzymatic conversion of amino acid alkyl esters or N-protected peptide alkyl esters.

38. The isolated or recombinant nucleic acid of claim 1, wherein the amidase retains activity under conditions comprising a temperature range of between about 37° C. to about 95° C.

39. The isolated or recombinant nucleic acid of claim 38, wherein the amidase retains activity under conditions comprising a temperature range of between about 55° C. to about 85° C.

40. The isolated or recombinant nucleic acid of claim 1, wherein the amidase activity is thermotolerant.

41. The isolated or recombinant nucleic acid of claim 40, wherein the polypeptide retains an amidase activity after exposure to a temperature in the range from greater than 37° C. to about 95° C.

42. A nucleic acid probe for identifying a nucleic acid encoding a polypeptide with an amidase activity, wherein the probe comprises at least 10, 20, 30, 40 or 50 consecutive bases of a sequence comprising SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, wherein the probe identifies the nucleic acid by binding or hybridization.

43. The nucleic acid probe of claim 42, wherein the probe comprises an oligonucleotide comprising at least about 10 to 50, about 20 to 60, about 30 to 70, about 40 to 80, or about 60 to 100, or about 70 to 150 consecutive bases of a sequence comprising SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113.

44. A nucleic acid probe for identifying a nucleic acid encoding a polypeptide having an amidase activity, wherein the probe comprises at least 10, 20, 30, 40 or 50 consecutive bases of a sequence as set forth in claim 1.

45. An amplification primer sequence pair for amplifying a nucleic acid encoding a polypeptide having an amidase activity, wherein the primer pair is capable of amplifying a nucleic acid as set forth in claim 1.

46. The amplification primer pair of claim 45, wherein each member of the amplification primer sequence pair comprises an oligonucleotide comprising at least between about 10 to 50 consecutive bases of the sequence.

47. A method of amplifying a nucleic acid encoding a polypeptide having an amidase activity comprising amplification of a template nucleic acid with an amplification primer sequence pair as set forth in claim 45.

48. An expression cassette comprising a nucleic acid as set forth in claim 1 or claim 22.

49. A vector comprising a nucleic acid comprising a nucleic acid as set forth in claim 1 or claim 22.

50. A cloning vehicle comprising a vector as set forth in claim 49, or a nucleic acid as set forth in claim 1 or claim 22, wherein the cloning vehicle comprises a viral vector, a plasmid, a phage, a phagemid, a cosmid, a fosmid, a bacteriophage or an artificial chromosome.

51. The cloning vehicle of claim 50, wherein the viral vector comprises an adenovirus vector, a retroviral vector or an adeno-associated viral vector.

52. The cloning vehicle of claim 50, comprising a bacterial artificial chromosome (BAC), a plasmid, a bacteriophage P1-derived vector (PAC), a yeast artificial chromosome (YAC), or a mammalian artificial chromosome (MAC).

53. A transformed cell comprising a vector as set forth in claim 49, or a nucleic acid as set forth in claim 1 or claim 22.

54. A transformed cell comprising a vector as set forth in claim 49, or a nucleic acid as set forth in claim 1 or claim 22.

55. The transformed cell of claim 54, wherein the cell is a bacterial cell, a mammalian cell, a fungal cell, a yeast cell, an insect cell or a plant cell.

56. A transgenic non-human animal comprising a vector as set forth in claim 49, or a nucleic acid as set forth in claim 1 or claim 22.

57. The transgenic non-human animal of claim 56, wherein the animal is a mouse.

58. A transgenic plant comprising a vector as set forth in claim 49, or a nucleic acid as set forth in claim 1 or claim 22.

59. The transgenic plant of claim 58, wherein the plant is a corn plant, a sorghum plant, a potato plant, a tomato plant, a wheat plant, an oilseed plant, a rapeseed plant, a soybean plant, a nice plant, a barley plant, a grass, or a tobacco plant.

60. A transgenic seed comprising a vector as set forth in claim 49, or a nucleic acid as set forth in claim 1 or claim 22.

61. The transgenic seed of claim 60, wherein the seed is a corn seed, a wheat kernel, an oilseed, a rapeseed, a soybean seed, a palm kernel, a sunflower seed, a sesame seed, a rice, a barley, a peanut or a tobacco plant seed.

62. An antisense oligonucleotide comprising a nucleic acid sequence complementary to or capable of hybridizing under stringent conditions to a vector as set forth in claim 49, or a nucleic acid as set forth in claim 1 or claim 22.

63. The antisense oligonucleotide of claim 62, wherein the antisense oligonucleotide is between about 10 to 50, about 20 to 60, about 30 to 70, about 40 to 80, or about 60 to 100 bases in length.

64. A method of inhibiting the translation of an amidase message in a cell comprising administering to the cell or expressing in the cell an antisense oligonucleotide comprising a nucleic acid sequence complementary to or capable of hybridizing under stringent conditions to a vector as set forth in claim 49, or a nucleic acid as set forth in claim 1 or claim 22.

65. An isolated or recombinant polypeptide comprising (a) a polypeptide comprising at least 50% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 32, SEQ ID NO: 34, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 76, SEQ ID NO: 78, SEQ ID NO: 80, SEQ ID NO: 82, SEQ ID NO: 85, SEQ ID NO: 87, SEQ ID NO: 88, SEQ ID NO: 92, SEQ ID NO: 94, SEQ ID NO: 96, SEQ ID NO: 98, SEQ ID NO: 102, SEQ ID NO: 104, SEQ ID NO: 106, SEQ ID NO: 108, SEQ ID NO: 110, SEQ ID NO: 112,
at least 55% sequence identity to SEQ ID NO: 36, SEQ I NO: 74, SEQ ID NO: 90, SEQ ID NO: 114,
at least 60% sequence identity to SEQ ID NO: 28, SEQ ID NO: 30, SEQ ID NO: 58,
at least 65% sequence identity to SEQ ID NO: 100, at least 90% sequence identity to SEQ ID NO: 56,
at least 99% sequence identity to SEQ ID NO: 38, over a region of at least about 100 residues; or
(b) a polypeptide encoded by a nucleic acid comprising a nucleic acid as set forth in claim 1 or claim 22.

66. The isolated or recombinant polypeptide of claim 65, wherein the polypeptide has an amidase activity.

67. The isolated or recombinant polypeptide of claim 65, wherein the polypeptide comprises an amino acid sequence having at least 50% identity a sequence region of at least about 150, 200 250, 300, 350, 400, 450 or 500 residues.

68. The isolated or recombinant polypeptide of claim 65, wherein the polypeptide comprises an amino acid sequence having at least 55%, 60%, 65%, 70%, 75%, 80%, 85%, 90%, 95% or 99% identity over a region of at least about 100 residues.

69. The isolated or recombinant polypeptide of claim 68, wherein the polypeptide comprises an amino acid sequence as set forth in SEQ ID NO: 2, SEQ ID NO: 4, SEQ ID NO: 6, ID NO: 8, SEQ ID NO: 10, SEQ ID NO: 12, SEQ BD NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ED NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 113, SEQ ID NO: 114.

70. The isolated or recombinant polypeptide of claim 66, wherein the amidase activity comprises hydrolyzing an amide bond.

71. The isolated or recombinant polypeptide of claim 66, wherein the amidase activity comprises hydrolyzing an amide bond.

72. The isolated or recombinant polypeptide of claim 66, wherein the amidase activity comprises a secondary amidase activity.

73. The isolated or recombinant polypeptide of claim 66, wherein the amidase activity comprises an internal amidase activity.

74. The isolated or recombinant polypeptide of claim 66, wherein the amidase activity comprises a C-terminal amidase activity.

75. The isolated or recombinant polypeptide of claim 66, wherein the amidase activity comprises an N-terminal amidase activity.

76. The isolated or recombinant polypeptide of claim 66, wherein the amidase activity comprises hydrolyzing amide bonds in a protein.

77. The isolated or recombinant polypeptide of claim 66, wherein the amidase activity comprises hydrolyzing an amide bond in a cephalosporin.

78. The isolated or recombinant polypeptide of claim 77, wherein the cephalosporin comprises cephalosporin C.

79. The isolated or recombinant polypeptide of claim 66, wherein the amidase activity comprises an hydrolyzing amide bond in cephalosporin C to produce 7-aminocephalosporanic acid (7-ACA).

80. The isolated or recombinant polypeptide of claim 66, wherein the amidase activity is enantioselective.

81. The isolated or recombinant polypeptide of claim 80, wherein the amidase generates enantiomerically pure L-amino acids from racemic mixtures.

82. The isolated or recombinant polypeptide of claim 66, wherein amidase generates peptides by the enzymatic conversion of amino acid alkyl esters or N-protected peptide alkyl esters.

83. The isolated or recombinant polypeptide of claim 66, wherein the amidase retains activity under conditions comprising a temperature range of between about 37° C. to about 95° C.

84. The isolated or recombinant polypeptide of claim 83, wherein the amidase retains activity under conditions comprising a temperature range of between about 55° C. to about 85° C.

85. The isolated or recombinant polypeptide of claim 66, wherein the amidase activity is thermotolerant.

86. The isolated or recombinant polypeptide of claim 85, wherein the polypeptide retains an amidase activity after exposure to a temperature in the range from greater than 37° C. to about 95° C.

87. An isolated or recombinant polypeptide comprising a polypeptide as set forth in claim 65 and lacking a signal sequence.

88. The isolated or recombinant polypeptide of claim 66, wherein the amidase activity comprises a specific activity at about 37° C. in the range from about 100 to about 1000 units per milligram of protein.

89. The isolated or recombinant polypeptide of claim 88, wherein the amidase activity comprises a specific activity from about 500 to about 750 units per milligram of protein.

90. The isolated or recombinant polypeptide of claim 65, wherein the polypeptide comprises at least one glycosylation site.

91. The isolated or recombinant polypeptide of claim 90, wherein glycosylation is an N-linked glycosylation.

92. The isolated or recombinant polypeptide of claim 90, wherein amidase is glycosylated after being expressed in a P. pastoris or a S. pombe.

93. The isolated or recombinant polypeptide of claim 66, wherein the polypeptide retains an amidase activity under conditions comprising about pH 5 or about pH 4.5.

94. The isolated or recombinant polypeptide of claim 71, wherein the polypeptide retains an amidase activity under conditions comprising about pH 8.0, about pH 8.5, about pH 9, about pH 9.5, about pH 10 or about pH 10.5.

95. A protein preparation comprising a polypeptide as set forth in claim 65, wherein the protein preparation comprises a liquid, a solid or a gel.

96. A heterodimer comprising a polypeptide as set forth in claim 65 and a second domain.

97. The heterodimer of claim 96, wherein the second domain is a polypeptide and the heterodimer is a fusion protein.

98. The heterodimer of claim 97, wherein the second domain is an epitope.

99. The heterodimer of claim 97, wherein the second domain is a tag.

100. A homodimer comprising a polypeptide as set forth in claim 65.

101. An immobilized polypeptide having an amidase activity, wherein the polypeptide comprises a sequence as set forth in claim 65 or claim 96.

102. The immobilized polypeptide of claim 101, wherein the polypeptide is immobilized on a cell, a metal, a resin, a polymer, a ceramic, a glass, a microelectrode, a graphitic particle, a bead, a gel, a plate, an array or a capillary tube.

103. An array comprising an immobilized polypeptide as set forth in claim 65 or claim 96.

104. An array comprising an immobilized nucleic acid as set forth in claim 1 or claim 22.

105. An isolated or recombinant antibody that specifically binds to a polypeptide as set forth in claim 65 or to a polypeptide encoded by a nucleic acid as set forth in claim 1 or claim 22.

106. The isolated or recombinant antibody of claim 105, wherein the antibody is a monoclonal or a polyclonal antibody.

107. A hybridoma comprising an antibody that specifically binds to a polypeptide as set forth in claim 65 or to a polypeptide encoded by a nucleic acid as set forth in claim 1 or claim 32.

108. A method of isolating or identifying a polypeptide with an amidase activity comprising the steps of:
(a) providing an antibody as set forth in claim 105;
(b) providing a sample comprising polypeptides; and
(c) contacting the sample of step (b) with the antibody of step (a) under conditions wherein the antibody can specifically bind to the polypeptide, thereby isolating or identifying a polypeptide having an amidase activity.

109. A method of making an anti-amidase antibody comprising administering to a non-human animal a nucleic acid as set forth in claim 1 or claim 32, a polypeptide as set forth in claim 65, or a polypeptide encoded by a nucleic acid as set forth in claim 1 or claim 22 in an amount sufficient to generate a humoral immune response, thereby making an anti-amidase antibody.

110. A method of producing a recombinant polypeptide comprising the steps of:
(a) providing a nucleic acid operably linked to a promoter, wherein the nucleic acid comprises a sequence as set forth in claim 1 or claim 22; and
(b) expressing the nucleic acid of step (a) under conditions that allow expression of the polypeptide, thereby producing a recombinant polypeptide.

111. The method of claim 110, further comprising transforming a host cell with the nucleic acid of step (a) followed by expressing the nucleic acid of step (a), thereby producing a recombinant polypeptide in a transformed cell.

112. A method for identifying a polypeptide having an amidase activity comprising the following steps:
(a) providing a polypeptide as set forth in claim 65; or a polypeptide encoded by a nucleic acid as set forth in claim 1 or claim 22;
(b) providing an amidase substrate; and
(c) contacting the polypeptide with the substrate of step (b) and detecting a decrease in the amount of substrate or an increase in the amount of a reaction product, wherein a decrease in the amount of the substrate or an increase in the amount of the reaction product detects a polypeptide having an amidase activity.

113. The method of claim 112, wherein the substrate is a protein.

114. The method of claim 113, wherein the substrate is a cephalosporin C.

115. A method for identifying an amidase substrate comprising the following steps:
(a) providing a polypeptide as set forth in claim 65; or a polypeptide encoded by a nucleic acid as set forth in claim 1 or claim 22;
(b) providing a test substrate; and
(c) contacting the polypeptide of step (a) with the test substrate of step (b) and detecting a decrease in the amount of substrate or an increase in the amount of reaction product, wherein a decrease in the amount of the substrate or an increase in the amount of a reaction product identifies the test substrate as an amidase substrate.

116. A method of determining whether a test compound specifically binds to a polypeptide comprising the following steps:
(a) expressing a nucleic acid or a vector comprising the nucleic acid under conditions permissive for translation of the nucleic acid to a polypeptide, wherein the nucleic acid has a sequence as set forth in claim 1 or claim 22, or, providing a polypeptide as set forth in claim 65;
(b) providing a test compound;
(c) contacting the polypeptide with the test compound; and
(d) determining whether the test compound of step (b) specifically binds to the polypeptide.

117. A method for identifying a modulator of an amidase activity comprising the following steps:
(a) providing a polypeptide as set forth in claim 66 or a polypeptide encoded by a nucleic acid as set forth in claim 1 or claim 22;
(b) providing a test compound;
(c) contacting the polypeptide of step (a) with the test compound of step (b) and measuring an activity of the amidase, wherein a change in the amidase activity measured in the presence of the test compound compared to the activity in the absence of the test compound provides a determination that the test compound modulates the amidase activity.

118. The method of claim 116, wherein the amidase activity is measured by providing an amidase substrate and detecting a decrease in the amount of the substrate or an increase in the amount of a reaction product, or, an increase in the amount of the substrate or a decrease in the amount of a reaction product.

119. The method of claim 117, wherein a decrease in the amount of the substrate or an increase in the amount of the reaction product with the test compound as compared to the amount of substrate or reaction product without the test compound identifies the test compound as an activator of amidase activity.

120. The method of claim 117, wherein an increase in the amount of the substrate or a decrease in the amount of the reaction product with the test compound as compared to the amount of substrate or reaction product without the test compound identifies the test compound as an inhibitor of amidase activity.

121. A computer system comprising a processor and a data storage device wherein said data storage device has stored thereon a polypeptide sequence or a nucleic acid sequence, wherein the polypeptide sequence comprises sequence as set forth in claim 65, or a polypeptide encoded by a nucleic acid as set forth in claim 1 or claim 22.

122. The computer system of claim 120, further comprising a sequence comparison algorithm and a data storage device having at least one reference sequence stored thereon.

123. The computer system of claim 121, wherein the sequence comparison algorithm comprises a computer program that indicates polymorphisms.

124. The computer system of claim 120, further comprising an identifier that identifies one or more features in said sequence.

125. A computer readable medium having stored thereon a polypeptide sequence or a nucleic acid sequence, wherein the polypeptide sequence comprises a polypeptide as set forth in claim 65; a polypeptide encoded by a nucleic acid as set forth in claim 1 or claim 22.

126. A method for identifying a feature in a sequence comprising the steps of:
(a) reading the sequence using a computer program which identifies one or more features in a sequence, wherein the sequence comprises a polypeptide sequence or a nucleic acid sequence, wherein the polypeptide sequence comprises a polypeptide as set forth in claim 65; a polypeptide encoded by a nucleic acid as set forth in claim 1 or claim 22; and
(b) identifying one or more features in the sequence with the computer program.

127. A method for comparing a first sequence to a second sequence comprising the steps of:
(a) reading the first sequence and the second sequence through use of a computer program which compares sequences, wherein the first sequence comprises a polypeptide sequence or a nucleic acid sequence, wherein the polypeptide sequence comprises a polypeptide as set forth in claim 65 or a polypeptide encoded by a nucleic acid as set forth in claim 1 or claim 22; and
(b) determining differences between the first sequence and the second sequence with the computer program.

128. The method of claim 126, wherein the step of determining differences between the first sequence and the second sequence further comprises the step of identifying polymorphisms.

129. The method of claim 126, further comprising an identifier that identifies one or more features in a sequence.

130. The method of claim 126, comprising reading the first sequence using a computer program and identifying one or more features in the sequence.

131. A method for isolating or recovering a nucleic acid encoding a polypeptide with an amidase activity from an environmental sample comprising the steps of:
(a) providing an amplification primer sequence pair as set forth in claim 44;
(b) isolating a nucleic acid from the environmental sample or treating the environmental sample such that nucleic acid in the sample is accessible for hybridization to the amplification primer pair; and,
(c) combining the nucleic acid of step (b) with the amplification primer pair of step (a) and amplifying nucleic acid from the environmental sample, thereby isolating or recovering a nucleic acid encoding a polypeptide with an amidase activity from an environmental sample.

132. The method of claim 130, wherein each member of the amplification primer sequence pair comprises an oligonucleotide comprising at least about 10 to 50 consecutive bases.

133. A method for isolating or recovering a nucleic acid encoding a polypeptide with an amidase activity from an environmental sample comprising the steps of:
(a) providing a polynucleotide probe comprising a sequence as set forth in claim 1 or claim 22;
(b) isolating a nucleic acid from the environmental sample or treating the environmental sample such that nucleic acid in the sample is accessible for hybridization to a polynucleotide probe of step (a);
(c) combining the isolated nucleic acid or the treated environmental sample of step (b) with the polynucleotide probe of step (a); and
(d) isolating a nucleic acid that specifically hybridizes with the polynucleotide probe of step (a), thereby isolating or recovering a nucleic acid encoding a polypeptide with an amidase activity from an environmental sample.

134. The method of claim 130 or claim 132, wherein the environmental sample comprises a water sample, a liquid sample, a soil sample, an air sample or a biological sample.

135. The method of claim 133, wherein the biological sample is derived from a bacterial cell, a protozoan cell, an insect cell, a yeast cell, a plant cell, a fungal cell or a mammalian cell.

136. A method of generating a variant of a nucleic acid encoding a polypeptide with an amidase activity comprising the steps of:
(a) providing a template nucleic acid comprising a sequence as set forth in claim 1 or claim 22; and
(b) modifying, deleting or adding one or more nucleotides in the template sequence, or a combination thereof, to generate a variant of the template nucleic acid.

137. The method of claim 135, further comprising expressing the variant nucleic acid to generate a variant amidase polypeptide.

138. The method of claim 135, wherein the modifications, additions or deletions are introduced by a method comprising error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturated mutagenesis (GSSM), synthetic ligation reassembly (SLR) and a combination thereof.

139. The method of claim 135, wherein the modifications, additions or deletions are introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof.

140. The method of claim 135, wherein the modifications, additions or deletions are introduced by error-prone PCR.

141. The method of claim 135, wherein the modifications, additions or deletions are introduced by shuffling.

142. The method of claim 135, wherein the modifications, additions or deletions are introduced by oligonucleotide-directed mutagenesis.

143. The method of claim 135, wherein the modifications, additions or deletions are introduced by assembly PCR.

144. The method of claim 135, wherein the modifications, additions or deletions are introduced by sexual PCR mutagenesis.

145. The method of claim 135, wherein the modifications, additions or deletions are introduced by in vivo mutagenesis.

146. The method of claim 135, wherein the modifications, additions or deletions are introduced by cassette mutagenesis.

147. The method of claim 135, wherein the modifications, additions or deletions are introduced by recursive ensemble mutagenesis.

148. The method of claim 135, wherein the modifications, additions or deletions are introduced by exponential ensemble mutagenesis.

149. The method of claim 135, wherein the modifications, additions or deletions are introduced by site-specific mutagenesis.

150. The method of claim 135, wherein the modifications, additions or deletions are introduced by gene reassembly.

151. The method of claim 135, wherein the modifications, additions or deletions are introduced by synthetic ligation reassembly (SLR).

152. The method of claim 135, wherein the modifications, additions or deletions are introduced by gene site saturated mutagenesis (GSSM).

153. The method of claim 135, wherein the method is iteratively repeated until an amidase having an altered or different activity or an altered or different stability from that of a polypeptide encoded by the template nucleic acid is produced.

154. The method of claim 152, wherein the variant amidase polypeptide is thermotolerant, and retains some activity after being exposed to an elevated temperature.

155. The method of claim 152, wherein the variant amidase polypeptide has increased glycosylation as compared to the amidase encoded by a template nucleic acid.

156. The method of claim 152, wherein the variant amidase polypeptide has an amidase activity under a high temperature, wherein the amidase encoded by the template nucleic acid is not active under the high temperature.

157. The method of claim 135, wherein the method is iteratively repeated until an amidase coding sequence having an altered codon usage from that of the template nucleic acid is produced.

158. The method of claim 135, wherein the method is iteratively repeated until an amidase gene having higher or lower level of message expression or stability from that of the template nucleic acid is produced.

159. A method for modifying codons in a nucleic acid encoding a polypeptide with an amidase activity to increase its expression in a host cell, the method comprising the following steps:
(a) providing a nucleic acid encoding a polypeptide with an amidase activity comprising a sequence as set forth in claim 1 or claim 22; and,
(b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon under-represented in coding sequences in genes in the host cell, thereby modifying the nucleic acid to increase its expression in a host cell.

160. A method for modifying codons in a nucleic acid encoding an amidase polypeptide, the method comprising the following steps:
(a) providing a nucleic acid encoding a polypeptide with an amidase activity comprising a sequence as set forth in claim 1 or claim 22; and,
(b) identifying a codon in the nucleic acid of step (a) and replacing it with a different codon encoding the same amino acid as the replaced codon, thereby modifying codons in a nucleic acid encoding an amidase.

161. A method for modifying codons in a nucleic acid encoding an amidase polypeptide to increase its expression in a host cell, the method comprising the following steps:
(a) providing a nucleic acid encoding an amidase polypeptide comprising a sequence as set forth in claim 1 or claim 22; and,
(b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon under-represented in coding sequences in genes in the host cell, thereby modifying the nucleic acid to increase its expression in a host cell.

162. A method for modifying a codon in a nucleic acid encoding a polypeptide having an amidase activity to decrease its expression in a host cell, the method comprising the following steps:
(a) providing a nucleic acid encoding an amidase polypeptide comprising a sequence as set forth in claim 1 or claim 22; and
(b) identifying at least one preferred codon in the nucleic acid of step (a) and replacing it with a non-preferred or less preferred codon encoding the same amino acid as the replaced codon, wherein a preferred codon is a codon over-represented in coding sequences in genes in a host cell and a non-preferred or less preferred codon is a codon under-represented in coding sequences in genes in the host cell, thereby modifying the nucleic acid to decrease its expression in a host cell.

163. The method of claim 160 or 161, wherein the host cell is a bacterial cell, a fungal cell, an insect cell, a yeast cell, a plant cell or a mammalian cell.

164. A method for producing a library of nucleic acids encoding a plurality of modified amidase active sites or substrate binding sites, wherein the modified active sites or substrate binding sites are derived from a first nucleic acid comprising a sequence encoding a first active site or a first substrate binding site the method comprising the following steps:
(a) providing a first nucleic acid encoding a first active site or first substrate binding site, wherein the first nucleic acid sequence comprises a sequence that hybridizes under stringent conditions to a sequence as set forth in claim 1 or claim 22, or a subsequence thereof, and the nucleic acid encodes an amidase active site or an amidase substrate binding site;
(b) providing a set of mutagenic oligonucleotides that encode naturally-occurring amino acid variants at a plurality of targeted codons in the first nucleic acid; and,
(c) using the set of mutagenic oligonucleotides to generate a set of active site-encoding or substrate binding site-encoding variant nucleic acids encoding a range of amino acid variations at each amino acid codon that was mutagenized, thereby producing a library of nucleic acids encoding a plurality of modified amidase active sites or substrate binding sites.

165. The method of claim 163, comprising mutagenizing the first nucleic acid of step (a) by a method comprising an optimized directed evolution system.

166. The method of claim 163, comprising mutagenizing the first nucleic acid of step (a) by a method comprising gene site-saturation mutagenesis (GSSM).

167. The method of claim 163, comprising mutagenizing the first nucleic acid of step (a) by a method comprising a synthetic ligation reassembly (SLR).

168. The method of claim 163, further comprising mutagenizing the first nucleic acid of step (a) or variants by a method comprising error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturated mutagenesis (GSSM), synthetic ligation reassembly (SLR) and a combination thereof.

169. The method of claim 163, further comprising mutagenizing the first nucleic acid of step (a) or variants by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof.

170. A method for making a small molecule comprising the following steps:
(a) providing a plurality of biosynthetic enzymes capable of synthesizing or modifying a small molecule, wherein one of the enzymes comprises an amidase enzyme encoded by a nucleic acid comprising a sequence as set forth in claim 1 or claim 22;
(b) providing a substrate for at least one of the enzymes of step (a); and
(c) reacting the substrate of step (b) with the enzymes under conditions that facilitate a plurality of biocatalytic reactions to generate a small molecule by a series of biocatalytic reactions.

171. A method for modifying a small molecule comprising the following steps:
(a) providing an amidase enzyme, wherein the enzyme comprises a polypeptide as set forth in claim 65 or a polypeptide encoded by a nucleic acid as set forth in claim 1 or claim 22;
(b) providing a small molecule; and
(c) reacting the enzyme of step (a) with the small molecule of step (b) under conditions that facilitate an enzymatic reaction catalyzed by the amidase enzyme, thereby modifying a small molecule by an amidase enzymatic reaction.

172. The method of claim 170, comprising a plurality of small molecule substrates for the enzyme of step (a), thereby generating a library of modified small molecules produced by at least one enzymatic reaction catalyzed by the amidase enzyme.

173. The method of claim 170, further comprising a plurality of additional enzymes under conditions that facilitate a plurality of biocatalytic reactions by the enzymes to form a library of modified small molecules produced by the plurality of enzymatic reactions.

174. The method of claim 170, further comprising the step of testing the library to determine if a particular modified small molecule which exhibits a desired activity is present within the library.

175. The method of claim 173, wherein the step of testing the library further comprises the steps of systematically eliminating all but one of the biocatalytic reactions used to produce a portion of the plurality of the modified small molecules within the library by testing the portion of the modified small molecule for the presence or absence of the particular modified small molecule with a desired activity, and identifying at least one specific biocatalytic reaction that produces the particular modified small molecule of desired activity.

176. A method for determining a functional fragment of an amidase enzyme comprising the steps of:
(a) providing an amidase enzyme, wherein the enzyme comprises a polypeptide as set forth in claim 65 or a polypeptide encoded by a nucleic acid as set forth in claim 1 or claim 22; and
(b) deleting a plurality of amino acid residues from the sequence of step (a) and testing the remaining subsequence for an amidase activity, thereby determining a functional fragment of an amidase enzyme.

177. The method of claim 175, wherein the amidase activity is measured by providing an amidase substrate and detecting a decrease in the amount of the substrate or an increase in the amount of a reaction product.

178. A method for whole cell engineering of new or modified phenotypes by using real-time metabolic flux analysis, the method comprising the following steps:
(a) making a modified cell by modifying the genetic composition of a cell, wherein the genetic composition is modified by addition to the cell of a nucleic acid comprising a sequence as set forth in claim 1 or claim 22;
(b) culturing the modified cell to generate a plurality of modified cells;
(c) measuring at least one metabolic parameter of the cell by monitoring the cell culture of step (b) in real time; and,
(d) analyzing the data of step (c) to determine if the measured parameter differs from a comparable measurement in an unmodified cell under similar conditions, thereby identifying an engineered phenotype in the cell using real-time metabolic flux analysis.

179. The method of claim 177, wherein the genetic composition of the cell is modified by a method comprising deletion of a sequence or modification of a sequence in the cell, or, knocking out the expression of a gene.

180. The method of claim 177, further comprising selecting a cell comprising a newly engineered phenotype.

181. The method of claim 179, further comprising culturing the selected cell, thereby generating a new cell strain comprising a newly engineered phenotype.

182. A method for hydrolyzing a amide bond comprising the following steps:
(a) providing a polypeptide having an amidase activity, wherein the polypeptide comprises a polypeptide as set forth in claim 65 or a polypeptide encoded by a nucleic acid as set forth in claim 1 or claim 22;
(b) providing a composition comprising an amide bond; and
(c) contacting the polypeptide of step (a) with the composition of step (b) under conditions wherein the polypeptide hydrolyzes the amide bond.

183. The method as set forth in claim 181, wherein the composition comprises an internal amide bond.

184. The method as set forth in claim 181, wherein the composition comprises a C-terminal amide bond.

185. The method as set forth in claim 181, wherein the composition comprises a N-terminal amide bond.

186. A method of increasing thermotolerance or thermostability of an amidase polypeptide, the method comprising glycosylating an amidase polypeptide, wherein the polypeptide comprises at least thirty contiguous amino acids of a polypeptide as set forth in claim 65 or a polypeptide encoded by a nucleic acid as set forth in claim 1 or claim 22, thereby increasing the thermotolerance or thermostability of the amidase polypeptide.

187. The method of claim 185, wherein the amidase specific activity is thermostable or thermotolerant at a temperature in the range from greater than about 37° C. to about 95° C.

188. A method for overexpressing a recombinant amidase polypeptide in a cell comprising expressing a vector comprising a nucleic acid of claim 1 or claim 22, wherein overexpression is effected by use of a high activity promoter, a dicistronic vector or by gene amplification of the vector.

189. A detergent composition comprising a polypeptide as set forth in claim 65 or a polypeptide encoded by a nucleic acid as set forth in claim 1 or claim 22, wherein the polypeptide comprises an amidase activity.

190. A method for resolution of racemic mixtures of optically active compounds comprising the following steps:
(a) providing a polypeptide comprising a polypeptide as set forth in claim 65 or a polypeptide encoded by a nucleic acid as set forth in claim 1 or claim 22, wherein the polypeptide is selective for one enantiomer of optically active compounds;
(b) providing a racemic mixture of optically active compounds, and
(c) contacting the polypeptide of step (a) with the mixture of step (b) under conditions wherein the polypeptide can selectively convert only one enantiomer of optically active compound thereby resulting in a resolution of racemic mixtures.

191. The method as set forth in claim 189 wherein the polypeptide is selective for a L-enantiomer.

192. The method as set forth in claim 189 wherein the polypeptide is selective for an R-enantiomer.

193. The method as set forth in claim 189 wherein the polypeptide is stereospecific.

194. A method for synthesizing a compound comprising an amide bond comprising the following steps:
(a) providing a polypeptide comprising a polypeptide as set forth in claim 65 or a polypeptide encoded by a nucleic acid as set forth in claim 1 or claim 22, wherein the polypeptide comprises an amidase activity;
(b) providing precursors; and
(c) contacting the polypeptide of step (a) with the precursor of step (b) under conditions wherein the polypeptide can catalyze the synthesis of the amide bond.

195. The method of claim 193 wherein the polypeptide is stereoselective or stereospecific and the compound comprising an amide bond is chiral.

196. The method of claim 193 wherein the precursors are poorly water-soluble.

197. The method of claim 193 wherein the precursors are achiral and the compound comprising an amide bond is chiral.

198. The method of claim 193 wherein the compound comprising an amide bond is an amino acid or amino amid.

199. The method of claim 193 wherein the compound is methyl dopa.

200. A method for hydrolysis of a penicillin comprising the following steps:
(a) providing a polypeptide comprising a polypeptide as set forth in claim 65 or a polypeptide encoded by a nucleic acid as set forth in claim 1 or claim 22;
(b) providing a composition comprising a penicillin;
(c) combining the polypeptide of step (a) with the composition of the step (b) under conditions wherein the polypeptide can hydrolyze the penicillin.

201. A method for hydrolysis of a cephalosporin comprising the following steps:
(a) providing a polypeptide comprising a polypeptide as set forth in claim 65 or a polypeptide encoded by a nucleic acid as set forth in claim 1 or claim 22;
(b) providing a composition comprising a cephalosporin;
(c) combining the polypeptide of step (a) with the composition of the step (b) under conditions wherein the polypeptide can hydrolyze the cephalosporin.

202. The method as set forth in claim 201, wherein the cephalosporin is cephalosporin C.

203. A method for synthesis of a 7-aminocephalosporanic acid (7-ACA) comprising the following steps:
(a) providing a polypeptide comprising a polypeptide as set forth in claim 65 or a polypeptide encoded by a nucleic acid as set forth in claim 1 or claim 22;
(b) providing a composition comprising a cephalosporin C;
(c) combining the polypeptide of step (a) with the composition of the step (b) under conditions wherein the polypeptide can convert the cephalosporin C to 7-aminocephalosporanic acid (7-ACA).

204. A method for cell wall hydrolysis comprising the following steps:
(a) providing a polypeptide comprising a polypeptide as set forth in claim 65 or a polypeptide encoded by a nucleic acid as set forth in claim 1 or claim 22;
(b) providing a composition comprising a cell wall; and
(c) contacting the polypeptide of step (a) with the composition of step (b) wherein the polypeptide can hydrolyze the cell wall.

205. A method for influencing fermentation in food processing comprising the following steps:
(a) providing a polypeptide comprising a polypeptide as set forth in claim 65 or a polypeptide encoded by a nucleic acid as set forth in claim 1 or claim 22;
(b) providing a composition comprising bacterial used in food processing;
(c) contacting the polypeptide of step (a) with the composition of step (b) under conditions wherein the polypeptide can change the fermentation characteristics of the bacteria.

206. The method as set forth in claim 204, wherein the fermentation characteristics of bacteria comprise speed of growth, acid production or survival.

207. A method for cheese ripening and flavor development comprising the following steps:
(a) providing a polypeptide comprising a polypeptide as set forth in claim 65 or a polypeptide encoded by a nucleic acid as set forth in claim 1 or claim 22;
(b) providing a composition comprising cheese;
(c) contacting the polypeptide of step (a) with the composition of step (b) under conditions wherein the polypeptide hydrolyzes milk casein thereby assisting in cheese ripening and the development of cheese flavor.

208. A method for promoting bacterial or fungal killing comprising providing a polypeptide comprising a polypeptide as set forth in claim 65 or a polypeptide encoded by a nucleic acid as set forth in claim 1 or claim 22, and contacting the polypeptide of step (a) with a composition, thereby promoting bacterial or fungal killing.

209. An antimicrobial composition comprising a polypeptide as set forth in claim 65 or a polypeptide encoded by a nucleic acid as set forth in claim 1 or claim 22.

210. The antimicrobial composition of claim 208, wherein the antimicrobial composition is a bacteriocide or a fungicide.

211. A food product comprising a polypeptide as set forth in claim 65 or a polypeptide encoded by a nucleic acid as set forth in claim 1 or claim 22.

212. A cheese comprising a polypeptide as set forth in claim 65 or a polypeptide encoded by a nucleic acid as set forth in claim 1 or claim 22.

213. A dairy product comprising a polypeptide as set forth in claim 65 or a polypeptide encoded by a nucleic acid as set forth in claim 1 or claim 22.

214. A pharmaceutical composition comprising a polypeptide as set forth in claim 65 or a polypeptide encoded by a nucleic acid as set forth in claim 1 or claim 22.

215. A fluorescent secondary amidase substrate comprising 7-(ε-D-2-aminoadipoylamido)-4-methylcoumarin.


SEQUENCE LISTING (114 sequences)

SEQ ID NO: 1 - DNA, 879 bp, Unknown
  Obtained from an environmental sample
atgaactcaaccttagcctacttcacggaacagggacccatgtctgacccgggaacctatcgttcgctttttgaagatcttcccacatccatcccagatctggtgaagcttgtgcagggagtcaccctacatatcttttggacggagcgatatggactcaaagttcccccgcaacgaatggaggaactgcagctccgttcgatggagaaacggctggcgcgcacgctcgaattagatccgcgtccacttgttgagccgcgtccgctagagaacaagttgctcggcaattgtcgggatcattctctattgcttaccgcgctgctgcgtcatcagggagttccggctcgcgcccgctgtgggtttggtgcctacttcctgccagaccattttgaggaccactgggtcgttgagtactggaatcaggagcaatcccgctgggtacttgtggacgcacagttggatgcctcacagcgcgaggtgttgaagatcgactttgacactttggatgtcccccgtgatcaattcatcgtcggcggcaaagcctggcaaatgtgccgttctggcgagcaagaccctggcaaattcggcattttcgatatgaatggattgggcttcgtgcggggggatcttgtacgtgatgtcgcctcgctcaataaaatggaattgctgccctgggattgctggggtgttattctcgttgagaaactcgatgacccggctgacctttccgtgcttgatcgagtcgcttcgctcaccgcgagagatgtccccgattttgaagtgctgcgcgcctgttatgagtctgatccgcgactgcgtgtgaacgactcattgctgagctacgtcaacgggaacatggtggaagtccagatcgcttaa

SEQ ID NO: 2 - protein, 292 aa, Unknown
  Obtained from an environmental sample
MNSTLAYFTEQGPMSDPGTYRSLFEDLPTSIPDLVKLVQGVTLHIFWTERYGLKVPPQRMEELQLRSMEKRLARTLELDPRPLVEPRPLENKLLGNCRDHSLLLTALLRHQGVPARARCGFGAYFLPDHFEDHWVVEYWNQEQSRWVLVDAQLDASQREVLKIDFDTLDVPRDQFIVGGKAWQMCRSGEQDPGKFGIFDMNGLGFVRGDLVRDVASLNKMELLPWDCWGVILVEKLDDPADLSVLDRVASLTARDVPDFEVLRACYESDPRLRVNDSLLSYVNGNMVEVQIA

SEQ ID NO: 3 - DNA, 936 bp, Unknown
  Obtained from an environmental sample
gtgccgagcctcgacgagtacgcgacccacagcgccttcaccgaccccggccggcaccgggacctgctcggcgcgaccgggacgtcgcccgacgacctgcaccgtgcggcgacaggcgtcgtcctgcactaccgcggccagcgcgaccggctcacggacgagcagctgcccgacgtcgacctgcgctggttctccgcccagctcgaggtcgttcggcaccgcgcggcgctcccgctcggcgcgcaccggacggacgcgcagcacctcgcggggtgctgccgcgaccacacgctgctcgccgtcgccgtcctgcgcgagcacggcatccccgcgcgcagccgcgtcggcttcgccgactacttcgagcccgacttccaccacgaccacgtcgtcgtcgagcggtgggacggcgcgcggtgggtgcgcttcgactcggcgctggacccggcggaccacctgttcgacgtggacgacatgccggcgggggagggcatgccgttcgagacggccgccgaggtctggctcgccgcgcgggcgggccgcgtcgacccccggcggtacggcgtggacaaggcgatgccgcacctgatcggcatcccgttcctgctcggcgaggtcttcctcgagctcgcgcaccggcagcgcgacgagatcctgctgtgggacgtgtggggcgtcggcatcccgccgttcgcgcggccggacggcctggcacccgtgaccatgtcggacgacgagatggcggagctcgccgacgaggtggcgcggctcgtcgtcgcggcggacgacggcgacgacgcggctgacgcggcgctcgacgcccgctacgccgccgatccccgcctgcggccgacggccaacccgctcgtggcgctctcgccgctcgaacgcatcggggacgtcgacctgacggcgcggacgacgacctggcggtga

SEQ ID NO: 4 - protein, 311 aa, Unknown
  Obtained from an environmental sample
VPSLDEYATHSAFTDPGRHRDLLGATGTSPDDLHRAATGVVLHYRGQRDRLTDEQLPDVDLRWFSAQLEVVRHRAALPLGAHRTDAQHLAGCCRDHTLLAVAVLREHGIPARSRVGFADYFEPDFHHDHVVVERWDGARWVRFDSALDPADHLFDVDDMPAGEGMPFETAAEVWLAARAGRVDPRRYGVDKAMPHLIGIPFLLGEVFLELAHRQRDEILLWDVWGVGIPPFARPDGLAPVTMSDDEMAELADEVARLVVAADDGDDAADAALDARYAADPRLRPTANPLVALSPLERIGDVDLTARTTTWR

SEQ ID NO: 5 - DNA, 909 bp, Unknown
  Obtained from an environmental sample
atgaccaatcagccggagcgcagcaccgcacggtcatactacgccgccccggcggcgatgaccgacttgagcgcgcatcgcgcgcgcttgcgcgacctgccgaccgatctggccgggctctgccgcgtcattcagggactgctggtgcatccctttctcgcgcacctctacggcctgccgtcgagcgcgctgcgcctcggcgagttggagttgcgccgcgcctcggcgatgctcgatcacgcgttgaccctcgacgcgcgcccgctcgtcgaggcgcgcccgccggagcgacgcctggtgggcaactgccgccacttttcggtgctgttctgcgccttactgcgcgcccagggcgttccggcgcgcgcccgctgcggattcggcgcctacttcaatccggcgcgtttcgaggatcactgggtcggcgaagtctgggactcgacgcgcggcgcctggcgcctcgtcgacgcacagctcgatgccgagcagcgccaggcgctgcgcatctcgttcgatccgctcgacgtgccgcgcagcgagttcgtggtagccggcgaggcgtggcgacggtgccggagcggcgcggccgctcccgaactgttcggcatcctcgatctgcgcggtctctggttcgtgcgcggcaacgtggtgcgcgacctcgccgcgttcagcaagcgcgaactgctgccgtgggacggctggggtctgatggcgacgcgcgaggacagcagtcctgccgagctggcgctactcgaccacgtcgccgagctgactctggccggcgacgagcgccacgacgagcgcctgcatctgcaggatgccgaacccggcctgcgcgtgcctcgcgtcgttctcagcttcaacctgaacggcgccgaggtcgatctcggccccggcgttgcgaactga

SEQ ID NO: 6 - protein, 302 aa, Unknown
  Obtained from an environmental sample
MTNQPERSTARSYYAAPAAMTDLSAHRARLRDLPTDLAGLCRVIQGLLVHPFLAHLYGLPSSALRLGELELRRASAMLDHALTLDARPLVEARPPERRLVGNCRHFSVLFCALLRAQGVPARARCGFGAYFNPARFEDHWVGEVWDSTRGAWRLVDAQLDAEQRQALRISFDPLDVPRSEFVVAGEAWRRCRSGAAAPELFGILDLRGLWFVRGNVVRDLAAFSKRELLPWDGWGLMATREDSSPAELALLDHVAELTLAGDERHDERLHLQDAEPGLRVPRVVLSFNLNGAEVDLGPGVAN

SEQ ID NO: 7 - DNA, 891 bp, Unknown
  Obtained from an environmental sample
atgcgcagcgacctcgcattctatcaaacacaggggatcatcaccgatcccggccaacatcacgacctgctgaccggcctgccgggcgacctgcccggcctggtcaaagtcgtccagggcctggtggtgcacgtcttctggctggagcgctacggcttgaagctgaaggagacgcgcaaggccgaggtgcagttgcgctgggctgaaaagcagctcgagcgcatccgcgcgctcgacccgcgcccgctggccgaagcccggcccctggagaagcgcctggtgggcaactgccgggatttcaccgtcctgctggtatgcctgctgcgcgcccggggcatcccggcccgcgcgcgctgcggtttcgccaagtacttcgaggcggggcggcacatggatcactgggtggccgaggtttggaacgccgagctgcaacgctggactttggtcgacgcacaactcgacgacctgcagcgcaaggcgctcgcgataccgttcaacccgctggacgtgccgcgcgtgcagttcctgaccggcggcgaagcctggctgcgctgccgcaaggggcaggccgaccccgagaccttcggcatcttcgacctgaaggggttgtggttcgtgcgcggggacttcgtgcgcgacgtggccgcgctcaacaaggttgagctgctgccctgggatgcatggggcatcgccgatgtgcaggaaaaggatatctccggggaagacctggttttcctggacgaggtggccgagctctcacatggcgacgtggagcgcttcgagcaggtgaaggggctgtatgaaaccgacccccggctgcacgtgccggaggtgatcaacagttacacacaggcaggggtgctgcgcgtcgatctccaagcacattcgtag

SEQ ID NO: 8 - protein, 296 aa, Unknown
  Obtained from an environmental sample
MRSDLAFYQTQGIITDPGQHHDLLTGLPGDLPGLVKVVQGLVVHVFWLERYGLKLKETRKAEVQLRWAEKQLERIRALDPRPLAEARPLEKRLVGNCRDFTVLLVCLLRARGIPARARCGFAKYFEAGRHMDHWVAEVWNAELQRWTLVDAQLDDLQRKALAIPFNPLDVPRVQFLTGGEAWLRCRKGQADPETFGIFDLKGLWFVRGDFVRDVAALNKVELLPWDAWGIADVQEKDISGEDLVFLDEVAELSHGDVERFEQVKGLYETDPRLHVPEVINSYTQAGVLRVDLQAHS

SEQ ID NO: 9 - DNA, 903 bp, Unknown
  Obtained from an environmental sample
atgaccgatcgtgcgccgtacgccgcccagagtcccatctccgatccgggcgatatgtccaggtggcttactggcttgccagcagatttcgcggccctgcgggcgctggccaggccgctggtcgcacactaccgggccgatgacctggcggcgttcggcattcccgaggagcgcgtggaggagatcgacacgcggtttgcggagcggatgctggcgcggctgcacgagatggagagcggtccgctcacgccggagcgcacgccggccaaccgcctcgtgggctgctgccgggacttcaccctgctctacctgaccatgctgcgccacgccggcatcccggcacggtcacgcgtgggctttgccggctactttgccgctggctggttcatcgaccacgtggtggctgaggtctgggacgaggccaacgggcgctggcgcctggtcgatccccagttggcggatgtgcgcactgaccccaacgacggcttccccatcgatacgctcgatatcccgcgcgaccgtttcctggttgcgggcatggcgtggcaggcttgccggagtgaggaactgcagccagagcagttcgtggttgacccagatctcgatatcccggtgacgcgcggctggctgcaactgcggcacaacctggtgcaggacctcgccgcactgacgaagcgggagatgatcctctgggatacgtggggcatcctgggtgacgagccggtggcggaggatacgctgcccttgctggacagcatcgcggctgtcaccgccgatcccgatgtcacgtacgcggacgccctcaatctctacgagcgggagccgggggtgcaggtgccgccagaggtgatgagcttcaacatgctggcgaacgagccaaggatggtggcgtcgggggtgtag

SEQ ID NO: 10 - protein, 300 aa, Unknown
  Obtained from an environmental sample
MTDRAPYAAQSPISDPGDMSRWLTGLPADFAALRALARPLVAHYRADDLAAFGIPEERVEEIDTRFAERMLARLHEMESGPLTPERTPANRLVGCCRDFTLLYLTMLRHAGIPARSRVGFAGYFAAGWFIDHVVAEVWDEANGRWRLVDPQLADVRTDPNDGFPIDTLDIPRDRFLVAGMAWQACRSEELQPEQFVVDPDLDIPVTRGWLQLRHNLVQDLAALTKREMILWDTWGILGDEPVAEDTLPLLDSIAAVTADPDVTYADALNLYEREPGVQVPPEVMSFNMLANEPRMVASGV

SEQ ID NO: 11 - DNA, 756 bp, Unknown
  Obtained from an environmental sample
atgcttgcagccggggtaccaggacgacttgtaggccttcaccggattgttgaactcgatctcgagcgtgaaacgctcgggcagctgcagcaggcacttcttcaggtcgccctgcagtgcctgcctgacgccctcgcggatctgcgcgcaggcggccttgggcgcgaggctgacggtcgacggcccgatgccctcgctgaccgcgacggcggtgatgttggggttggtctccttgatgtcggtgcagagctgccagtcgccggagacgaacaccaccgggacgccgaccatggcggcggcataggcgtgcagcaggaattccgaagtcgccacgccgttgatgcgcatgcgcatgacctcacccgtcagcgtgtgcgccaagggattggtctcgtcgccggccttcgagtgatagccgatgaacatcgcggcatcgaagctcttgtccagctcctgcaccatgctcatcggatggccgctccagccgcggatcaggcgcacattctccggcaggtcggcctgcaggatgttgcgcccggtcgcgtgtgcgtccttgatcaggatctccttggcccccgccgcgttggcgccgtcgcacgccgccagcacttcgcgcgtcatctgctcgcgatgctcgggatagtcggcgtgcggcttgcgcgcctcgtcccagttggtgatgccggcggtgccctcgatgtcggcgctgatgaagatcttcatgccactcctctttgcaaacgcgcgccactctag

SEQ ID NO: 12 - protein, 251 aa, Unknown
  Obtained from an environmental sample
MLAAGVPGRLVGLHRIVELDLERETLGQLQQALLQVALQCLPDALADLRAGGLGREADGRRPDALADRDGGDVGVGLLDVGAELPVAGDEHHRDADHGGGIGVQQEFRSRHAVDAHAHDLTRQRVRQGIGLVAGLRVIADEHRGIEALVQLLHHAHRMAAPAADQAHILRQVGLQDVAPGRVCVLDQDLLGPRRVGAVARRQHFARHLLAMLGIVGVRLARLVPVGDAGGALDVGADEDLHATPLCKRAPL

SEQ ID NO: 13 - DNA, 960 bp, Unknown
  Obtained from an environmental sample
ttgccgcaaggcgtgtgcgccgcttcactgcgtcggtatcggcaacgaaaggaacagtacctcatgacgatacaccaacagattctcgacttctatacgcgccctgccgggatgacgtccgccggccaattcgcgcccttattcgacgcgctgccgagcgacgtgggcgaactcgtccgcatcatccagggccttggggtgtatgaccttgtggcgtccggcttctacggcttcacgatcccggacgagcgccagggcgagatccacctccgccccgtagagaaaatgctgggccgcctcctcgccctcgacgaccggccgctccgtgtcgcccggccggtcgacaggcgtctggtcggccgctgccgtcacttcgtgctgctactcgtcgccatgttgcgggccaagggtgttccggcgcgggcgcgctgcgggttcggctcctactttagacgcgggttctttgaggaccactgggtgtgcgagtactggaacgccgccgaagcccgctgggtgcttgtcgatccacagttcgacgaggtttggcgggagacactacagatagatcacgacattcttgatgtgccgcgcgaccgtttcctggtagcgggcgacgcctgggcgcaatgccgcgcgggtgcggccgacccggcgaagttcgaaatcgttttcgccgacctgagcggactgtggttcatcgccgggaacctggtgcgcgacgtggcggcgctcaacaagacggagatgctgccgtgggacgtctggggcgcccagccccgcccgcacgaagcgctcgacgacgaccaactgaccttcttcgacaaactcgccgcgctcacgcgcgagcctgacgcgtcgttcgcggaactgcgcaccctctacgaaggagatgatcgcctgcgtgtgccggcgaccgtcttcaacgcgatgcgcaacgcgcccgaaacgatcgcgggctga

SEQ ID NO: 14 - protein, 319 aa, Unknown
  Obtained from an environmental sample
MPQGVCAASLRRYRQRKEQYLMTIHQQILDFYTRPAGMTSAGQFAPLFDALPSDVGELVRIIQGLGVYDLVASGFYGFTIPDERQGEIHLRPVEKMLGRLLALDDRPLRVARPVDRRLVGRCRHFVLLLVAMLRAKGVPARARCGFGSYFRRGFFEDHWVCEYWNAAEARWVLVDPQFDEVWRETLQIDHDILDVPRDRFLVAGDAWAQCRAGAADPAKFEIVFADLSGLWFIAGNLVRDVAALNKTEMLPWDVWGAQPRPHEALDDDQLTFFDKLAALTREPDASFAELRTLYEGDDRLRVPATVFNAMRNAPETIAG

SEQ ID NO: 15 - DNA, 1182 bp, Unknown
  Obtained from an environmental sample
gtggaccaaaccggagcaaatgacgcactggtggggcatggccggcggcccgcgtccgccggtcgccgagaccgacctgcgcgtcggcggccgcttcaaggtgcagttctgggatcccaagaacgagcatcacagcgtcagcggcatctacaaggaggtcgtgcccaaccggaagctcgccttctcgtgggcctggcagagcacgcccgagcgcgaatcgctggtgacgatcgagctcaacccggtcaccgagggcaccatgctgacgctgacccacgagcagttcttcgacgagaaggcgcgcgacgaccacggccgcggctggaacgtcgccctcgaccgcctggagagcttcctcacatgacccccggccaggccgtggaccgggcgttcgccggcttgccgggcgatcccgcgtcgctggccggcgtcgtgcagggccttttgatgcacgagcatatcgcgccggcctacggcctcaccctgagcgaggcccagcacgcggaggcgcacacccggccggtcgaggagatcgtgcgccagatcgtggcgcacgatcctcgtccgctcgccgagccgcgcgcgcccggcgaacgccaggtcggcaattgccggcacttcaccctgctgcacgtcacgatgctgcgccgcgccggcgtgcgggcgcgcgcccgctgcggcttcggcggctacttcgagccgggcaagttcctcgaccactgggtcaccgaatactggaacgagcggcgccaggcgtgggttctggtcgatgcccagctcgatgcccgccagcgcgagctcttcaagatcgccttcgaccccctcgacgtgccgcgcgacaagttcctggtcgcgggcgacgcctggcagcgctgccgcgccggcaccgccgatccgaacgcgttcggcatcctcgacatgcacgggctgtggttcgtcgccggcaatttgatccgcgacgtcgccgcgctcaacgaccacgtgatgctgccgtgggacgtgtggggcgcgatgacccagaacgacgcggagctcgaccaaccgttcctcgacaagctggccgcgctgaccgtcgagcccgaccgccatttcggcgagctgcgcgccgtctaccaggatccgcgcgtgaaagtgccggcgaccgtgttcaacgccatccgcaaccgccccgaaaccctttga

SEQ ID NO: 16 - protein, 393 aa, Unknown
  Obtained from an environmental sample
VDQTGANDALVGHGRRPASAGRRDRPARRRPLQGAVLGSQERASQRQRHLQGGRAQPEARLLVGLAEHARARIAGDDRAQPGHRGHHADADPRAVLRREGARRPRPRLERRPRPPGELPHMTPGQAVDRAFAGLPGDPASLAGVVQGLLMHEHIAPAYGLTLSEAQHAEAHTRPVEEIVRQIVAHDPRPLAEPRAPGERQVGNCRHFTLLHVTMLRRAGVRARARCGFGGYFEPGKFLDHWVTEYWNERRQAWVLVDAQLDARQRELFKIAFDPLDVPRDKFLVAGDAWQRCRAGTADPNAFGILDMHGLWFVAGNLIRDVAALNDHVMLPWDVWGAMTQNDAELDQPFLDKLAALTVEPDRHFGELRAVYQDPRVKVPATVFNAIRNRPETL

SEQ ID NO: 17 - DNA, 1032 bp, Unknown
  Obtained from an environmental sample
gtgcgatcctctcgcgcgatgtcgctcctcgatcgtccccacgccgagctgcgcgcggccctcgcgaccggcgcgcccgtctacctccccgtcaatcccgtcgagtaccacgggccgcacctctcgctgcacaacgacgcgctcgtcagcgccgggttgatgcgcgatctccacgcggccctgaacgcacgtcgacgcgcgcgcggcgaagaagagtggccgttcctcgtcgcgtcggacctcgagatcggcgtcgagccgacccccgggctcgggtcgcgccacacgccgttccccctcgcgcgcgcgctcgttcgcgaggccgcgcgtgcgatcgccgagctcggcgcgcggaaggtcgtcttcgcgacgttccacggatcgccccttcacaacgtcgcgatcgaggcgggcgtcgagctccttcaggcgcgcggcgtcctcgcgttgaacccgttcaatctggtcatgagcgagctcgtcgacgcgttcgcgctccccggcgagccccccgcgaacgacgtgacgacgctcgccgacgcgtacgcgcacatcgagccgtcgctgcgcgcgtcgatgatgcgcgaggccgcgagcgacttccacgcagggttcttcgagacgtcgatcacgctgcactacgcgccgcactcggtctcgccgtcctacgtcgacgtgccgccgtgcccgccgatcgtgccggagcgcgtgtggatcgccctcgcggcaggcgcgcgaatcgccggaaaaaaacgcctcgcgcgcgagctcaccctcgcggcgtacgggatggggtggcacgcgctcgatccattccctggttatacggggcggccatcgctcgcgcgcgccgagtccggggcgatcctcgcgcgcctctccgtcgagcgatacgtcgacgtcgccgaggcggtcttcgcggggcgcgcgtgcccaccgaagccgatcctcgcgtggacagggtcgctcagcttgaacgggcgcctctcgccgaagcccccgtcgctccgcgcgctcgaagcgctttcctga

SEQ ID NO: 18 - protein, 343 aa, Unknown
  Obtained from an environmental sample
MRSSRAMSLLDRPHAELRAALATGAPVYLPVNPVEYHGPHLSLHNDALVSAGLMRDLHAALNARRRARGEEEWPFLVASDLEIGVEPTPGLGSRHTPFPLARALVREAARAIAELGARKVVFATFHGSPLHNVAIEAGVELLQARGVLALNPFNLVMSELVDAFALPGEPPANDVTTLADAYAHIEPSLRASMMREAASDFHAGFFETSITLHYAPHSVSPSYVDVPPCPPIVPERVWIALAAGARIAGKKRLARELTLAAYGMGWHALDPFPGYTGRPSLARAESGAILARLSVERYVDVAEAVFAGRACPPKPILAWTGSLSLNGRLSPKPPSLRALEALS

SEQ ID NO: 19 - DNA, 945 bp, Unknown
  Obtained from an environmental sample
atgactgccccgtcggttcaggtctggtcacgtcagacccggttcagcgatcccggcccgcacgcgcagctgctcgccgcggctccggacgacattcgcggcctggccgcgacggtgcgcaacacgatcacgcactaccgggccggtccggcgctgccgccggggcggatgtcggagattgacaaccggtgggtggagcggattctcgccacggaccagacacgtcacgccgagccgctgagcgtgtcgcggccaccgcaggagagggtcgccggctgctgccgcgacttcacactgctgaccgtggccgcgctgcgtcagcggggcgtgccggcgcgtagtcgggtcgggttcgccggctacttctacgacaccggattccatgccgatcatgtgatcgtcgactggtgggatggtgaccgctgggtgttcaccgacgctcagcttgacccggcccacgactggggcttcgacccggccgacatgcgccggacggtgggtgaccgagtgccgggtccgttcgcgacggcagcccaggtgtggacggcgtaccggcgcggggagatcgacggtcagaactacggcgtggacccggagctgccgctgcggggcggcccgttcctgcgcgactacgtgatcatggagctggcccatcggcgcggcgacgaactcctgctctgggaccgttggggcgccgggtcgaccaacccggtgggcgaactgaccgatgaggtggacttcatcgacgaggtggccgcccttctgctgcgggccgacgagggggacgaacgggccgaggttgagctggtcgagcggtacgcggccgacgatcggctgcaccccggcgccgaggtgacctgcgtgtcaccgaccggcgtcgtgtcctcggtcgacctggcgacgcgtaccagccgagaggtcggaaagatagatctttcccactga

SEQ ID NO: 20 - protein, 314 aa, Unknown
  Obtained from an environmental sample
MTAPSVQVWSRQTRFSDPGPHAQLLAAAPDDIRGLAATVRNTITHYRAGPALPPGRMSEIDNRWVERILATDQTRHAEPLSVSRPPQERVAGCCRDFTLLTVAALRQRGVPARSRVGFAGYFYDTGFHADHVIVDWWDGDRWVFTDAQLDPAHDWGFDPADMRRTVGDRVPGPFATAAQVWTAYRRGEIDGQNYGVDPELPLRGGPFLRDYVIMELAHRRGDELLLWDRWGAGSTNPVGELTDEVDFIDEVAALLLRADEGDERAEVELVERYAADDRLHPGAEVTCVSPTGVVSSVDLATRTSREVGKIDLSH

SEQ ID NO: 21 - DNA, 2139 bp, Unknown
  Obtained from an environmental sample
atgaaaattcactcccgtttaccctttctgcttgtgctcttagttacgtgtgttctctcgatcgatagcaacgttatcgcgcaatcaaatggaggagcgggcgcgcctcaaccaccaatggcagagaagaaaacaaagactacaaatattcacggcgacacgatggtcgacgactatttttggctgcgtgaaaagacgaacccttcggtgcaggcgtatctcaagtcagaggacgaatacgcactatcggcgatgaagcacaccaccggacttcaggaaaagctgtacaacgaaatgctcagtcatatcaaacagactgacgagaacgtgccgtatcgttcaggaaactatttttattacagcagaaccgagcaaggtaaacagtatccgatcttttgccgcaagaaaggcaccaaggatgctgctgaagaaattattctcgacgtgaatgaattggccaaaggccagaagttcatgagcgtcggcatgttcataccgagtgacgacggaaacctgctcgcctatagcaccgacaatacgggctatcgccagtttttacttcaattcaaggatctgcgcacgggccaggttttgcctgaaaagattgaacgcgtgactggtctcgcatgggccaacgacaacaagacgtacttctatgtgacggaagatccagtcacaaaacgaagcgacaagtttttccgtcgcgttctcggaggagacaaggcggatctggtttacgaagagaaggatgagttgtttgatatcggaaccggtcgatcgctcgataagaaagtaatctttctcgagtcgttcagtaaaacctcgaccgaggtttattacctgctcgcggccgagccgaacggtgcgatgaagatgattcttccgcgacaagcggagcacgagtacgacgtaaatcatcgcaatggccttttctatatccgaacaaacaaaggcgcgaagaacttccgcatcgtgactgcgccggtcagcgatccctcagagaagaactggaaagagtttgtggcccatcggccggcggtgaaggtcgaagggatcagtctcttcgccgatcacgctgtgctttctgaatgggaaaacggcctgcaacaactcgaagtcatcgatttcaaatcaaacaaacgtcataacattaagtttcctgaaccggtttactcagcgaatttgacttcaaatcgcgaatacgacacgagcatagtcaggtacggctacaattcgctcgtcacgccgttttcagtgtttgattacgacatgaacacgggcaagtcgatcctgctgaaacaaaccgaagtgcccggaggttttgacagagccaactaccaggccgagcgcgtgtttgcaacggcgtccgatggaacaaaggttccgatgtcgatggtctatcgaaaaggagtcaagctcgacggcacagcgcctctacttctttacgggtatggttcatacggcgcatcgattccgccgaccttttcgtcgagtcgactcagtcttctcgatcgcggagtgatctacgtgattgctcacattcgtggtggtggcgagttgggagaagaatggcggcaagccggcaggatgatgaacaagatgaatacgttcaccgacttcatcgcctgtgccgaagcactgatcaagcagaaatacacgtcagcggatcgactcgtgatcacgggtggaagcgccggaggtttgttgatgggcgctgtatcaaacatgcgtccggatcttttcaaagcagtcgtttcgcacgtgccgtttgttgacgtgctaaacacgatgctcgatgcaacgttgccgctcaccacgagtgaatacatcgagtggggcaatccaaatgaaaagccggcgtatgaatacatgaagaagtattcgccgtacgacaatgttcagaagaaaaattacccggcaacgctggtgaaggtttccgtgaacgatagtcaggtgccgtattgggaaggtgcgaaactcgtcgccaagatgcgcgcgttgaaaaccgattcgaacccgctgctattgaaggtaaacttcggcgcgggccatggtggctcatcgggccgctacgacgcgctacgcgaaaatgcgtttgactatgcgttcgttctctggcaaatgggtattgcggaatag

SEQ ID NO: 22 - protein, 712 aa, Unknown
  Obtained from an environmental sample
MKIHSRLPFLLVLLVTCVLSIDSNVIAQSNGGAGAPQPPMAEKKTKTTNIHGDTMVDDYFWLREKTNPSVQAYLKSEDEYALSAMKHTTGLQEKLYNEMLSHIKQTDENVPYRSGNYFYYSRTEQGKQYPIFCRKKGTKDAAEEIILDVNELAKGQKFMSVGMFIPSDDGNLLAYSTDNTGYRQFLLQFKDLRTGQVLPEKIERVTGLAWANDNKTYFYVTEDPVTKRSDKFFRRVLGGDKADLVYEEKDELFDIGTGRSLDKKVIFLESFSKTSTEVYYLLAAEPNGAMKMILPRQAEHEYDVNHRNGLFYIRTNKGAKNFRIVTAPVSDPSEKNWKEFVAHRPAVKVEGISLFADHAVLSEWENGLQQLEVIDFKSNKRHNIKFPEPVYSANLTSNREYDTSIVRYGYNSLVTPFSVFDYDMNTGKSILLKQTEVPGGFDRANYQAERVFATASDGTKVPMSMVYRKGVKLDGTAPLLLYGYGSYGASIPPTFSSSRLSLLDRGVIYVIAHIRGGGELGEEWRQAGRMMNKMNTFTDFIACAEALIKQKYTSADRLVITGGSAGGLLMGAVSNMRPDLFKAVVSHVPFVDVLNTMLDATLPLTTSEYIEWGNPNEKPAYEYMKKYSPYDNVQKKNYPATLVKVSVNDSQVPYWEGAKLVAKMRALKTDSNPLLLKVNFGAGHGGSSGRYDALRENAFDYAFVLWQMGIAE

SEQ ID NO: 23 - DNA, 696 bp, Unknown
  Obtained from an environmental sample
atgcgactggaagacctgaattggatggacgtcgaatcctatcttcagaccgacgaccgcatcatcctcatcaccggcgcggctgaacagcacgcctacctgagcttactgaccgatattctgatcccgtcaaagatcgctctggccgcagccgaacgcgagcacgtactggtcgctccgccgctgaacttcggcatgagcgactattttctggcctttcccggcaccatcagcctgagcgaagccacgttccacgccgtcgtgaccgaggtcgtcaacagtctgcgccggcacggcttccgccggttcttcgtgctcaacggccacggcggcaacaccgtcccgccggcgctccaggcgctgcgcgaccgcggcgaaatcggcctgatctggtataactggtggcagcagaccgccgcccgcgcctttgaagcccactttcagacgcgcatcaaccacgccaactggggcgaaaatttccccttcaaccgggtagccgagtcgccggcggggtcaaaggccgaggtgaacctggccggcgtcaccgacccggcggcgatgcgggcggcgctgggcgatggcagctttggcggcccctaccaggccggcgacgaccagatgcagacgctgttcaccatggtggtggacgacgtgaccgacctgctgcgcgatatgccgcccgtcagccgcggctga

SEQ ID NO: 24 - protein, 231 aa, Unknown
  Obtained from an environmental sample
MRLEDLNWMDVESYLQTDDRIILITGAAEQHAYLSLLTDILIPSKIALAAAEREHVLVAPPLNFGMSDYFLAFPGTISLSEATFHAVVTEVVNSLRRHGFRRFFVLNGHGGNTVPPALQALRDRGEIGLIWYNWWQQTAARAFEAHFQTRINHANWGENFPFNRVAESPAGSKAEVNLAGVTDPAAMRAALGDGSFGGPYQAGDDQMQTLFTMVVDDVTDLLRDMPPVSRG

SEQ ID NO: 25 - DNA, 870 bp, Unknown
  Obtained from an environmental sample
atgaagttttttatttccgcagatctggaaggggcctcctgggtgaccagcccccggcaatgctttcccgagctggatttgcaaggataccagatggcggtacagcaactggccctggaagtggaagccgtcgtgcagaccattctcgcccatgggcatgcccacaaccagacccaccacatcattgtcaacgattctcacgggtatatgaccaatttaaccctggcgcatttttccccggagattcggggcaagttctcgctgctgtcgggcaagcccaaaacctgtgcaatggcggcggggctggatgaaacctgtgatgccgccctgtatattggctatcatgccaaggcgggtacccaccaggggattttaaaccacacgtttcaccccaaactgtatgatgtccgagtgaacggtgtctcctacggcgaagggggcattaacgctttccatgcgagtgtaagctttggggtgcccgtcattctggccagcggagacgaggccttctgccgggaaatccgcggggtgattccccgcatcagcaccgtctgcacgaagcattccctgtcgtttgccgccgcccttagtcgcccggtggatgaagtgttggcagagtaccaggccaagaccacggcgttgttaaacgccccgcaccagtggaaggaaaatggcctggccttgaaccccccatatgtcctggaggtcgaatttatcaattccctgtgtgccgatgtggcctgcaccatgccgtggttgcaacgaatagacggcagaacggtgacctactccgcccagtcgttccaggagctttaccaggtgctgcaatccagctatgccattctggcctacagcggctacatggaatga

SEQ ID NO: 26 - protein, 289 aa, Unknown
  Obtained from an environmental sample
MKFFISADLEGASWVTSPRQCFPELDLQGYQMAVQQLALEVEAVVQTILAHGHAHNQTHHIIVNDSHGYMTNLTLAHFSPEIRGKFSLLSGKPKTCAMAAGLDETCDAALYIGYHAKAGTHQGILNHTFHPKLYDVRVNGVSYGEGGINAFHASVSFGVPVILASGDEAFCREIRGVIPRISTVCTKHSLSFAAALSRPVDEVLAEYQAKTTALLNAPHQWKENGLALNPPYVLEVEFINSLCADVACTMPWLQRIDGRTVTYSAQSFQELYQVLQSSYAILAYSGYME

SEQ ID NO: 27 - DNA, 816 bp, Unknown
  Obtained from an environmental sample
atgaagatcgaagtggtacagacgacaacccgcgacggcgacaccgcccacaacctgcgcggtatcctcgaggccctggcccagtgcgcgccggacaccgatatcctgctgttccccgagtcgcacctgaccggttttctcaccgcggacgacgtcggcatgctgtccgaagccatcgacggcgatgccgtgcaggcggtggtcgcggcggcgcgccagcgcagtatcgcggtggtgctggggctgtacgagaacgacggtggcaccttctacaacaccaccctgttcatcgcccctgaaggcatcctgctcacctaccgcaagacccacctgtggttgccggagcacggcatcgtcgttccgggcaatcgcttcgccaccgtcgaatggcgcggcgtgcgcctgggtctgttgatctgctacgacagcgaattccccgagacggcgcgggcactgaaggccttgggcgcgcagttgatgctgatcaccgatggcctggccgaaccggaggatcatgtgcaccgggtgtcggtgatggccagggcgatggagaaccaggtgttcgccgtggtcgccaaccgcgtgggcgatgggccggatggctgcgtgttcgtgggggcgagcctggcggtggacccgtatggccgcacgctgttcgaggctgggcgcggcgagtcgaggcatagcgtgaccctggacctgtcgctgatcggcaaggcccgcgccttccatgactacagcgccaaccagcgcatccagttgccgggtgagcggatcgagcatgccagtggcgtatgggagctggttatcgattaa

SEQ ID NO: 28 - protein, 271 aa, Unknown
  Obtained from an environmental sample
MKIEVVQTTTRDGDTAHNLRGILEALAQCAPDTDILLFPESHLTGFLTADDVGMLSEAIDGDAVQAVVAAARQRSIAVVLGLYENDGGTFYNTTLFIAPEGILLTYRKTHLWLPEHGIVVPGNRFATVEWRGVRLGLLICYDSEFPETARALKALGAQLMLITDGLAEPEDHVHRVSVMARAMENQVFAVVANRVGDGPDGCVFVGASLAVDPYGRTLFEAGRGESRHSVTLDLSLIGKARAFHDYSANQRIQLPGERIEHASGVWELVID

SEQ ID NO: 29 - DNA, 798 bp, Unknown
  Obtained from an environmental sample
atgaagatcttcatcagcgccgacatcgagggcaccgccggcatcaccaactgggacgaggcgcgcaagccgcacgccgactatcccgagcatcgcgagcagatgacgcgcgaagtgctggcggcgtgcgacggcgccaacgcggcgggggccaaggagatcctgatcaaggacgcacacgcgaccgggcgcaacatcctgcaggccgacctgccggagaatgtgcgcctgatccgcggctggagcggccatccgatgagcatggtgcaggagctggacaagagcttcgatgccgcgatgttcatcggctatcactcgaaggccggcgacgagaccaatcccttggcgcacacgctgacgggtgaggtcatgcgcatgcgcatcaacggcgtggcgacttcggaattcctgctgcacgcctatgccgccgccatggtcggcgtcccggtggtgttcgtctccggcgactggcagctctgcaccgacatcaaggagaccaaccccaacatcaccgccgtcgcggtcagcgagggcatcgggccgtcgaccgtcagcctcgcgcccaaggccgcctgcgcgcagatccgcgagggcgtcaggcaggcactgcagggcgacctgaagaagtgcctgctgcagctgcccgagcgtttcacgctcgagatcgagttcaacaatccggtgaaggcctacaagtcgtcctggtaccccggctgcaagcatatcggcgagcgcaccatccggttcgagaccagcgactatttcgaggtcatgcgggccctgaagttcgtggtatag

SEQ ID NO: 30 - protein, 265 aa, Unknown
  Obtained from an environmental sample
MKIFISADIEGTAGITNWDEARKPHADYPEHREQMTREVLAACDGANAAGAKEILIKDAHATGRNILQADLPENVRLIRGWSGHPMSMVQELDKSFDAAMFIGYHSKAGDETNPLAHTLTGEVMRMRINGVATSEFLLHAYAAAMVGVPVVFVSGDWQLCTDIKETNPNITAVAVSEGIGPSTVSLAPKAACAQIREGVRQALQGDLKKCLLQLPERFTLEIEFNNPVKAYKSSWYPGCKHIGERTIRFETSDYFEVMRALKFVV

SEQ ID NO: 31 - DNA, 1425 bp, Unknown
  Obtained from an environmental sample
atgggcaagacggaactgtggcagtgggatgccgtcgacctggcgaaggcgatccgcctcaggaagatctccagttacgaggcgaccaaggcgtgcctggatcggctcgaggcggtgaatccgaagctcaacgccatcacgttccagatcgccgcgcaggcgttgcgcgacgccgaggctgccgatgcggcggtgaaggccggcgaggcgctcggcccactgcacggcgtgccggtcagcaccaaggagaatatcgaccagaaggacctgccgacccccaacggcgtggtcgcctacaaggatgtaatcgcgccggccgactcgccggtggtcgccaactggcgcaaggccggcgccgtcttcatcggccgcaccaacgcgccggcctattcgctgcgctgggacaccgacaatgcgctccgcggccgcacctataatccgtgggccaagggacggacgccgggcggttcctccggcggcgccggcgcggcggtcgccgccggcatcgggccgatctcccacggcaacgactatggcggatcgatccgctatccggcctattgctgcggcgtcgccggcatccgcccgacccagggccgcgttccggccttcaatccatcggcgcaggtcgagcggccgccgaccatcgcgatgttcgcggtgcaagggccgctggcgcgccgcatcaaggacgtgcgcctcggcctccatgccatgagcgggcgcgacgcgcgcgatccgtggtgggtgcctgcgcatcatcacggccgctcgccgcaacgaccgatcaaggtcgggatcatcacggaagcgccctatgttcatccgaagatcgcggaagcagtgcgcaaagccggcgtcgccctgggcaatgccggctatgcggtggaggaggtgacgccgccgtcgatcgaagccgcgcgcgagctgtgggtgaagctggtcgccgccgacatccgcgagctgctgtgggagaccatcgaggccaatgccgacccgctcggcgtcaaggcggtgaagctgtggcgcgacgccctgccggcgatctccatgggcgaatatctgaaaggcttcgcgcagatcaccggacatcggcgcgaatggtcgctgttcctggagcaatacccgctgctggtcggccccaattgcggcgacctgccattcgagatcggcttcgacaccaccgacatggaacgcacgctgcatgtgcttgccagccaggcgctgatggtgacggtcaacctgctcggcttccccgccgccgcggtgccggtcggaaccacgccggcggacggcgcgccgaaaggcctgccgctcggcgtccaggtgatcgccggccgctaccgcgaagacctcgccctcgacgcggcggaggtgatcgaagcgcaacacggattggagacgccgatcgatccggtgtggtag

SEQ ID NO: 32 - protein, 474 aa, Unknown
  Obtained from an environmental sample
MGKTELWQWDAVDLAKAIRLRKISSYEATKACLDRLEAVNPKLNAITFQIAAQALRDAEAADAAVKAGEALGPLHGVPVSTKENIDQKDLPTPNGVVAYKDVIAPADSPVVANWRKAGAVFIGRTNAPAYSLRWDTDNALRGRTYNPWAKGRTPGGSSGGAGAAVAAGIGPISHGNDYGGSIRYPAYCCGVAGIRPTQGRVPAFNPSAQVERPPTIAMFAVQGPLARRIKDVRLGLHAMSGRDARDPWWVPAHHHGRSPQRPIKVGIITEAPYVHPKIAEAVRKAGVALGNAGYAVEEVTPPSIEAARELWVKLVAADIRELLWETIEANADPLGVKAVKLWRDALPAISMGEYLKGFAQITGHRREWSLFLEQYPLLVGPNCGDLPFEIGFDTTDMERTLHVLASQALMVTVNLLGFPAAAVPVGTTPADGAPKGLPLGVQVIAGRYREDLALDAAEVIEAQHGLETPIDPVW

SEQ ID NO: 33 - DNA, 858 bp, Unknown
  Obtained from an environmental sample
atgaataccgatgcctttcgcgcgccgatctcgatgagcgacgccggagccaaagccgccttgctcgacgggctgccgcgcgatgtcggcgggctggccaagatcgtgcagggcgtgctgatccacgagcatgtcgcgccggcctacggcgtcaccctgtcggctgagcagcatgcgcaggcgcatatgcgtggcgtcgaatcgatgctcgactgcatcacccgccaggacggtcggcccttgccgcaggcgcgcccgccaggcgagcgcgtggtcggcgtctgccggcatttcacgctcctgcacgtcgccatgctgcgccgacagggcttcgccgcgcgggcgcgctgtggcttcggcgcctacttcgagaagggcaagttcatcgaccactgggtcactgaatactggagcgagagcgccaggcgttgggtcctggtcgacgcccagcttgaccagcgccagcgcgagctgttcggcatcgccttcgatccgctcgacgtgccgcgcgaccagttcgtggtggcgggcgatgcctggcagctctgccgcagcggcaaggccgacccgggcgcgttcggcatcctcgacatgcacggtctgtggttcatcgccggcaacgtcatccgcgacgtggccgcgctcaacaatcacgagatgctgccatgggacgtgtggggcggcatggcgcagaccgacgccgagctcgacctcgccttcatcgatcgtctggccaggctctcgcatgccccggatcgtcacttcggcgacttgcgcgccatctacaaggacaagcgcgtcgccgtgcccggcacggtgttcaacgccgtcctcaaccgtcccgaccctgtccactga

SEQ ID NO: 34 - protein, 285 aa, Unknown
  Obtained from an environmental sample
MNTDAFRAPISMSDAGAKAALLDGLPRDVGGLAKIVQGVLIHEHVAPAYGVTLSAEQHAQAHMRGVESMLDCITRQDGRPLPQARPPGERVVGVCRHFTLLHVAMLRRQGFAARARCGFGAYFEKGKFIDHWVTEYWSESARRWVLVDAQLDQRQRELFGIAFDPLDVPRDQFVVAGDAWQLCRSGKADPGAFGILDMHGLWFIAGNVIRDVAALNNHEMLPWDVWGGMAQTDAELDLAFIDRLARLSHAPDRHFGDLRAIYKDKRVAVPGTVFNAVLNRPDPVH

SEQ ID NO: 35 - DNA, 1458 bp, Unknown
  Obtained from an environmental sample
atgaacgacatcgcctaccgctcggccaccgaactcgccggcttgctcggcgcgcggcaaataggctcacgcgaactgctcgagcacctgctgcggcggctcgaggcattcaatccccgcatcaacgccgtcgtcacgctcgacctggagcgcgcacgcgcccgcgccgatgccgccgacgcggcgctcgcacgcggcgagagctggggcccgctgcatggcctgcccatgacgatcaaggactccatcgagacggccggcctgcgcacgacctcgggtgccgcgcactacgcgaagcacgtacccaagtcgaacgccccagccgtgcagcggctggtggacgcgggggccgtcgtgttcggcaagaccaacttgccggccttcgcaatggacacccagtcgtacaacgagctcttcggcaccaccaacaacccgtgggacctctcgcggacctcggggggctcctctggcggggcggccgcggcgctggcaaccggcatcacgccgctggagctgggcagcgacatcggcgggtcgatccgcaacccggcgcactactgcggcgtgtacgggcacaaatcgagccacggcatcgtgcccgggcgcggccacattccggggccgccgggctcgctgtcggagcccgacatcgccgtgctggggccgatggcgcgcagcgccgacgacctcgcgcttgcactggaagtcctggccggccccgacgacgaccgagccgtggcctggcacctgacgctgccgccgtctcgtcatcggcatctgcgcgagttcagggtggccgcgtggctgggagacgatgccgcgccgatcgaccgcgaggtggccgcatgcctgcgcaagacagtggacctgctgcgcgagcacggcgtcagtgtcgatgagcgcgcgcggccggccgtcgacatgcccaaggcgctcgacgcctattggcggctgttgttcggcgtgacctgtgcgtcgcccccaccagagaccttcgcggccctgtgcaaggccgccgagcagctgccgcccgggcaaggcggcgccgacgcgcgcttcgtgcgcggcacgacacagcgccaccgagactgggtctacaccaacgaagcccgcatgcgaatgcgcgccgcctgggcggcattcttcaacgaccacgatgtgttgctgtgtccggtcgcgccgacggccgccttcgcgcacgaccattccgagccgatgggtgctcgggtgatcgcgatcgacggcgagtcgcggccctacatcgaccagcttgcgtgggccggcttcgtcggcatggcatacctgccgtccaccagcgctccggtgggccgcacgcccgccgggctgcccgtgggcatccagatcgtggggccctatctcgaagacctgacgaccatcggctttgcggcgcagctcgcgcagctcaccggcgggttcgtcgcgcccccggggtactga

SEQ ID NO: 36 - protein, 485 aa, Unknown
  Obtained from an environmental sample
MNDIAYRSATELAGLLGARQIGSRELLEHLLRRLEAFNPRINAVVTLDLERARARADAADAALARGESWGPLHGLPMTIKDSIETAGLRTTSGAAHYAKHVPKSNAPAVQRLVDAGAVVFGKTNLPAFAMDTQSYNELFGTTNNPWDLSRTSGGSSGGAAAALATGITPLELGSDIGGSIRNPAHYCGVYGHKSSHGIVPGRGHIPGPPGSLSEPDIAVLGPMARSADDLALALEVLAGPDDDRAVAWHLTLPPSRHRHLREFRVAAWLGDDAAPIDREVAACLRKTVDLLREHGVSVDERARPAVDMPKALDAYWRLLFGVTCASPPPETFAALCKAAEQLPPGQGGADARFVRGTTQRHRDWVYTNEARMRMRAAWAAFFNDHDVLLCPVAPTAAFAHDHSEPMGARVIAIDGESRPYIDQLAWAGFVGMAYLPSTSAPVGRTPAGLPVGIQIVGPYLEDLTTIGFAAQLAQLTGGFVAPPGY

SEQ ID NO: 37 - DNA, 795 bp, Unknown
  Obtained from an environmental sample
atgaaagtttatattagtgcagatttagaaggagttgcatgctcaacaacttggattgagacggaaaaaaatacaccggattatgccccttttcaaaagcaaatgacaaaagaagttgaagccgcaatcgaaggagcaatagcgggtggagcaactgaaatcgtattaaaagatgcccatgactctgcaaggaatattgatttttcaaacttaccagaaaatgtgaaaataattcgtggctggacaggggatccaatgtgtatggttgctggattagatgaaagttttgatcgcgccattttcatcggttatcacagtaaaggtggaagtcatcgaaatccattggcccacacattggtcgtgaatgccgatgtgaaaatcaacggtgaatatgcaagtgaatttttaattaatgcttatgcggcagcactacatggagttcctgttgcatttgtcagtggagatgttggacttacagaggaaatcaagtctgttaacgaaaatattgttacgcttcctatcaaagagggaattggtggagctactgtaagtattagccctaaattagcaattagcgaaacaaagagattggtagaagaagcgatgaaggttgatagagattcaattaaaatagagcttccaaagcatttcaatgtcgaaattatttacagagatcatacacgtgcatatcgaaattctttttatcctggagcaaaatttaagcctcataatacagtggaatatgaaacagataactattttgacgttttacgaatattgcagtttttaacttaa

SEQ ID NO: 38 - protein, 264 aa, Unknown
  Obtained from an environmental sample
MKVYISADLEGVACSTTWIETEKNTPDYAPFQKQMTKEVEAAIEGAIAGGATEIVLKDAHDSARNIDFSNLPENVKIIRGWTGDPMCMVAGLDESFDRAIFIGYHSKGGSHRNPLAHTLVVNADVKINGEYASEFLINAYAAALHGVPVAFVSGDVGLTEEIKSVNENIVTLPIKEGIGGATVSISPKLAISETKRLVEEAMKVDRDSIKIELPKHFNVEIIYRDHTRAYRNSFYPGAKFKPHNTVEYETDNYFDVLRILQFLT

SEQ ID NO: 39 - DNA, 903 bp, Unknown
  Obtained from an environmental sample
gtgacctcgtcgctgttcgacctgccgcacatcgaagcgcggcgccgattggacaccggcgcgcccgtctacctgaccatcaatccggtcgagtatcacgggccgcacctgtcgctgcacaacgaccggctgatctcgcaggcgctggtggagcggctgcacgcgcgcttgcagaagacctggccggaccggcctttgctgctcgcagccgatctcgaaattggggtcgagccgtgctcgggcgtcgggacgcgccatacgttgttcgcgaccgctcgcgagctggtgtgcgaggcctgccgcgcgctcgccgagctgggcgcgcagcgggtcgtgctgatgacctttcacggatcgccgctccacaacctggcgctcgaggccggcgtggagctgctgcgcgagctcggggtcgcggcggtctcgccgttcaattggatcttgcgggagctactgctgctcgacgatccgtcgccgtacgcggccgcgttcgcacacgtcgaagacccggccgagcggagcgcgatgatggccgagctgcgcgaggactttcacgccggcttcttcgagacttcggtggcgctccatctgatccccgatcacgtcgcgtcgatgcaccgcgagcttccgccgtgccccgagctgccgcgcgacccgtcgctgctgttggccgcgcgggcgatgcgggcgatcgggcgggaccaactcgcgcgcgagctggcgttcgcggccgcgggaaagggctggtatgcgatgcggccgttctatggctacaccggtcggccgcaccgggcatcggcgtcggcgggggaagtgttcaccaacgccatcctcgatcgttacgaagcgggcgttccgcgggtgctcgaaggacaagagcggtcgccggagccgatcatgagc

SEQ ID NO: 40 - protein, 301 aa, Unknown
  Obtained from an environmental sample
MTSSLFDLPHIEARRRLDTGAPVYLTINPVEYHGPHLSLHNDRLISQALVERLHARLQKTWPDRPLLLAADLEIGVEPCSGVGTRHTLFATARELVCEACRALAELGAQRVVLMTFHGSPLHNLALEAGVELLRELGVAAVSPFNWILRELLLLDDPSPYAAAFAHVEDPAERSAMMAELREDFHAGFFETSVALHLIPDHVASMHRELPPCPELPRDPSLLLAARAMRAIGRDQLARELAFAAAGKGWYAMRPFYGYTGRPHRASASAGEVFTNAILDRYEAGVPRVLEGQERSPEPIMS

SEQ ID NO: 41 - DNA, 837 bp, Unknown
  Obtained from an environmental sample
atgcgcgtgctcatctcggcagacatggaaggtacggccggcattgccgtctggcagcaggtcattcctcctgatctggccgataaggggggtgccccagcactctatgaggccggccagcgactgatgacggctgaggtcaatgccgcaattcgtggggctcgagcggccggagcgaccgagatcatcgtgaacgactcccacgacggtatgcggaacatactgccgtcggaactcgatcccgatgcctggctgattaccggcaaccagaagccactggtgatggtctgtgatgtcgataaggacatcgatgcgctggtcttcaccgggtaccacgcccgtgccggaacccagaacggtgtgctggcacattcgtttaccggctggatcatggacatgtggatcggtgatatccaggtcggtgagcacggtatgaacgccgcgactgccggccactggaatgtcccgctcgtggccgttaccggtgatgaagctgccgttcttcagacgcaagagctcgttggtgatgcctgtgccggagcgatcgtcaagtacggccgggggcgtcactctgccaaacatatgcacccgacccgtgcctgtgaactgatcgaggccacagtcaaggatggggtctcccgtgccagcgagatctcaccgttcaagccgccattccctgcgacagtcactgttcgagtcgatacggccgaccgggccgacaagatggccaaagttccgggaaccaaccgaccggatggcgataccgtgagctacgaagcacgagacggcaagcacctgatggagcactttctggcaatgatgggctgcgctggatag

SEQ ID NO: 42 - protein, 278 aa, Unknown
  Obtained from an environmental sample
MRVLISADMEGTAGIAVWQQVIPPDLADKGGAPALYEAGQRLMTAEVNAAIRGARAAGATEIIVNDSHDGMRNILPSELDPDAWLITGNQKPLVMVCDVDKDIDALVFTGYHARAGTQNGVLAHSFTGWIMDMWIGDIQVGEHGMNAATAGHWNVPLVAVTGDEAAVLQTQELVGDACAGAIVKYGRGRHSAKHMHPTRACELIEATVKDGVSRASEISPFKPPFPATVTVRVDTADRADKMAKVPGTNRPDGDTVSYEARDGKHLMEHFLAMMGCAG

SEQ ID NO: 43 - DNA, 822 bp, Unknown
  Obtained from an environmental sample
atgaagatattaatcgcagtggacatggaagggatcaccggcatcacgacctgggaccaggtcacgccggggcatgccgagtacgggcgctttcgcaggctcatgacccaggacgtgaacgctgccatccgcggcgcgtacgacgccggcgccgaggaagtcttggtcgcggatggacactggttcggctcgaatatcctgatcgaggagctcgaccagcgggcgcgcctgaacaccggcagcccttccccattttccatgatgcagggtatcgaggaaaacgtgaacggtgtgatgttcgtgggctaccatgcccgtaacggatcgcctaacgcggttctcgaccacacctggtcctcgaagacggtagcgaacgtctggctgaacgatatcctcactggcgagtatggcttgaacgcggccgtggcgggtcattttggcgcgccggtcattatggtctcgggagatcaaacagcctgtgcacaggttacggatttgatcggcgcggtggagaccgccattgtcaaacaggcttccagccggtttgctgctgaatgcctgcctccgcaggttacacaggacatgatctgtgccagcgcacggcgcgcggtggaacggctcgcccagggcgacgcaccacatccattcgtgctggaaccaccgatcacggtcacagtggggttcttcacctccgacatggcagaccgcgccacgcgcatgccgttcaccaaacgtgaggggaggcgcgtttcactcactgtgccagagatggactccgcttataacggcttccgcgccatggtcatgctggcatcgggctag

SEQ ID NO: 44 - protein, 273 aa, Unknown
  Obtained from an environmental sample
MKILIAVDMEGITGITTWDQVTPGHAEYGRFRRLMTQDVNAAIRGAYDAGAEEVLVADGHWFGSNILIEELDQRARLNTGSPSPFSMMQGIEENVNGVMFVGYHARNGSPNAVLDHTWSSKTVANVWLNDILTGEYGLNAAVAGHFGAPVIMVSGDQTACAQVTDLIGAVETAIVKQASSRFAAECLPPQVTQDMICASARRAVERLAQGDAPHPFVLEPPITVTVGFFTSDMADRATRMPFTKREGRRVSLTVPEMDSAYNGFRAMVMLASG

SEQ ID NO: 45 - DNA, 687 bp, Unknown
  Obtained from an environmental sample
atgcgatttgaagaactgaactggatggacgtggaacgataccttgaaaccgatgaccgtttgatattggtcactggtgcgtgcgagcaacatgcctatttgagtttgctgaccgatatcaaaattccgctggcgatggcagatgccgcctcaaacgtgaccggggtgctggtcgctccgcccgtgaactttggcaactctccatactttcgcgattaccccggcacgctgaccttgcgagtctctactttgaatgcagtggtcgaagacattatccgctcggcgtatcatcagggctttcggcgcattttaattctcaacggacatggcggtaacaagggattgaacgttcatttgaatgacgtcatcaatgatatgcccggtttgaagttgaactggtatgactggtggacgtctcattctgtggagaaagtggcgctcaaatacaacctgaaaccgaaacatgctaactggctggaggcgtttccgttcacggtggtcagcgagatgccgaaagagccgaaactttcgccgatggttcccagctccatcatggactccaaaactgcccgtgaagtctacggcgacggttccttcggcggcgaatattttgttagcaatgagatcatgaccgagatgttcaatgcagcagtggaagatattttgatgatgttgaaatttgagtag

SEQ ID NO: 46 - protein, 228 aa, Unknown
  Obtained from an environmental sample
MRFEELNWMDVERYLETDDRLILVTGACEQHAYLSLLTDIKIPLAMADAASNVTGVLVAPPVNFGNSPYFRDYPGTLTLRVSTLNAVVEDIIRSAYHQGFRRILILNGHGGNKGLNVHLNDVINDMPGLKLNWYDWWTSHSVEKVALKYNLKPKHANWLEAFPFTVVSEMPKEPKLSPMVPSSIMDSKTAREVYGDGSFGGEYFVSNEIMTEMFNAAVEDILMMLKFE

SEQ ID NO: 47 - DNA, 1455 bp, Unknown
  Obtained from an environmental sample
atggatgtgttgatgcgcccggccaccgaaattctcgcggcgctgggacgccgggagatgtcggcggttgaactgctggacgccacgctggcgcgcatcgcggcggtcaacccggcgttgaacgccgtcgtcgcgctcgacgccgaggccgcccggcgcgcggcccaggcctccgacgcccgcagagcgcgcggcgaggatggcctgctcgaaggtctgccgatgacgatcaaggacgcgttcgacactgccgggctggtcacgaccggcggcgcgccggcatggcgcgagcgcgtgccggagagcgacgccagcgccgtggggcgcctgcgggcggccggcgcggtgatcctcggcaagaccaacgtcccgccctattcgggagactggatcgctgacaacgccgtgtacggccgcaccaacaatccgtgggacgtgacgcgcagccccggcggctcgtcgggcggcgcggccgccgccgtcgccacgggcatgagcgcgctggagctgggctcggacctcggcggctcgatccgctggccggcccatgcctgcggcctcttcgcccacaagccgacctggggcctcgtctcgacgcgcggccatcgcccgccgccgcccggcatcgacatcgaggccgacctgtccgtcgccggccccctcgcccgctcggcgcgtgacctcgacctcatgctgtccgtgatcagcggccccgccgcgctcgacggcgtgaagccgcgcctcgatccgccgcgccgaacctccgccggcggcctgcgcgtggcgctgtgggcgagcgagcccttcgcgcccgtcgatcacgaggtctcggcagccgtgcgccgcgccgccgcgctactggcgggcgcgggcgcggaaatcgacgatgaagcgcgcccggccttccgcttcgaggagagcttcgaggtctatgccctgatgaaccacgccatcgtcgccgccggcctgccggagaagatccgcgaccggctggccgccgaagcggcgaattatccgccgggcgacctctcccaccgcgccctgcaggcgcgcggcgcgcggctcgatgtcgcgaccttccgtgcgctgcaggatcgccgcgccgcgctgaaacgcgcctgggacgcgttcttcacacggttcgacgtggtgctgatgccctgcgcgcccacgcccgccgtgccgcacccgccgaccgacatgaaggaccggcggctcgtcgtcgacggcatggagcggccctggttcgatttcctgctctggtcgggccccgccaccgtcgcgcacctgccggcttccgtcgcgcccgtgatgcgcggcgagtcgggcctgccgctcggggtgcagatcgtggccgccgaaggcgcggaccgcaccgccatcgcggtcgcggcgatgctggagggactgggctgtcgttttgtcgccccacctctgccgtccgctgggggctga

SEQ ID NO: 48 - protein, 484 aa, Unknown
  Obtained from an environmental sample
MDVLMRPATEILAALGRREMSAVELLDATLARIAAVNPALNAVVALDAEAARRAAQASDARRARGEDGLLEGLPMTIKDAFDTAGLVTTGGAPAWRERVPESDASAVGRLRAAGAVILGKTNVPPYSGDWIADNAVYGRTNNPWDVTRSPGGSSGGAAAAVATGMSALELGSDLGGSIRWPAHACGLFAHKPTWGLVSTRGHRPPPPGIDIEADLSVAGPLARSARDLDLMLSVISGPAALDGVKPRLDPPRRTSAGGLRVALWASEPFAPVDHEVSAAVRRAAALLAGAGAEIDDEARPAFRFEESFEVYALMNHAIVAAGLPEKIRDRLAAEAANYPPGDLSHRALQARGARLDVATFRALQDRRAALKRAWDAFFTRFDVVLMPCAPTPAVPHPPTDMKDRRLVVDGMERPWFDFLLWSGPATVAHLPASVAPVMRGESGLPLGVQIVAAEGADRTAIAVAAMLEGLGCRFVAPPLPSAGG

SEQ ID NO: 49 - DNA, 1779 bp, Unknown
  Obtained from an environmental sample
atgaaaagcaacctccgcccgtttctactctcacttttgctgatcggaatcttcctcgtcgcgtcctgctctggcgggacagaaacgcctccgccgccccccagcctgccgcgacccaccccggcctcgtcccagcccgcgcggactctcctcaccgccgccgcgttgacggacggtttcgagttcgacagcccgttggatgaatctgccctcacgccgcccgaagaatccgcgccgcctgcgcatatcttcgaaggctgcctggaactgatcggcgaacaggatggtggacacatccaaattctgcgcggcgagctggggacggaatacgcctccctgccggagttcgatttcgagttcgtgcaggcggacggctggctgattcccgttcgacgcggcaacctcattgccgaccaccctgtctggaacatcatccttgagccggggcgcgtctggcaggaaaccggggatgccggtttgtcacgtgcttctttgccatttgccctcacggtcaagggcgggaatgccaccttcaacggcacattgactttccttttcgatgatcaacatgtttcaaaggtctggtatcaggtcacacaggagaccaccagtcatacccgtgccaatatgtgggggttgctggatgcggtttatcgtccgggagcagtgaccgggtccgaaaagatccgtgcggactttgccgcagagttgtctgcgcggatgcaggtcaagcctatcgaagtactggcagaggattatcccggcgtggatgtctccgcttttgggcgcggcgtgactccagaacacatgacctggtatggcgtcgtggtggatggtgtcaattacatcggcggatgcgagacgcgcttcggtcgttatccctattgcgaatcgatgcgcgccacatcctactcaacggcgaaatccgcctttgccagcgtggcattgatgcggctggcgcaaatatatggcgtggacgtgccaggcttgctcatcagggactacgtgcccgagtatgccgccagcgcaggtgattgggagcaggtgaccttcaatgacacactggatatgtccacgggcaactatgtttcagcaggatttatggtggatgatgacagtgacaagatgggggagtttttcggcgcccagccttacactgaacggatcaatgaagcgttcagcgcgccccatcaggctgatccgggcaaccgttgggtttaccgcaccagcgacacgttcatcctgagccgtgccatgcaaaactacctgcaatcccagcagggatcgaatgccgacatctacgagttcgtggtggatgaagtgtatcgtccgctcagacttggtcccggtgccttcaccaccatgcgcaccgccgatgataactggcagggacaagccgagggcggctatggtcaatggtggattcccgatgatatcgccaaggtcgggaccttcctgattcacgacgatggagcaatcaacggtgaacagatcctgcatccaaacctgttggcggcatcactgcaacaggatcccgatgaccgcggcgtgcgcatcgacagccagcgcatgtacaacaactcgttctgggcgaatcactacaccacaataaacggattcgactgtgagttctgggtgccgcagatgctcggcgtcagcgggaatgtggtggcgttattccccaacgggatcacctattactatttcagcgacaatcgggaatttacctgggatgcggcgttgcgagagtcagacaagctcattccactttgccgttga

SEQ ID NO: 50 - protein, 592 aa, Unknown
  Obtained from an environmental sample
MKSNLRPFLLSLLLIGIFLVASCSGGTETPPPPPSLPRPTPASSQPARTLLTAAALTDGFEFDSPLDESALTPPEESAPPAHIFEGCLELIGEQDGGHIQILRGELGTEYASLPEFDFEFVQADGWLIPVRRGNLIADHPVWNIILEPGRVWQETGDAGLSRASLPFALTVKGGNATFNGTLTFLFDDQHVSKVWYQVTQETTSHTRANMWGLLDAVYRPGAVTGSEKIRADFAAELSARMQVKPIEVLAEDYPGVDVSAFGRGVTPEHMTWYGVVVDGVNYIGGCETRFGRYPYCESMRATSYSTAKSAFASVALMRLAQIYGVDVPGLLIRDYVPEYAASAGDWEQVTFNDTLDMSTGNYVSAGFMVDDDSDKMGEFFGAQPYTERINEAFSAPHQADPGNRWVYRTSDTFILSRAMQNYLQSQQGSNADIYEFVVDEVYRPLRLGPGAFTTMRTADDNWQGQAEGGYGQWWIPDDIAKVGTFLIHDDGAINGEQILHPNLLAASLQQDPDDRGVRIDSQRMYNNSFWANHYTTINGFDCEFWVPQMLGVSGNVVALFPNGITYYYFSDNREFTWDAALRESDKLIPLCR

SEQ ID NO: 51 - DNA, 801 bp, Unknown
  Obtained from an environmental sample
atgaagaagattttcatgtcctcggatatcgagggtacctgcggtattgcccattgggacgaaacggagaagggcaaggtggactatcctgctttctccacccagatgagccgcgaggtggcggctgcctgccagggtgcgctggatggcggcgcggtggatgtgtttgtccgggattcccacgattccgccaggaacattcagcctggtatgctgccggaggacatcaagatcttccgaggctgggggcgcgatccctacagcatgatgagcggcatcgacgggagctttgagggtgtgatcttcactggctatcattccgctgccagctgggatggcaatcccctcagccacaccatgaacacccagaacaattatgtgaaggtgaatggtgaactctgctctgaactgatgatgaacagcctcaccgccgccatgttcggggtacctgtgctgatggtgaccggtgacatgatgctgtgcgaatggttcaagaccaaggttcccgatgccatcactgtgccggtgagctatggcattggcaacggcagcgtgtccatccatcctgacaaggccgtgaagcagattcagaaggccgccaagaaggctgtgcatctggacggcaaaaagtgcatgttccccatgccggagtattttactgtggaggtatgctacaagctgcaccacaaggctcgttctgccagctggtatcccggtgcgcatctggaaaacagcaccacagtggtgtatgaaagcgacagctggatggatgtactgaccttcttccactattgcctgtga

SEQ ID NO: 52 - protein, 266 aa, Unknown
  Obtained from an environmental sample
MKKIFMSSDIEGTCGIAHWDETEKGKVDYPAFSTQMSREVAAACQGALDGGAVDVFVRDSHDSARNIQPGMLPEDIKIFRGWGRDPYSMMSGIDGSFEGVIFTGYHSAASWDGNPLSHTMNTQNNYVKVNGELCSELMMNSLTAAMFGVPVLMVTGDMMLCEWFKTKVPDAITVPVSYGIGNGSVSIHPDKAVKQIQKAAKKAVHLDGKKCMFPMPEYFTVEVCYKLHHKARSASWYPGAHLENSTTVVYESDSWMDVLTFFHYCL

SEQ ID NO: 53 - DNA, 1623 bp, Unknown
  Obtained from an environmental sample
atgaatcgacgcgactttcttgctaccgtgccgctggctcttgcggcgtccactctgcccgcttctgaaatgtttggcgcgaccgctgctgcgaagccggatactgccgcgcaaagcttcttaccttctgcatggcagaacaaagtaggcgggacacccgccgcgctcgcttcgcatgaactcgacttcgccacggctttgcaggcggcggaagccattcgcacaaagaaagtctcgtccgtcgaactcacggagcgcatgtttgcccgcattgatcattacaatccgcagctcaacgtctttgcctatcagttgcgagaagacgcgctggcgcaggcaagaaaggccgatgctgccatggcgcagggcaagacgcacggcgttttccatggtgttcctgttacggtgaaagaaagttttgccgtggcgggacatccatgcacgtggggcttccctccactccgcgactccaaagcgccccaaaatgctgaagtggtaaaccgtttactctcggatgccggcgctgtgctgctgggcgctacgaatgtgccggtggcgctctctgattggcaaagctacaacccgatttatggccagaccaataacccatgggacgtgaagcgatctcccggcggatcttcgggcggaagcgctgcagggctcgccgcgggcctgggatatctgagcgttggcagtgacattggcggatccattcgcgtgccgtcgcatttctgtgggctctttgggcacaagccgacgctcgatctggtgagcacaagtggtcaaattcccggcggtaatcccggtttgccggatttctcaacgctattggcggttgccggaccgctggcccgcagcgctcaagatctgctggtggcgctgaaagttcttggcggtccaacaggctgggacgccaaagcatggaagtggcaaatgcccgagccgcgcgcacacagtttgaaagatttccgcgtgggttatgtgattgacgatcccattgctccaccaacaccggaagtaaaagccatgctggaaaatgcgattgagaagcttggccgcgctggcgcaaaaatgaagcccgggtggcctgagggtttcaagcctgcggaactgctggccaattatgtattcatgctggaagctttcttgtacagcacagcgcctcctgaggagcaagagcgcctgagaaaaacagttggcaacatggttgtcggcaagaactccggagccatgagctcatttgccgagtggcagcagcagaattttcgccggctggcgtaccgcgcgcaatggcaggcatatttcaagcaggtggatgttttcctttcgcctgtggctttcaccgcagctttcccccacgaccacagcgagccgcaagaccagcgcaccatcgccacctcagccggcccgcgccactatatggatatgctcaactggatcgctccggccacgctgacgggctgtccggcgacagtcgctccggtcggtcgcacgccgcagggacttccggtgggaatccagatcatggggccgttctgggaagacgccacgccgatcacgtttgccgacctgctggcagtagaagttggcggctttgtgccgccttctggatataaagaataa

SEQ ID NO: 54 - protein, 540 aa, Unknown
  Obtained from an environmental sample
MNRRDFLATVPLALAASTLPASEMFGATAAAKPDTAAQSFLPSAWQNKVGGTPAALASHELDFATALQAAEAIRTKKVSSVELTERMFARIDHYNPQLNVFAYQLREDALAQARKADAAMAQGKTHGVFHGVPVTVKESFAVAGHPCTWGFPPLRDSKAPQNAEVVNRLLSDAGAVLLGATNVPVALSDWQSYNPIYGQTNNPWDVKRSPGGSSGGSAAGLAAGLGYLSVGSDIGGSIRVPSHFCGLFGHKPTLDLVSTSGQIPGGNPGLPDFSTLLAVAGPLARSAQDLLVALKVLGGPTGWDAKAWKWQMPEPRAHSLKDFRVGYVIDDPIAPPTPEVKAMLENAIEKLGRAGAKMKPGWPEGFKPAELLANYVFMLEAFLYSTAPPEEQERLRKTVGNMVVGKNSGAMSSFAEWQQQNFRRLAYRAQWQAYFKQVDVFLSPVAFTAAFPHDHSEPQDQRTIATSAGPRHYMDMLNWIAPATLTGCPATVAPVGRTPQGLPVGIQIMGPFWEDATPITFADLLAVEVGGFVPPSGYKE

SEQ ID NO: 55 - DNA, 798 bp, Unknown
  Obtained from an environmental sample
atgaaagtcttcatcagctccgacatcgagggcaccgccgggatcacggcgtgggacgaggccaagaagggcaatcccgaatacctggagtttcgggaatatatgacggacgaactcgtcgccgcatgcgagggcgccagagcggccggagcaaccgaagtggtggtgaaagatgcccattccacggctcgcaacctcattctgtcgaagctgccggactatgttcgcatcgtccgtggctggagcggccatcccgacatgatgatgttcggaattgattcgagcttcgcggctgcactctatatcggctatcacaacaaggctggaacggacaccaatccccttgcccataccctgaccggtaccgtctcgcgtctgctgatcaatggcgaggttgcgtccgaattcacgatcaatgccctctgtgccgcgcgctatggcgttccctcagtcttccttgccggcgataagggcatgtgcgcagaggctaagggactggttgccgaaatcggcaccgtggccaccagcgagggcttcgggccggcaaccgcctccctgtcgccgggcgcgtcgcggcgtgtgatccgggacggtgtcgagaaggctctgtcccgtaatttctcaatgtgtctgccggagctcgccgaacggttcgagctcatcgtcgaatatacgaccccgacggaggcctatcgcggcagctggtaccccggtgtcgaacacatcgcaccgcgaacgctccgtttcaatgccagggatttcttcgacatccagcgcgcgatccgcttcatcgtttga

SEQ ID NO: 56 - protein, 265 aa, Unknown
  Obtained from an environmental sample
MKVFISSDIEGTAGITAWDEAKKGNPEYLEFREYMTDELVAACEGARAAGATEVVVKDAHSTARNLILSKLPDYVRIVRGWSGHPDMMMFGIDSSFAAALYIGYHNKAGTDTNPLAHTLTGTVSRLLINGEVASEFTINALCAARYGVPSVFLAGDKGMCAEAKGLVAEIGTVATSEGFGPATASLSPGASRRVIRDGVEKALSRNFSMCLPELAERFELIVEYTTPTEAYRGSWYPGVEHIAPRTLRFNARDFFDIQRAIRFIV

SEQ ID NO: 57 - DNA, 798 bp, Unknown
  Obtained from an environmental sample
atgaaagtctacatcagcgccgacatcgaaggcacggcgggcatcaccgtctggccggaggcggaaaagaaccacgccgactatcccgagcatcgccaggaaatgactcgcgaagtgctcgccgcttgcgagggcgcgacgcgcgccggcgccaaggaaatcctgatcaaggacgcgcacgacagcggccgcaacatcctgcaggcggacctgccggaacacgcgcggctgatccgcggctggagcgggcatccgatgagcatggtgcaggagctggacaagagctttgatgcggcgatgttcgtcggctatcactccaaggccggcgacgaaaccaatccgctggcgcacacgctgaccctgcgggtcatgcgcatgcgcatcaacggcgaggcggtctcggagttcgtcttgtacggctacgccgccgcctatgtcggcgtgccggtggtgttcgtctccggcgacaagcagctctgcgccgacatcaagggctacaatcccaacatcgtcaccgtgaccaccgccgaggggaaggggccgtcgaccatctcgctgtcgcccaaggccgcttgtgccaagatccgcgagggcgtggagaaggcgctgaagggcaacctgaagaaatcgatgctgaagctgccgaagaagttcacgctggaagtcgaatattccaatccggtcaacgcctacaaatcctcctggtatcccggcgtcgaacacatcggcaaccgcaccctgcgcttcgagaccaaggactatttcgaggtgctgcgggcgctgaagttcatcgcctag

SEQ ID NO: 58 - protein, 265 aa, Unknown
  Obtained from an environmental sample
MKVYISADIEGTAGITVWPEAEKNHADYPEHRQEMTREVLAACEGATRAGAKEILIKDAHDSGRNILQADLPEHARLIRGWSGHPMSMVQELDKSFDAAMFVGYHSKAGDETNPLAHTLTLRVMRMRINGEAVSEFVLYGYAAAYVGVPVVFVSGDKQLCADIKGYNPNIVTVTTAEGKGPSTISLSPKAACAKIREGVEKALKGNLKKSMLKLPKKFTLEVEYSNPVNAYKSSWYPGVEHIGNRTLRFETKDYFEVLRALKFIA

SEQ ID NO: 59 - DNA, 858 bp, Unknown
  Obtained from an environmental sample
atgaacctcgccccctaccgcagcccggtggacatgagcgatgccggcgcctggtccggcctgctcgaaggcctgccgcgggatcccgcgcgcctcgcgggattaatccagggcctgatgatccatcaacacatcgcacccgcctacggcgtgacgctgactgggtcgcaacaggccgaaccgcatgtacgcgccgtcgagagcatgctcgagcgcatcgccgcgcacgatgcacgtccgctcgaccaggcccggccggtcggcgagcgcctggtcggcgtgtgccgccatttcaccttgctccatgtcgcgatgctgcgccgccacggcgtggccgcccgcgcgcgctgcggcttcggcgcctatttcgagcggggcaaattcctcgatcactgggtcaccgagtactggaacgaagcggagagacgctgggtgctgttcgacgcccagatcgacgcgcgccagcgcgagctcttcgggatcgatttcgatcccgcggacgtgccgcgcgatcggttcctggtggccgggcaggcctggcagatgtgccgcctgggcgcggccgatgccggcgccttcgggatcctcgacatgtatggcctgtggttcatcgccggcaacgtcatccgcgacgtcgctgcgctgaacaacaaggagatgctgccatgggacgtgtggggcggcatggccggacccgacggtgacctcgacgtgccgttcatcgacgggctggcgccactcagccgcgatccggatggtcacgaacaagccttgcgcgacgcctacgacgacaagcgggtcgccgtgcccgacaccgtgttcaacgccgttctcaaccgacccgatcgggtcaattga

SEQ ID NO: 60 - protein, 285 aa, Unknown
  Obtained from an environmental sample
MNLAPYRSPVDMSDAGAWSGLLEGLPRDPARLAGLIQGLMIHQHIAPAYGVTLTGSQQAEPHVRAVESMLERIAAHDARPLDQARPVGERLVGVCRHFTLLHVAMLRRHGVAARARCGFGAYFERGKFLDHWVTEYWNEAERRWVLFDAQIDARQRELFGIDFDPADVPRDRFLVAGQAWQMCRLGAADAGAFGILDMYGLWFIAGNVIRDVAALNNKEMLPWDVWGGMAGPDGDLDVPFIDGLAPLSRDPDGHEQALRDAYDDKRVAVPDTVFNAVLNRPDRVN

SEQ ID NO: 61 - DNA, 2064 bp, Unknown
  Obtained from an environmental sample
atgaccgatcttcccaagccgcccctcgccgccacccgcccccacagcttcgccgcgcatggcgtcacgatcgaggacccctgggcgtggctcaaggaccccgcctatccgaccgtcaccgacaaggacgtgctcgcctatctcgaggaggagaatggctatttcgagaaggtgatggagccgctcaagccgctctccgaggccctgttcaaggagatgcgcgggcggatcaaggaagacgaatcgaccgtaccgcagaaggacggcgactggctctactggaccgatttcgagaccggcggcgaatatccgcgctggtggcggcggcatgtggacggtggcgagggcgcgctgatcctcgacgagccggcgctggcggcgggcaaggagtatttccggctcggcgccttctcggtcagccccgacggcaagctgctcgcctatgcctatgacgacaatggatcggagcggttcgaggtccgggtgaaggacctggacagcggcgaactgctgcccgacgtgatcccgggcatgctctccgagatcgtctggaccgcggactccaagggctttctctacggcctggcgaacgagaactggcggaccgacaatgcccgctggcacatattgggcgagccggtggacagcgaccggctgctctacaaggaagccgatgagggctaccgcgtcgcggtgggcgagacccagtcgcgcaagtatctgctggtctcgaccggggaccatgtgaccagcgaagtctatctgctcgatcccgccgacccttcgaagccgatggtttgtgtaagcccgcgcaggccggggcgcgaatatgatgtcgacgagcatgacggtacgctgttcatccacaccaacgacgtcgacccgcagttccggctggtgaccgcttcggtcgacaatcccggtgattgggccgagcggatcgcaccatcgaagcatttctacatgaccggcgtcacctgcttcgccgacttcttcatcgtcgaggggcgcgaggacgggctcgatcaaatcgagctgcaccgctacgatcccgcaatcgccccggtgcggctgggcttccccgaggcgagctatgtcgcggggctgggcgacaatcccgaataccagatggaaaagctgcgcatcggctatgagtcgatggtcacgccgggcacagtctatgactacaccgtcgccgacggcacgctcgagacgctgaaggtgcaggagatcccgtcgggctatgacgggagcaagtaccgcaccgagcggctgaagatcgcggcccgcgacgggacggaggtaccggtgtcggtggtctatcccaaggacttccctagagacggcagcggcaagctctatctctatgcctatggcgcctatggctatgcgatcccgccgggcttctcgaccagccggatctcgttcctcgatcgcggcgtcgccttcgccatcgcccatatccgcggcggcgacgatctcggccagcaatggtatctcgacggcaagctcgagaagcggaccaacaccttcaacgatttcgtcgatgtggcgaaggggctgatcgaccggggcttcacccacaagggcggcatcgccatcgccgggcgctcggcgggcggcgaactgatgggcgcggtggtcaattccgatcccgatctgtggggcgttgtcgtcgccgatgtgccgttcgtcgacgtgctcaacaccatgctcgacgagagcctgccgctcacccccggcgaatggcccgaatggggcaatccggtggaggacgccgaggcgttcgcgctgatccgctcctattcgccctacgacaatgtcactgcccaggcctatccgcccttgttcatctcgggcgggctcaacgatccgcgggtgacctattgggagccggccaaatgggcggcgaagctgcgggcgaccaagaccgacgatcatgtcctcgttctcaagacgaacatgggcgccgggcatggcggcaagtcgggccgttgggagagcctgaaggaagcggcggacgagatggcgttcgtgctgtggcaactgggagtgacgcaatga

SEQ ID NO: 62 - protein, 687 aa, Unknown
  Obtained from an environmental sample
MTDLPKPPLAATRPHSFAAHGVTIEDPWAWLKDPAYPTVTDKDVLAYLEEENGYFEKVMEPLKPLSEALFKEMRGRIKEDESTVPQKDGDWLYWTDFETGGEYPRWWRRHVDGGEGALILDEPALAAGKEYFRLGAFSVSPDGKLLAYAYDDNGSERFEVRVKDLDSGELLPDVIPGMLSEIVWTADSKGFLYGLANENWRTDNARWHILGEPVDSDRLLYKEADEGYRVAVGETQSRKYLLVSTGDHVTSEVYLLDPADPSKPMVCVSPRRPGREYDVDEHDGTLFIHTNDVDPQFRLVTASVDNPGDWAERIAPSKHFYMTGVTCFADFFIVEGREDGLDQIELHRYDPAIAPVRLGFPEASYVAGLGDNPEYQMEKLRIGYESMVTPGTVYDYTVADGTLETLKVQEIPSGYDGSKYRTERLKIAARDGTEVPVSVVYPKDFPRDGSGKLYLYAYGAYGYAIPPGFSTSRISFLDRGVAFAIAHIRGGDDLGQQWYLDGKLEKRTNTFNDFVDVAKGLIDRGFTHKGGIAIAGRSAGGELMGAVVNSDPDLWGVVVADVPFVDVLNTMLDESLPLTPGEWPEWGNPVEDAEAFALIRSYSPYDNVTAQAYPPLFISGGLNDPRVTYWEPAKWAAKLRATKTDDHVLVLKTNMGAGHGGKSGRWESLKEAADEMAFVLWQLGVTQ

SEQ ID NO: 63 - DNA, 2142 bp, Unknown
  Obtained from an environmental sample
atgaaaaagcgtcaccttctgctggcatcccccgccctggtcgccggcctgctcaaccctccgcccgttctggcgcagggcgcgcgtccgccgctcgcggagcggaagccgcatccgacgaagatccacggctatacgctcgaggacgactacttctggctgcgggacaagcagaaccccgaggtcatcaagtatctcgaggccgagaacgcgtataccgacgagcagatgcggcagaccaaggatctgcaggaaaagctgtatgcggaaatgctcggccgtatcaagcagacggacctgagcgtcccctcgcgcattggcgagtacttctattactcgcgcacggaagagggaaagcagtacccctacatgtgccgccgcaagggcaacatgcaggcgcccgaagagatcctgctcgatctcaaccagctcgcccagggccacaagtttctcggcctcggcgcttattcggtgagcgacgatggcaacctgctggcgttctctacggataccaccggctatcgacagttcacgatgcaggtcaaggatctccgcaccggtgagctgctgaaggagaagatcgagcggaccggctcgatcgtgtgggcgaacgacagcaagacgttgttctattcgaccgaagatccggtgtcgaagcggtcagacaagatctggcgccacgccgtcggcgccgatggcagcgatctggtctacgaagaaaaggacgaacagttcgacgtcggcgtgggacgatcgctcgacaagaaagtacttttcatcgccgcctacgccaagacgtcacgcgagttccgctatcttcccgcgggcgatccgcgaggcaccttcacggtcgtgctgccgcgcgaggcggagcacgaatacgacgtcgatcactacaacggcctcttctacatcacgaccaatcgcggcgcgaagaacttccgcgtagtgacagcgccgatgaacgatccttccgaggagaactggaaaccattcatcgatcacaacccggcgatcaagatcgacggtttgacgttcttcaaggggcacctcgtcgtctccgagcgcgaaggcgggctcacgtacctgcgcgtgaccgacatggcgacgaagcagtctcaccggatcgccaccgacgaaccggactatgcgctcttcctcagcggcaacccggagttcgacaccgagaccgtccgctactcgtatcaatcgatggtcacgccatcgtcggtctacgagtacgacctgaacacacgcaagcgcacgctgctgaaacaacaggacgtcctcggtggctacgaccccaaggcctacgagagcaagcgcgtgtgggtcacggcgcgcgacggcacgaaggtcccgatgtcactcgtgtatcggaagggcgtcaaagtcgacggcagcgcgccgctgctgctctacgggtatgggtcgtacggtgcctcgatgacgccgacgttctcctccagccgattgagcctgctcgatcgtggcgtgatttatgcgctcgcttatatccgcggcggcggcgagctgggcgaagagtggcgccaggcggggcgcatgatgcagaagatgaacacgttcaacgacttcatcgactgcgccgatttcctggtcaaggaaaagtacacgtcatcgaaccgcctggtgatccagggcggcagcgccggcggcttgctggttggcgccgtcacgaacatgcggcccgacctgttcaaggccgtcgtcgcccaggtgcccttcgtcgacgtcatgaacacgatgctcgatgcgtccctgccgttgacgacgagcgagtacaccgagtggggaaacccgaacgagaaagccgcgttcgactacatgatcaagtactcgccgtacgacaacatcaaggcccagaactatcccgccatcctcatccaggtctcgctcaacgacagtcaggtcccctactgggaaggcgcgaaattcgccgccaaactgcgcgtgaccaagaccgacagcaaccctctgctgctgaaaacgaacatgggcgccggccacggcggagcgtcaggccgttacgacgccctgcgcgaaaccgcattcagctacgcattcatgttgtggcagatgggcttactaccgccagccgccgtttcggcggctggacggtag

SEQ ID NO: 64 - protein, 713 aa, Unknown
  Obtained from an environmental sample
MKKRHLLLASPALVAGLLNPPPVLAQGARPPLAERKPHPTKIHGYTLEDDYFWLRDKQNPEVIKYLEAENAYTDEQMRQTKDLQEKLYAEMLGRIKQTDLSVPSRIGEYFYYSRTEEGKQYPYMCRRKGNMQAPEEILLDLNQLAQGHKFLGLGAYSVSDDGNLLAFSTDTTGYRQFTMQVKDLRTGELLKEKIERTGSIVWANDSKTLFYSTEDPVSKRSDKIWRHAVGADGSDLVYEEKDEQFDVGVGRSLDKKVLFIAAYAKTSREFRYLPAGDPRGTFTVVLPREAEHEYDVDHYNGLFYITTNRGAKNFRVVTAPMNDPSEENWKPFIDHNPAIKIDGLTFFKGHLVVSEREGGLTYLRVTDMATKQSHRIATDEPDYALFLSGNPEFDTETVRYSYQSMVTPSSVYEYDLNTRKRTLLKQQDVLGGYDPKAYESKRVWVTARDGTKVPMSLVYRKGVKVDGSAPLLLYGYGSYGASMTPTFSSSRLSLLDRGVIYALAYIRGGGELGEEWRQAGRMMQKMNTFNDFIDCADFLVKEKYTSSNRLVIQGGSAGGLLVGAVTNMRPDLFKAVVAQVPFVDVMNTMLDASLPLTTSEYTEWGNPNEKAAFDYMIKYSPYDNIKAQNYPAILIQVSLNDSQVPYWEGAKFAAKLRVTKTDSNPLLLKTNMGAGHGGASGRYDALRETAFSYAFMLWQMGLLPPAAVSAAGR

SEQ ID NO: 65 - DNA, 1476 bp, Unknown
  Obtained from an environmental sample
gtgaacaacgagtggttgtactgcaatgcgaagcaactggttcagggtatcaagaaaggtgagatcagcagccgggcgttgctggaaacctacattgcccgcatccaggaaaagaaccctgggctgaatgcagtggtggccacggattttgacaaggcgcggcagcgggcggatgaggcggatcaggcgaccgcccggggtgaaagctgggggccgctgcacggcctgccgctgaccatcaaggacacttacgaggtgcccggcatgccctgtactgccggtgcgccccgtttgcgccagcatatgccggcaaaaccggcggcggctattgcgcgcctgcaggaggccggtgccatcattatcggcaaaaccaacgtgcccctgttcgcatcggatctgcagagttacaacaagatttacggcaccaccaacaatccctggaacgtggaatgcacccctggtggttcatcgggtggtgcggcagcggccctggcggcgggtttcacgccgctggaactgggcagtgatattggcggttccattcgcattccggcccatttctgcggcgtctacggccacaaggtaagccatggcatcgtgtcgttgcggggccatatacccgggccgccgggcagcctgagtgaaccggatctggcagtgggtggccccatggcccgttgcgccgacgatctgcaactgatgctggatgtggtggcgggcccatcacccctgcaacagcccggctggcaactgaaactgccggcacccagccagaagaaactggaagattttcgcgtgctgctgtggatggacgatcccctgtgtcccatcgacagtcagatggccagcctctacctgcagttgaaatccgccctgactggtgcgggggtgtcggttaccagcggttcgcccctggggatggggcttgatgatttctatcctttgtatctgaacctgctgggcagcatcatgggtgcatcacagaaaaaaatcgaacgccgggtgatgggattggcggcgccgttatggcaacgcatggggcagtacctggatctgcccaaacgcttcgagaattttctggaaggcgcagcccagagtcatgtggactggttgcgtatggatgaaaagcggcgccgcattcgccagaaattcctggcggtgttcgatcagtacgatgtgatcctgatgccaccggcggcgactaccgctctgcgtcaccagcatcatccggaaatgcccctgcgcaagatcaccatcaacggtgaaaagcgtaattacgcagatatgctgatctggatatccccggccaccctgatggggctgccctcgaccagtgcgccgattggtaagaccaccgccggcatgccggtgaacgtgcagatcatgggcgcgccgtatcaggaccgcactaccatccgtttcgcgtcattgatgagcaaggtgatgggtgggtttaccttgccgccggggtgttga

SEQ ID NO: 66 - protein, 491 aa, Unknown
  Obtained from an environmental sample
MNNEWLYCNAKQLVQGIKKGEISSRALLETYIARIQEKNPGLNAVVATDFDKARQRADEADQATARGESWGPLHGLPLTIKDTYEVPGMPCTAGAPRLRQHMPAKPAAAIARLQEAGAIIIGKTNVPLFASDLQSYNKIYGTTNNPWNVECTPGGSSGGAAAALAAGFTPLELGSDIGGSIRIPAHFCGVYGHKVSHGIVSLRGHIPGPPGSLSEPDLAVGGPMARCADDLQLMLDVVAGPSPLQQPGWQLKLPAPSQKKLEDFRVLLWMDDPLCPIDSQMASLYLQLKSALTGAGVSVTSGSPLGMGLDDFYPLYLNLLGSIMGASQKKIERRVMGLAAPLWQRMGQYLDLPKRFENFLEGAAQSHVDWLRMDEKRRRIRQKFLAVFDQYDVILMPPAATTALRHQHHPEMPLRKITINGEKRNYADMLIWISPATLMGLPSTSAPIGKTTAGMPVNVQIMGAPYQDRTTIRFASLMSKVMGGFTLPPGC

SEQ ID NO: 67 - DNA, 813 bp, Unknown
  Obtained from an environmental sample
atgaaaatcctgattgcagcagacatggaaggggtaaccggtgtggtccactgggatcacgtaagcagcagccatgccgaatattcccgtttccgcaagctgatgaccggcgacatcaacgccgctgtgcgcggcgccttcgacagcggcgccgacgaggtgctggttaccgatgggcatggcaacgcccgaaatgtcctgatcgaagaattggacgtccgcgcccggctgaacagcggcaacacccgcccgctggcgatggtgcagggcgtcgatacgggcgtggcggcggcgattttcatcggttatcacgcgcgggcgggcactgtcaacgccatcctcgatcacatctggactggcggagtgaacaatgtatggctgaacggtgtgccggtcggcgagatcggcctcaatgcggcggtgtgcggccatttcgatgtcccggtgatcgcgctgtctggctgtcagggggcctgcgacgaagcggcggctctgctgggggagatcgagaccgcaccggtcaaatttcccagtgggcgcatggcggcccagtgcctgccgccggcagcggctcagcaggtcatctacgaagccgtacggcgagccgtgcaacgcctggcccggggacaggcgcccggaccgttctgcgtggagacgccggtcgaactgcaggtcgagttcaacagttctgacatggccgacggcgcctcgattttgccgggtgtgcagcgcgccgaccggacgctcaccttgacggctgaggacatgccgaccgcctaccgactattccgcgttgcggcttccctggcgtga

SEQ ID NO: 68 - protein, 270 aa, Unknown
  Obtained from an environmental sample
MKILIAADMEGVTGVVHWDHVSSSHAEYSRFRKLMTGDINAAVRGAFDSGADEVLVTDGHGNARNVLIEELDVRARLNSGNTRPLAMVQGVDTGVAAAIFIGYHARAGTVNAILDHIWTGGVNNVWLNGVPVGEIGLNAAVCGHFDVPVIALSGCQGACDEAAALLGEIETAPVKFPSGRMAAQCLPPAAAQQVIYEAVRRAVQRLARGQAPGPFCVETPVELQVEFNSSDMADGASILPGVQRADRTLTLTAEDMPTAYRLFRVAASLA

SEQ ID NO: 69 - DNA, 822 bp, Unknown
  Obtained from an environmental sample
atgaaaattttgatcgcaaccgatatggaaggtattactggcgtcacaacatgggatcaagtcacaccaggacatgcagagtatgcgcgttttcgcaagttaatgacccaagatgtcaatgcggccatccgtggagcaatggacgcgggtgcagatgaagtcatcattgcagatgggcattggaatgggtcaaatattctggttgaagaacttgactcacgcgcgcggctgaacacgggttcaccttctccattttcgatgatgcaggggattgatgaaagtgtggatggcgtgatgttcgttggttatcatgcccgtaatggaactccgaacgcaattctcgatcacacctggtcttccaaaacagtcgtaaatgtttggctgaacgatattttgacaggtgagtacggcttgaatgcggcggtagctggtcattttggcgcgcccgtcctcatggtttccggtgatcaaacagcctgtgcgcaggtgactgagcttctgggcgatatcgaaatggctatcgtcaaacaggcaacgagtcgtttcgcggcggaatgtctgactccgcaagcctcacaagaattgatctgtatgactgcaatgcgtgcggtggaacgtttgtctgaaggtgatgtacctgacccgttcgtgttggatacacctgtccgtgtaacagtcgaattcttcacctccgacatggcggatcgagcaacgcgtatcccatttacggagcgagagggcacgcgcgtctctctcaccgtacaggagatggcttcggcctacagcggtttccgcgcgatgataatgctggcactggggtga

SEQ ID NO: 70 - protein, 273 aa, Unknown
  Obtained from an environmental sample
MKILIATDMEGITGVTTWDQVTPGHAEYARFRKLMTQDVNAAIRGAMDAGADEVIIADGHWNGSNILVEELDSRARLNTGSPSPFSMMQGIDESVDGVMFVGYHARNGTPNAILDHTWSSKTVVNVWLNDILTGEYGLNAAVAGHFGAPVLMVSGDQTACAQVTELLGDIEMAIVKQATSRFAAECLTPQASQELICMTAMRAVERLSEGDVPDPFVLDTPVRVTVEFFTSDMADRATRIPFTEREGTRVSLTVQEMASAYSGFRAMIMLALG

SEQ ID NO: 71 - DNA, 798 bp, Unknown
  Obtained from an environmental sample
atgaagatttatatttccgcggatatggaaggcatcaccggggctgcaacctgggaagaggttgataaatcaaagccggactacacggagtttcgtcttaatatgtcgcgcgaagtggcagctgcctgtcaaggcgccagagatgctggcgcgaccgaggtttttgtcaaggacgcgcactggaccggcagaaatatcctgcatgaattgctcccccagggcacacatctgattcggggttggtcgggtcatcccatctccatggtgcagcacctggataaaacctttgcggctgtagtgatggtgggctaccactcacgggcgggggccaacggcaatcctctcgcgcacacgatgagcagcagcaagtatgcgagcatccgaatcaatgaccgtttcgcgtccgaatttctgctccatggttacaccgccgcgaccatgggggtcccggtctgctgcgtgtccggagacgccggcctcatggacgaagtgcgaagcgtcaatgaatgcattaccaccgtagcaacacatgaaggtgttggcgcctcggtcattgccccccatcccgttgacacgcaacaaaagcttcgccaggccgttcagatttcattgtcaggcgatcgaagcaaatgtttgctgccgctgccccgcaactttcaggttctcgtgactttcaaagatcacatgaaagcctacaaggcgtcgttctatcccggcgcctcacaggtggatgagatgagcattaagtttgaaacaaaggagcactttgaagtaatgcgccttttgagcttcatttga

SEQ ID NO: 72 - protein, 265 aa, Unknown
  Obtained from an environmental sample
MKIYISADMEGITGAATWEEVDKSKPDYTEFRLNMSREVAAACQGARDAGATEVFVKDAHWTGRNILHELLPQGTHLIRGWSGHPISMVQHLDKTFAAVVMVGYHSRAGANGNPLAHTMSSSKYASIRINDRFASEFLLHGYTAATMGVPVCCVSGDAGLMDEVRSVNECITTVATHEGVGASVIAPHPVDTQQKLRQAVQISLSGDRSKCLLPLPRNFQVLVTFKDHMKAYKASFYPGASQVDEMSIKFETKEHFEVMRLLSFI

SEQ ID NO: 73 - DNA, 2133 bp, Unknown
  Obtained from an environmental sample
atgaaaaaacgatttaatttcagtttaaaaggaaaaacgctgttggttgccgcgctggcattggcttttgccgcctgcacccaaaaagcggaagtaaaaccacccgttgccaaaaaagttcccaaagaattgaccattcatggcgacacccgcatcgataattattactggctgaatgagcgtgaaaatcaggaagtgattgcttatctggaagcagaaaatgcttataaagatgcgatgatggcgcacacaaaaccgttgcaggacaagctctttgaagaaataaaatcgaaaatcaaacagcaggacgagtcggtgccgtacaaaaagaatggttactattattacacacgcacggtagctgaaaaagagtactatgtttcgtgccggaaaaaggaaagtctggaagcaccggaagaagttttgctggatgtaaacgcgatggccgaaggttttgattattatgcaatcggtggaagctcggtgagtaccgataacaaaattcttgcttatggcgtggatacggtaagccgccgaaattatacgatttactttaaaaacctcgaaaccggcgaattgctggccgaccagattccgatgacaagcggcggtgcaacctgggccaacgacaacaaaactgttttttatatgctgaaagacagcgtgacactgcgttcggaaaaagtgatgaaacatgttttgggaacccctgtttcaagcgatgtggaggttttttatgaagccgacgaaacttttaacgcttttgtgtacaaaaccaaatcggagaaatacctgattatcggcagcgagagtacgctcacttcggaatttcgttttctggatgcaaatacaccaaatggcgagtttaaaatcattcagccacggacccgcggactggaatatgcagttgaccattttgacgactacttttatatccgcacgaacctggatgcactcaattttaaactggtaaaaactccggttgccaaaaccaccaaagaaaactgggtggatgtgattccgcaccgtgctgatgtttattttgaagagttcgatattttccagaactacctggtggcatctgaaaaagtggaaggaatcaccaaattgcgcgtgatgccttggcagggaggagaatattacattgattttgaagaggaagtttacaccgcctactcaggcacaaacgttgatttcaatactgaagtgttccgttttggttatacttcgatgaccactccgacttcaacttttgacttcaatatgaaaaccaaagaacgcacccttctgaaacagcaggaagtgttgggtggtttcgacaaaagcaattacgaaacaaaacgtatttatgccactgccggcgatggaacaaaaattccgatatccattgtttacaaaaaaggtatggagaaaaatggtaataatccggcactgatttatggttacggttcttatggttacgaaacggaacccgctttcagctcatcacgtttacctttgttagacaggggttttgtgtatgccattgcacacattcgtggtggtcagatttatggccgccaatggtacgaggatggaaaactgctgaaaaagatgaacacttttaccgattttaacgactgtgcccagtttttaattgacgaaaaatataccaatgccggaaaactttttgccatgggcggcagcgccggtggtttgctgatgggcgccatcatcaatctgaaacctgaattgtacaaaggggtaattgcagccgttccgtttgtggatgtggtgaccaccatgctcgacgaaagcattccgctcaccaccggcgagtttgatgaatggggcaatccgaaagattcgacctactatttctacatgaaatcgtattcgccatacgacaatgtggaagccaaaaactacccggctttgctagttacaacggggttgcacgattcgcaggtgcagtattttgagccagcaaaatgggtggctaaactccgcgaaatgaaaaccgacaccaatccgctgattttccatatcaatatggaattcggtcatggcggggcatccgggcgttttgattatatcaaagacattgcattggaatacgcgtttatttttgaccagattgggattaaggaataa

SEQ ID NO: 74 - protein, 710 aa, Unknown
  Obtained from an environmental sample
MKKRFNFSLKGKTLLVAALALAFAACTQKAEVKPPVAKKVPKELTIHGDTRIDNYYWLNERENQEVIAYLEAENAYKDAMMAHTKPLQDKLFEEIKSKIKQQDESVPYKKNGYYYYTRTVAEKEYYVSCRKKESLEAPEEVLLDVNAMAEGFDYYAIGGSSVSTDNKILAYGVDTVSRRNYTIYFKNLETGELLADQIPMTSGGATWANDNKTVFYMLKDSVTLRSEKVMKHVLGTPVSSDVEVFYEADETFNAFVYKTKSEKYLIIGSESTLTSEFRFLDANTPNGEFKIIQPRTRGLEYAVDHFDDYFYIRTNLDALNFKLVKTPVAKTTKENWVDVIPHRADVYFEEFDIFQNYLVASEKVEGITKLRVMPWQGGEYYIDFEEEVYTAYSGTNVDFNTEVFRFGYTSMTTPTSTFDFNMKTKERTLLKQQEVLGGFDKSNYETKRIYATAGDGTKIPISIVYKKGMEKNGNNPALIYGYGSYGYETEPAFSSSRLPLLDRGFVYAIAHIRGGQIYGRQWYEDGKLLKKMNTFTDFNDCAQFLIDEKYTNAGKLFAMGGSAGGLLMGAIINLKPELYKGVIAAVPFVDVVTTMLDESIPLTTGEFDEWGNPKDSTYYFYMKSYSPYDNVEAKNYPALLVTTGLHDSQVQYFEPAKWVAKLREMKTDTNPLIFHINMEFGHGGASGRFDYIKDIALEYAFIFDQIGIKE

SEQ ID NO: 75 - DNA, 2148 bp, Unknown
  Obtained from an environmental sample
atgcacaagaaacacctgaggttcgtgccgcctgtactcgtgggtgctcttctcgcgccggcaccagcgcccgcgcaggaaagcagcaaaccgccggtcgcagcgaggaaaccgcacaccaccgagattcacggctacacgctgacggacgactacttctggctgcgcgagaagggcagcacggacgtcacgaagtacctggaagccgagaacgcctacaccgatgcggtgatgaagccgttcaagccgctcgaggaggcgctgtacaaggaaatgctcggccgcatcaagcagacggacctcagcgtgccataccggctcggcgagtacttctattactcgcgcacggaagaaggaaagcagtatcccatcctgtgccgcaagaagggcagcatggacgcgctcgaagaagtgatgctcgatctgaacaagctcgcggagggccacaagttcctcggcctcggcgcggtgacggtgagcgacgacgggaactggctggcatattcgaccgatacgaccggctaccggcagtacacgctgaaggtgaagaacctgcgcaccggcgaaacgctcaaggacaacatcgaacgcgtcggctcggtggtgtgggccaacgacaacaagacgttgttctactcgactgaagacgcggtgtccaagcgatccgacaaggtgtggcggcacgccgtcggcgctgacggcagcgagctgatttacgaagagaaggacgagaacttcgacgtcggcgcgggccgatccctcgaccgcaagatcatcttcatcgcgtcctacgcgaagacgtcgcgcgaataccgctacctgcgcgccgacgatcccgcaggcacgccgaagctcgtgctcccgcgcgagccgggacacgagttcgacgtcgaccactacaacggcgacttctacatcaacacgaaccgcggcgcgaaaaacttccgagtcgtgaaggcgccgctttccgatccgtccgagaagaactggcagccgttcatcgaccacaaccccgccgtcaagatcgacgggatcacgttcttcaccggccacgcggtcgtctcggaacgcgagaacggcctcaactacctgcgcgtgatcgacatgaagtccagggcgtcacatcgcattgcgaccgacgagccggactacgcgatgttccttgggaccaatcccgagttcaacacgacgtcggtccgttacagctatcagtcgatggtgaccccaagcactgtctacgagtacgacctgaacacgaaggagcggaagctcctgaagcagcagccggttctcggcggctacgatccgaagcagtacgaggccaaacgggtgtgggcggtcgcgcgcgacggcacgaaggtgccgatctcggtcgtgtacaagaaaggtgtgaagcaggacggcacggcgccgctgctcctttatgcttacggatcttacggcgcctcgatggcgccgacgttctcgtcggggcgtctgagcctgctcgatcgcggcgccgcctatgcgatcgcctacatcaggggcggcggcgagctcggcgaggaatggcgggaacagggtcgcatgatgcagaagatgacgaccttcaccgacttcatcgactgcgccgagtacctcgtcaaaaacaagtatacgtcgtccgaccggctggtcatcaatggcggaagtgccggtggcctgctgatgggcgcggtgacgaacatgcgtccggatctgttcaaggcggtcgtcgccgaagtgccgttcgtcgacgtgatcaacacgatgctcgacgcgtcgctgcccctcaccacgagcgagtacacggagtgggggaacccgaacgagaaagcggcgttcgaatacatgttgaagtactcgccgtacgacaacgtcaaggccgtcaaatacccggcgatgctcgtgaaagtgtccgtgaacgacagccaggtgccgtactgggaaggcgccaagttcgttgcgaagctccgggcgacgaagaccgacaacaatccgctgctgctgaaggtcaacatgggcgccgggcacggaggcgcgtcaggacgatacgacgccatccgtgaaacggcatttacctacgccttcgtgctgtggcagatgggcttggctccgtccgcggccggaacgaacgcgcaaaaacactga

SEQ ID NO: 76 - protein, 715 aa, Unknown
  Obtained from an environmental sample
MHKKHLRFVPPVLVGALLAPAPAPAQESSKPPVAARKPHTTEIHGYTLTDDYFWLREKGSTDVTKYLEAENAYTDAVMKPFKPLEEALYKEMLGRIKQTDLSVPYRLGEYFYYSRTEEGKQYPILCRKKGSMDALEEVMLDLNKLAEGHKFLGLGAVTVSDDGNWLAYSTDTTGYRQYTLKVKNLRTGETLKDNIERVGSVVWANDNKTLFYSTEDAVSKRSDKVWRHAVGADGSELIYEEKDENFDVGAGRSLDRKIIFIASYAKTSREYRYLRADDPAGTPKLVLPREPGHEFDVDHYNGDFYINTNRGAKNFRVVKAPLSDPSEKNWQPFIDHNPAVKIDGITFFTGHAVVSERENGLNYLRVIDMKSRASHRIATDEPDYAMFLGTNPEFNTTSVRYSYQSMVTPSTVYEYDLNTKERKLLKQQPVLGGYDPKQYEAKRVWAVARDGTKVPISVVYKKGVKQDGTAPLLLYAYGSYGASMAPTFSSGRLSLLDRGAAYAIAYIRGGGELGEEWREQGRMMQKMTTFTDFIDCAEYLVKNKYTSSDRLVINGGSAGGLLMGAVTNMRPDLFKAVVAEVPFVDVINTMLDASLPLTTSEYTEWGNPNEKAAFEYMLKYSPYDNVKAVKYPAMLVKVSVNDSQVPYWEGAKFVAKLRATKTDNNPLLLKVNMGAGHGGASGRYDAIRETAFTYAFVLWQMGLAPSAAGTNAQKH

SEQ ID NO: 77 - DNA, 870 bp, Unknown
  Obtained from an environmental sample
atgtcgagaagaacaatcctgataagcgccgtcctgctgatcctcgtcgcgggcggcggtttcggatggaggcgattcaatcgcgcggccgtcggcatcccagacttctacgtctggaaattcctttccgggcagtcgcataacggacatcgcgctgccgttaatggcattagcatctactacgagacctacggccacggacagccggttctcctgctgcatggcgcgaccggatttctcgaaacgatgcattatttcatcaccggacttgcccccgcgcatttcgtgatcgccgtggacagtcgcgcgcagggacgctcgaccgattccgatgcgccgatcagctacgcactaatgggtgatgacatgatcaagctgcttgatacgatgcatatccgatcgacggatgttgtcggctggagcgacggcgggatcataggcctcgacatggcgatgaagcatcctgaccgtgtgcgccgcctggtcgccatcggcgccaattacgatgtcgacgggatcgatcccgagcatttccacccgaacgagaacgaacagcctctcccggtcgtcaagcagttctatgagagagtcgcgcccgacccgtctcattttccgattatgttcaagaaggtcaggacgatggtgcgcacacagccgcactacacgctggccgagctgagccagattcgaagccccacgctaatcgtggccggtcaacacgatctgatcctcccggagcatacagcaaagctcgccagcgccattgcgggcgcacgcaagatcattgttcccggcgcgagtcattttggtccactcgaacagccggaaacgtacaatgagatggtgctgaaatttctcgaggcgccgtaa

SEQ ID NO: 78 - protein, 289 aa, Unknown
  Obtained from an environmental sample
MSRRTILISAVLLILVAGGGFGWRRFNRAAVGIPDFYVWKFLSGQSHNGHRAAVNGISIYYETYGHGQPVLLLHGATGFLETMHYFITGLAPAHFVIAVDSRAQGRSTDSDAPISYALMGDDMIKLLDTMHIRSTDVVGWSDGGIIGLDMAMKHPDRVRRLVAIGANYDVDGIDPEHFHPNENEQPLPVVKQFYERVAPDPSHFPIMFKKVRTMVRTQPHYTLAELSQIRSPTLIVAGQHDLILPEHTAKLASAIAGARKIIVPGASHFGPLEQPETYNEMVLKFLEAP

SEQ ID NO: 79 - DNA, 825 bp, Unknown
  Obtained from an environmental sample
atgaagattctgattgcggccgatatggaaggtgttaccggtgtgatgaactgggaccaggtcacgcctggccagaccgagtatgaccgttttcgtcgcctgatgaccggcgacgtcaacgcggccattcgcggcgcggctgcggccggcgcaactgaattcatcgtcagcgacggccacgccgccaaccgcaacctgctgatcgaggaccttgacccccgcgttcgcttgaactgcggcagcccggcgcccctgtcgatggtccagggggtggatgagccgggcgtcgccgcggcgctgtttattggttaccacgcccgggccggggcgcagccggccgtgctagaccatacctggtcattgagcgtgctcaatttgtggctgaacggccaacgcgttggcgagattgggctgaacgcggccatctgcggctattttggcgtgccagtggcgctggtctccggcgaccagaccgccgcggccgaggccgttgagctgttgggccaggtggagatagccgtggtcaagcaggcccgcggccagcaggcggccgactgcctgccgccggaggtggcgcagtcccatatattcggggccgctcaacgagccatcgagcgggtgcaggccggccaggcccctccaccatttcagcttgagccgcctatcaccgtaacggttgagttcaccgagtcggtgttggccgaccgggccgcgctgttgccgggcagccggcggctccaagagcggcgcgttgagtttgtggcggcggatatgcccaccgcttaccgggcttttcgaactttggcggtgttggccgatggctaa

SEQ ID NO: 80 - protein, 274 aa, Unknown
  Obtained from an environmental sample
MKILIAADMEGVTGVMNWDQVTPGQTEYDRFRRLMTGDVNAAIRGAAAAGATEFIVSDGHAANRNLLIEDLDPRVRLNCGSPAPLSMVQGVDEPGVAAALFIGYHARAGAQPAVLDHTWSLSVLNLWLNGQRVGEIGLNAAICGYFGVPVALVSGDQTAAAEAVELLGQVEIAVVKQARGQQAADCLPPEVAQSHIFGAAQRAIERVQAGQAPPPFQLEPPITVTVEFTESVLADRAALLPGSRRLQERRVEFVAADMPTAYRAFRTLAVLADG

SEQ ID NO: 81 - DNA, 1461 bp, Unknown
  Obtained from an environmental sample
atgcccgaccacctaactgctactgatgaattactccaagcacacgcacaagacccagggattatcgaagaaacaattgtatcaagcttgacgagcattaaggaaaaaaatcctaatctcaatgcggttatctggcaaaacgaagacttcacaaacgatgagattaaaaaacttctgctgagcatacgacgaaaccaagtcgaaaacaataacaacacgcaagctctacttgaagagtttccacttttaggtattccagttttagtcaaagagatcgactgtgcaataaatggcaccgaaaattcatggggaaatatagagctaaaggaagctggatacaaagacacttacactgcaacaaaagtaaaacttcttcaaaaagcgggcgcgattattgttggcaaaacaaacaattctgagttagctggcgcactagtgacgaactctgatgcacatggaccttgtattaacccgctcgattcaaaacgaaattcgctgggatcatctggtggatcagcttgtgctgttgcaagtgcaatgactaatttcgcgactggtggagatggtggaggaagtatacgcatgccagctgctgcttgtggcgtatttggcttcaaaccttcacgtggccgtataagtcatggaccaatggtcagcgaaagttggtcaggaattgcttcttctggagcgttcactaaaaaccttcctgatatggcgagaatcatagatgtgttaagtgcgttcgatgcaggtgacacctatagcgccagcccacatcaaactaatttctcaaatgaattagagaactgtccagataatctgaaaattggaatccgtacagaaatactggattcaagattcaaagtcattccagaaataaaagaagctgtacacaaaatcgctgattttttacagttgcaaggctgcattatagaaactagtagcccttcgacattcaatgaaacatggatcacagaaaaatttgaaactttaattgcggttcacaccaagcatgatttcaattatgtttccaaaaggctagggcgcaattttgatatcaacagagtcactaaaactccgaaatactttttagatatcgcaagtaaaataaatctagagggctttctttatgccaaggaagactttgaatactacgcaagagaaaccgctcgatggtttgagaactttgatatcctaatcacccccactatcggtgacttcgcaccgggccacgactatcttaatgaagaagcttatatctattccggaaatacatttccaggaaatattgcaggattaccaggactcagtattccagtaaagacatctcattcgcttccttgcggaatacagttatattcaaaacgagataatgatttactggtcctacaactaggccgtctcatcgaaagggactattcggacatttttgttaccgaaaatcataatattactaattag

SEQ ID NO: 82 - protein, 486 aa, Unknown
  Obtained from an environmental sample
MPDHLTATDELLQAHAQDPGIIEETIVSSLTSIKEKNPNLNAVIWQNEDFTNDEIKKLLLSIRRNQVENNNNTQALLEEFPLLGIPVLVKEIDCAINGTENSWGNIELKEAGYKDTYTATKVKLLQKAGAIIVGKTNNSELAGALVTNSDAHGPCINPLDSKRNSLGSSGGSACAVASAMTNFATGGDGGGSIRMPAAACGVFGFKPSRGRISHGPMVSESWSGIASSGAFTKNLPDMARIIDVLSAFDAGDTYSASPHQTNFSNELENCPDNLKIGIRTEILDSRFKVIPEIKEAVHKIADFLQLQGCIIETSSPSTFNETWITEKFETLIAVHTKHDFNYVSKRLGRNFDINRVTKTPKYFLDIASKINLEGFLYAKEDFEYYARETARWFENFDILITPTIGDFAPGHDYLNEEAYIYSGNTFPGNIAGLPGLSIPVKTSHSLPCGIQLYSKRDNDLLVLQLGRLIERDYSDIFVTENHNITN

SEQ ID NO: 83 - DNA, 981 bp, Unknown
  Obtained from an environmental sample
atgattccacccattggcggcaaatcggacccgatcgacacttccagcacctggcagtccagcctgtcgcaggctgccaacaccaaccagccgcagcagcaatacaatgcttccggtctgtttggtccctctggcggcccgcgccccgacgacatcgtccagggcccgatcggcgactgctacttcctcgcggcgctcgcctccttcgcgcagcagcagccccagctgatccgcaacgcgatcgccttcgatgccaacaccggaaacttcaacgtgacgctgcacaaggatgcgcaaccctggaatccgttcaatcgccacgagaaggtgaccgtccgggtcacgcagcaggaaatcgacagccatgtcatgaacgacaagggcgccaggctgggcaacgacggcgcgcggtggccggtggtcatggagatcgcgcgcgccaagatgctcgacagcaatccgaagaacggcttggacgaaggctacaatgcactcgagcatcagacgccgttcggcttgcttcagggcggcatgccgtcatcggccatggagaccatctccggccaacccggcgacacggcatacacgacgccgctcggcatcttcggagacgcgacgcaaggacccttgcagccgtggctctcgctcgtctcgcccgggcaggcgttagcgccggaggcggcaaaccatcactatagccaggtcaaagccgcgctggaggacggacggcccgtgacgctgggtaccggcttgtccagcccgcaggacggcctcgttcgcgggcacgcctatcaggtcgaggatatccagcgcaacgccgacggcagcgttaacgtcacggttcgcaatccatggggaaccaatcagggtgtcggcgaaggcaccaatccggccgacccgcgtgtcaccatcaggatgggagccgcgggcctgtcgctgttcgcaatcggttcaagcgcgcaggagaccagcgccggatag

SEQ ID NO: 84 - protein, 326 aa, Unknown
  Obtained from an environmental sample
MIPPIGGKSDPIDTSSTWQSSLSQAANTNQPQQQYNASGLFGPSGGPRPDDIVQGPIGDCYFLAALASFAQQQPQLIRNAIAFDANTGNFNVTLHKDAQPWNPFNRHEKVTVRVTQQEIDSHVMNDKGARLGNDGARWPVVMEIARAKMLDSNPKNGLDEGYNALEHQTPFGLLQGGMPSSAMETISGQPGDTAYTTPLGIFGDATQGPLQPWLSLVSPGQALAPEAANHHYSQVKAALEDGRPVTLGTGLSSPQDGLVRGHAYQVEDIQRNADGSVNVTVRNPWGTNQGVGEGTNPADPRVTIRMGAAGLSLFAIGSSAQETSAG

SEQ ID NO: 85 - DNA, 831 bp, Unknown
  Obtained from an environmental sample
atgaagatctttatctcaaccgacatggaaggcaccgcaggagttgttgactggagtcaatgtattccaggtaatcccgaatacggctactaccgcgaactccttcaaaatgaagtaaatgcaagtatcaagggagcaatggcagctggagcgaaggagttccttgtcaatgattcccactcttcgatgcagaatcttcgaccgcattcacttgaaggaaatgctcgctacctttcaggtaaacacaagccgatgtacatgatgcaaggactcgatagttcttatgatgcaatctttttcgtctcatatcacggttcgatgagcgctgaagcgtcagttctttctcacacatacaatccgcgcgccatttcagatatccgactaaacggcgaattcgtatctgaggctgggattaacgcgcttgttgcgcaggcctacggagtgcctattgtgcttgttaccggtgatgagaccacagaagttgagaccaagatgtggtcacctggtgtcaaagcagcagtcgttaagaagagtattactcgctttgcagccgacagcatgcacccaaatcaagcgtgcgacttgatcttcgaaaaggcaaaggtgtctattgccgaactcaaatcaattccggtacctgcaatcgaacttccagcaacacttgaaatcaccttccataacaatgattttgcagatatggctgcttggattccaggcaacctgaagactggtacaaagaaagttgggattacagataaggaccctctggcgctcttccgtcgtttcatcacaaccgtcatcctcactcgcagtatcgttgagtggagctaa

SEQ ID NO: 86 - protein, 276 aa, Unknown
  Obtained from an environmental sample
MKIFISTDMEGTAGVVDWSQCIPGNPEYGYYRELLQNEVNASIKGAMAAGAKEFLVNDSHSSMQNLRPHSLEGNARYLSGKHKPMYMMQGLDSSYDAIFFVSYHGSMSAEASVLSHTYNPRAISDIRLNGEFVSEAGINALVAQAYGVPIVLVTGDETTEVETKMWSPGVKAAVVKKSITRFAADSMHPNQACDLIFEKAKVSIAELKSIPVPAIELPATLEITFHNNDFADMAAWIPGNLKTGTKKVGITDKDPLALFRRFITTVILTRSIVEWS

SEQ ID NO: 87 - DNA, 1926 bp, Unknown
  Obtained from an environmental sample
atgagcaaaacaacatcgcactatggaagctggagatccccgatcacagccgacctcattaccgccaaaaccatttcacctgttgaagtgatcgtcgatggcgacgatatctattggatcgaatcccgcccgctcgaaaaaggtcgctgtgtcatcatgcggcgcgcttcagatgggaacatcaccgaatgcacaccgccagagttcaatgtgcgcacttccgtacacgaatacggcaatggcgcattcacagttttcaaaggcatcatttactttatcaattacaaagaccagcacatctatcgtcaagccccgggtgaaacacccgttccgctcactccaatgaacggctatcgttacgccgatcttcgagtggatgaatctcgtaaccgcattatctgcattcgcgaagaccataccggcgaaggcgaagcggtcaacaccattgtgagtgttaatatcaatggcaacgacgatggaaccgttctcgtttcaggcaacaatttttattcttcgcctcgcctcgaccccaacggaaccaaactcgcctatctgacttggaatcaccctaacatgccctgggatggatgcgaactctggctggcagatatccacgaagacggcacgcttcacaacgcggaccttgtagctggttctgccaacgaatccatttaccaacccgaatggtctcccacaggcgtcctgcacttcgtcgcggaaccgaatggctggtggaatttgtaccgcttgaacgatggagccatcgaatccctacaccccatggaagcagagttcggtgaaccgcagtgggtcttcaatatggcaaagtatggcttcgtctcagaacacatcatcttttgtgcctacacccaaaacggactttggcatcttgccctgctcgatacccaaaccaaaaccttctcccccatttcttcacctttcacctacgccgccgacgtccattgcggtaagggctttagcgcattcattggcggttcattctctcaaccgctttctgtgatacggtatgatgtcacatcaaaccgctttgaaaccatcaaacaagcgtttgaagtgaccgtcgatgcgcagtatttatccactccgcaacccatcgccttctcaactacaaatgggaaggtcgcgcacgccatttactatctaccttgcaataaagacttctcagccccagagggagaacgcccgccgctgatggtcatcagccatggcggaccgaccgcacaaaccgctgtgaccttgcaatacaaaatccaatattggaccagtcgcggctttgctgttgccgatgtcaactacggcggttcaacgggctatgggcgggaataccgtcagcgcctcaacggcaactggggcatcgtggacgtggacgattgctgcaacgccgcgctttatctcgtagagcaggggctcgccgacccagaacgtctcgccattcgcggcggcagcgcaggtgggtacaccacgtttgcctgccttgccttccgcaatgtattcaaagccggagctggtcattatggcgtcagcgaccttgaagttcttgccaaagacacccacaagtttgagtcaagataccccgactcactgatcggtccataccctgaacggaaagatctgtattacgaacgttccccgatccatttttcgcacaagatcacttgtccgctgattctctttcagggagatgaagataaggtcgtgccgcccagccaatctcaatccatgtttgaggcggtacgggcaagggagattcccaccgcatacattctctttgaaggcgagcagcacgggttccgtaaagcggagtccatcaaacgcgctctagaaggcgagttgtatttttattcgaagatattcaagttcgagttggcagaagatatcccacccatcgagatcgaaaatctataa

SEQ ID NO: 88 - protein, 641 aa, Unknown
  Obtained from an environmental sample
MSKTTSHYGSWRSPITADLITAKTISPVEVIVDGDDIYWIESRPLEKGRCVIMRRASDGNITECTPPEFNVRTSVHEYGNGAFTVFKGIIYFINYKDQHIYRQAPGETPVPLTPMNGYRYADLRVDESRNRIICIREDHTGEGEAVNTIVSVNINGNDDGTVLVSGNNFYSSPRLDPNGTKLAYLTWNHPNMPWDGCELWLADIHEDGTLHNADLVAGSANESIYQPEWSPTGVLHFVAEPNGWWNLYRLNDGAIESLHPMEAEFGEPQWVFNMAKYGFVSEHIIFCAYTQNGLWHLALLDTQTKTFSPISSPFTYAADVHCGKGFSAFIGGSFSQPLSVIRYDVTSNRFETIKQAFEVTVDAQYLSTPQPIAFSTTNGKVAHAIYYLPCNKDFSAPEGERPPLMVISHGGPTAQTAVTLQYKIQYWTSRGFAVADVNYGGSTGYGREYRQRLNGNWGIVDVDDCCNAALYLVEQGLADPERLAIRGGSAGGYTTFACLAFRNVFKAGAGHYGVSDLEVLAKDTHKFESRYPDSLIGPYPERKDLYYERSPIHFSHKITCPLILFQGDEDKVVPPSQSQSMFEAVRAREIPTAYILFEGEQHGFRKAESIKRALEGELYFYSKIFKFELAEDIPPIEIENL

SEQ ID NO: 89 - DNA, 2130 bp, Unknown
  Obtained from an environmental sample
atgaagcgaatcccttacgttcttgctatccttgcgctaattggttccgtcacaatgctccttaacgcccaaagtttatctacttccgaaaccgatccggcgccgccaacggcagccaggaatcctaaaaagactgaaatacacggcgagacactcgtggatgattacttttggctgcgcgagaaatcgagcccggctgtgatctcgtatctggaggccgaaaatgcctacacagattcagtcatgaagccaactgatgcgctgcaagacaagctgtacaaagagatgctttcgcgcatcaaagagaccgacgtcaatgtccctttcaaagacggcaactactattactattcccgcacagaacagggaaagcagtatgccatctacgcgcgcaaacgtaagcttgatgctcaggaagagatcacgctcgatttgaacgagatcgccaaagggcagaagtttactgcgctgggcaactattcagtcagcgaggacggcaacctgcttgcctactcgatcgacaacacaggcttcagggaatacatcctttacataaaagatcttcgtaccggcgagctgttgcccgatcagctcggcaaagtcagaagcgccttctgggctaacgacaacaagacgctcttctacgtgattgaggatcgtgccaagcgtccgtaccagatttatcgccacacgctcggacagaagaaagaagacgataagctggtattcgaggacaaagacgagctgtttcgggtcttcgcattcaagtcacgaagcaaggattacatcttcttcggctcggctagctttacctcgactgaattcagctacgttccagccgcaaggcccaatgaagcgccaaaggtgcttctgcggcgcgaagccagtcacgagtactacccggatcatcacgggaaccatttttacatccgcacgaatgatgcgggccgcaatttcaggcttgtctctgctcctgtttccgatccgcagaaaaagaactggagcgagatactgccgcaccgcaaggatgtcatgctcgagagcgtagacatcttcaaagactactttgtggcaagcgagcgcgaaggcggtctgccgaggttccgcgtgatggttccgaagtcctggaaacatcactatgtggagttcaaggagcccgcctacacggcgtctgccgcacagaaccgagaattcgatacagcgaagttccggttcaactatcagtcgctcgtaacgcccagctcggtctttgattacgatatgaaaacccgcgaacagaccttgctgaagcagtacgaggttctgggtggctacgatcagtcgcaatatcaaacggagcgcatctacgctgctgcgtccgatggcacgcgaataccaatctcactcgtctataagaaagcgctcgttcgagatggaaagcggcccatgctgttgaacgggtacggctcgtatggaatatcgagcaatccgacgttcaattccaaccggctcagcctgattgatcggggcgtcatctttgcgatcgcgcacatacgcgggggcggcgagcttggaacgccctggcacgatcagggcaagatgatgaacaagaagaataccttcacggactttatcgccgtggccgagcacctggtcaaagagaagtacacatcttcggatcgtctggtgatcacaggtggcagcgccggcggcctgttgatgggcgccgtcgtgaacatgcgacccgacctcttcaaggcggtcgttacgtacgtgccattcgtggacgtcatcaacaccatgcttgatacttctttgccgctgaccgtcggtgaatacgaggagtggggcaatccgaacgacaaagatgcgtatctctacatgaagacctattcgccttacgacaatctggagaagaagagctatccggcgatgctggtgaagacttctctgaatgacagccaagtcatgtactgggagcctgccaaatacgtggccaagctcagaactctgaaggcagacaacaacgttcttctattcaagacaaacatggctgccgggcacggcggcgcttctggaagatacgattcgtggaaagagcttgcgttcgactatgcttttatagtcacgcaactcggactaaacgactag

SEQ ID NO: 90 - protein, 709 aa, Unknown
  Obtained from an environmental sample
MKRIPYVLAILALIGSVTMLLNAQSLSTSETDPAPPTAARNPKKTEIHGETLVDDYFWLREKSSPAVISYLEAENAYTDSVMKPTDALQDKLYKEMLSRIKETDVNVPFKDGNYYYYSRTEQGKQYAIYARKRKLDAQEEITLDLNEIAKGQKFTALGNYSVSEDGNLLAYSIDNTGFREYILYIKDLRTGELLPDQLGKVRSAFWANDNKTLFYVIEDRAKRPYQIYRHTLGQKKEDDKLVFEDKDELFRVFAFKSRSKDYIFFGSASFTSTEFSYVPAARPNEAPKVLLRREASHEYYPDHHGNHFYIRTNDAGRNFRLVSAPVSDPQKKNWSEILPHRKDVMLESVDIFKDYFVASEREGGLPRFRVMVPKSWKHHYVEFKEPAYTASAAQNREFDTAKFRFNYQSLVTPSSVFDYDMKTREQTLLKQYEVLGGYDQSQYQTERIYAAASDGTRIPISLVYKKALVRDGKRPMLLNGYGSYGISSNPTFNSNRLSLIDRGVIFAIAHIRGGGELGTPWHDQGKMMNKKNTFTDFIAVAEHLVKEKYTSSDRLVITGGSAGGLLMGAVVNMRPDLFKAVVTYVPFVDVINTMLDTSLPLTVGEYEEWGNPNDKDAYLYMKTYSPYDNLEKKSYPAMLVKTSLNDSQVMYWEPAKYVAKLRTLKADNNVLLFKTNMAAGHGGASGRYDSWKELAFDYAFIVTQLGLND

SEQ ID NO: 91 - DNA, 825 bp, Unknown
  Obtained from an environmental sample
atgaaggttttcctgtccagcgacatggagggcacggcgggcgtcgtcgactgggaccagtgcgtcggcgacgggccggaggcggcggccgggcgcgcgctgctgctcgccgaggtgaacgcggccatcgagggcgcggtggccggcggcgccaccgagatcgtggtcaacgactcgcactacaaaatgcgcaacctcccggccggcgcgctggcgggcggggcgtcctacatctccggcagccacaagccgctgtacatgatgcagggcctggacggcagcttcgacgcggtgctgttcgtgtcctaccacgggtcgatcggggcaccggccgggctgtcgcacacctacaacccgggcgcggtgatggaggccaggctcggtggcgtggtgaccggggaagcgggcatcaacgcgctggtggccgcgcactacggggtgccggtcgtgctggtcaccggggaccggtgcgcgtgcgaggaaaccgccgcgctgatccccggcgtgcacaccgcggtggtcaaggagcacgtcagccggctcgccgcgcacagcctgcacccggcccgcgcctgcgcgctgatccgggagaccgcgcagcaggcggtcgcgggggcgggcgccgccgcgtcgccgccggtgggctcggccccgctggaggtcgcggtccgcaccaccgacatcgcggaagcggccagctgggtccgcggcgtggagcgcaccggcccccgcgaacttcgcttccacggcgacgacccgctcgccgtctaccgcagtttttgcgcggcgatcctgctgacccgcgcggttgcggaggtggtttga

SEQ ID NO: 92 - protein, 274 aa, Unknown
  Obtained from an environmental sample
MKVFLSSDMEGTAGVVDWDQCVGDGPEAAAGRALLLAEVNAAIEGAVAGGATEIVVNDSHYKMRNLPAGALAGGASYISGSHKPLYMMQGLDGSFDAVLFVSYHGSIGAPAGLSHTYNPGAVMEARLGGVVTGEAGINALVAAHYGVPVVLVTGDRCACEETAALIPGVHTAVVKEHVSRLAAHSLHPARACALIRETAQQAVAGAGAAASPPVGSAPLEVAVRTTDIAEAASWVRGVERTGPRELRFHGDDPLAVYRSFCAAILLTRAVAEVV

SEQ ID NO: 93 - DNA, 822 bp, Unknown
  Obtained from an environmental sample
atgaaaattctcatcgcagccgatatggaaggaataacaggcgtcaccacctgggaccaggtcaccgccgggcattttgaatatccgcgcttccgccgcctgatgacggaagatgtcaatgccgccgtgcgtggcgcgttcgatggcggcgcaaaggaagtttttgtcaccgatggtcacgcggtgggttccaacatcttgatcgaagaactcgatccacgtgcctcgctcaacaccggttcgccctcgccgctctcgatggtccagggtgtggatgaagtggacgggctgatctttgtcggctatcacgcccgggccggttcgccggacgctatcctggaccacacctggtcttcaaaaacggtggcaaacgtctggttgaacgacatcctggtgggggagtatggtttgaacgcggcggtggccggtcacttcgatgtgccggtcctgatgattaccggggaccagacggcctgcgcccaggcggttgaactgctgggggatttggagaccgtcatagtcaagcgcgcggccggtcgctatgccgcggagtgcctgccgcccaaagtcacacataccatgatccagcgtgcagcagcgcaggcggttaccaggttcgcccggggtaaagcgcctgtgcccttcatcctggaggaaccgatttccttatcggttgagttccacacatccgtcatggccgatcatgccatgcgcatgccgggcgccgagcgcgatgggacctgtgtgactctatccctgccagatatggtcacgacgtatcatgccttcagggcgatcgtggcgctggcgagtatgggatag

SEQ ID NO: 94 - protein, 273 aa, Unknown
  Obtained from an environmental sample
MKILIAADMEGITGVTTWDQVTAGHFEYPRFRRLMTEDVNAAVRGAFDGGAKEVFVTDGHAVGSNILIEELDPRASLNTGSPSPLSMVQGVDEVDGLIFVGYHARAGSPDAILDHTWSSKTVANVWLNDILVGEYGLNAAVAGHFDVPVLMITGDQTACAQAVELLGDLETVIVKRAAGRYAAECLPPKVTHTMIQRAAAQAVTRFARGKAPVPFILEEPISLSVEFHTSVMADHAMRMPGAERDGTCVTLSLPDMVTTYHAFRAIVALASMG

SEQ ID NO: 95 - DNA, 1185 bp, Unknown
  Obtained from an environmental sample
atgagcgcaatctcgcgtactgcgatgtggactgctattgcagcttctttgtacgtcgtatcggcacgcgcggagctcgcgccgttgccgccacagcccgcggatgtcccgtggccgacgaacgattggccgaccggtccgcttcccgaaggtgtcgaccgtgccgcgctggaggcggcgatgacggacgcggtcgacaacaaggtgcctggactcggcgagacgcgcgaagtcgcgatcattctcggcggcaagctcgtctacgagcagtacggggccggctacaaatccgacatgaagctcgtgtcgtggtcgatggcgaagtcgatcacccaggcgctggtcggcgtcgcctcgcagcaaggcaaggtgaatgtcgacgagccgatgggcaacccgcattggcctgcgaccgacaagcgcgcacagataccgtggcgcacgtggttgcagatgacggacggcatgcgctacctggagatcgaagcaaaaacgatcgccgacagcgacgcgtcacgcaaactgttcggcccgggccgcctcgacgtcgcaaactactgcgccggcctgccgctgctcttcgagcccggcacgcactggaactacaactcgtgcggcatcgtactcacggccgacgcgttgacgcgcgcgatcgtgccgaaccccgcgtcacccgaggcgcgccgcgcagacatgctgaagtggatgcacgaaagtctgttcgacgtgatcggcatgaaggcacagccggaattcgacgcgcgcggcctgtactacggcagcgcgctcatctatgcctccgcgcgcgacttcgcgaagttcggactgctctatctgcgcgatggcatgtgggacggccagcgcgtcctgcctgaaggctgggtcgactttgcgcgcaccgccggcaagagcgacaacgccgacatctacggcgcaggctggtgggtcacgccggcggacggcgacggcaaaccgtataaatctcgaatcgatacgggcacgcaacgcgacgcgttcagcgccgaaggcttcgagggccaattcacgctgatcgtgccgtcgaaggatctgatcatcgtgcgcctcggcttcacgccggagaaagatctgcgcaccggcgcgatccgggtgtggctcggccgtgtcgcacgtgcattcccggtggcggggcaagggacggagtag

SEQ ID NO: 96 - protein, 394 aa, Unknown
  Obtained from an environmental sample
MSAISRTAMWTAIAASLYVVSARAELAPLPPQPADVPWPTNDWPTGPLPEGVDRAALEAAMTDAVDNKVPGLGETREVAIILGGKLVYEQYGAGYKSDMKLVSWSMAKSITQALVGVASQQGKVNVDEPMGNPHWPATDKRAQIPWRTWLQMTDGMRYLEIEAKTIADSDASRKLFGPGRLDVANYCAGLPLLFEPGTHWNYNSCGIVLTADALTRAIVPNPASPEARRADMLKWMHESLFDVIGMKAQPEFDARGLYYGSALIYASARDFAKFGLLYLRDGMWDGQRVLPEGWVDFARTAGKSDNADIYGAGWWVTPADGDGKPYKSRIDTGTQRDAFSAEGFEGQFTLIVPSKDLIIVRLGFTPEKDLRTGAIRVWLGRVARAFPVAGQGTE

SEQ ID NO: 97 - DNA, 2124 bp, Unknown
  Obtained from an environmental sample
atgaatagatgcctgctgttggcggctgccatttttctaaacgggctgccattatccgccattgctcaaaccgatatgaaaccaccagtgcccaagaaggttccccatgttaccaagatccacggttacgaacgagtggataactaccattggatgcgtgaccgcaatgagccgcgcgctccggagatcatcaaatatctcgaagaggaaaatgcctacacagatgcgtatatgaagcctcattcggggctagtcgattctctttacaaagaaatgctcggacggatcaagcaagacgacacctcagtacccgtcaaatatggcgatttctggtatttcaatcgtaccgaagagggaaagcaatatcccgtttataagcgtagcgagtcccgcgacggaaaagatgccgaagtcctgctcgaccaaaacgagatggcgaagggcttccagtttttcgccataatgggtgcctcaaacgtcagcgacgatggcaatatgctcgccttttctaccgacaccacgggctaccggcagtacaccctgcaattcaagaaccttaagtccggcgagatactccctcacaaattcgaacgggtcacctcggtcgtatgggctgcggataacaagaccattttcttcgttaccgaagacgaggtcgataagcgtccaaatattctctggcgatacgtgatcggcgggcccgcacccgtcaagctctacgaagaaaaagatgttttataccgcatcggtgcaggccgcacccgcgacaaaaagtacataatgatgggctcgtttgccgcgaccatgcgggaataccggtacattcccgcagataagcccactgaagaattcaagatcatcctcccccgtgagaaagaccacgagtattcggtcgatcattggaacggcgagtttttcattacgaccaataagaatgctgagaattcacgggtcgttagggcgccggttagcgatccgagcgaaaagaattggaaggagtttattccccacaaccccgcgataaagatcgaagcgatcgaggtttttaaagatttcgccgttgtatccgaactcgagaacggcctggaatacttgaaggttctcgaccttcgcacgcgtcgggccccggtcaggatccccaccgccgaagaagtttttacaatggggacgatgggcaaccgcgagttcgacaccgacgttgtccggtactactactcgtcgatgatcactccgaattcgacctatgaatataacttcaagacgggcaagagcgaacttctcaaacggcaggaaattccgagcggacacgatatttcaaaatatgagacgaagcgactatgggctaccgccagagacggcgtaaaggtgcctatttcgctcatgatgctcaaaggtacagagcttgacggcaagtcaccgatgctgctctacgcatacggctcttatggtgcctctatgacgccgaatttctcatcaaatcgccttagcctcgtcgatcgcggaatgatctatgccatcgcccacattcgcggcggctcggaactcggtgagaagtggcggcaagacggccgtatgttcaagaaacttaacaccttccacgattttgtcgattccgcaaaatggctcattgacgagaaatatacctcttcggaccgtcttgttattcagggtggctcagcaggcgggctgttaatgggtgcggtgatgaatatgtcccctgagacgttcaaggctgcgttagtgcaagtgccgtttgtcgatgtactcagcacaatgcttgatgaatcgctgcctcttaccacgggcgaatggatagaatggggcaatcctaatgagaaagaggcttttgattatatggcgaagtattcgccttatgacaacattaagaagcaagcctatccgaacatgatggtgcagatctcgctatacgatagccaggttctatattgggagggagcaaaatacgccgctaaagttcgcgaacacaattccggcgattcaaccatcctattaaagaccaacatgggcgcgggccacggcggttcttcgggacgctacgaccgcctcaaggaaatagctttcgattacgcttacgcactaagccaggtgggcattacaaaatag

SEQ ID NO: 98 - protein, 707 aa, Unknown
  Obtained from an environmental sample
MNRCLLLAAAIFLNGLPLSAIAQTDMKPPVPKKVPHVTKIHGYERVDNYHWMRDRNEPRAPEIIKYLEEENAYTDAYMKPHSGLVDSLYKEMLGRIKQDDTSVPVKYGDFWYFNRTEEGKQYPVYKRSESRDGKDAEVLLDQNEMAKGFQFFAIMGASNVSDDGNMLAFSTDTTGYRQYTLQFKNLKSGEILPHKFERVTSVVWAADNKTIFFVTEDEVDKRPNILWRYVIGGPAPVKLYEEKDVLYRIGAGRTRDKKYIMMGSFAATMREYRYIPADKPTEEFKIILPREKDHEYSVDHWNGEFFITTNKNAENSRVVRAPVSDPSEKNWKEFIPHNPAIKIEAIEVFKDFAVVSELENGLEYLKVLDLRTRRAPVRIPTAEEVFTMGTMGNREFDTDVVRYYYSSMITPNSTYEYNFKTGKSELLKRQEIPSGHDISKYETKRLWATARDGVKVPISLMMLKGTELDGKSPMLLYAYGSYGASMTPNFSSNRLSLVDRGMIYAIAHIRGGSELGEKWRQDGRMFKKLNTFHDFVDSAKWLIDEKYTSSDRLVIQGGSAGGLLMGAVMNMSPETFKAALVQVPFVDVLSTMLDESLPLTTGEWIEWGNPNEKEAFDYMAKYSPYDNIKKQAYPNMMVQISLYDSQVLYWEGAKYAAKVREHNSGDSTILLKTNMGAGHGGSSGRYDRLKEIAFDYAYALSQVGITK

SEQ ID NO: 99 - DNA, 2109 bp, Unknown
  Obtained from an environmental sample
atgaagcgtttcgttgttgcagcggcgatcgccacggcagtgccggccaatagccttgcgcagaccgttccggcaccgccggtcgccgagcaaaagcctcataccgtcaaggggcccaaagagcgaaacgatccctattactggctccgcgacgatacccgcaaagacccggcgatgctcgcctatctcaaagccgagaacgcttatgcggacgcggtgcttgccgacaccaggccgcttcgcgacaaattgttcagcgagttcgtcgggcgcatcaagcaggacgattccaccgtccccgcccgtcagcgcggctattattattattcacggttcgaaaccggcggagattatccgatcatcgctcgcaagcagggcaatctggaagcggcggagcaggtgcttctcgacatacccgcaatggccaaagggcacggctttttctcgatcggcgaatggatggtcagccaggacaaccggctgctcgcctttgcgcaggataatgtcggccgacgccaatattcgctgaaggtgaaggatatcgcgactggcgagatccacgccgacgccgtcgagaatgtcgagccgatcttcgtttgggccgacgacaaccgcacaatcttttacatcgagaaagatccggtgacgcttctgagcaagcgcgtgaaggcgcatgtgctcggcacccctgcaacggccgacgagctggtatacgaggaggaggacgacagcttctacctggcgctcggccgaaccagcgacgacaagtacatctgcatcatcgtacagagcacggtcagcaacgagcagcactgtacgagtgcagccaatccgggggagtggcgcgtgatcgcgccgcgcgagcgggacttcctttacagggccgatcacatcggcaaccgctggatcatccgcaccaactggaatgcgccgaactacaggctgatgacggtcgccgacgcaaatgcggcccaagcccgctccggctggaccgatctcgttcctcacgatgcaaacgtgttcatcgaggacttcaagccgttcaacagcttcatcgcgatcgagcagcgctccggcggcaacaagaagattcggctactatccaatgagggaaagagcagcgaagtcgtgtcggatgagccggcctatgcgatggcgctgagtaccaacctggagccggacacgacaaagcttcgctatgtctatgattcgctaacgactccgcgcatctggtacgaggtcgacgccaccaccggcgagcgcacgctgttgaagcaaacacccacccccaattacgattcctccgattacgtgacggagcgggtgtgggcgacggctcgcgacggaacgaggattccggtgtcgctggtctatcgtaaaggcttcaatcgggatggcacggcggcaatgctccagtacggctatggcagctacggcctctcgcaggatccgaggtggtcgccaatggttccaagccttctcgaccgcggaatggtctatgcgctcgcgcatattcgcggcgggcaggagatgggacgcgcctggtacgaccaaggtaagctcctcaacaagaagaacacgttcaccgacttcattgacgtgacacggtatctcgttgcgcagcgttatgctgcgaaggaccgagtggcagcgatgggtggaagcgcaggaggcctgctgatgggagctatcgcaaacatggccccgcaggactatcgagtgatcatcagccaggttccgttcgtcgacgtggtgacgacgatgctcgatgcgtcgatcccgctcaccaccaatgaatatgacgaatggggtaacccgtccgaacagcaattttacgattacatgctgtcttactcgccctacgaccagatcgaggcgaaggcatatccggccatgttcatcggcaccggcctgcatgacagccaggtgcagtatttcgagccgacaaagtatgtcgccaggcttcgagcaatgaagatcgacagcaacccactggtttaccgtgtgaatatggaagccggtcacggcggaaaatctggtcgcttcgagcaatatcgctcgagggcggaatattatgcgttcatgctcgaccagctgggggtcgagtag

SEQ ID NO: 100 - protein, 702 aa, Unknown
  Obtained from an environmental sample
MKRFVVAAAIATAVPANSLAQTVPAPPVAEQKPHTVKGPKERNDPYYWLRDDTRKDPAMLAYLKAENAYADAVLADTRPLRDKLFSEFVGRIKQDDSTVPARQRGYYYYSRFETGGDYPIIARKQGNLEAAEQVLLDIPAMAKGHGFFSIGEWMVSQDNRLLAFAQDNVGRRQYSLKVKDIATGEIHADAVENVEPIFVWADDNRTIFYIEKDPVTLLSKRVKAHVLGTPATADELVYEEEDDSFYLALGRTSDDKYICIIVQSTVSNEQHCTSAANPGEWRVIAPRERDFLYRADHIGNRWIIRTNWNAPNYRLMTVADANAAQARSGWTDLVPHDANVFIEDFKPFNSFIAIEQRSGGNKKIRLLSNEGKSSEVVSDEPAYAMALSTNLEPDTTKLRYVYDSLTTPRIWYEVDATTGERTLLKQTPTPNYDSSDYVTERVWATARDGTRIPVSLVYRKGFNRDGTAAMLQYGYGSYGLSQDPRWSPMVPSLLDRGMVYALAHIRGGQEMGRAWYDQGKLLNKKNTFTDFIDVTRYLVAQRYAAKDRVAAMGGSAGGLLMGAIANMAPQDYRVIISQVPFVDVVTTMLDASIPLTTNEYDEWGNPSEQQFYDYMLSYSPYDQIEAKAYPAMFIGTGLHDSQVQYFEPTKYVARLRAMKIDSNPLVYRVNMEAGHGGKSGRFEQYRSRAEYYAFMLDQLGVE

SEQ ID NO: 101 - DNA, 1449 bp, Unknown
  Obtained from an environmental sample
atgtctgacgcagcgggcctggacggcctggacctcgccgaactggtacggcggcggcaggtttcgccggaggaattgctgcaggcggcagtagcgcgggcggaggcggcgcagatgcagatcaactgtttcgcggcgatttatccggagattgccggcgcgcagattgcggcaggcggcctcgaagggccatatgcgggcgtgccgtttgtggtgaaggacctcggtgtcgaagtgaaaggcgcgccaatgaccggcggcagccgggcgtatcgcggcaatgtggcgagccgggactcgacacttgtggagcggttccgcaaggcaggcctggtgttttttgcctcgacgaccacgcccgagttcggtctgacgctgacaacggaatcgaccctgtacggacaaacgcgcaatccgtgggatgtgacgcgcacgaccggcggttcgtccggcggcgcggcagcggccgtggcggcgggcgtgttgccggtggcgcatgcgagcgatggcggcgggtcgatccgcattccggcggcgtgctgcggcgtgttcgggctgaaaccctcgcgcggtcggatgccgatggggccggcaaagacggaaggctggaacgggctctcgaccgtgggcgtggtcagccgcacggtgcgcgatacggcggcgatgctggacctgacgcacgggccggaacaaggtagccgttacaccgcgccggcaccggtacggccttacctgtctgaacttgagcgcgatccgaagccgctgcgcgtggcactgtggcggtccgccccgaacgggacgaagccggatgcggacgccgaagcggggctgatggcgacggcaaagctactcgaagcgcttggccacaccgtggttgaggcgggaccggtcctggacggcgaggcgctcagcaaggcggcgctgttcacgatctctgcgaacgtcgcggcgatggtagaagaacgcggcgcggcacgcggtgctccggtcacggacgatgagctggagccggtgacggcctcgatggtgcggttggggcggaccgtgccgatggtggagctggcgaaggccaacaacgcgttcattaccgcggcgatccagtatgaacatttcctggacgcgggcgggtttgacatgacgctgtcgccgacgatccatcgcgcgccggacccgctgggcacgatggcgctgacaaccaatccgcaagcgatgggcgctgcgatcgcaggctttgcgccgcactgcggggtgttcaaccagatgggcttcccggcgatgagcgttccattgcactggaccgggccgaagccgggcgcgccggcagggctgcctatcggtatgatgttcggcgcgcggttcggacaggaaggcctgctgctggcccttgcagggcaactggagcgcgcggcaccctgggcacaccggaggccgccggtatgggtggggtga

SEQ ID NO: 102 - protein, 482 aa, Unknown
  Obtained from an environmental sample
MSDAAGLDGLDLAELVRRRQVSPEELLQAAVARAEAAQMQINCFAAIYPEIAGAQIAAGGLEGPYAGVPFVVKDLGVEVKGAPMTGGSRAYRGNVASRDSTLVERFRKAGLVFFASTTTPEFGLTLTTESTLYGQTRNPWDVTRTTGGSSGGAAAAVAAGVLPVAHASDGGGSIRIPAACCGVFGLKPSRGRMPMGPAKTEGWNGLSTVGVVSRTVRDTAAMLDLTHGPEQGSRYTAPAPVRPYLSELERDPKPLRVALWRSAPNGTKPDADAEAGLMATAKLLEALGHTVVEAGPVLDGEALSKAALFTISANVAAMVEERGAARGAPVTDDELEPVTASMVRLGRTVPMVELAKANNAFITAAIQYEHFLDAGGFDMTLSPTIHRAPDPLGTMALTTNPQAMGAAIAGFAPHCGVFNQMGFPAMSVPLHWTGPKPGAPAGLPIGMMFGARFGQEGLLLALAGQLERAAPWAHRRPPVWVG

SEQ ID NO: 103 - DNA, 1131 bp, Unknown
  Obtained from an environmental sample
atgaagtccgatctggatcgtttgatgcagacccgcgacctggtcgcgctgctggtggtcggcgacggccacgataacccaccccgcgcctacctgaccggcggcgtccatatcactggcgggctggtgctgaaacggcatggcgaagcgccgctgctggtcgtcaaccccatggagaccgaagaagccgcccgcagcggcctggccgtatattctttccacgacctggactgggcgcagctggtggaggctgcccagggcgaccggagtcaggcggcgatcgccctgtggggccgctgcctggaacgcttcggcgtgccgccgggcaaagtcggcgtgtatgggtcgggcagcatcaacgtctacctggagatgacgcgacgggccgccgatgcctaccccgcctaccagttcgtcggcgagttaggcatgaccctgttcgacgaagcctacgtcaccaaggatgccgccgagatcgcccgcatccaggcggtggcggcggcgaccagcgccgtgctgcgcgcgacctgggatttcatcgccggccaccgcgccgacggcgaccgggtggtgaacggcgccggccaaccgctgatcatcggcgccgtgaaacgtttcgtgcggcgggcgctgctggactatgatttagaggataccggcatgatcttcgcccaggggcgcgacgcgggcttcccccacagccggggcgacgacggccaggcgctgcggttgggacagtccatcatcttcgacttattcccgcgggcgctgggcgggggctaccatcacgacgccacccgcacctggtgcatcggccatgcgccggccgaggtgcaggctgcctatgacgacgtgctggccgcctttgaaacggcgttggtcgcctatgccgagcccggccaaccggcgcacgaactccaggacgcggtgctggactacttcgaaagccgggggcatcccacctcgcgcagccagcccagcaccaccgagggctacgtccacagcctgggacacggcgtcgggctgaacatccatgaacggccgtcgctcagccacctggccaaagacgaccggctgcaaatcggcaacctgatccaaagaattcaaaaagcttctcgagagtacttctag

SEQ ID NO: 104 - protein, 376 aa, Unknown
  Obtained from an environmental sample
MKSDLDRLMQTRDLVALLVVGDGHDNPPRAYLTGGVHITGGLVLKRHGEAPLLVVNPMETEEAARSGLAVYSFHDLDWAQLVEAAQGDRSQAAIALWGRCLERFGVPPGKVGVYGSGSINVYLEMTRRAADAYPAYQFVGELGMTLFDEAYVTKDAAEIARIQAVAAATSAVLRATWDFIAGHRADGDRVVNGAGQPLIIGAVKRFVRRALLDYDLEDTGMIFAQGRDAGFPHSRGDDGQALRLGQSIIFDLFPRALGGGYHHDATRTWCIGHAPAEVQAAYDDVLAAFETALVAYAEPGQPAHELQDAVLDYFESRGHPTSRSQPSTTEGYVHSLGHGVGLNIHERPSLSHLAKDDRLQIGNLIQRIQKASREYF

SEQ ID NO: 105 - DNA, 1440 bp, Unknown
  Obtained from an environmental sample
atgaacgagcggcttgcagtcctggagcggcgcttccgcggtgaaatttctgccgcgcaggctgtcgagcactacgcgtcccgccacgcgcgtctccacagccgagtcaacgccatcgtgacacccgatttcgagcgggcgctcgagcaggcgaggcgacaagacgccgcgcgcccggaagcggcgcgtccactgggcggactgccgatcaccatcaaggacggcatccacgtcgcgggcctgccgtcgaccggaggaatgatcgaaccggacctcgcgatcgccgacgaggacgcccccaatgtcgcgcgcctgcgcgcggcgggcgcgatcatcctcggcaagaccaatgtgccagtcgcgaacgccgactggcaggccgtgaacaagctcttcggccgctcgctgaatccctggaaccccgcgctcacaccgggcggcagcacgggcggaggagcggcggccgtggcggccgggctctcggccgcggaactcggcagcgacatcggtggctcgatccgcatcccggccgccttctgcgggctcttcgcgcacaagccgagcgccggcgccgtctcgaacgccggacatttcccttggcgtggcaacgtcccgaatcccgcgctcgatcttgcggcgcaggggccgctcacgcgcagcgccgaagacctcgacctgttcctcgacgtgctggccggcgccaacggactcgacgcgaaggcgtggcgactcgagctgccgccgctgcgattcgactcactggagcggtgtcgcgtcggcctcctgcgcctgcccgcgtggatcgcggtggagcaatccatcctcgacgcgcagcaggagctcgtcgacggtctcgcccggcggggcgcaattgtccgcgatatcgaactgtccgggacctttggcgacctgacgcagtactaccggcaatacctgacgagcctgcagtgcgtcctcggcggcgcaactcctccgcacgctcgcgcgaaggctgcggcgaagatgcggacgtacgacgacgcgttcctcgcggccgtcgccgacgggcttgagggcagcgcggcgctcctgatcgagatgctggaggccgggcagcgctatcgccggcaatgggagacggtgttccgcgacgtcgacgtgattctctcgccggtgtgtaacgtcaacgcatttgcgcacgacgacgcgttcttctacgaccgcaggctcatgatcgacggcgtgccgtatccgtattaccgcctgagcgcgatcccgtcgctcgcctcgctagcagggctcccggtcaccgtgttcccgaccggtcggcgcgcggcgaccgggtccccgatcgggctgcaggtgatgggggccttcctcgaggaccggacgacgatccgctttgcgcgcctcgtggaagcggagttcggaggcttcacgccaccgcccggcttcgaatag

SEQ ID NO: 106 - protein, 479 aa, Unknown
  Obtained from an environmental sample
MNERLAVLERRFRGEISAAQAVEHYASRHARLHSRVNAIVTPDFERALEQARRQDAARPEAARPLGGLPITIKDGIHVAGLPSTGGMIEPDLAIADEDAPNVARLRAAGAIILGKTNVPVANADWQAVNKLFGRSLNPWNPALTPGGSTGGGAAAVAAGLSAAELGSDIGGSIRIPAAFCGLFAHKPSAGAVSNAGHFPWRGNVPNPALDLAAQGPLTRSAEDLDLFLDVLAGANGLDAKAWRLELPPLRFDSLERCRVGLLRLPAWIAVEQSILDAQQELVDGLARRGAIVRDIELSGTFGDLTQYYRQYLTSLQCVLGGATPPHARAKAAAKMRTYDDAFLAAVADGLEGSAALLIEMLEAGQRYRRQWETVFRDVDVILSPVCNVNAFAHDDAFFYDRRLMIDGVPYPYYRLSAIPSLASLAGLPVTVFPTGRRAATGSPIGLQVMGAFLEDRTTIRFARLVEAEFGGFTPPPGFE

SEQ ID NO: 107 - DNA, 885 bp, Archaeon
gtgcttgggagggtttacgctactcgagggcccatagcgttgccaagcgctctcatctctttagactttgaggggctaccctacatcgtctcgtcaaggcaccagctaggacccgatggaaggctatggggcgaggcaaggcggatagcaacgaaactagcgaagctcgtctcccagaagctcatcgaagccgggtatgagaaggtgatagtggccgatagtcacggtcttatgataaacatagagcctttagaattcccgcgtggagttgtgcttgtgaccgggttccctagggcctcgcccgccatggtccctctggttgagagtgttgacgttgccatctttctcgggtaccatgcgcgtgctggtgagcgctccgttctggctcatacctatgccgggaggtacgtggaggaggtagtagtgaacggtatgccagcgtccgagtacctgttgaacgcgttgtacctaggcgagaatggtatcccggttggtcttgtggctggctcggccgagctaatggaagaagtggagagatacacgccctgggcagaaagagtagtgctcaagaggagtctaggcttccttgctgccgtgtctccctcgctacaggatctcgaggaagcgttggtcaaagcattgcaagccatgaacgagaagcttgagaagggtgaattgaagccattgaaaccaaagacaccagtagaagttactatccggctaacgagcccgctgtacgccgatgtgtctgagttgcttccgggagcccgcagggtagacgggcgcaccattgcatacaaagctgaaaccatggagcaagctatgaggatgctagagctgctgctctatgcgtgggttggcgctgctagcatagcaggcaaatag

SEQ ID NO: 108 - protein, 294 aa, Archaeon
MLGRVYATRGPIALPSALISLDFEGLPYIVSSRHQLGPDGRLWGEARRIATKLAKLVSQKLIEAGYEKVIVADSHGLMINIEPLEFPRGVVLVTGFPRASPAMVPLVESVDVAIFLGYHARAGERSVLAHTYAGRYVEEVVVNGMPASEYLLNALYLGENGIPVGLVAGSAELMEEVERYTPWAERVVLKRSLGFLAAVSPSLQDLEEALVKALQAMNEKLEKGELKPLKPKTPVEVTIRLTSPLYADVSELLPGARRVDGRTIAYKAETMEQAMRMLELLLYAWVGAASIAGK

SEQ ID NO: 109 - DNA, 798 bp, Unknown; identical to SEQ ID NO: 71
  Obtained from an environmental sample

SEQ ID NO: 110 - protein, 265 aa, Unknown; identical to SEQ ID NO: 72
  Obtained from an environmental sample

SEQ ID NO: 111 - DNA, 1320 bp, Unknown
  Obtained from an environmental sample
atgttgccggtttatgccgccagcctggctgcagggggaatggcgttgtatcatgccgggacttcgcgcaagaagacaaggaacgccgcctcggacatgagcgataatccgcgcccacggcttcgcggattcaagcgtttgatgatcgctttggtattatgcctgtatgccatcctgtcggctgccctgccgtcgcttctgccggtgttcacgtttgccgaaccgcaaggaacgtacaaggtcggaacaacgacattgatgctggaggataaacgaagggaagagccggctacggaggatcgggcggatcggcgcaggctcatgattcaaatctggtatccggcggaagccggcacaggcagcggcaaggcgccatacatcgagcatctgccgattgttctggaaggcctgcgccagtcgatttcaatgcctccgttcctgctgagccagctaaagtatgtgcagccttatgccatgcaggatgcggaagtatcccgggatcaggagcgatatcccgtgctgctcttctctcacgggctttccggctttcggaaccagaacacgtttcaggtaacggagcttgccagccggggatacatcgtggtcggcatcgaccatgcctatgacgccgccgccgtcgtgttcccggaccggaccgccatgctcaagcttggcaatttgtcgggcttcgatgcctacgaggagaagagccggctctgggtggaggatgcgaaatttgtcctggacgagctggagcggatgagcgggcctgccgcttcagggttcttgtccgggcggctcgacatggacaagatcggcatgttcggccactcgttcggcggggcgaccgcggcgcaaatgctgctggcggacagccgggtgaaggccgcactgaatatggacggaaccctatacggacagccggtgccggaaggcggctttggcaagccgtatatgcagatgaatgctgagctaagcatcgaccgtgccgtatttgaacaatcgctggaccgggcgatggcgtcttccggccgcagccgggaggattacgagcagttctgggccgagtcggtcgaacgccgaagccgcgccgagtcgggtgaagcgcataccatcatatttgcccatgcgaaccatatgagctttacggatttttacctgttctccccgctgcttccgcccaagggtgcggagccaaggcagatgcacagctcgatcaacgcgctgtccgcggcctttttcgatcaatatgtgaaaggcgatccgggcgcctcggttgaaaagaccgcgctcagcctgccgggactgatccgaccgtaa

SEQ ID NO: 112 - protein, 439 aa, Unknown
  Obtained from an environmental sample
MLPVYAASLAAGGMALYHAGTSRKKTRNAASDMSDNPRPRLRGFKRLMIALVLCLYAILSAALPSLLPVFTFAEPQGTYKVGTTTLMLEDKRREEPATEDRADRRRLMIQIWYPAEAGTGSGKAPYIEHLPIVLEGLRQSISMPPFLLSQLKYVQPYAMQDAEVSRDQERYPVLLFSHGLSGFRNQNTFQVTELASRGYIVVGIDHAYDAAAVVFPDRTAMLKLGNLSGFDAYEEKSRLWVEDAKFVLDELERMSGPAASGFLSGRLDMDKIGMFGHSFGGATAAQMLLADSRVKAALNMDGTLYGQPVPEGGFGKPYMQMNAELSIDRAVFEQSLDRAMASSGRSREDYEQFWAESVERRSRAESGEAHTIIFAHANHMSFTDFYLFSPLLPPKGAEPRQMHSSINALSAAFFDQYVKGDPGASVEKTALSLPGLIRP

SEQ ID NO: 113 - DNA, 1008 bp, Unknown
  Obtained from an environmental sample
atgaaagcgaattgtttcggcctggccgtcagcatctttttcgtcctctcgatatttccattgatggtgacctctcaaagcaagccgcgcgcccgcgatcttggcgttccgtttgaaggcacgccgggaccgctcaacgcgattacggatgtcaaaggcatcgaggtcggacacgcgacgctcgtttccggcgaagggaaattgcaagtcggcgccggcccggtgcgcaccggagtgaccgccgttctgccgcgcggccggggatcgaatgagccggtgtttgccggctggttctcgctcaacggcaacggcgagatgaccggcacaacgtggatcgaagaatcgggtttcctcgaagggccggttatgattaccaatacgcacagcgtcggcgtagtgcgcgatgcggtcatcgcctggcaagcgaagcaatacggcaaaatggctcagccgtggatgctgccggtagtcgcggaaacctacgacggattcctgaacgacatcaacggatttcacgtcaaagagaatcacgtctttgatgcgctgagcagcgccaggtcggggccggtagcggaaggcaatgtcggcggcggcacggggatgatctgtcacggcttcaaaggcggcatcggcacagcgtcgagaaagctgagtgaaggagcgggcggctacacggttggtgtgctcgtgcaagccaattacggatcgcgaagtcagctccgcattgcgggcgtgccagtgggacaggagatcacggacctgactatgatctctcaaacgaataatccatccggcaattctttcgctgatttttctggtgaaactggctcgatcatcatcgtggtggcgaccgatgcgccactcctgccgcatcagatgaaacggctggcgcggcgcgcggcgttggggctggcccgcaacggcagcacggcgggtaacggttcgggcgacatcttcatcgctttctcgacggccaacgcgggagcggcgaaggcatcaggcaacgcg

SEQ ID NO: 114 - protein, 336 aa, Unknown
  Obtained from an environmental sample
MKANCFGLAVSIFFVLSIFPLMVTSQSKPRARDLGVPFEGTPGPLNAITDVKGIEVGHATLVSGEGKLQVGAGPVRTGVTAVLPRGRGSNEPVFAGWFSLNGNGEMTGTTWIEESGFLEGPVMITNTHSVGVVRDAVIAWQAKQYGKMAQPWMLPVVAETYDGFLNDINGFHVKENHVFDALSSARSGPVAEGNVGGGTGMICHGFKGGIGTASRKLSEGAGGYTVGVLVQANYGSRSQLRIAGVPVGQEITDLTMISQTNNPSGNSFADFSGETGSIIIVVATDAPLLPHQMKRLARRAALGLARNGSTAGNGSGDIFIAFSTANAGAAKASGNA